(19) United States
(12) Patent Application Publication
(10) Pub. No.: US 2025/0059604 A1
(43) Pub. Date: Feb. 20, 2025

(54) BIOMARKERS AND METHODS RELATED TO FRAGILE X SYNDROME

(71) Applicants: University of Massachusetts, Boston, MA (US); Rush University Medical Center, Chicago, IL (US)

(72) Inventors: Joel D. Richter, Wellesley, MA (US); Sneha Shah, Worcester, MA (US); Elizabeth Berry-Kravis, DesPlaines, IL (US)

(21) Appl. No.: 18/723,148

(22) PCT Filed: Dec. 23, 2022
(86) PCT No.: PCT/US2022/082382

§ 371 Date: Jun. 21, 2024

Related U.S. Application Data
(60) Provisional application No. 63/265,994, filed on Dec. 23, 2021.
(60) Provisional application No. 63/334,634, filed on Apr. 25, 2022.

(51) Int. Cl. C12Q 1/6883

(52) U.S. Cl. CPC C12Q 1/6883; C12Q 2600/156; C12Q 2600/158; G01N 2800/50; G01N 2800/52

(57) ABSTRACT

Provided herein, in various embodiments, are methods of diagnosing a subject as having, or having a propensity to develop, a fragile X-associated disorder (e.g., FXS), methods of prognosing a fragile X-associated disorder (e.g., FXS) in a subject, methods of predicting a treatment outcome of a fragile X-associated disorder (e.g., FXS) in a subject, methods of stratifying a set of subjects having a fragile X-associated disorder, methods of stratifying a population of subjects having, or having a propensity to develop, FXS, and method for assessing the efficacy of a drug for treatment of FXS. Also provided herein, in various embodiments, are assays and systems useful for performing the disclosed methods. The present disclosure also provides methods of treating a subject having FXS.

### RELATED APPLICATIONS

This application claims the benefit of U.S. Provisional Applications No. 63/265,994 filed on Dec. 23, 2021, and 63/334,634 filed on Apr. 25, 2022. The entire teachings of the above applications are incorporated herein by reference.

### GOVERNMENT SUPPORT

This invention was made with government support under GM046779 and GM135087 from National Institutes of Health. The government has certain rights in the invention.

### INCORPORATION BY REFERENCE OF MATERIAL IN ASCII TEXT FILE

This application incorporates by reference the Sequence Listing contained in the following eXtensible Markup Language (XML) file being submitted concurrently herewith:
a) File name: 54391026002.xml; created Dec. 22, 2022, 13,968 Bytes in size.

### BACKGROUND

Fragile X Syndrome (FXS) lies on the autism spectrum and is the most frequent inherited form of intellectual impairment. FXS afflicts 1 in 4000 boys and 1 in 7000 girls. In addition to intellectual impairment, children with FXS present a range of symptoms, including speech and developmental delays, perseveration, hyperactivity, aggression, and epilepsy, among other maladies. FXS is caused by a CGG triplet repeat expansion in a single gene, FMR1, which resides on the X chromosome. When the CGG triplet expands to 200 or more, the FMR1 gene is methylated and thereby transcriptionally inactivated. The loss of the FMR1 gene product, the protein fragile X messenger ribonucleotide protein (FMRP), is the cause of the disorder.

### SUMMARY

In one aspect, the present disclosure provides a method of diagnosing a subject as having, or having a propensity to develop, a fragile X-associated disorder, the method comprises assaying at least one biomarker in a biological sample (e.g., a non-neural biological sample) from the subject, wherein the level and/or splicing of the at least one biomarker in the biological sample is indicative of the subject as having, or having a propensity to develop, a fragile X-associated disorder.

In another aspect, the present disclosure provides a method of prognosing a fragile X-associated disorder in a subject, comprising assaying at least one biomarker in a biological sample (e.g., a non-neural biological sample) from the subject, wherein the level and/or splicing of the at least one biomarker in the biological sample is indicative of the subject as having a propensity to have a poorer prognosis of the fragile X-associated disorder.

In another aspect, the present disclosure provides a method of predicting a treatment outcome of a fragile X-associated disorder in a subject, comprising assaying at least one biomarker in a biological sample (e.g., a non-neural biological sample) from the subject, wherein the level and/or splicing of the at least one biomarker in the biological sample is indicative of the subject as having a propensity to have a poorer treatment outcome.

In certain embodiments, the at least one biomarker is a RNA biomarker.

In some embodiments, the fragile X-associated disorder is FXS.

In some embodiments, the biological sample is a non-brain tissue sample. In certain embodiments, the biological sample is a non-neural biological sample, i.e., a sample that does not comprise any neurons.

In some embodiments, the at least one RNA biomarker is selected from the group consisting of AGAP1, RAB25, FAM3B, XKR3, MAP3K15, LEP, RP11-706O15.3, GCOM1, CXCL6, RGL3, NECAB2, TGM3, LRRC6, MAB21L3, RP11-36B15.1, AC091878.1, RP11-154H23.3, NOV, AC093495.4, RP11-455F5.6, RGPD2, COL9A3, CLEC18A, RP11-256L6.2, LINC01127, SLC38A11, EFCAB12, LA16c-380H5.5, CXCL1, RP11-1334A24.5, AC100793.2, ANKDD1A, AVIL, RP11-44F14.8, RP11-290F20.1, AC116366.5, EPHB4, ST6GALNAC3, PANX2, CREB5, KIAA0319, HECW2, ADCY4, LINC00173, RP11-59D5_B.2, RP11-274B18.2, RP11-213H15.3, CORO7-PAM16, HAL, DPEP3, AC002467.7, MGAM, PNMA8A, FMR1, S100B, RP11-885N19.6, RP11-545I5.3, AC091814.2, KLRC2, L1TD1, PGBD5, MXRA7, CROCC2, SEMA5A, PLA2G4C, RP11-1008C21.1, TANC1, C4orf50, NUAK1, AC104809.4, RGS17, KCNS1, DRAXIN, B3GAT1, ARHGEF28, KIF19, APOL4, GZMH, GAS1, SCD5, GLB1L2, IGHA1, KNVDC1, RP11-383H13.1, FGFR2, TFCP2L1, PDGFRB, LAG3, GPR153, PODN, CKB, CERCAM, ZNF365, JUP, TRNP1, JAKMIP1, CPXM1, SLC1A7, LGR6, FCRL6, MORN4, TUBB2A, PRSS23, BFSP1, NCALD, ZNF573, PAK1, MIR4435-2HG, CD8B, PDGFC, TRAPPC2L, AC006504.5, ZNF512, FAM228B, NEIL2, FAM78A, FYB1, RNF216P1, ZCWPW1, DTX2, ATP5MD, MX2, LYRM1, GUF1, DPH7, NSFL1C, MTMR1, GTPBP10, RGS3, DRAM2, RHOH, LAIR2, GBP3, GTF2H1, XPNPEP3, ZNF888, TBC1D5, AC060780.1, SDHAP2, KMT2A, SH3BP2, CSNK1G2, NSUN5P1, LINC01128, RNF19A, SNHG8, TOP1MT, AL135818.1, CR1, CRIM1, NAP1L1, AC004593.2, SEC61A2, PCNX2, TPT1-AS1, HLA-A, LUCAT1, PTPN2, SEC31B, POLR2J3, POLR2J4, CAST, POLR2J4, NUMBL, PRMT7, ATF7IP2, TIMM23B-AGAP6, ADGRE2, GTF2H2B, MICA, SLC29A2, ZBTB10, NLGN3, METTL25, ADAM15, SSH1, SIRPB1, PARP2, PACRGL, ENTPD1-AS1, FUZ, SDR39U1, EPOR, ZSCAN26, SNHG17, GPS2, NECAP1, MRPL11, DNAJC19, ANKZF1, C1orf162, PIGT, SLC25A37, AP1G1, CIC, ITGB7, ATG16L2, BECN1, ARHGEF40, BANP, PIGA, RAD52, IRF3, CEP78, SPINT1, TMEM156, NT5C3B, PLD2, ANKRD12, CASP8, PACS2, HLA-DMA, DHPS, PDCD6, SNX5, MPPE1, AC016394.2, DPM1, E2F5, PTPN7, MTFP1, TOR1AIP1, POT1, JOSD2, NLRX1, FDXR, ZDHHC16, ALKBH4, RPS9, ZNF302, TENT4B, TKT, CARD8, RBM26, WSB1, DDX60L, ATP11A, SRGAP2, CEACAM21, COX18, WDR47, PATZ1, POLM, CC2D1B, CLK4, MIB2, PHF1, KANSL1, TCF3, and combinations thereof.

In another aspect, the present disclosure provides a method of stratifying a population of subjects having, or having a propensity to develop, a fragile X-associated disorder (e.g., FXS), comprising assaying non-neural biological samples from the subjects for the presence of FMR1 RNA isoform 12.

In another aspect, the present disclosure provides a method for assessing the efficacy of a drug for treatment of a fragile X-associated disorder (e.g., FXS), comprising stratifying a population of subjects to create a stratified population comprising a subpopulation who has the FMR1 RNA isoform 12 and a subpopulation who does not have the FMR1 RNA isoform 12, and administering the drug to the subpopulation who has FMR1 RNA isoform 12, or to both subpopulations.

In another aspect, the present disclosure provides a method of stratifying a set of subjects having a fragile X-associated disorder (e.g., FXS), comprising assaying FMR1 RNA in a biological sample from the subject, and stratifying the set of subjects for treatment based on the presence and/or level of the FMR1 RNA isoform 12 in the biological sample.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a volcano plot of log 2FC of RNA levels (FXS vs typically developing (TD)). Statistically significant changes (P value<0.0002) are shown as black dots (down-regulated) and white dots (up-regulated). Gray dots refer to unchanged RNAs.

FIG. 2 is a heat map for RNA clustering based on z-scores of differentially expressed genes in leukocytes from FXS (N=29) vs TD (N=13) individuals.

FIG. 3 shows histograms for transcripts per million (TPM) values for RNAs that are up or down-regulated in FXS vs TD. *p<0.05; **p<0.01).

FIG. 4 shows histograms of RT-qPCR validations of up or down-regulated RNAs in FXS (N=7) and TD (N=5) leukocytes.

FIG. 5 shows histograms of RT-qPCR analysis of S100B, RAB25, and GAPDH RNAs in FXS2 LCLs compared to TD1 LCLS. The amounts of S100B and RAB25 were made relative to GAPDH. (*P<0.05, **P<0.01, t test).

FIG. 6A shows a summary table for changes in alternative splicing events in FXS vs TD leukocytes detected by rMATS at an FDR<5% and a difference in the exon inclusion levels (PSI, Percent spliced-in) between the genotypes (deltaPSI) of ≥5%. Schematic for the splicing event categories is shown at the left of the table. FIG. 6B shows violin plots of alternative splicing in FXS vs TD leukocytes indicating PSI for each type event.

FIG. 7A shows inclusion levels for exon 3 of LAIR2 RNA in FXS vs TD samples from rMATS analysis. FIG. 7B shows RT-PCR exon 3 skipping of the LAIR2 RNA in TD (N=3) and FXS (N=9) samples.

FIG. 8 is a volcano plot showing statistically significant changes (P_{adj}<0.05) of RNA markers with increased (428, “x”) or reduced (305, “Δ”) expression levels in the white blood cells (WBCs) of fragile X syndrome (FXS) individuals (patients) versus WBCs from typically developing (TD) individuals. Gray dots refer to unchanged RNAs.

FIG. 9 is a heat map for RNA clustering based on z-scores of differentially expressed genes in leukocytes from FXS (N=7) vs TD (N=10) individuals.

FIG. 10 shows metagene profiles using deepTools 2 for distribution of H3K4me3 marks along gene lengths. A similar increase in ChIP signal irrespective of genotype was seen at the transcription start site (TSS) for the H3K4me3 ChIP.

FIG. 11 shows metagene profiles using deepTools 2 for distribution of H3K36me3 marks along gene lengths. A similar increase in ChIP signal irrespective of genotype was seen in the gene body and transcription end site (TES) for H3K36me3 ChIP.

FIG. 12 shows changes in the alternative polyadenylation site (Ln-long 3′UTR-distal polyA site usage, Sh-Short 3′UTR-proximal polyA site usage) between the genotypes (P<0.05, average reads count>5 in each replicate in each region (aUTR and cUTR)).

FIG. 13 shows changes in intronic polyadenylation (IPA) site usage between the genotypes (P<0.05, average reads count>5 in each replicate in each region (aUTR and cUTR)).

FIG. 14 shows a genome browser view of PNMA8A RNA, showing exons and introns. PNMA8A RNA was strongly expressed in the white blood cells of FXS individuals and virtually absent in the white blood cells of typically developing individuals.

FIG. 15 shows a genome browser view of XKR3 RNA, showing exons and introns. XKR3 RNA was strongly expressed in the white blood cells of FXS individuals and virtually absent in the white blood cells of typically developing individuals.

FIG. 16 shows a genome browser view of S100B, showing exons and introns. S100B RNA expression was reduced in the white blood cells of FXS individuals, compared to typically developing individuals.

FIG. 17 is a scatter plot of percent spliced in (PSI) of skipped exons (SE) in white blood cells of FXS individuals versus typically developing individuals. The symbol “x” represents RNA markers having an increased exon skipping in FXS individuals relative to typically developing individuals. The symbol “▴” represents RNA markers having a reduced exon skipping in FXS individuals relative to typically developing individuals. All data are statistically significant (p<0.05 and FDR<0.05). NC: No change in alternative exons in 17064 RNAs, AS UP: Increased alternative exon inclusion in 419 RNAs, AS DOWN: Decreased alternative exon inclusion in 705 RNAs.

FIG. 18 is a scatter plot of percent spliced in (PSI) of mutually excluded exons (MXE) in white blood cells of FXS individuals versus typically developing individuals. 689 RNA markers had a decreased mutually exclusive exon switching in FXS individuals relative to typically developing individuals (“x”, less inclusion of mutually excluded exon) and 571 RNA markers had an increased mutually exclusive exon switching in FXS individuals relative to typically developing individuals (“▴”, more inclusion of mutually excluded exon). All data are statistically significant (p<0.05 and FDR<0.05).

FIG. 19 shows gene ontology terms for biological processes using clusterProfiler (v3.12.0) (Yu et al., 2012) enriched in RNAs up-regulated (top) and down-regulated (bottom) in FXS leukocytes relative to TD (P_{adj} value<0.05).

FIG. 20 summarizes changes in alternative splicing events in FXS vs TD leukocytes detected by rMATS (Shen et al., 2014) at an FDR<5% and a difference in the exon inclusion levels (PSI, Percent spliced-in) between the genotypes (deltaPSI) of ≥5% and read counts >1 in each sample. Schematic for the splicing event categories is shown at the left of the table.

FIG. 21 shows normalized gene counts (transcripts per million, TPM) obtained from RNA-seq data analysis for total FMR1 (all isoforms), FMR1-205 (encodes full-length 632 amino acid FMRP), FMR1-217 (a mis-spliced RNA), and FXR2, a paralogue of FMR1. The scale from light grey to dark gray denotes highest to lowest gene counts.

FIG. 22 shows a genome browser view of RNA-seq data for FXS and TD individuals for the FMR1 gene. FMR1 RNA is detected in all TD individuals (top 13 reads) and FXS individuals 1-21 show (bottom 29 reads). The black box marked on the FMR1 gene illustrated at the bottom shows the region of intron 1 with differential reads between TD (1-13) and FXS (1-21) individuals.

FIG. 23 shows an expanded view of FMR1 exon 1 and intron 1. The reads displayed here map to an exon that comprises the annotated FMR1-217 isoform. All annotated FMR1 isoforms and sequence data for FMR1-217 PCR fragments from FXS RNA sample are shown in FIG. 24. H refers to high and L refers to low FMR1.

FIG. 24 shows FMR1 isoforms annotated in the GRCh38.p13 genome assembly. The FMR1-217 isoform (ENST00000621447.1) is marked with a grey box.

FIG. 25 shows changes in intronic polyadenylation (IPA) site usage between the genotypes (P<0.05, average reads count>5 in each replicate in each region (aUTR and cUTR)).

FIG. 26 shows the full length FMR1 RNA and the FMR1-217 isoform illustrated with the CGG repeats in the 5′ UTR. The proportion of full length FMR1 to FMR1-217 was quantified by RT-qPCR in TD, H FMR1 and L FMR1 individuals. The forward (F) and reverse (R) primers used for q-PCR are shown. The total FMR1 RNA relative to GAPDH RNA levels was significantly reduced in H FMR1 and L FMR1 vs TD (* represents P<0.05, t test). Bar graphs indicate mean, Error bars indicate+/−SEM.

FIG. 27 is a volcano plot of log 2FC of RNA level changes (L FMR1 vs H FMR1). Statistically significant changes (P_{adj} value<0.05) are enlarged black dots circled by a thick line (down-regulated) and enlarged black dots left un-circled (up-regulated) relative to unchanged RNAs (smaller light gray dots).

FIGS. 28A-28B are scatter plots of percent spliced in (PSI) of skipped exon (SE) or mutually excluded exon (MXE) in the white blood cells of FXS individuals who expressed FMR1 RNA isoform 12 versus those who did not. FIG. 29A. The symbol “x” represents RNA markers having an increased exon skipping in isoform 12-expressing FXS individuals relative to those who did not. The symbol “▴” represents RNA markers having a reduced exon skipping in isoform 12-expressing FXS individuals relative to those who did not. FIG. 29B. The symbol “x” represents RNA markers having a reduced mutually exclusive exon switching in isoform 12-expressing FXS individuals relative to those who do not. The symbol “▴” represents RNA markers having an increased mutually exclusive exon switching in isoform 12-expressing FXS individuals relative to those who did not.

FIG. 29 is a summary table of changes in alternative splicing events from L FMR1 vs H FMR1 samples detected by rMATS (Shen et al., 2014) at an FDR<5% and a difference in the exon inclusion levels (PSI, Percent spliced-in) between the genotypes (deltaPSI) of ≥5%. Read counts >1 in each sample are depicted. Schematic for the splicing event categories is shown at the left of the table.

FIG. 30 shows changes in the alternative polyadenylation site (Ln-long 3′UTR-distal polyA site usage, Sh-Short 3′UTR-proximal polyA site usage) between the genotypes (P<0.05, average reads count>5 in each replicate in each region (aUTR and cUTR)).

FIG. 31 depicts sample information for postmortem FXS frontal cortex and premutation FXS carriers and TD individuals (derived from (Tran et al., 2019)). RNA-seq datasets GSE107867 (NIH samples) and GSE117776 were reanalyzed for DGE, DAS and APA. The TPM for FMR1 RNA in the samples is shown.

FIG. 32A shows Integrative Genomics Viewer (IGV) tracks of RNA-seq data (Tran et al., Widespread RNA editing dysregulation in brains from autistic individuals, Nat. Neurosci. (2019)) for FXS and TD individuals for the FMR1 gene. FIG. 32B IGV tracks of selected regions of FMR1 re-analyzed from the RNA-seq data of Vershkov et al., FMR1 Reactivating Treatments in Fragile X iPSC-Derived Neural Progenitors In Vitro and In Vivo, Cell Rep. 26: 2531-39 (2019), who deleted the FMR1 CGG expansion by CRISPR/Cas9 gene editing. Biologic duplicate of iPSC-derived neural stem cells (NSCs) from FXS individuals (FXS-NSC) treated with vehicle or 5-aza-2-deoxycytidine (5-azadC) as well as isogenic CGG-edited samples are shown. FMR1-217 reads are detected only in the 5-azadC-treated samples. FIG. 32C IGV tracks of selected regions of FMR1 re-analyzed from the RNA-seq data of Liu et al. Rescue of Fragile X Syndrome Neurons by DNA Methylation Editing of the FMR1 Gene, Cell 172: 979-91 (2018), who performed targeted FMR1 gene demethylation in FXS iPSCs and iPSC-derived neurons. iPSCs derived from FXS individuals were incubated with viruses expressing a mock guide RNA (i_mock), or an FMR1 guide RNA and catalytically inactive Cas9 fused to the Tet1 demethylase (i_Tet1). iPSC-derived neurons from to FXS individuals were treated with a mock guide RNA (N1_mock, N2_mock), or an FMR1 guide RNA and catalytically inactive Cas9 fused to the Tet1 demethylase (N1_Tet1, N2_Tet1, N3_Tet1). All cells were incubated with an FMR1 guide RNA and catalytically inactive Cas9 fused to the Tet1 demethylase express FMR1-217.

FIG. 33 is a volcano plot of log 2FC of RNA level changes (FXS vs TD (UCD), N=2/genotype). Statistically significant changes (P_{adj} value<0.05) are shown in enlarged black dots circled by a thick line (down-regulated) and enlarged black dots left un-circled (up-regulated) relative to unchanged RNAs (smaller light gray dots).

FIG. 34 is a summary table of changes in alternative splicing events FXS vs TD (Univ of California at Davis) and FXS vs FXS carriers (NIH NeuroBioBank) detected by rMATS (Shen et al., 2014) at an FDR<5% and a difference in the exon inclusion levels (PSI, Percent spliced-in) between the genotypes (deltaPSI) at ≥5%. Read counts >1 in each sample are depicted. Schematic for the splicing event categories is shown at the left of the table.

FIG. 35 depicts the experimental design for RNA extraction from post-mortem cortical tissue obtained from 6 FXS males (F1-F6) and 5 typically developing (T1-T5) age-matched males. RT-qPCR data for cortical tissue-derived RNA samples representing abundance for FMR1 and FMR1-217 isoforms relative to GAPDH RNA. Each sample was analyzed in duplicate. Primers used for amplification are represented in FIG. 26 (P<0.01**, t test).

FIG. 36 depicts a schematic diagram of fibroblast generated from skin biopsies obtained from three male premutation carriers (P1-P3) and three male TD individuals (T1-T3). The table shows patient de-identified designation, genotypes, and CGG repeat numbers in the 5′UTR in the FMR1 gene. ND, not determined. qPCR data for fibroblast-derived RNA samples representing abundance for FMR1 and FMR1-217 isoforms relative to GAPDH RNA. Each sample was analyzed in duplicate. Primers used for amplification are represented in FIG. 26.

FIG. 37 shows sample information for lymphoblast cell lines (LCLs) (Coriell Institute, NJ) from two FXS and two TD members of a family are shown. FMRP and GAPDH (loading control) levels were determined by western blots. Ratios of FMRP/GAPDH normalized to FXS1 are shown below the blot.

FIG. 38 shows the proportion of full length FMR1 to FMR1-217 quantified using RT-qPCR in the TD and FXS2 LCLs relative to GAPDH RNA levels. Primers used for q-PCR are shown in the gene illustration. The total FMR1 RNA was unchanged but the proportion of FMR1-217 was significantly higher in FXS2 LCL compared to TD LCL.

FIG. 39 shows the proportion of full length FMR1 to FMR1-217 quantified using RT-qPCR in the FXS1 and FXS2 LCLs treated with 5-AzadC relative to vehicle, normalized to GAPDH RNA levels (** represents P<0.001, t test). FMRP levels were determined using western blots relative to GAPDH in FXS1 and FXS2 LCLs treated with DMSO or 5-AzadC. Ratios of FMRP/GAPDH are shown below the blots. Histograms indicate mean values; error bars indicate+/−SEM.

FIG. 40 shows changes in the alternative polyadenylation site (Ln-long 3′UTR-distal poly(A) site usage, Sh-Short 3′UTR-proximal poly(A) site usage) between the genotypes (P<0.05, average reads count>5 in each replicate in each region (aUTR and cUTR))

FIG. 41 is a volcano plot of log 2FC of RNA level change (FXS vs pre-mutation carriers (NIH), N=2/genotype). Statistically significant changes (P_{adj} value<0.05) are shown in enlarged black dots circled by a thick line (down-regulated), and enlarged black dots left un-circled (up-regulated) relative to unchanged RNAs (smaller light gray dots).

FIG. 42 shows changes in the alternative polyadenylation site (Ln-long 3′UTR-distal poly(A) site usage, Sh-Short 3′UTR-proximal poly(A) site usage) between the genotypes (P<0.05, average reads count>5 in each replicate in each region (aUTR and cUTR)).

FIG. 43 shows a summary of statistically significant RNA events distinguishing FXS individuals from typically developing individuals, and FXS individuals who express FMR1 RNA isoform 12 from those who did not.

FIG. 44 shows correlation of FXS molecular parameters with IQ. Three-dimensional comparison of indicated parameters. The inset shows samples with 100% methylation. The increasing size of the dots represent increase in FMRP levels, and the darkness from low to high represent increase in IQ levels.

### DETAILED DESCRIPTION

A description of example embodiments follows.

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an” and “the” should be understood to include plural reference unless the context clearly indicates otherwise.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

“About” means within an acceptable error range for the particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of ±20%, e.g., ±10%, ±5% or ±1% of a given value. It is to be understood that the term “about” can precede any particular value specified herein, except for particular values used in the Exemplification. When “about” precedes a range, as in “about 24-96 hours,” the term “about” should be read as applying to both of the given values of the range, such that “about 24-96 hours” means about 24 hours to about 96 hours.

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the invention, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

When introducing elements disclosed herein, the articles “a,” “an,” “the,” and “said” are intended to mean that there are one or more of the elements. Further, the one or more elements may be the same or different. Thus, for example, unless the context clearly indicates otherwise, “a biomarker” includes a single biomarker, and two or more biomarkers. Further the two or more biomarkers can be the same or different as, for example, in embodiments wherein a first biomarker has a reduced expression and a second biomarker has an increased alternative 5′ splicing.

The term “poor” or “poorer” refers to greater degree of fragile X-associated disorder (e.g., FXS) symptoms, increased extent of disease, decreased (i.e., worsening) state of disease, increased or enhanced state of disease progression, deterioration or worsening of the disease state, whether detectable or undetectable.

### Methods and Compositions for Diagnosing/Prognosing Fragile X-Associated Disorders

In one aspect, the present disclosure provides a method of diagnosing a subject as having, or having a propensity to develop, a fragile X-associated disorder, the method comprising assaying at least one biomarker (e.g., RNA biomarker) in a biological sample from the subject, wherein the level and/or splicing of the at least one RNA biomarker in the biological sample is indicative of the subject as having, or having a propensity to develop, a fragile X-associated disorder.

In another aspect, the present disclosure provides a method of prognosing a fragile X-associated disorder in a subject, comprising assaying at least one biomarker (e.g., RNA biomarker) in a biological sample from the subject, wherein the level and/or splicing of the at least one RNA biomarker in the biological sample is indicative of the subject as having a propensity to have a poorer prognosis of the fragile X-associated disorder.

In another aspect, the present disclosure provides a method of predicting a treatment outcome of a fragile X-associated disorder in a subject, comprising assaying at least one biomarker (e.g., RNA biomarker) in a biological sample from the subject, wherein the level and/or splicing of the at least one RNA biomarker in the biological sample is indicative of the subject as having a propensity to have a poorer treatment outcome.

### Fragile X-Associated Disorders

Fragile X-associated disorders are caused by mutation of the fragile X messenger ribonucleoprotein 1 (FMR1, previously known as fragile X mental retardation 1) gene, located in the q27.3 loci of the X chromosome. The expansion of the trinucleotide CGG above the normal range (greater than 54 repeats) in the non-coding region of the FMR1 gene has been associated with the development of the fragile X-associated disorders in those carrying the premutation (55-200 CGG repeats). Non-limiting examples of fragile X-associated disorders include fragile-X associated tremor/ataxia syndrome (FXTAS), fragile X-associated primary ovarian insufficiency (FXPOI), fragile X-associated neuropsychiatric disorders (FXAND), and fragile X syndrome (FXS). In some embodiments, the fragile X-associated disorder is FXS.

### Biological Samples

As used herein, “biological sample” refers to any sample that can be from or derived from a human subject. The methods disclosed herein can be performed using RNA molecules obtained from a variety of possible biological sample types. For example, a single cell or cell lysate, a population of cells, a cell culture, a tissue, or a biological fluid.

In some embodiments, the biological sample is a non-brain sample. In certain embodiments, the biological sample is a non-neural biological sample. In some embodiments, the biological sample is a bodily fluid sample, a hair sample (e.g., from hair follicles), nasal (e.g., nasal swab) sample, buccal (e.g., buccal swab) sample or a skin sample. Non-limiting examples of biological fluids (bodily fluids) include blood (e.g., whole blood and derivatives and fractions of blood, such as plasma or serum), bone marrow aspirates, cerebrospinal fluid, extracted galls, GCF gingival crevicular fluid, milk, prostate fluid, pus, saliva (including whole saliva, individual gland secretions, oral rinse), skin scrapes, sputum, surface washings, tears (liquid secreted by lacrimal glands), and urine. In certain embodiments, the bodily fluid comprises blood, saliva, sputum, tears, urine or semen, or a combination thereof. In particular embodiments, the bodily fluid comprises white blood cells.

In other embodiments, the biological sample is a brain sample.

In certain embodiments, the biological sample comprises a fetal cell (e.g., circulating fetal cell), a blastomere, a trophectoderm cell, a stem cell (e.g., induced pluripotent stem cell (iPSC) or derived stem cell), a fibroblast (e.g., a dermal derived fibroblast cell or lung-derived fibroblast cell), a modified fibroblast, a leukocyte, a pluripotent cell, or a cultured cell.

### Biomarkers

As used herein, “biomarker” refers to a nucleotide sequence (e.g., RNA) or encoded product thereof (e.g., a protein) used as a point of reference when identifying altered RNA splicing or expression. A marker can be derived from expressed nucleotide sequences (e.g., from an RNA, mRNA, a cDNA, etc.), or from an encoded polypeptide.

In some embodiments, a biomarker disclosed herein comprises at least one RNA biomarker.

In some embodiments, at least one RNA biomarker having an increased expression, having a reduced expression, having an increased exon skipping, having a reduced exon skipping, having an increased mutually exclusive exon switching, having a reduced mutually exclusive exon switching, having an increased alternative 5′ splicing, having a reduced alternative 5′ splicing, having an increased alternative 3′ splicing or having a reduced alternative 3′ splicing, in the biological sample, relative to a control sample, is indicative of the subject as having, or having a propensity to develop, a fragile X-associated disorder (e.g., FXS).

In certain embodiments, at least one RNA biomarker having an increased expression, having a reduced expression, having an increased exon skipping, having a reduced exon skipping, having an increased mutually exclusive exon switching, having a reduced mutually exclusive exon switching, having an increased alternative 5′ splicing, having a reduced alternative 5′ splicing, having an increased alternative 3′ splicing or having a reduced alternative 3′ splicing, relative to a control sample, is indicative of the subject as having a propensity to have a poorer prognosis of a fragile X-associated disorder (e.g., FXS).

In certain embodiments, at least one RNA biomarker having an increased expression, having a reduced expression, having an increased exon skipping, having a reduced exon skipping, having an increased mutually exclusive exon switching, having a reduced mutually exclusive exon switching, having an increased alternative 5′ splicing, having a reduced alternative 5′ splicing, having an increased alternative 3′ splicing or having a reduced alternative 3′ splicing, relative to a control sample, is indicative of the subject as having a propensity to have a poorer treatment outcome for a fragile X-associated disorder (e.g., FXS).

In some embodiments, the at least one RNA biomarker of the disclosure is AGAP1, RAB25, FAM3B, XKR3, MAP3K15, LEP, RP11-706O15.3, GCOM1, CXCL6, RGL3, NECAB2, TGM3, LRRC6, MAB21L3, RP11-36B15.1, AC091878.1, RP11-154H23.3, NOV, AC093495.4, RP11-455F5.6, RGPD2, COL9A3, CLEC18A, RP11-256L6.2, LINC01127, SLC38A11, EFCAB12, LA16c-380H5.5, CXCL1, RP11-1334A24.5, AC100793.2, ANKDD1A, AVIL, RP11-44F14.8, RP11-290F20.1, AC116366.5, EPHB4, ST6GALNAC3, PANX2, CREB5, KIAA0319, HECW2, ADCY4, LINC00173, RP11-59D5_B.2, RP11-274B18.2, RP11-213H15.3, CORO7-PAM16, HAL, DPEP3, AC002467.7, MGAM, PNMA8A, FMR1, S100B, RP11-885N19.6, RP11-545I5.3, AC091814.2, KLRC2, L1TD1, PGBD5, MXRA7, CROCC2, SEMA5A, PLA2G4C, RP11-1008C21.1, TANC1, C4orf50, NUAK1, AC104809.4, RGS17, KCNS1, DRAXIN, B3GAT1, ARHGEF28, KIF19, APOL4, GZMH, GAS1, SCD5, GLB1L2, IGHA1, KNDC1, RP11-383H13.1, FGFR2, TFCP2L1, PDGFRB, LAG3, GPR153, PODN, CKB, CERCAM, ZNF365, JUP, TRNP1, JAKMIP1, CPXM1, SLC1A7, LGR6, FCRL6, MORN4, TUBB2A, PRSS23, BFSP1, NCALD, ZNF573, PAK1, MIR4435-2HG, CD8B, PDGFC, TRAPPC2L, AC006504.5, ZNF512, FAM228B, NEIL2, FAM78A, FYB1, RNF216P1, ZCWPW1, DTX2, ATP5MD, MX2, LYRM1, GUF1, DPH7, NSFL1C, MTMR1, GTPBP10, RGS3, DRAM2, RHOH, LAIR2, GBP3, GTF2H1, XPNPEP3, ZNF888, TBC1D5, AC060780.1, SDHAP2, KMT2A, SH3BP2, CSNK1G2, NSUN5P1, LINC01128, RNF19A, SNHG8, TOP1MT, AL135818.1, CR1, CRIM1, NAP1L1, AC004593.2, SEC61A2, PCNX2, TPT1-AS1, HLA-A, LUCAT1, PTPN2, SEC31B, POLR2J3, POLR2J4, CAST, POLR2J4, NUMBL, PRMT7, ATF7IP2, TIMM23B-AGAP6, ADGRE2, GTF2H2B, MICA, SLC29A2, ZBTB10, NLGN3, METTL25, ADAM15, SSH1, SIRPB1, PARP2, PACRGL, ENTPD1-AS1, FUZ, SDR39U1, EPOR, ZSCAN26, SNHG17, GPS2, NECAP1, MRPL11, DNAJC19, ANKZF1, C1orf162, PIGT, SLC25A37, AP1G1, CIC, ITGB7, ATG16L2, BECN1, ARHGEF40, BANP, PIGA, RAD52, IRF3, CEP78, SPINT1, TMEM156, NT5C3B, PLD2, ANKRD12, CASP8, PACS2, HLA-DMA, DHPS, PDCD6, SNX5, MPPE1, AC016394.2, DPM1, E2F5, PTPN7, MTFP1, TOR1AIP1, POT1, JOSD2, NLRX1, FDXR, ZDHHC16, ALKBH4, RPS9, ZNF302, TENT4B, TKT, CARD8, RBM26, WSB1, DDX60L, ATP11A, SRGAP2, CEACAM21, COX18, WDR47, PATZ1, POLM, CC2D1B, CLK4, MIB2, PHF1, KANSL1 or TCF3, or a combination thereof.

In particular embodiments, the at least one RNA biomarker comprises fragile X messenger ribonucleoprotein 1 (FMR1).

In some embodiments, the method comprises assaying at least 2, 3, 4, 5, 10, 15, 20, 25, 30, 35, 40 or 45 RNA markers in the biological sample from the subject. Non-limiting examples of RNA biomarkers having an increased expression, having a reduced expression, having an increased exon skipping, having a reduced exon skipping, having an increased mutually exclusive exon switching, having a reduced mutually exclusive exon switching, having an increased alternative 5′ splicing, having a reduced alternative 5′ splicing, having an increased alternative 3′ splicing or having a reduced alternative 3′ splicing, or a combination thereof, can be found in Tables 1-10.

### Increased RNA Expression

In some embodiments, at least one RNA biomarker (e.g., at least: 2, 3, 4, 5, 6, 7, 8, 9, 10, 15, 20 or 25 RNA biomarkers) having an increased expression in the biological sample, relative to a control sample, is indicative of the subject as having, or having a propensity to develop, a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having an increased expression in the biological sample, relative to a control sample, is indicative of the subject as having a propensity to have a poorer prognosis of a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having an increased expression in the biological sample, relative to a control sample, is indicative of the subject as having a propensity to have a poorer treatment outcome for a fragile X-associated disorder (e.g., FXS).

In certain embodiments, expression of the at least one RNA biomarker has a log 2 fold increase of at least about 0.50 in the biological sample, relative to a control sample, for example, the log 2 fold increase is at least about 0.55, 0.60, 0.65, 0.70, 0.75, 0.80, 0.85, 0.90, 0.95, 1.00, 1.25, 1.50, 1.75, 2.00, 2.25, 2.50, 2.75, 3.00, 3.25, 31.50, 3.75, 4.00, 4.25, 4.50, 4.75, 5.00, 5.25, 5.50, 5.75, or 6.00. In particular embodiments, expression of the at least one RNA biomarker has a log 2 fold increase of ≥0.80 in the biological sample, relative to a control sample, optionally, wherein the log 2 fold increase is ≥0.95.

In some embodiments, expression of the at least one RNA biomarker has a log 2 fold increase of about 0.50-10.00 in the biological sample, relative to a control sample, for example, about: 0.55-10.00, 0.55-9.50, 0.60-9.50, 0.60-9.00, 0.65-9.00, 0.65-8.50, 0.70-8.50, 0.70-8.00, 0.75-8.00, 0.75-7.50, 0.80-7.50, 0.80-7.00, 0.85-7.00, 0.85-6.50, 0.90-6.50, 0.90-6.00, 0.95-6.00 or 0.95-5.95.

Non-limiting examples of RNA biomarkers having increased expression in a biological sample, relative to a control sample, can be found in Table 1.

In some embodiments, the at least one RNA biomarker is AGAP1, RAB25, FAM3B, XKR3, MAP3K15, LEP, RP11-706O15.3, GCOM1, CXCL6, RGL3, NECAB2, TGM3, LRRC6, MAB21L3, RP11-36B15.1, AC091878.1, RP11-154H23.3, NOV, AC093495.4, RP11-455F5.6, RGPD2, COL9A3, CLEC18A, RP11-256L6.2, LINC01127, SLC38A11, EFCAB12, LA16c-380H5.5, CXCL1, RP11-1334A24.5, AC100793.2, ANKDD1A, AVIL, RP11-44F14.8, RP11-290F20.1, AC116366.5, EPHB4, ST6GALNAC3, PANX2, CREB5, KIAA0319, HECW2, ADCY4, LINC00173, RP11-59D5_B.2, RP11-274B18.2, RP11-213H15.3, CORO7-PAM16, HAL, DPEP3, AC002467.7, MGAM or PNMA8A, or a combination thereof.

In particular embodiments, the at least one RNA biomarker comprises isoform 12 of FMR1.

### Reduced RNA Expression

In some embodiments, at least one RNA biomarker (e.g., at least: 2, 3, 4, 5, 6, 7, 8, 9, 10, 15, 20 or 25 RNA biomarkers) having a reduced expression in the biological sample, relative to a control sample, is indicative of the subject as having, or having a propensity to develop, a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having a reduced expression in the biological sample, relative to a control sample, is indicative of the subject as having a propensity to have a poorer prognosis of a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having a reduced expression in the biological sample, relative to a control sample, is indicative of the subject as having a propensity to have a poorer treatment outcome for a fragile X-associated disorder (e.g., FXS).

In certain embodiments, expression of the at least one RNA biomarker has a log 2 fold reduction of at least about 0.50 in the biological sample, relative to a control sample, for example, the log 2 fold reduction is at least about 0.55, 0.60, 0.65, 0.70, 0.75, 0.80, 0.85, 0.90, 0.95, 1.00, 1.25, 1.50, 1.75, 2.00, 2.25, 2.50, 2.75, 3.00, 3.25, 31.50, 3.75, 4.00, 4.25, 4.50, 4.75, 5.00, 5.25, 5.50, 5.75, or 6.00. In particular embodiments, expression of the at least one RNA biomarker has a log 2 fold reduction of ≥1.00 in the biological sample, relative to a control sample, optionally, wherein the log 2 fold reduction is ≥1.16.

In some embodiments, expression of the at least one RNA biomarker has a log 2 fold reduction of about 0.50-7.00 in the biological sample, relative to a control sample, for example, about: 0.50-6.50, 0.55-6.50, 0.55-6.00, 0.60-6.00, 0.60-5.50, 0.65-5.50, 0.65-5.00, 0.70-5.00, 0.70-4.60, 0.75-4.60, 0.75-4.40, 0.80-4.40, 0.80-4.20, 0.85-4.20, 0.85-4.10, 0.90-4.10, 0.90-4.00, 0.95-4.00, 0.95-3.90, 1.00-3.90, 1.00-3.80, 1.05-3.80, 1.05-3.70, 1.10-3.70, 1.10-3.60, 1.15-3.60 or 1.15-3.00.

Non-limiting examples of RNA biomarkers having reduced expression in a biological sample, relative to a control sample, can be found in Table 2.

In some embodiments, the at least one RNA biomarker is FMR1, S100B, RP11-885N19.6, RP11-545I5.3, AC091814.2, KLRC2, L1TD1, PGBD5, MXRA7, CROCC2, SEMA5A, PLA2G4C, RP11-1008C21.1, TANC1, C4orf50, NUAK1, AC104809.4, RGS17, KCNS1, DRAXIN, B3GAT1, ARHGEF28, KIF19, APOL4, GZMH, GAS1, SCD5, GLB1L2, IGHA1, KNDC1, RP11-383H13.1, FGFR2, TFCP2L1, PDGFRB, LAG3, GPR153, PODN, CKB, CERCAM, ZNF365, JUP, TRNP1, JAKMIP1, CPXM1, SLC1A7, LGR6, FCRL6, MORN4, TUBB2A, PRSS23 or BFSP1, or a combination thereof.

In particular embodiments, the at least one RNA biomarker comprises isoform 1 of FMR1.

### Increased Exon Skipping

In some embodiments, at least one RNA biomarker (e.g., at least: 2, 3, 4, 5, 6, 7, 8, 9, 10, 15, 20 or 25 RNA biomarkers) having an increased exon skipping in the biological sample, relative to a control sample, is indicative of the subject as having, or having a propensity to develop, a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having an increased exon skipping in the biological sample, relative to a control sample, is indicative of the subject as having a propensity to have a poorer prognosis of a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having an increased exon skipping in the biological sample, relative to a control sample, is indicative of the subject as having a propensity to have a poorer treatment outcome for a fragile X-associated disorder (e.g., FXS).

In certain embodiments, exon skipping of the at least one RNA biomarker is increased by at least about 5% in the biological sample, relative to a control sample, for example, at least about: 6%, 7%, 8%, 9%, 10%, 12%, 15%, 18%, 20%, 25%, 30%, 35%, 40%, 45%, 50%, 55%, 60%, 65%, 70%, 75%, 80%, 85% or 90%. In particular embodiments, the increase is at least about 13.0%, optionally, the increase is at least about 16.0%.

In some embodiments, exon skipping of the at least one RNA biomarker is increased by about 5-90% in the biological sample, relative to a control sample, for example, about: 6-90%, 6-85%, 7-85%, 7-80%, 8-80%, 8-75%, 9-75%, 9-70%, 10-70%, 10-68%, 11-68%, 11-65%, 12-65%, 12-62%, 13-62%, 13-60%, 14-60%, 14-58%, 15-58%, 15-55% or 16-55%.

Non-limiting examples of RNA biomarkers having increased exon skipping in a biological sample, relative to a control sample, can be found in Table 4.

In some embodiments, the at least one RNA biomarker is NCALD, ZNF573, PAK1, MIR4435-2HG, CD8B, PDGFC, TRAPPC2L, AC006504.5, ZNF512, FAM228B, NEIL2, FAM78A, FYB1, RNF216P1, ZCWPW1, DTX2, ATP5MD, MX2, LYRM1, GUF1, DPH7, NSFL1C, MTMR1, GTPBP10 or RGS3, or a combination thereof.

### Reduced Exon Skipping

In some embodiments, at least one RNA biomarker (e.g., at least: 2, 3, 4, 5, 6, 7, 8, 9, 10, 15, 20 or 25 RNA biomarkers) having a reduced exon skipping in the biological sample, relative to a control sample, is indicative of the subject as having, or having a propensity to develop, a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having a reduced exon skipping in the biological sample, relative to a control sample, is indicative of the subject as having a propensity to have a poorer prognosis of a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having a reduced exon skipping in the biological sample, relative to a control sample, is indicative of the subject as having a propensity to have a poorer treatment outcome for a fragile X-associated disorder (e.g., FXS).

In certain embodiments, exon skipping of the at least one RNA biomarker is reduced by at least about 5% in the biological sample, relative to a control sample, for example, at least about: 6%, 7%, 8%, 9%, 10%, 12%, 15%, 18%, 20%, 25%, 30%, 35%, 40%, 45%, 50%, 55%, 60%, 65%, 70%, 75%, 80%, 85% or 90%. In particular embodiments, the reduction is at least about 13.0%, optionally, the reduction is at least about 17.0%.

In some embodiments, exon skipping of the at least one RNA biomarker is reduced by about 5-90% in the biological sample, relative to a control sample, for example, about: 6-90%, 6-85%, 7-85%, 7-80%, 8-80%, 8-75%, 9-75%, 9-70%, 10-70%, 10-68%, 11-68%, 11-65%, 12-65%, 12-62%, 13-62%, 13-60%, 14-60%, 14-58%, 15-58%, 15-55%, 16-55% or 17-55%.

Non-limiting examples of RNA biomarkers having reduced exon skipping in a biological sample, relative to a control sample, can be found in Table 3.

In some embodiments, the at least one RNA biomarker is NCALD, DRAM2, RHOH, LAIR2, GBP3, GTF2H1, XPNPEP3, ZNF888, TBC1D5, AC060780.1, SDHAP2, KMT2A, SH3BP2, CSNK1G2, ATP5MD, NSUN5P1, LINC01128, RNF19A, SNHG8, TOP1MT or AL135818.1, or a combination thereof.

### Increased Mutually Exclusive Exon Switching

In some embodiments, at least one RNA biomarker (e.g., at least: 2, 3, 4, 5, 6, 7, 8, 9, 10, 15, 20 or 25 RNA biomarkers) having an increased mutually exclusive exon switching in the biological sample, relative to a control sample, is indicative of the subject as having, or having a propensity to develop, a fragile X-associated disorder (e.g., FXS). Mutually exclusive splicing generates alternative isoforms by retaining only one exon of a cluster of neighboring internal exons in the mature transcript and is a way to modulate protein function. See, e.g., Hatje et al., Mol Syst Biol. 13(12):959 (2017), Letunic et al., Hum Mol Genet. 11(13):1561-7 (2002), Meijers et al., Nature 449(7161):487-91 (2007), Pohl et al., Biosystems 114(1):31-8 (2013) and Tress et al., Trends Biochem Sci. 42(2):98-110 (2017).

In certain embodiments, at least one RNA biomarker (e.g., at least: 2, 3, 4, 5, 6, 7, 8, 9, 10, 15, 20 or 25 RNA biomarkers) having an increased mutually exclusive exon switching in the biological sample, relative to a control sample, is indicative of the subject as having a propensity to have a poorer prognosis of a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having an increased mutually exclusive exon switching in the biological sample, relative to a control sample, is indicative of the subject as having a propensity to have a poorer treatment outcome for a fragile X-associated disorder (e.g., FXS).

In certain embodiments, mutually exclusive exon switching of the at least one RNA biomarker is increased by at least about 5% in the biological sample, relative to a control sample, for example, at least about: 6%, 7%, 8%, 9%, 10%, 12%, 15%, 18%, 20%, 25%, 30%, 35%, 40%, 45%, 50%, 55%, 60%, 65%, 70%, 75%, 80%, 85% or 90%. In particular embodiments, the increase is at least about 10.0%, optionally, the increase is at least about 13.0%.

In some embodiments, mutually exclusive exon switching of the at least one RNA biomarker is increased by about 5-90% in the biological sample, relative to a control sample, for example, about: 5-85%, 6-85%, 6-80%, 7-80%, 7-75%, 8-75%, 8-70%, 9-70%, 9-65%, 10-65%, 10-60%, 11-60%, 11-55%, 12-55%, 12-50%, 13-50%, 13-45%, 14-45% or 14-40%.

Non-limiting examples of RNA biomarkers having increased mutually exclusive exon switching in a biological sample, relative to a control sample, can be found in Table 6.

In some embodiments, the at least one RNA biomarker is CR1, CRIM1, ZCWPW1, NAP1L1, TBC1D5, MIR4435-2HG, AC004593.2, GBP3, SEC61A2, PCNX2, TPT1-AS1, HLA-A, LUCAT1, PTPN2, SEC31B, POLR2J3, POLR2J4, CAST, NUMBL, PRMT7, ATF7IP2 or TIMM23B-AGAP6, or a combination thereof.

### Reduced Mutually Exclusive Exon Switching

In some embodiments, at least one RNA biomarker (e.g., at least: 2, 3, 4, 5, 6, 7, 8, 9, 10, 15, 20 or 25 RNA biomarkers) having a reduced mutually exclusive exon switching in the biological sample, relative to a control sample, is indicative of the subject as having, or having a propensity to develop, a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having a reduced mutually exclusive exon switching in the biological sample, relative to a control sample, is indicative of the subject as having a propensity to have a poorer prognosis of a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having a reduced mutually exclusive exon switching in the biological sample, relative to a control sample, is indicative of the subject as having a propensity to have a poorer treatment outcome for a fragile X-associated disorder (e.g., FXS).

In certain embodiments, mutually exclusive exon switching of the at least one RNA biomarker is reduced by at least about 5% in the biological sample, relative to a control sample, for example, at least about: 6%, 7%, 8%, 9%, 10%, 12%, 15%, 18%, 20%, 25%, 30%, 35%, 40%, 45%, 50%, 55%, 60%, 65%, 70%, 75%, 80%, 85% or 90%. In particular embodiments, the reduction is at least about 12.0%, optionally, the increase is at least about 15.0%.

In some embodiments, mutually exclusive exon switching of the at least one RNA biomarker is reduced by about 5-90% in the biological sample, relative to a control sample, for example, about: 5-88%, 6-88%, 6-85%, 7-85%, 7-82%, 8-82%, 8-80%, 9-78%, 9-75%, 10-75%, 10-72%, 11-72%, 11-70%, 12-70%, 12-68%, 13-68%, 13-65%, 14-65%, 14-62%, 15-62% or 15-60%.

Non-limiting examples of RNA biomarkers having reduced mutually exclusive exon switching in a biological sample, relative to a control sample, can be found in Table 5.

In some embodiments, the at least one RNA biomarker is HLA-A, ADGRE2, PAK1, TBC1D5, GTF2H2B, MICA, SLC29A2, ZBTB10, NLGN3, CAST, METTL25, ADAM15, LUCAT1, SSH1, SIRPB1 or GBP3, or a combination thereof.

### Increased Alternative 5′ Splicing

In some embodiments, at least one RNA biomarker (e.g., at least: 2, 3, 4, 5, 6, 7, 8, 9, 10, 15, 20 or 25 RNA biomarkers) having an increased alternative 5′ splicing in the biological sample, relative to a control sample, is indicative of the subject as having, or having a propensity to develop, a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having an increased alternative 5′ splicing in the biological sample, relative to a control sample, is indicative of the subject as having a propensity to have a poorer prognosis of a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having an increased alternative 5′ splicing in the biological sample, relative to a control sample, is indicative of the subject as having a propensity to have a poorer treatment outcome for a fragile X-associated disorder (e.g., FXS).

In certain embodiments, alternative 5′ splicing of the at least one RNA biomarker is increased by at least about 2.0% in the biological sample, relative to a control sample, for example, at least about: 2.5%, 3.0%, 3.5%, 4.0%, 4.5%, 5.0%, 52.5%, 6.0%, 6.5%, 7.0%, 7.5%, 8.0%, 8.5%, 9.0%, 9.5%, 10%, 12%, 15%, 18%, 20%, 25%, 30%, 35%, 40%, 45%, 50%, 55%, 60%, 65%, 70%, 75%, 80%, 85% or 90%. In particular embodiments, the increase is at least about 4.5%, optionally, the increase is at least about 5.0%.

In some embodiments, alternative 5′ splicing of the at least one RNA biomarker is increased by about 2.0-65% in the biological sample, relative to a control sample, for example, about: 2.5-65%, 2.5-60%, 3.0-60%, 3.0-55%, 3.5-55%, 3.5-50%, 4.0-50%, 4.0-45%, 4.5-45%, 4.5-40%, 5.0-40% or 5.0-35%.

Non-limiting examples of RNA biomarkers having increased alternative 5′ splicing in a biological sample, relative to a control sample, can be found in Table 8.

In some embodiments, the at least one RNA biomarker is PARP2, PACRGL, ENTPD1-AS1, NEIL2, FUZ, SDR39U1, ADAM15, EPOR, ZSCAN26, SNHG17, GPS2, NECAP1, MRPL11, DNAJC19, ANKZF1, C1orf162, PIGT, SLC25A37, AP1G1, CIC, ITGB7, ATG16L2, BECN1 or ARHGEF40, or a combination thereof.

### Reduced Alternative 5′ Splicing

In some embodiments, at least one RNA biomarker (e.g., at least: 2, 3, 4, 5, 6, 7, 8, 9, 10, 15, 20 or 25 RNA biomarkers) having a reduced alternative 5′ splicing in the biological sample, relative to a control sample, is indicative of the subject as having, or having a propensity to develop, a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having a reduced alternative 5′ splicing in the biological sample, relative to a control sample, is indicative of the subject as having a propensity to have a poorer prognosis of a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having a reduced alternative 5′ splicing in the biological sample, relative to a control sample, is indicative of the subject as having a propensity to have a poorer treatment outcome for a fragile X-associated disorder (e.g., FXS).

In certain embodiments, alternative 5′ splicing of the at least one RNA biomarker is reduced by at least about 2.0% in the biological sample, relative to a control sample, for example, at least about: 2.5%, 3.0%, 3.5%, 4.0%, 4.5%, 5.0%, 52.5%, 6.0%, 6.5%, 7.0%, 7.5%, 8.0%, 8.5%, 9.0%, 9.5%, 10%, 12%, 15%, 18%, 20%, 25%, 30%, 35%, 40%, 45%, 50%, 55%, 60%, 65%, 70%, 75%, 80%, 85% or 90%. In particular embodiments, the reduction is at least about 4.5%, optionally, the increase is at least about 5.5%.

In some embodiments, alternative 5′ splicing of the at least one RNA biomarker is reduced by about 2.0-65% in the biological sample, relative to a control sample, for example, about: 2.5-65%, 2.5-60%, 3.0-60%, 3.0-55%, 3.5-55%, 3.5-50%, 4.0-50%, 4.0-45%, 4.5-45%, 4.5-40%, 5.0-40%, 5.0-35%, 5.5-35% or 5.5-30%.

Non-limiting examples of RNA biomarkers having reduced alternative 5′ splicing in a biological sample, relative to a control sample, can be found in Table 7.

In some embodiments, the at least one RNA biomarker is BANP, PIGA, SNHG8, RAD52, IRF3, CEP78, SPINT1, TMEM156, NT5C3B, PLD2, HLA-A, ANKRD12, CASP8, PACS2, HLA-DMA, DHPS or PDCD6, or a combination thereof.

### Increased Alternative 3′ Splicing

In some embodiments, at least one RNA biomarker (e.g., at least: 2, 3, 4, 5, 6, 7, 8, 9, 10, 15, 20 or 25 RNA biomarkers) having an increased alternative 3′ splicing in the biological sample, relative to a control sample, is indicative of the subject as having, or having a propensity to develop, a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having an increased alternative 3′ splicing in the biological sample, relative to a control sample, is indicative of the subject as having a propensity to have a poorer prognosis of a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having an increased alternative 3′ splicing in the biological sample, relative to a control sample, is indicative of the subject as having a propensity to have a poorer treatment outcome for a fragile X-associated disorder (e.g., FXS).

In certain embodiments, alternative 3′ splicing of the at least one RNA biomarker is increased by at least about 2.0% in the biological sample, relative to a control sample, for example, at least about: 2.5%, 3.0%, 3.5%, 4.0%, 4.5%, 5.0%, 52.5%, 6.0%, 6.5%, 7.0%, 7.5%, 8.0%, 8.5%, 9.0%, 9.5%, 10%, 12%, 15%, 18%, 20%, 25%, 30%, 35%, 40%, 45%, 50%, 55%, 60%, 65%, 70%, 75%, 80%, 85% or 90%. In particular embodiments, the increase is at least about 6.5%, optionally, the increase is at least about 7.5%.

In some embodiments, alternative 3′ splicing of the at least one RNA biomarker is increased by about 5.0-90% in the biological sample, relative to a control sample, for example, about: 5.0-85%, 5.2-85%, 5.2-80%, 5.5-80%, 5.5-75%, 5.8-75%, 5.8-70%, 6.0-70%, 6.0-65%, 6.2-65%, 6.2-60%, 6.5-60%, 6.5-55%, 6.8-55%, 6.8-50%, 7.0-50%, 7.0-45%, 7.2-45%, 7.2-40% or 7.5-40%.

Non-limiting examples of RNA biomarkers having increased alternative 3′ splicing in a biological sample, relative to a control sample, can be found in Table 10.

In some embodiments, the at least one RNA biomarker is SNX5, POLR2J3, MPPE1, AC016394.2, DPM1, E2F5, PTPN7, MTFP1, TOR1AIP1, POT1, JOSD2, NLRX1, FDXR, ZDHHC16, ALKBH4, RPS9, ZNF302, TENT4B, ADGRE2, TKT, CARD8, RBM26 or WSB1, or a combination thereof.

### Reduced Alternative 3′ Splicing

In some embodiments, at least one RNA biomarker (e.g., at least: 2, 3, 4, 5, 6, 7, 8, 9, 10, 15, 20 or 25 RNA biomarkers) having a reduced alternative 3′ splicing in the biological sample, relative to a control sample, is indicative of the subject as having, or having a propensity to develop, a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having a reduced alternative 3′ splicing in the biological sample, relative to a control sample, is indicative of the subject as having a propensity to have a poorer prognosis of a fragile X-associated disorder (e.g., FXS).

In certain embodiments, at least one RNA biomarker (e.g., at least: 2, 3, 4, 5, 6, 7, 8, 9, 10, 15, 20 or 25 RNA biomarkers) having a reduced alternative 3′ splicing in the biological sample, relative to a control sample, is indicative of the subject as having a propensity to have a poorer treatment outcome for a fragile X-associated disorder (e.g., FXS).

In certain embodiments, alternative 3′ splicing of the at least one RNA biomarker is reduced by at least about 2.0% in the biological sample, relative to a control sample, for example, at least about: 2.5%, 3.0%, 3.5%, 4.0%, 4.5%, 5.0%, 52.5%, 6.0%, 6.5%, 7.0%, 7.5%, 8.0%, 8.5%, 9.0%, 9.5%, 10%, 12%, 15%, 18%, 20%, 25%, 30%, 35%, 40%, 45%, 50%, 55%, 60%, 65%, 70%, 75%, 80%, 85% or 90%. In particular embodiments, the reduction is at least about 4.0%, optionally, the increase is at least about 5.0%.

In some embodiments, alternative 3′ splicing of the at least one RNA biomarker is reduced by about 2.0-65% in the biological sample, relative to a control sample, for example, about: 2.5-65%, 2.5-60%, 3.0-60%, 3.0-55%, 3.5-55%, 3.5-50%, 4.0-50%, 4.0-45%, 4.5-45%, 4.5-40%, 5.0-40% or 5.0-35%.

Non-limiting examples of RNA biomarkers having reduced alternative 3′ splicing in a biological sample, relative to a control sample, can be found in Table 9.

In some embodiments, the at least one RNA biomarker is DDX60L, ATP11A, SRGAP2, CEACAM21, COX18, WDR47, PATZ1, POLM, CC2D1B, CLK4, MIB2, PHF1, KANSL1 or TCF3, or a combination thereof.

### Assaying Biomarkers

The level or splicing of a RNA biomarker can be measured using any technique suitable for detecting RNA expression level or expression pattern in a biological sample. For example, by performing northern blot analysis, in situ hybridization, quantitative reverse transcriptase polymerase chain reaction (RT-qPCR), a microarray assay, cDNA sequencing (RNA-Seq, Drop-Seq, CEL-seq2, MARS-seq, SCRB-seq, Smart-seq, and Smart-seq2), flow cytometry, or a combination thereof. See, e.g., www.illumina.com/science/sequencing-method-explorer/kits-and-arrays/drop-seq.html, Macosko et al., Cell 161(5):1202-14 (2015) and Ziegenhain et al., Mol Cell 65(4):631-43 (2017). In some embodiments, the level or splicing of the at least one RNA biomarker is measured using a microarray assay.

Primers for amplifying and/or sequencing biomarkers of the disclosure and suitable probes for detecting such biomarkers can be designed using conventional methodology by those skilled in the art.

In particular embodiments, the level or splicing of a RNA biomarker is measured indirectly, at the protein level, using any technique known in the art. For example, by performing enzyme-linked immunoassay (ELISA) or Western blotting.

In some embodiments, the level and/or splicing of the at least one RNA biomarker in the sample is compared to that in a control sample or a reference standard.

In some embodiments, the control sample comprises tissue or blood from an unaffected subject or a population of unaffected subjects. An unaffected subject is a healthy subject, a subject who is not diagnosed with a fragile X-associated disorder (e.g., FXS) or a subject who does not have a fragile X-associated disorder (e.g., FXS). In some embodiments, the control sample (e.g., tissue or blood sample) is processed along with the sample from the subject. In other embodiments, the control sample is processed separately (e.g., at an earlier or a later time) from the test sample.

The term “reference standard” can be, for example, a mean, an average, a numerical mean or range of numerical means, a numerical pattern, a graphical pattern or the corresponding RNA expression or splicing level derived from a reference subject (e.g., an unaffected subject) or reference population (e.g., a population of unaffected subjects).

In some embodiments, the control sample is from a sample from a typically developing subject, e.g., from an age-matched sample from a typically developing subject. In certain embodiments, the control sample is a theoretical value calculated from the general population. In particular embodiments, the control sample is a baseline sample of the subject, e.g., at an earlier age or before treatment.

### Subjects

The term “subject” refers to a mammalian subject, preferably human, diagnosed with or suspected of having a fragile X-associated disorder (e.g., FXS).

In some embodiments, the subject has one X chromosome and one Y chromosome. In some embodiments, the subject has two X chromosomes. In certain embodiments, the subject has two X chromosomes and one Y chromosome. In particular embodiments, the subject has one X chromosome and two Y chromosomes.

In some embodiments, the subject is a human male. In some embodiments the subject is human female.

In some embodiments, the subject is at least about 1 month of age, for example, at least about 2, 3, 4, 5, 6, 7, 8, 9, 10, 11, 12, 15, 18 or 21 months of age, or at least about: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 years of age. In some embodiments, the subject is about: 1-100, 1-80, 1-60, 1-30, 1-24, 1-20, 1-18, 1-12, 1-10, 1-8, 1-6, 2-100, 2-80, 2-60, 2-30, 2-24, 2-20, 2-18, 2-12, 2-10, 2-8, 2-6, 3-100, 3-80, 3-60, 3-30, 3-24, 3-20, 3-18, 3-12, 3-10, 3-8, 3-6, 4-100, 4-80, 4-60, 4-30, 4-24, 4-20, 4-18, 4-12, 4-10, 4-8, 4-6, 5-100, 5-80, 5-60, 5-30, 5-24, 5-20, 5-18, 5-12, 5-10, 5-8, 6-100, 6-80, 6-60, 6-30, 6-24, 6-20, 6-18, 6-12, 6-10, 8-100, 8-80, 8-60, 8-30, 8-24, 8-20, 8-18, 8-12, 10-100, 10-80, 10-60, 10-30, 10-24, 10-20, 10-18, 12-100, 12-80, 12-38, 12-60, 12-50, 12-40, 12-30, 12-24, 12-20, 12-18, 18-100, 18-80, 18-60, 18-50, 18-40, 18-30, 18-24, 20-100, 20-80, 20-60, 20-50, 20-40, 20-30, 20-25, 30-100, 30-80, 30-60, 30-55, 30-50, 30-45, 30-40, 40-100, 40-80, 40-60, 40-55 or 40-50 years of age. In some embodiments, the subject is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80 or 100 years of age. In other embodiments, the subject is a fetus. In some embodiments, the subject is a neonatal subject.

In some embodiments, the subject is 18 years of age or older, e.g., 18 to less than 40 years of age, 18 to less than 45 years of age, 18 to less than 50 years of age, 18 to less than 55 years of age, 18 to less than 60 years of age, 18 to less than 65 years of age, 18 to less than 70 years of age, 18 to less than 75 years of age, 40 to less than 75 years of age, 45 to less than 75 years of age, 50 to less than 75 years of age, 55 to less than 75 years of age, 60 to less than 75 years of age, 65 to less than 75 years of age, 60 to less than 75 years of age, 40 years of age or older, 45 years of age or older, 50 years of age or older, 55 years of age or older, 60 years of age or older, 65 years of age or older, 70 years of age or older, 75 years of age or older or 100 years of age or older. In some embodiments, the subject is a child. In some embodiments, the subject is 18 years of age or younger, e.g., 0-18 years of age, 0-12 years of age, 0-16 years of age, 0-17 years of age, 2-12 years of age, 2-16 years of age, 2-17 years of age, 2-18 years of age, 3-12 years of age, 3-16 years of age, 3-17 years of age, 3-18 years of age, 4-12 years of age, 4-16 years of age, 4-17 years of age, 4-18 years of age, 6-12 years of age, 6-16 years of age, 6-17 years of age, 6-18 years of age, 9-12 years of age, 9-16 years of age, 9-17 years of age, 9-18 years of age, 12-16 years of age, 12-17 years of age or 12-18 years of age.

In some embodiments, the subject has one or more of the physical and/or medical features associated with a fragile X-associated disorder (e.g., FXS). Non-limiting examples of physical features associated with FXS include a long face, prominent ears and chin, arched palate, large testicles at puberty, low muscle tone, flat feet, and hyperextensible joints. Non-limiting examples of medical or behavioral features associated with FXS include sleep problems, seizures, recurrent ear infections, mitral valve prolapse, behaviors of hyperactivity, short attention span, hand biting or hand flapping, poor eye contact and social skills, shyness, anxiety, autism, epilepsy, aggression, delayed speech and motor development, repetitive speech, sensitivity to sensory stimulation (including a hypersensitivity to being touched, to light or to sound). In certain embodiments, the subject is a female with an IQ score of less than 115, 110, 105, 100, 95 or 90. In particular embodiments, the subject is a male with an IQ score of less than 60, 55, 50 or 45.

In certain embodiments, the subject has one or more of the following: irregular menses, fertility problem, elevated FSH (follicle-stimulating hormone) level, premature ovarian failure, primary ovarian insufficiency, and vasomotor symptoms (e.g., “hot flash”). In some embodiments, the subject has one or more of the following: intention tremor, parkinsonism, ataxia, memory loss, white matter lesion involving middle cerebellar peduncles, and cognitive decline.

### Treatments

In some embodiments, the method further comprises treating the subject if the subject is diagnosed to have, or has a propensity to develop, a fragile X-associated disorder (e.g., FXS).

“Treat,” “treating” or “treatment” refers to therapeutic treatment wherein the objective is to slow down (lessen) an undesired physiological change or disease, such as the development or progression of the fragile X-associated disorder (e.g., FXS), or to provide a beneficial or desired clinical outcome during treatment. Beneficial or desired clinical outcomes include alleviation of symptoms, diminishment of extent of disease, stabilized (i.e., not worsening) state of disease, delay or slowing of disease progression, amelioration or palliation of the disease state, whether detectable or undetectable.

Non-limiting examples of symptoms include speech and motor development symptoms, cognitive disabilities, including learning and intellectual disabilities, hyperactivity, short attention span, anxiety, sensitivity to sensory stimulation, sleep problems, seizures, recurrent ear infections, and mitral valve prolapse.

In some embodiments, treating the subject comprises administering a therapeutic, providing the subject with a specific diet, or a combination thereof. Non-limiting examples of the therapeutics include metabotropic glutamate receptor 5 (mGluR5) modulator (e.g., Basimglurant or Mavoglurant), GABAB receptor activator (e.g., arbaclofen), GABAA or GABAB receptor activator (e.g., acamprosate), AMPAkine (e.g., AX516), CB1 inhibitor (e.g., rimonabant), RAS signaling inhibitor (e.g., lovastatin), STEP inhibitor, S6K inhibitor, PAK inhibitor (e.g., FRAX486), MMP9 inhibitor (e.g., minocycline), and GSK3β inhibitor (e.g., lithium). In particular embodiments, treating the subject comprises providing the subject with a ketogenic (“keto”) diet.

### Systems

In another aspect, the present disclosure provides a system, comprising one or more polynucleotide probes and/or one or more polynucleotide primers configured to detect, in a biological sample, the level and/or splicing of the at least one biomarker associated with fragile X syndrome (FXS).

In some embodiments, the biomarker is a RNA biomarker. In certain embodiments, the at least one RNA biomarker is selected from the group consisting of AGAP1, RAB25, FAM3B, XKR3, MAP3K15, LEP, RP11-706O15.3, GCOM1, CXCL6, RGL3, NECAB2, TGM3, LRRC6, MAB21L3, RP11-36B15.1, AC091878.1, RP11-154H23.3, NOV, AC093495.4, RP11-455F5.6, RGPD2, COL9A3, CLEC18A, RP11-256L6.2, LINC01127, SLC38A11, EFCAB12, LA16c-380H5.5, CXCL1, RP11-1334A24.5, AC100793.2, ANKDD1A, AVIL, RP11-44F14.8, RP11-290F20.1, AC116366.5, EPHB4, ST6GALNAC3, PANX2, CREB5, KIAA0319, HECW2, ADCY4, LINC00173, RP11-59D5_B.2, RP11-274B18.2, RP11-213H15.3, CORO7-PAM16, HAL, DPEP3, AC002467.7, MGAM, PNMA8A, FMR1, S100B, RP11-885N19.6, RP11-545I5.3, AC091814.2, KLRC2, L1TD1, PGBD5, MXRA7, CROCC2, SEMA5A, PLA2G4C, RP11-1008C21.1, TANC1, C4orf50, NUAK1, AC104809.4, RGS17, KCNS1, DRAXIN, B3GAT1, ARHGEF28, KIF19, APOL4, GZMH, GAS1, SCD5, GLB1L2, IGHA1, KNDC1, RP11-383H13.1, FGFR2, TFCP2L1, PDGFRB, LAG3, GPR153, PODN, CKB, CERCAM, ZNF365, JUP, TRNP1, JAKMIP1, CPXM1, SLC1A7, LGR6, FCRL6, MORN4, TUBB2A, PRSS23, BFSP1, NCALD, ZNF573, PAK1, MIR4435-2HG, CD8B, PDGFC, TRAPPC2L, AC006504.5, ZNF512, FAM228B, NEIL2, FAM78A, FYB1, RNF216P1, ZCWPW1, DTX2, ATP5MD, MX2, LYRM1, GUF1, DPH7, NSFL1C, MTMR1, GTPBP10, RGS3, DRAM2, RHOH, LAIR2, GBP3, GTF2H1, XPNPEP3, ZNF888, TBC1D5, AC060780.1, SDHAP2, KMT2A, SH3BP2, CSNK1G2, NSUN5P1, LINC01128, RNF19A, SNHG8, TOP1MT, AL135818.1, CR1, CRIM1, NAP1L1, AC004593.2, SEC61A2, PCNX2, TPT1-AS1, HLA-A, LUCAT1, PTPN2, SEC31B, POLR2J3, POLR2J4, CAST, POLR2J4, NUMBL, PRMT7, ATF7IP2, TIMM23B-AGAP6, ADGRE2, GTF2H2B, MICA, SLC29A2, ZBTB10, NLGN3, METTL25, ADAM15, SSH1, SIRPB1, PARP2, PACRGL, ENTPD1-AS1, FUZ, SDR39U1, EPOR, ZSCAN26, SNHG17, GPS2, NECAP1, MRPL11, DNAJC19, ANKZF1, C1orf162, PIGT, SLC25A37, AP1G1, CIC, ITGB7, ATG16L2, BECN1, ARHGEF40, BANP, PIGA, RAD52, IRF3, CEP78, SPINT1, TMEM156, NT5C3B, PLD2, ANKRD12, CASP8, PACS2, HLA-DMA, DHPS, PDCD6, SNX5, MPPE1, AC016394.2, DPM1, E2F5, PTPN7, MTFP1, TOR1AIP1, POT1, JOSD2, NLRX1, FDXR, ZDHHC16, ALKBH4, RPS9, ZNF302, TENT4B, TKT, CARD8, RBM26, WSB1, DDX60L, ATP11A, SRGAP2, CEACAM21, COX18, WDR47, PATZ1, POLM, CC2D1B, CLK4, MIB2, PHF1, KANSL1 or TCF3, or a combination thereof.

Non-limiting examples of hybridization formats include solution phase, solid phase, and mixed phase. In some embodiments, the one or more polynucleotide probes are immobilized on a solid substrate.

In particular embodiments, the system is a microarray. Array-based detection can be performed using commercially available arrays, e.g., from Affymetrix/Thermo Fisher Scientific or other manufacturers. See, e.g., Schena et al., Science 270(5235):467-70 (1995) and Barbulovic-Nad et al., Crit Rev Biotechnol 26(4):237-59 (2006), the contents of which are incorporated herein by reference.

Primers and/or probes for detecting and/or quantifying RNA biomarkers of the disclosure can be designed using conventional methodology by those skilled in the art, for example, using custom probe designing tools available through commercial vendors.

In some embodiments, the primer is a DNA polynucleotide. In some embodiments, the primer has a length of at least about 12 nucleotides, for example, at least about: 13, 14, 15, 16, 17, 18, 19, 20, 21, 22, 23, 24, 25, 26, 27, 28, 29 or 30 nucleotides. In some embodiments, the primer has a length of about 12-40 nucleotides, for example, about: 12-35, 12-30, 12-25, 13-40, 13-35, 13-30, 13-25, 14-40, 14-35, 14-30, 14-25, 15-40, 15-35, 15-30 or 15-25 nucleotides. In certain embodiments, the primer has a length of about 15-25 nucleotides. In particular embodiments, the primer has a length of about: 12, 13, 14, 15, 16, 17, 18, 19, 20, 21, 22, 23, 24, 25, 26, 27, 28, 29, 30, 35 or 40 nucleotides. In some embodiments, the primer is an oligonucleotide.

In some embodiments, the primer is complementary to at least a portion of an RNA biomarker that has an altered (e.g., increased or reduced) expression in the biological sample, relative to a control sample. In certain embodiments, the primer is complementary to at least a portion of an exon that has an altered (e.g., increased or reduced) exon skipping in the biological sample, relative to a control sample. In particular embodiments, the primer is complementary to at least a portion of an exon that has an altered (e.g., increased or reduced) mutually exclusive exon switching in the biological sample, relative to a control sample. In some embodiments, the primer is complementary to an alternative 5′ splice site that has an altered (e.g., increased or reduced) splicing in the biological sample, relative to a control sample. In certain embodiments, the primer is complementary to an alternative 3′ splice site that has an altered (e.g., increased or reduced) splicing in the biological sample, relative to a control sample. In particular embodiments, methods of the disclosure also include using a control primer that is complementary to a sequence that is not altered in its expression and/or splicing in the biological sample, relative to a control sample.

In some embodiments, the probe is a DNA polynucleotide. In some embodiments, the probe has a length of at least about 12 nucleotides, for example, at least about: 13, 14, 15, 16, 17, 18, 19, 20, 21, 22, 23, 24, 25, 26, 27, 28, 29 or 30 nucleotides. In some embodiments, the probe has a length of about 12-40 nucleotides, for example, about: 12-35, 12-30, 12-25, 13-40, 13-35, 13-30, 13-25, 14-40, 14-35, 14-30, 14-25, 15-40, 15-35, 15-30 or 15-25 nucleotides. In certain embodiments, the probe has a length of about 15-25 nucleotides. In particular embodiments, the probe has a length of about: 12, 13, 14, 15, 16, 17, 18, 19, 20, 21, 22, 23, 24, 25, 26, 27, 28, 29, 30, 35 or 40 nucleotides. In some embodiments, the probe is an oligonucleotide.

In some embodiments, the probe is complementary to at least a portion of an RNA biomarker that has an altered (e.g., increased or reduced) expression in the biological sample, relative to a control sample. In certain embodiments, the probe is complementary to at least a portion of an exon that has an altered (e.g., increased or reduced) exon skipping in the biological sample, relative to a control sample. In particular embodiments, the probe is complementary to at least a portion of an exon that has an altered (e.g., increased or reduced) mutually exclusive exon switching in the biological sample, relative to a control sample. In some embodiments, the probe is complementary to an alternative 5′ splice site that has an altered (e.g., increased or reduced) splicing in the biological sample, relative to a control sample. In certain embodiments, the probe is complementary to an alternative 3′ splice site that has an altered (e.g., increased or reduced) splicing in the biological sample, relative to a control sample. In particular embodiments, methods of the disclosure also include using a control probe that is complementary to a sequence that is not altered in its expression and/or splicing in the biological sample, relative to a control sample.

### Methods and Compositions for Stratifying Fragile X-Associated Disorder Patients

In another aspect, the present disclosure provides a method of stratifying a population of subjects having, or having a propensity to develop, fragile X-associated disorder (e.g., FXS), wherein the method comprises assaying biological samples from the subjects for the presence of FMR1 RNA isoform 12.

In another aspect, the present disclosure provides a method of stratifying a set of subjects having fragile X-associated disorder (e.g., FXS), e.g., wherein the method comprises assaying FMR1 RNA in a biological sample from the subject, and stratifying the set of subjects for treatment based on the level of the FMR1 RNA in the biological sample.

In another aspect, the present disclosure provides a method for assessing the efficacy of a drug (outcome measure) for treatment of fragile X-associated disorder (e.g., FXS), comprising stratifying a population of subjects to create a stratified population comprising a subpopulation who has the FMR1 RNA isoform 12 and a subpopulation who does not have the FMR1 RNA isoform 12, and administering the drug to the subpopulation who has FMR1 RNA isoform 12, or to both subpopulations.

The FMR1 gene is located within chromosome band Xq27.3 between base pairs 147,911,919 and 147,951,125. The assembly of FMR1 gene transcript that comprises 17 exons (corresponding to the UniProtKB reference number Q06787) is known as the normal FMR1 RNA splicing. That is, the first exon (between base pairs 147,911,919 and 147,912,230, SEQ ID NO: 7) is spliced to the second exon (between base pairs 147,921,933 and 147,921,985, SEQ ID NO: 8) to produce “isoform 1” or “isol.” FMR1 isoform 1 is produced in typical developing individuals and a subpopulation of FXS subjects.

In a subpopulation of FXS subjects, the first exon (between base pairs 147,911,919 and 147,912,230, SEQ ID NO: 7) is spliced to a pseudo exon (between base pairs 147,912,728 and 147,914,451, SEQ ID NO: 9) to produce “isoform 12” or “iso12.” This predicted isoform is also annotated as FMR1-217 or ENST00000621447.1.

(SEQ ID NO: 7)
CTCAGTCAGGCGCTCAGCTCCGTTTCGGTTTCACTTCCGGTGGAG
GGCCGCCTCTGAGCGGGCGGCGGGCCGACGGCGAGCGCGGGCGGC
GGCGGTGACGGAGGCGCCGCTGCCAGGGGGCGTGCGGCAGCGCGG
CGGCGGCGGCGGCGGCGGCGGCGGCGGAGGCGGCGGCGGCGGCGG
CGGCGGCGGCGGCTGGGCCTCGAGCGCCCGCAGCCCACCTCTCGG
GGGCGGGCTCCCGGCGCTAGCAGGGCTGAAGAGAAGATGGAGGAG
CTGGTGGTGGAAGTGCGGGGCTCCAATGGCGCTTTCTACAAG
(SEQ ID NO: 8)
GCATTTGTAAAGGATGTTCATGAAGATTCAATAACAGTTGCATTT
GAAAACAA
(SEQ ID NO: 9)
CATTGGGACTTCGGAGAGCTCCACTGTTCTGGGCGAGGGCTGTGA
AGAAAGAGTAGTAAGAAGCGGTAGTCGGCACCAAATCACAATGGC
AACTGATTTTTAGTGGCTTCTCTTTGTGGATTTCGGAGGAGATTT
TAGATCCAAAAGTTTCAGGAAGACCCTAACATGGCCCAGCAGTGC
ATTGAAGAAGTTGATCATCGTGAATATTCGCGTCCCCCTTTTTGT
TAAACGGGGTAAATTCAGGAATGCACATGCTTCAGCGTCTAAAAC
CATTAGCAGCGCTGCTACTTAAAAATTGTGTGTGTGTGTTTAAGT
TTCCAAAGACCTAAATATATGCCATGAAACTTCAGGTAATTAACT
GAGAGTATATTATTACTAGGGCATTTTTTTTTTAACTGAGCGAAA
ATATTTTTGTGCCCCTAAGAACTTGACCACATTTCCTTTGAATTT
GTGGTGTTGCAGTGGACTGAATTGTTGAGGCTTTATATAGGCATT
CATGGGTTTACTGTGCTTTTTAAAGTTACACCATTGCAGATCAAC
TAACACCTTTCAGTTTTAAAAGGAAGATTTACAAATTTGATGTAG
CAGTAGTGCGTTTGTTGGTATGTAGGTGCTGTATAAATTCATCTA
TAAATTCTCATTTCCTTTTGAATGTCTATAACCTCTTTCAATAAT
ATCCCACCTTACTACAGTATTTTGGCAATAGAAGGTGCGTGTGGA
AGGAAGGCTGGAAAATAGCTATTAGCAGTGTCCAACACAATTCTT
AAATGTATTGTAGAATGGCTTGAATGTTTCAGACAGGACACGTTT
GGCTATAGGAAAATAAACAATTGACTTTATTCTGTGTTTACCAAT
TTTATGAAGACATTTGGAGATCAGTATATTTCATAAATGAGTAAA
GTATGTAAACTGTTCCATACTTTGAGCACAAAGATAAAGCCTTTT
GCTGTAAAAGGAGGCAAAAGGTAACCCCGCGTTTATGTTCTTAAC
AGTCTCATGAATATGAAATTGTTTCAGTTGACTCTGCAGTCAAAA
TTTTAATTTCATTGATTTTATTGATCCATAATTTCTTCTGGTGAG
TTTGCGTAGAATCGTTCACGGTCCTAGATTAGTGGTTTTGGTCAC
TAGATTTCTGGCACTAATAACTATAATACATATACATATATATGT
GTGAGTAACGGCTAATGGTTAGGCAAGATTTTGATTGACCTGTGA
TATAAACTTAGATTGGATGCCACTAAAGTTTGCTTATCACAGAGG
GCAAGTAGCACATTATGGCCTTGAAGTACTTATTGTTCTCTTCCA
GCAACTTATGATTTGCTCCAGTGATTTTGCTTGCACACTGACTGG
AATATAAGAAATGCCTTCTATTTTTGCTATTAATTCCCTCCTTTT
TTGTTTTGTTTTGTAACGAAGTTGTTTAACTTGAAGGTGAATGAA
GAATAGGTTGGTTGCCCCTTAGTTCCCTGAGGAGAAATGTTAATA
CTTGAACAAGTGTGTGTCAGACAAATTGCTGTTATGTTTATTTAA
TTAAGTTTGATTTCTAAGAAAATCTCAAATGGTCTGCACTGATGG
AAGAACAGTTTCTGTAACAAAAAAGCTTGAAATTTTTATATGACT
TATAATACTGCTGTGAGTTTTAAAAGTAAAGCAAAAGTAAACTGA
GTTGCTTGTCCAGTGGGATGGACAGGAAAGATGTGAAATAAAAAC
CAATGAAAAATGAA

Additional information on Exon 1, the pseudo exon, and isoform 12 can be found at: useast.ensembl.org/Homo_sapiens/Transcript/Summary?db=core;g=ENSG00000102081;r=X :147911951-147951125;t=ENST00000370475; and useast.ensembl.org/Homo_sapiens/Transcript/Summary?db=core;g=ENSG00000102081;r=X :147911951-147951125;t=ENST00000621447, the contents of both of which are incorporated herein by reference in their entirety.

In some embodiments, the presence of FMR1 RNA isoform 12 in the biological sample is assayed before, during, and/or after a therapeutic treatment for evaluating therapeutic efficacy (outcome measure).

The subject can be any one of the subjects disclosed herein.

### EXEMPLIFICATION

Most FXS studies focus on Fmr1 knockout (KO) mouse models. Shah et al. show, for the first time, that Fmr1 KO mice have dysregulated pre-mRNA splicing in the hippocampus part of the brain (Shah et al., Cell Rep. 30(13):4459-72 (2020)). The present disclosure shows that missplicing in the Fmr1 KO mouse occurs in all brain regions tested, as well as all peripheral tissues tested. Because FMRP is likely present in all cells, missplicing probably also occurs in all cells.

Based on the mouse FMRP knockout data, it is surmised that RNA missplicing would also occur in human cells, and possibly white blood cells (WBCs) (red blood cells and platelets are anucleate). It is believed that RNA biomarkers from biological samples comprising such cells would be more easily obtainable than biomarkers from brain tissues, and can be used for FXS diagnosis, prognosis, and patient stratification. The methods disclosed herein would be a useful platform for testing drug efficacy and perhaps stratification of individuals (e.g., individuals with FXS), and would be useful for personalized medicine for individuals with FXS.

### Example 1. Methods

### Human FXS Participant Studies

All samples collected for the study were based on voluntary informed consent provided by the participants in accordance with Rush University Medical Center IRB regulations. All participants were Caucasian males with a FMR1 full mutation (CGG repeats >200) or typically developing individuals (CGG repeats <55) as confirmed by DNA analysis. Intelligence quotient (IQ) scores were obtained using the Stanford-Binet Scale—Fifth Edition (SB5) (Roid and Pomplun, 2012). The adaptive skills of participants was determined using an semi-structured interview and measured using the Vineland Adaptive Behavior skills (Vineland-3). The Adaptive Behavior Composite (ABC) standard score (SS) is the measure of overall adaptive functioning based on scores assessing the following domains: communication, daily living skills, and socialization. All FXS males in the study were diagnosed with Autism Spectrum Disorders (ASD) based on both Autism Diagnostic Observation Schedule (ADOS) assessments and the Diagnostic and Statistical Manual-5th Edition criteria (DSM-5) (ref) by clinicians with expertise in idiopathic ASD, and ASD in FXS. FXS patients were aged 16-38 years with FXS phenotypes, an IQ range of 20-52 and ABC standard score range of 20-41 (Table 1). Age matched TD individuals for the study were aged 22-29 with a normal IQ and no known neuropsychiatric conditions (Table 1).

For CGG repeat size determination in the 5′ UTR of the FMR1 gene, DNA isolated from whole blood was analyzed using the Asuragen FMR1 Amplidex PCR Kit. Methylation status was determined using the Asuragen FMR1 methylation PCR Kit and/or Southern blot analysis. FMRP levels were quantified by generating dried blood spots (DBS) from the samples. FMRP levels were quantified by generating dried blood spots (DBS) from the samples. To generate DBS, 12-50 μl spots were put on each blood card and allowed to dry. The blood cards were then stored at−80. Discs were punched using a 6 mm punch and incubated in lysis buffer. Extracted sample was centrifuged and FMRP quantified using the Luminex Microplex immunochemistry assay. FMRP levels were normalized to 1000 WBCs per sample. Additionally, FMRP levels were also quantified by using peripheral blood mononuclear cells (PBMC) samples. PBMC were isolated from whole blood using a Cell Preparation (CPT) blood tubes. Isolated PBMC were lysed and quantified for total protein concentration using a spectrophotometer and FMRP quantified using a Luminex Microplex immunochemistry assay. FMRP levels were normalized to total protein. Both methods produced comparable levels of FMRP in the samples assessed.

Frozen post-mortem brain tissues were obtained from University of California at Davis Brain Repository from FXS male individuals (N=6) and age-matched typically developing (TD) males (N=5).

### RNA Extraction and Sequencing

RNA was extracted from patient leukocytes using the LeukoLOCK™ total RNA isolation system (AM1923, Thermo Fisher Scientific, Waltham, MA). Ten mL fresh blood was collected from FXS male patients (N=10) and age-matched typically developing males (N=7) (controls) in an anti-coagulant containing tube, and RNA was extracted using a LeukoLOCK™ fractionation & stabilization kit (AM1933, Thermo Fisher Scientific, Waltham, MA), per the manufacturer's instructions. Briefly, the blood sample was passed through a LeukoLOCK™ filter and 3 mL phosphate buffered saline (PBS) was used to rinse the filter followed by 3 mL of RNAlater® RNA Stabilization Solution (Thermo Fisher Scientific, Waltham, MA). The residual RNAlater® was expelled from the LeukoLOCK™ filter and the filters were capped and stored at −80° C.

To extract RNA, the filters were thawed at room temperature for 5 mins and then the remaining RNAlater® was removed. The filter was flushed with 4 ml of TRI Reagent, and the lysate was collected in a 15-ml tube. 800 μl 1-Bromo-3-chloropropane (BCP) was added to each tube and vortexed vigorously for 30 seconds. The tube was then incubated at room temperature for 5 minutes. After centrifugation for 10 minutes at 4° C. at ˜2,000×g, the aqueous phase was recovered. To recover long RNA fractions, 0.5 volumes of 100% ethanol were added and mixed well. The RNA was then recovered using the RNA clean and concentrator kit. DNase treatment was performed using Turbo™ DNase (Thermo Fisher Scientific, Waltham, MA), and the RNA obtained was resuspended in RNAse free water and stored at −80° C. 1 μg of the RNA was used for cDNA synthesis using the QuantiTect® reverse transcription kit (Qiagen, Hilden, Germany) to assess for depletion of the Globin mRNA using qPCR, to confirm exclusion of red blood cells from the prep. 3 μg of RNA sample was sent to Novogene (Beijing, China) for a directional mRNA library preparation using polyA enrichment. The libraries were sequenced on the NovaSeq platform to generate paired end, 150 bp reads.

The post-mortem frozen cortical tissues from FXS male individuals (N=6) and age-matched typically developing (TD) males (N=5), were powdered in liquid nitrogen using a mortar and pestle. The fine powder was then homogenized on ice in a dounce homogenizer using TRIzol™ Reagent (ThermoFisher Scientific #15596026) and the lysate collected. Total RNA was extracted using BCP and recovered as above and stored at −80° C.

### Brain Tissue

The post-mortem frozen cortical tissues from FXS male individuals (N=6) and age-matched typically developing (TD) males (N=5) were powdered in liquid nitrogen using a mortar and pestle. The fine powder was then homogenized on ice in a Dounce homogenizer using TRIzol™ Reagent (ThermoFisher Scientific #15596026), and the lysates were collected. Total RNA was extracted using BCP, recovered as described above, and stored at −80° C.

cDNA Synthesis and qPCR

One μg of total RNA was primed with oligo(dT)20 to generate cDNA with a QuantiTect cDNA synthesis kit (Qiagen, #205311) using random hexamers. qPCR was performed using the iTaq™ Universal SYBR® Green Supermix (BIO-RAD #1725122) on a QuantStudio 3 qPCR machine in duplicate.

### RNA-Seq Data Analysis

Fastq files were uploaded to the DolphinNext platform (Yukselen et al., BMC Genomics 21(1):310 (2020)) at the UMMS Bioinformatics Core for mapping and quantification. The reads were subjected to FastQC (v0.11.8) analysis, and the quality of reads was assessed. 9-nt molecular labels were trimmed from both 5′ ends of the pair-end reads and quality-filtered with Trimmomatic (0.32). Reads mapped to human rRNA by Bowtie2 (2.1.0) were filtered out. Cleaned reads were next mapped to the Refseq (V38) human transcriptome and quantified by RSEM (1.2.11). Gene Ontology (GO) enrichment analysis was done using the clusterProfiler package (Yu et al., 2012) for biological processes enriched in the differentially expressed genes. Estimated counts on each gene were used for the differential gene expression analysis by DESeq2 (1.16.1). After the normalization by median of ratios method, only the genes with minimal 5 counts average across all samples were kept for the Differential Gene expression analysis. The FDR (padj) cut-off <5% was used. The TDF files generated were uploaded on the Integrative Genomics Viewer for visualization.

The ratio between reads including or excluding exons, also known as “Percent Spliced In” (PSI), indicates how efficiently sequences of interest are spliced into transcripts. The False Discovery Rate (FDR) is a method of conceptualizing the rate of type I errors in null hypothesis testing when conducting multiple comparisons.

### Alternative Splicing Analysis

RNA-seq data generated from leukocytes from FXS male patients (N=10) and age-matched typically developing males (N=7) was used to analyze alternative splicing (AS) using the rMATS package v3.2.5 (Shen et al., Proc Natl Acad Sci USA. 111(51):E5593-601 (2014)) with default parameters. The Percent Spliced In (PSI) levels or the exon inclusion levels were calculated by rMATS using a hierarchical framework. To calculate the difference in PSI between genotypes, a likelihood-ratio test was used. AS events with an FDR<5% and |deltaPSI|≥5% as identified using rMATS were used for further analysis.

### Primers Sets for Detecting FMR1 Isoforms

Iso12_1Forward:
(SEQ ID NO: 1)
5′ AGAAGATGGAGGAGCTGGTG 3′
Iso12_1Reverse:
(SEQ ID NO: 2)
5′ CAGTGGAGCTCTCCGAAGTC 3′
Iso12_2Forward:
(SEQ ID NO: 3)
5′ CCAGCAGTGCATTGAAGAAG 3′
Iso12_2Reverse:
(SEQ ID NO: 4)
5′ CTGAAGCATGTGCATTCCTG 3′
Iso1_1 Forward:
(SEQ ID NO: 5)
5′ AGAAGATGGAGGAGCTGGTG 3′
Iso1_1 Reverse:
(SEQ ID NO: 6)
5′ TTCATGAACATCCTTTACAAAT
GC 3′

Differential gene expression analysis: DESeq2 (v3.9) was used to obtain differentially expressed genes from the estimated counts table. After normalization by the median of ratios method, genes with minimal 5 counts average across all samples were kept for the Differential Gene expression analysis. The Padj<5% was used as a cutoff. The TDF files generated were uploaded on the Integrative Genomics Viewer (2.6.2) for visualization.

Alternative splicing analysis: To analyze differential alternative splicing (AS), the rMATS package v3.2.5 (Shen et al., 2014) was used with default parameters. The Percent Spliced In (PSI) levels or the exon inclusion levels calculated by rMATS using a hierarchical framework. To calculate the difference in PSI between genotypes a likelihood-ratio test was used. AS events with an FDR<5% and |deltaPSI|≥5% as identified using rMATS were used for further analysis The genes with significant skipped exons were used for validation using RT-qPCR analysis. One ug of RNA was used to generate cDNA using the QuantiTect cDNA synthesis kit. Primers were designed to overlap skipped/inclusion exon junctions and qPCR was performed using the Bio-Rad SYBR reagent on a Quantstudio3 instrument.

Alternative polyadenylation (APA) analysis: Differential polyadenylation site usage was assessed using the APAlyzer (Wang and Tian, 2020). The RNA-seq read density between the last exon and the proximal (Sh-Short) polyadenylation site and for the distal (Ln-long) polyadenylation site was calculated, which determine the constitutive (cUTR) and the alternative (aUTR) 3′UTR, respectively. The difference in APA for a gene is calculated using a Relative-Expression(RE) score−log 2(RDaUTR/RDcUTR). The RE difference and the P value<0.05 was used to determine 3′UTR lengthening ‘UP’ and 3′UTR shortening; ‘DN’. ‘NC’ indicates no significant change. To assess intronic polyadenylation (IPA), the read density upstream and downstream of the intronic polyadenylation site was calculated and genes with activation or use of the IPA site are indicated by ‘UP’ and suppression of the IPA site use between the genotypes is indicated by ‘DN’. ‘NC’ indicates no significant change. Average reads count >5 in each replicate in each region (aUTR and cUTR) were used as a cutoff.

### Chromatin Immunoprecipitation Sequencing (ChIP-Seq)

Eight mL fresh blood was collected from FXS male (N=10) and age-matched typically developing males (N=7) individuals (See Supplemental Table 1) in a BD vacutainer CPT (Cell Preparation Tube with sodium citrate-blue top tube, Becton Dickinson #REF362761). The tube was gently inverted 5 times and the sample was centrifuged for 25 minutes at 1500-1800 RCF at room temperature. The tubes were then inverted to collect the lymphocytes and other mononuclear cells resuspended in the upper liquid phase in a new 15 mL tube. The samples were centrifuged again for 10 minutes at 300 RCF to obtain the PBMC (peripheral blood mononuclear cells) pellet. The PBMCs were rinsed with 1× Dublecco's phosphate buffered saline w/o calcium or magnesium (D-PBS) (Invitrogen #14190-094). The PBMC pellet was resuspended in 250 uL ice-cold D-PBS with protease inhibitors. Chromatin isolation and sequencing was performed as previously described (Shah et al., 2020). Briefly the cells were cross-linked with 1% formaldehyde and quenched with 150 mM glycine. After centrifugation at 2000 g for 10 min at 4° C. the cells were lysed. After homogenization the nuclei were harvested by centrifugation at 2000 g for 5 min at 40 C The nuclei were lysed by incubating for 20 mins on ice in nuclear lysis buffer (10 mM Tris (pH 8.0), 1 mM EDTA, 0.5 mM EGTA). 0.5% SDS was added and the samples sonicated on a Bioruptor® sonicator at high power settings for 9 cycles (sonication: 30 sec on, 90 sec off) of 15 min each at 4° C. The samples were centrifuged and diluted to adjust the SDS concentration to <0.1%. 10% of each sample was used as input. The remainder of the samples were divided into two and incubated with protein G dynabeads coupled overnight at 40 C with antibodies against H3K36me3 (Abcam ab9050, 5 μg per ChIP) or H3K4me3 (Active Motif-39159, 5 g per ChIP). After IP, the beads were washed and chromatin and de-crosslinked overnight at 65° C. After RNAse and proteinase K treatment the DNA was purified. ChIP-Seq libraries were prepared by performing the following steps: ends repair using T4 DNA polymerase, A′ base addition by Klenow polymerase and Illumina adapter ligation using T4 Polynucleotide kinase from New England Biolabs (NEB). The library was PCR amplified using multiplexing barcoded primers. The libraries were pooled with equal molar ratios, denatured, diluted, and sequenced with NextSeq 500/550 High Output Kit v2.5 (Illumina, 75 bp paired-end runs) on a Nextseq500 sequencer (Illumina).

### ChIP-Seq Analysis

For ChIP-seq data analysis, alignments were performed with Bowtie2 (2.1.0) using the GRCh38 (hg38) version 34 genome, duplicates were removed with Picard and TDF files for IGV viewing were generated using a ChIP-seq pipeline from DolphinNext (Yukselen et al., 2019). The broad peaks for H3K36me3 ChIP-Seq were called using the broad peak parameter MACS2. Narrow peaks for H3K4me3 ChIP were called using the narrow parameter in MACS2. deepTools (Ramirez et al., 2016) was used to plot heatmaps and profiles for genic distribution of H3K36me3 and H3K4me3 ChIP signals over input. IGV tools (2.6.2) were used for visualizing TDF files and all tracks shown were normalized for total read coverage.

### Cell Culture

### Lymphoblast Cell Lines

Lymphoblast cell lines (LCL) were obtained from Coriell Institute from two FXS individuals (GM07365 (FXS1), GM06897 (FXS2)) and two typically developing control males (GM07174 (WT3), GM06890 (WT4)). Cells were cultured in RPMI 1640 medium (Sigma-Aldrich), supplemented with 15% fetal bovine serum (FBS) and 2.5% L-glutamine at 37° C. with 5% CO₂ in T25 flasks.

### Fibroblast Cells

Skin biopsies from participants were collected in a 15 cc tube with transfer culture media (DMEM with 5% Gentamicin). The biopsy was then removed from the transfer media with tweezers onto a sterile tissue culture dish and dissected into approximately 6-7 pieces using sterile tweezers and scissors in the culture hood. Three to four pieces of skin explants were kept on the bottom of a T25 flask and 3 ml CHANG AMNIO culture media was added. The flask was then incubated at 37° C. with 5% CO₂ for 10 days. The culture media was changed after cells started growing out from the skin explants. After the cells had grown to 5-6 layers around the skin explants, the skin explants were removed from the culture flask and fibroblasts were trypsinized and spread evenly in the flask. The media were changed after overnight incubation with trypsin. Fibroblast culture medium was added (complete media-(500 ml DMEM (15-017-CV) with 10% FBS and 1× antibiotic-antimitotic, 1×L-glutamine 5 ml)) twice a week to cells in a T25 culture flasks at 37° C. with 5% CO₂.

### 5-AzadC Treatment

For each cell culture, 30×105 cells/ml were added to a final volume of 20 ml media (RPMI1640 medium (Sigma-Aldrich) supplemented with 15% fetal bovine serum (FBS) and 2.5% L-glutamine at 37° C. with 5% C02) per T25 flask. 5-Aza-2′-deoxycytidine (5-AzadC) (Sigma-Aldrich, A3656) was added to the cell cultures (final concentration 1 μM) for 7 consecutive days. A 2 mM stock of 5-AzadC was made in DMSO. For each cell line, two independent treatments were performed (n=2). For the no treatment controls for each cell line, DMSO was added to the flasks. For samples with both 5-AzadC and ASO treatment, 80 nM or 160 nM ASOs or vehicle were added on Day 1 and either 5-AzadC or DMSO was added each day from Day 2 up to Day 9 at a final concentration of 1 μM. On Day 9 the cells were collected in 1× phosphate buffered saline to proceed with RNA extraction or Western blotting.

### Western Blotting

Cells were homogenized at 4° C. in RIPA buffer, with incubation on ice for 10 minutes and dissociation by pipetting. The extract was centrifuged at 13,200 rpm for 10 minutes at 4° C., and the supernatant collected. Protein concentration was determined using BCA reagent. Proteins (10 g) were diluted in SDS-bromophenol blue reducing buffer with 40 mM DTT and analyzed using western blotting with the following antibodies: FMRP (Millipore, mAb2160, 1:1,000), FMRP (Abcam, ab17722, 1:1,000) and GAPDH (14C10, Cell Signaling Technology, mAb 2118, 1:2,000), diluted in 1× TBST with 5% non-fat milk. Membranes were washed three times for 10 minutes with 1×TBST and incubated with anti-rabbit or anti-mouse secondary antibodies (Jackson, 1:10,000) at room temperature for 1 hour. Membranes were washed three times for 10 minutes with 1×TBST, developed with ECL-Plus (Piece), and scanned with GE Amersham Imager.

### Quantification and Statistical Analysis

All grouped data are presented as mean±s.e.m. All tests used to compare the samples are mentioned in the respective figure. legends and corresponding text. When exact P values are not indicated, they are represented as follows: *, p<0.05; **, p<0.01; ***, p<0.001; ****, P value<0.0001; n.s., p>0.05.

### Example 2. Gene Expression Changes in Leukocytes from FXS Individuals

To investigate whether mis-splicing of mRNAs also occurs in blood samples from individuals with Fragile X Syndrome (FXS), deep (60-90 million reads) and long read (150PE) RNA-seq on freshly obtained leukocytes from 29 FXS males and 13 age-matched typically developing (TD) males was performed. CGG repeat expansion (>200) for all samples and FMR1 promoter methylation status for FXS samples when available was confirmed by either southern blot or methylation PCR assays.

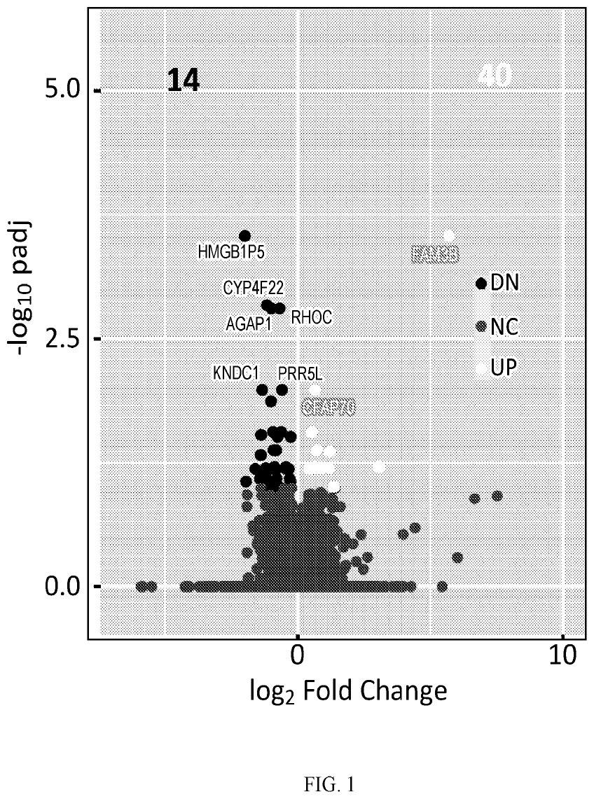

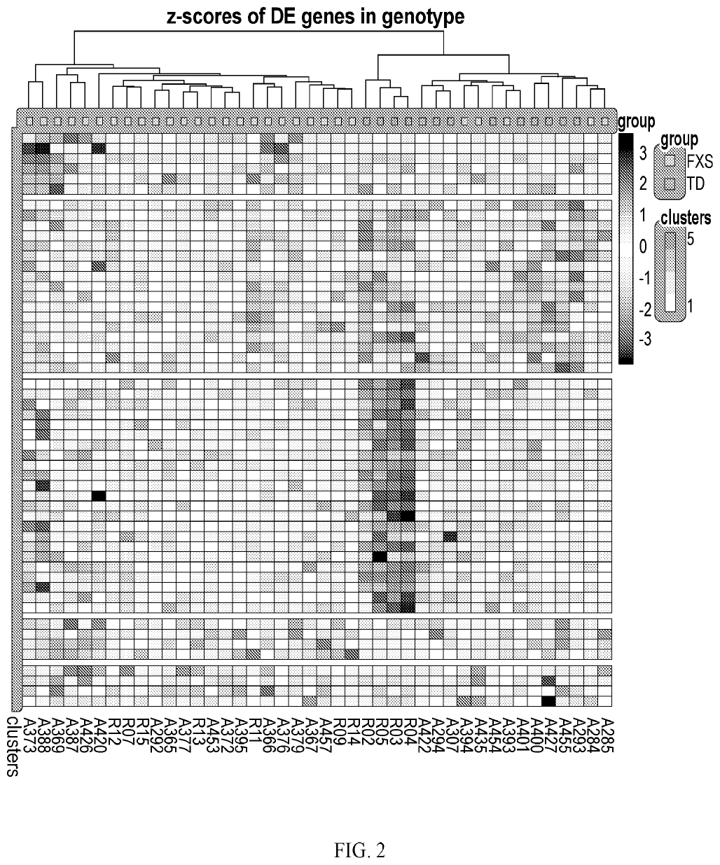

Differential gene expression (DGE) and differential alternative splicing (DAS) were conducted. DGE analysis revealed that ˜50 RNAs were up- or down-regulated in FXS leukocytes relative to TD (P value<0.0002) (FIG. 1) and were clustered based on their z-scores (FIG. 2). FXS individuals are indicated with black squares, while TD individuals are indicated with white squares.

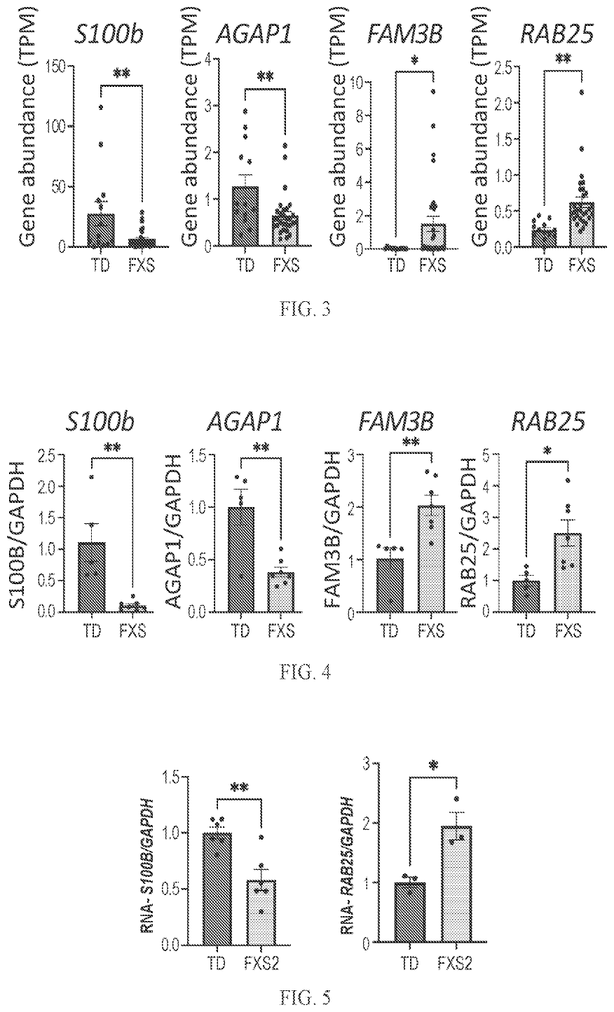

S100B (S100 calcium-binding protein B), AGAP1 (ArfGAP With GTPase Domain, Ankyrin Repeat And PH Domain 1), FAM3B (FAM3 Metabolism Regulating Signaling Molecule B), and RAB25 (RAS oncogene family member 25) are examples of RNAs that were depleted or up-regulated in the FXS samples relative to TD (log 2FC, P value<0.0002) (FIG. 3). The differential expression of these RNAs in FXS leukocytes was confirmed by RT-qPCR (FIG. 4). The decreased levels of S100B and increase in RAB25 levels in FXS cells were also confirmed in a lymphoblastoid cell line from a FXS individual (FXS2, GM06897, Coriell Institute) using qPCR assays (FIG. 5).

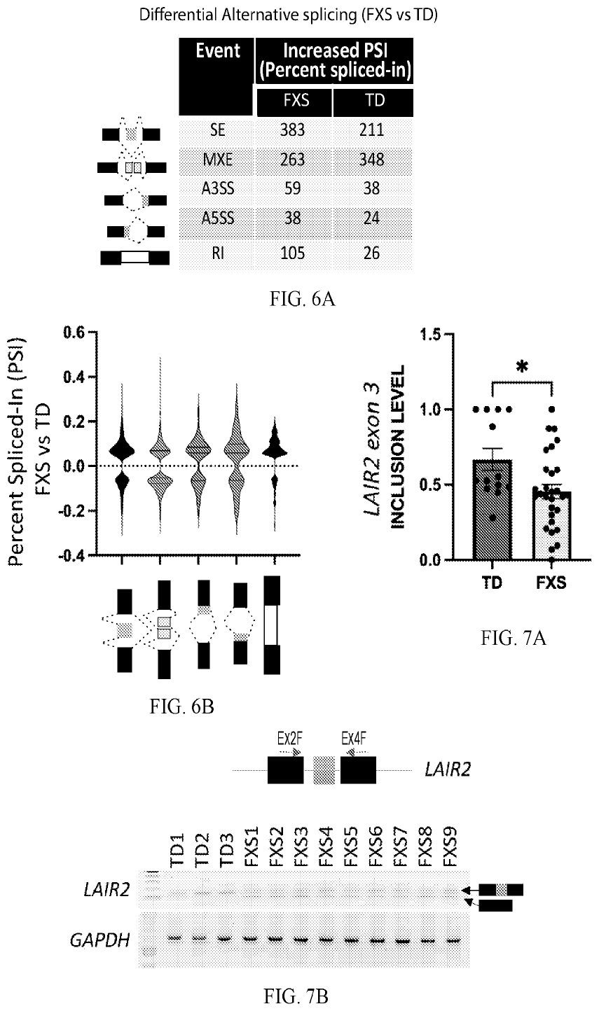

Finally, differential splicing using rMATS was assessed, finding hundreds of statistically significant events that were altered between genotypes (FXS vs. TD) (using an FDR<5% and a difference in the exon inclusion levels (PSI, Percent spliced-in) between the genotypes (deltaPSI) of ≥5% (FIG. 6A). A violin plot of the DAS demonstrates that most significant splicing changes were −/+˜10% in FXS vs TD with some changes near 30-40% (FIG. 6B). Using RT-PCR the increased skipping of exon 3 in the LAIR2 (leukocyte associated immunoglobulin like receptor 2) RNA was confirmed, which shows ˜20% increased skipping in FXS samples in the DAS analysis (FIGS. 7A and 7B).

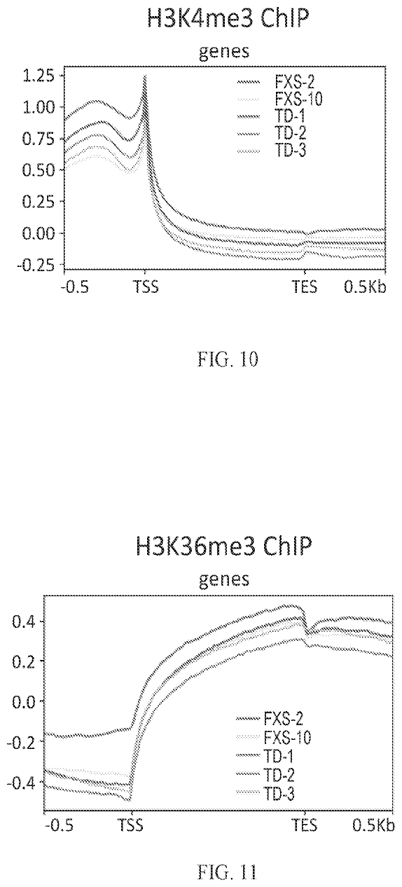

A previous study showed that Fmr1-dependent changes in the epigenetic mark H3K36me3 (histone H3 trimethylation at lysine 36) were correlated with aberrant alternative splicing in mouse hippocampus (Shah et al., Cell Rep. 30(13):4459-72 (2020)). Fmr1-dependent changes in RNA levels were also correlated with the chromatin mark H3K4me3 (histone H3 trimethylation at lysine 4) in cultured mouse neurons. Consequently, ChIP-Seq was performed to determine whether similar changes in chromatin marks occur in FXS cells. However, results from FXS (n=2) and TD (n=3) leukocyte samples showed no genotype-specific changes in H3K36me3 or H3K4me3 (FIG. 10 and FIG. 11).

### Example 3. FXS-Specific RNA Biomarkers

White blood cells were isolated from freshly drawn blood from 10 FXS individuals (males, ˜12-38 yrs) and 7 age-matched typically developing individuals (males, “TD” or “control”). RNA was extracted from the white blood cells, and deep, paired-end large-read length sequencing and analysis were performed. Greater than 1,000 misregulated RNA “events,” all having statistical significance (p<0.05), were detected in the FXS samples relative to the control samples.

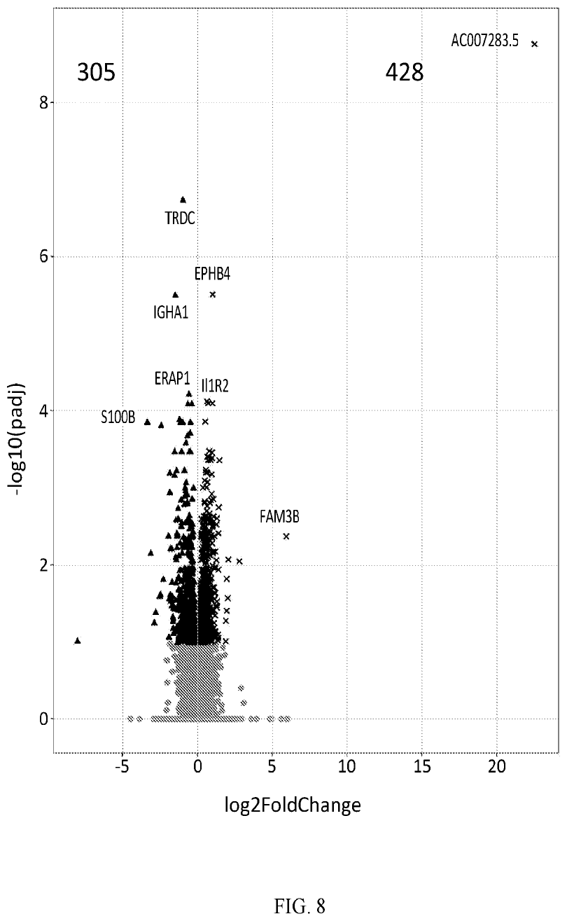

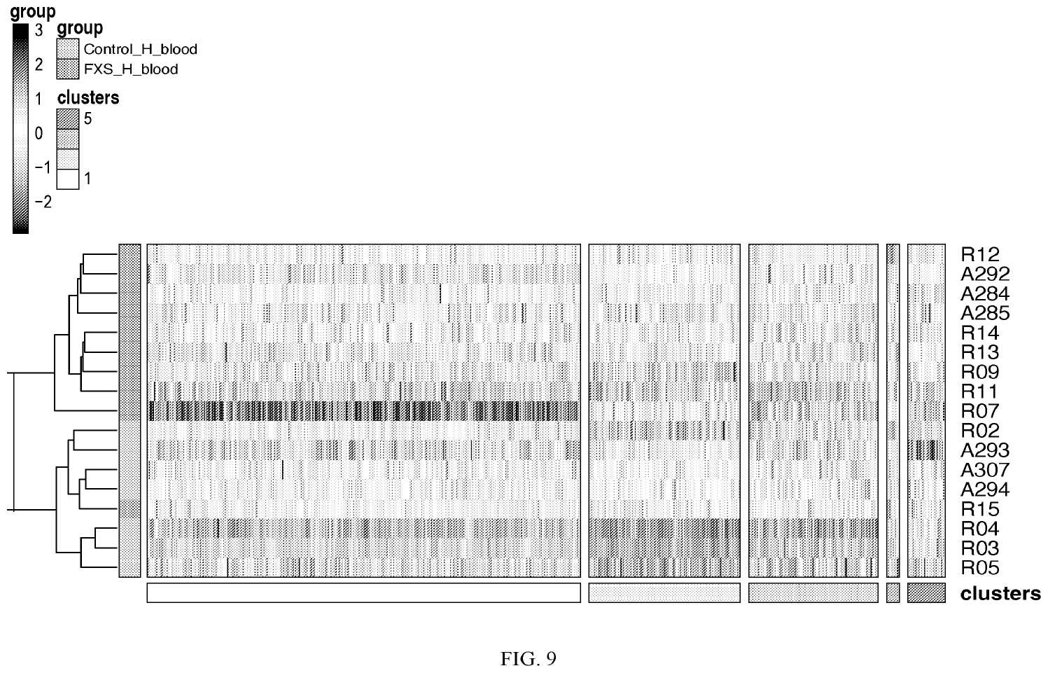

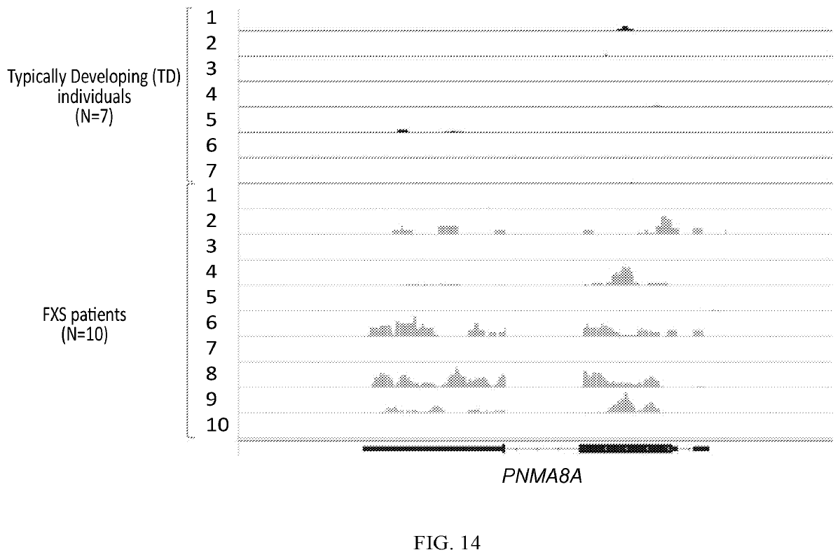

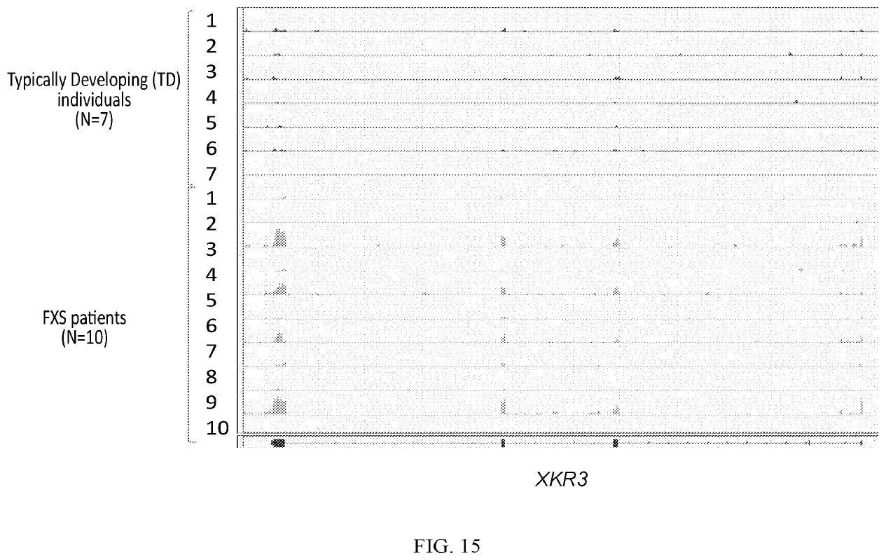

428 RNA markers were upregulated in the white blood cells of the FXS individuals compared to the typically developing individuals (FIG. 8, “x”). A genome browser view shows that the PNMA8A RNA is strongly expressed in FXS individuals but virtually absent in all typically developing individuals (FIG. 14). Non-limiting examples of RNA markers with increased expression in FXS individuals, relative to typically developing individuals, are listed in Table 1.

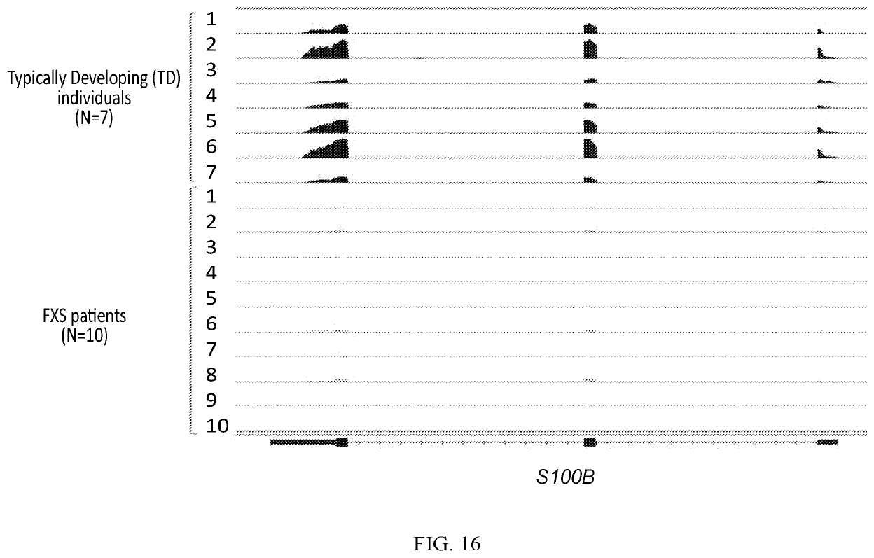

305 RNA markers were down regulated in the white blood cells of FXS individuals compared to typically developing individuals (FIG. 8, “Δ”). A genome browser view shows that expression of the S100B RNA is reduced in the FXS individuals, compared to typically developing individuals (FIG. 16). Non-limiting examples of RNA markers with reduced expression in FXS individuals, relative to typically developing individuals, are listed in Table 2.

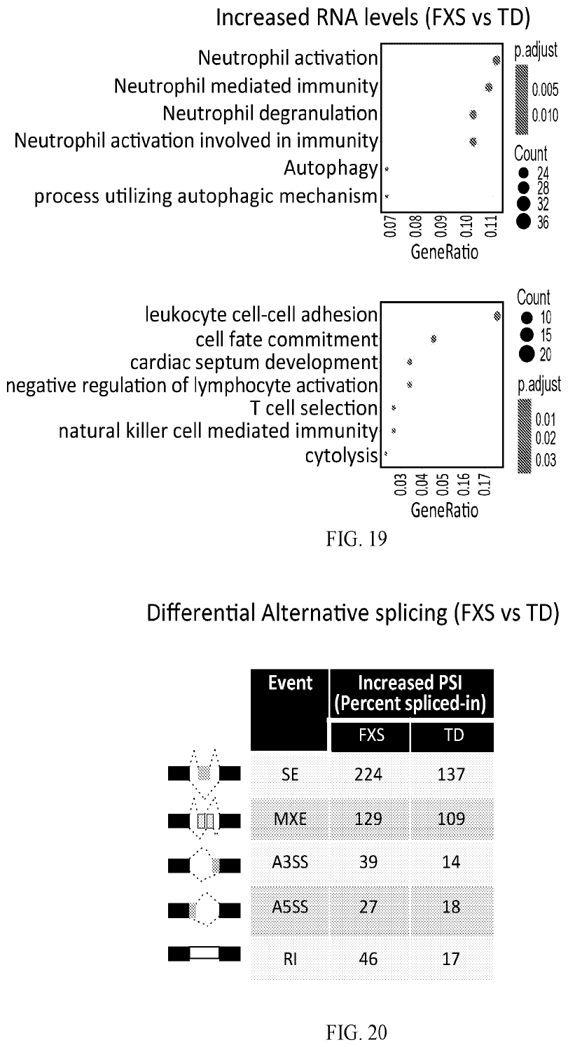

GO (Gene ontology) analysis indicates that RNAs enriched in the FXS samples encode proteins involved in biological processes such as neutrophil activation and immunity-related functions while the RNAs depleted in FXS encode proteins involved T cell and natural killer cell function (FIG. 19). We next assessed differential splicing using rMATS (Shen et al., 2014) and find hundreds of statistically significant events that are altered between genotypes (FXS vs. TD) (using an FDR<5% and a difference in the exon inclusion levels (PSI, Percent spliced-in) between the genotypes (deltaPSI) of ≥5% and read count >1 in each sample) (FIG. 20).

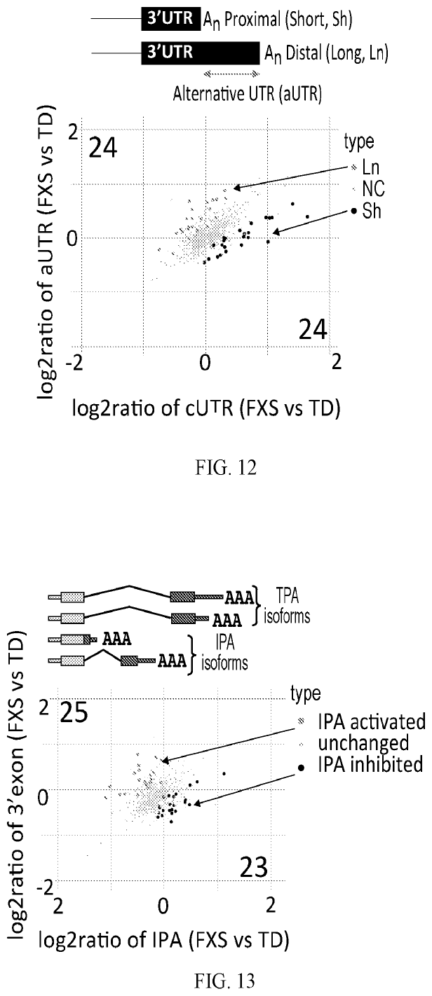

To determine whether alternative splicing or RNA level changes in FXS leukocytes correlate with FXS-dependent alterations in H3K36me3 or H3K4me3, ChIP-seq was performed for chromatin marks. FIGS. 10 and 11 demonstrate that the distribution and levels of these marks were similar in FXS (n=2) and TD (n=3) leukocytes. These results suggest that leukocytes from FXS individuals do not regulate histone modifications in a manner similar to Fmr1-deficient mouse brain or neurons. Finally, genotype-specific changes in the usage of distal or proximal polyadenylation sites were detected in RNA 3′UTRs (FIG. 12) as well as usage of intronic polyadenylation sites (FIG. 13), which add to the repertoire of altered RNA processing events that depend on FMR1.

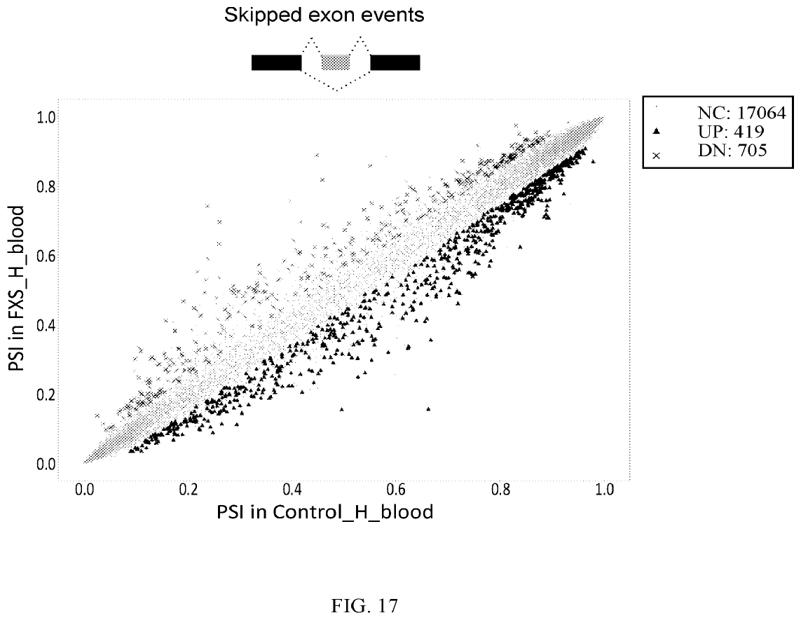

Most missplicing events were exon skipping or mutually exclusive exon switching. 705 RNA markers had increased exon skipping in the white blood cells of the FXS individuals, relative to the typically developing individuals (FIG. 17, “x”, less inclusion of skipped exon). Non-limiting examples of RNA markers with increased exon skipping in FXS individuals, relative to typically developing individuals, are listed in Table 4. All data are statistically significant (p<0.05 and FDR<0.05).

419 RNAs had reduced exon skipping in the white blood cells of the FXS individuals, relative to the typically developing individuals (FIG. 17, “▴”, more inclusion of skipped exon). Non-limiting examples of RNA markers with reduced exon skipping in FXS individuals, relative to typically developing individuals, are listed in Table 3. All data are statistically significant (p<0.05 and FDR<0.05).

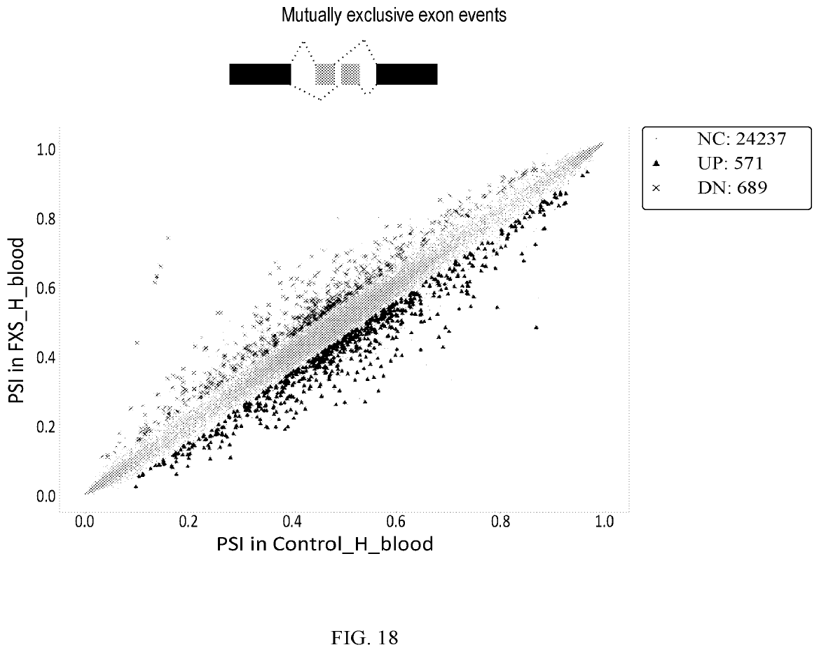

A similar pattern occurs with mutually exclusive exon switching. 571 RNAs had increased mutually exclusive exon switching in the white blood cells of the FXS individuals, relative to the typically developing individuals (FIG. 18, “▴”, more inclusion of mutually excluded exon). Non-limiting examples of RNA markers with increased mutually exclusive exon switching in FXS individuals, relative to typically developing individuals, are listed in Table 6. All data are statistically significant (p<0.05 and FDR<0.05).

689 RNAs had reduced mutually exclusive exon switching in the white blood cells of the FXS individuals, relative to the typically developing individuals (FIG. 18, “x”, less inclusion of mutually excluded exon). Non-limiting examples of RNA markers with reduced mutually exclusive exon switching in FXS individuals, relative to typically developing individuals, are listed in Table 5. All data are statistically significant (p<0.05 and FDR<0.05).

Some RNA markers showed altered 5′ or 3′ splice sites. The top 25 RNA markers with increased alternative 5′ splice site (ASSS) in FXS individuals, relative to typically developing individuals, are listed in Table 8. Non-limiting examples of RNA markers with reduced alternative 5′ splice site in FXS individuals, relative to typically developing individuals, are listed in Table 7. Non-limiting examples of RNA markers with increased alternative 3′ splice site (A3SS) in FXS individuals, relative to typically developing individuals, are listed in Table 10. Non-limiting examples of RNA markers with reduced alternative 3′ splice site in FXS individuals, relative to typically developing individuals, are listed in Table 9. All data are statistically significant (p<0.05 and FDR<0.05).

These findings suggest that RNA markers can be used for diagnosing an individual as having FXS, or having a propensity to develop FXS.

### Example 4. FMR1 Isoform 12 Detected in a Subpopulation of FXS Patients

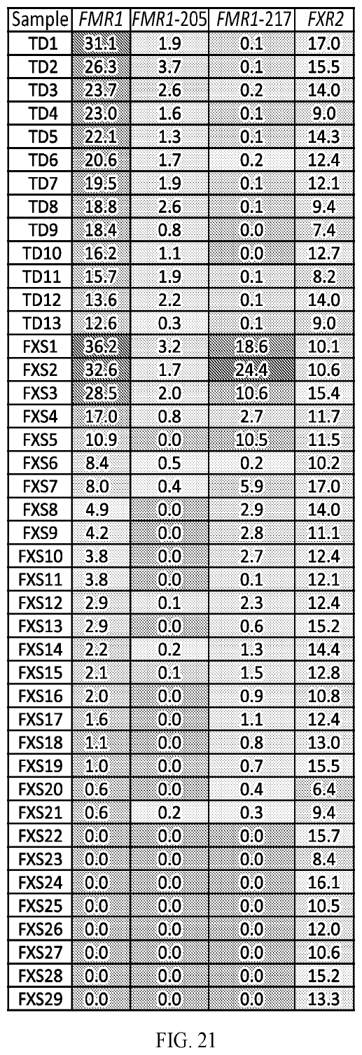

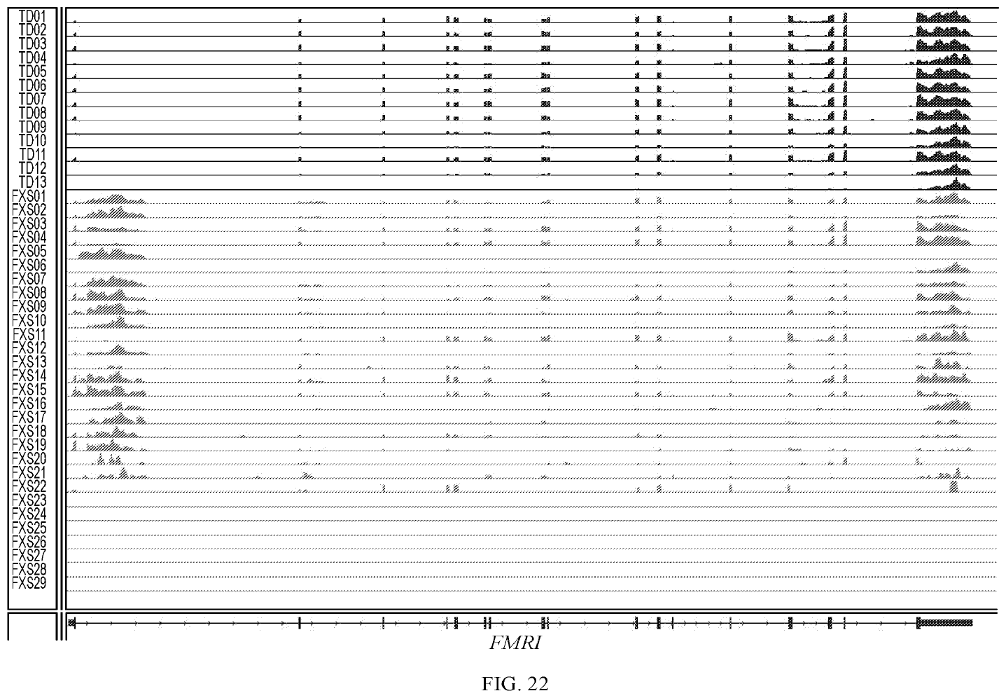

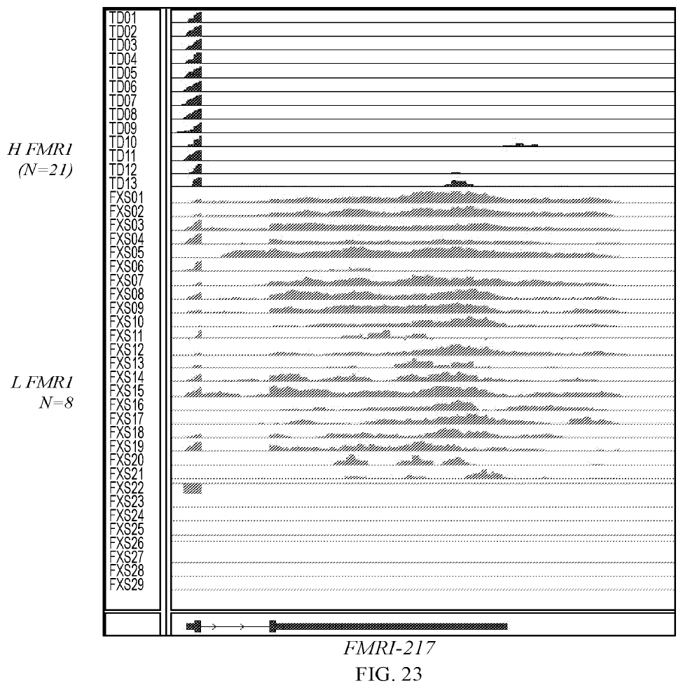

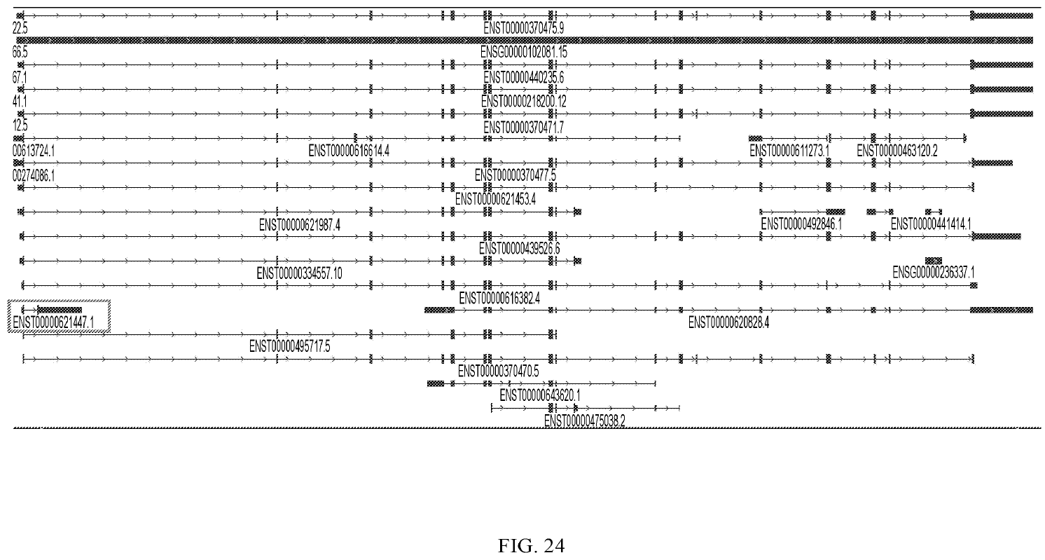

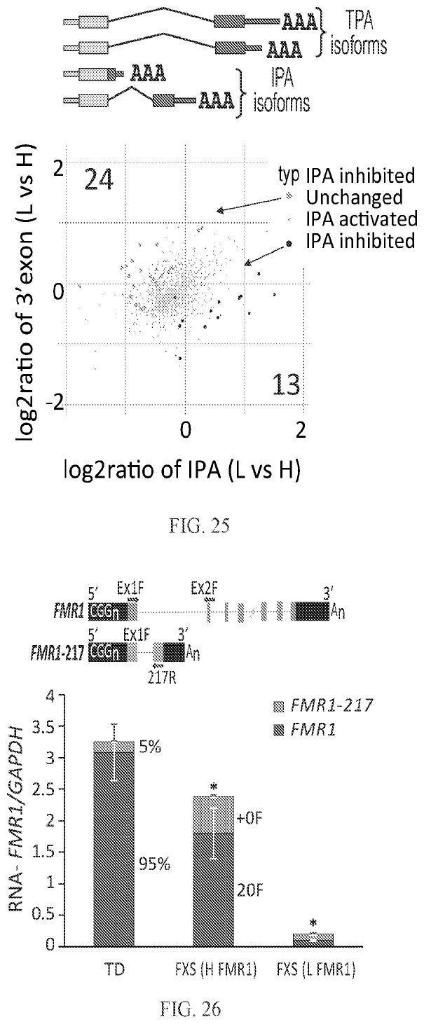

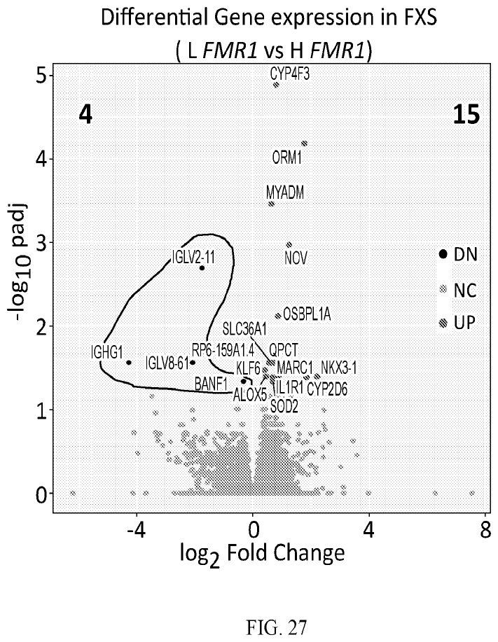

Expansion of >200 CGG repeats in FMR1 induces gene methylation, transcriptional silencing, loss of FMRP, and FXS. It was therefore surprising that in leukocytes of 21 of 29 FXS individuals, FMR1 RNA was detected, and in four individuals, the level of all isoforms of this RNA were similar to or even higher than those in the TD individuals (FIG. 21, FMR1 RNA TPM levels). When only full-length FMR1 encoding 632 amino acid FMRP (FMR1-205) was examined (FIG. 24), WBCs from 6 individuals had levels of this transcript that were similar to those of TD (FIG. 21). For comparison, the levels of the FMR1 paralog FXR2 were similar in all individuals (FIG. 21). Visualizing the RNA reads at the FMR1 locus with the Integrated Genome Viewer (IGV) make it evident that exonic reads were detected at robust levels in TD individuals (top 13 reads) and that the exonic reads were also detected in FXS individuals (bottom 29 reads) (FIGS. 22 and 23). FXS individuals 1-18 expressed relatively high FMR1 levels (with a cutoff of 0.6 TPM) (H FMR1) compared to FXS individuals 19-29 who expressed low or undetectable FMR1 levels (L FMR1) (FIGS. 22, 23, and 24). Remarkably, the H-FMR1 FXS individuals displayed strong RNA reads in intron 1 of FMR1 (black box FIG. 22, enlarged in FIG. 23). Notably, RNA reads in this intronic region were not detected in any TD individuals even though FMR1 RNA was strongly expressed (FIGS. 22 and 23).

The FMR1 locus expresses multiple alternatively spliced RNA isoforms (FIG. 24). The RNA reads detected in FMR1 intron 1 correspond to the second exon of the FMR1-217 RNA isoform (FIG. 24, grey box). FMR1-217 (ENST00000621447.1) is a 1.8 kb transcript comprised of two exons and is predicted to encode a 3 kDa truncated protein. RT-PCR was used to detect the FMR1-217 isoform in the FXS leukocyte samples (reverse transcription primed with oligodT(20)) and sequenced the amplified product using primers specific to the FMR1-217 exon-exon junction. Aligning this sequence to FMR1 showed that it is a spliced product of FMR1 exon one and FMR1-217 exon 2 (FIG. 24).

(SEQ ID NO: 10)
MEELVVEVRGSNGAFYKHWDFGELHCSGRGL

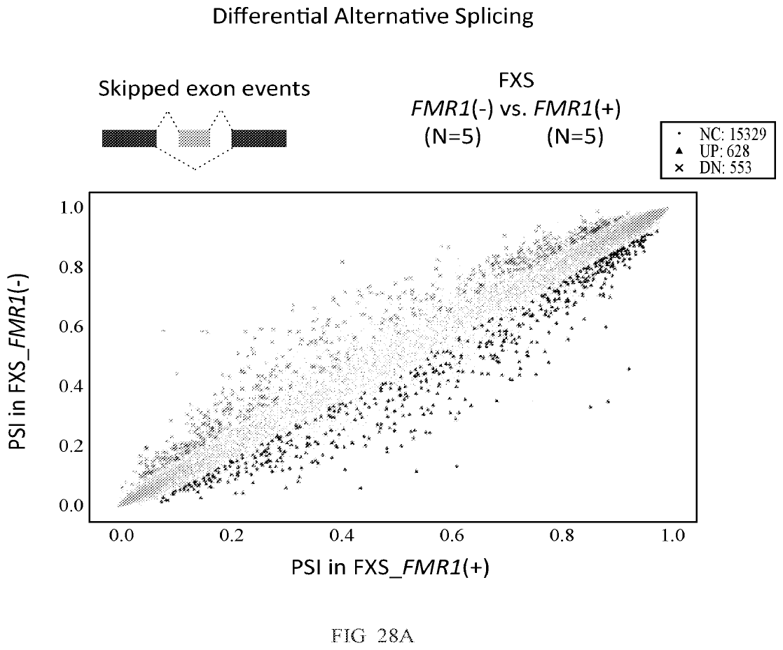

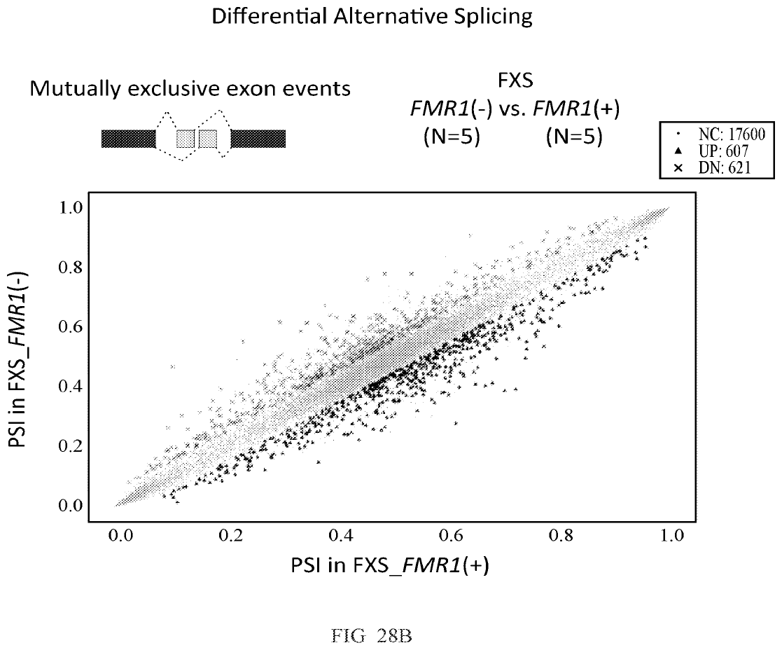

A scatter plot shows that overall splicing was differentially regulated in FXS individuals who expressed FMR1 RNA isoform 12, versus those who did not (FIGS. 28A-28B). 628 RNA markers had reduced exon skipping in the white blood cells of FXS individuals who expressed FMR1 RNA isoform 12 relative to those who did not (FIG. 28A, “▴”). 553 RNA markers had increased exon skipping in the white blood cells of FXS individuals who expressed FMR1 RNA isoform 12 relative to those who did not (FIG. 28A, ccx).

607 RNA markers had increased mutually exclusive exon switching in the white blood cells of FXS individuals who expressed FMR1 RNA isoform 12 relative to those who did not (FIG. 28B, “▴”). 621 RNA markers had reduced mutually exclusive exon switching in the white blood cells of FXS individuals who expressed FMR1 RNA isoform 12 relative to those who did not (FIG. 28B, “x”).

These findings suggest that RNA markers such as FMR1 RNA not only can be used for diagnosing an individual as having FXS, or having a propensity to develop FXS, but also can be used for stratifying FXS individuals. The identification FMR1 RNA isoform 12 enables stratification of FXS individuals into two subpopulations, those who express isoform 12 and those who do not. FXS individuals expressing some FMR1 RNA differ in their overall cellular splicing pattern compared to FXS individuals who do not express FMR1 RNA, thus, providing a robust basis for distinguishing the two subpopulations of FXS individuals via splicing data.

Next, the proportion of full-length FMR1 RNA to FMR1-217 RNA in TD or FXS leukocytes was assessed. In the TD samples, 95% of the total FMR1 RNA (primers Ex1F and Ex1R) represented full-length molecules (primers Ex1F and Ex2R), whereas in the H FMR1 samples, 75% of the total FMR1 RNA was full-length and 25% was FMR1-217 (primers Ex1F and 217R) (FIG. 26). In the L FMR1 samples, both isoforms were just barely detected. The total FMR1 RNA levels in all the samples were normalized to GAPDH RNA expression (* denotes P values<0.05). Importantly, all FXS individuals in this study, irrespective of FMR1 expression, displayed typical FXS symptoms, suggesting that even in patients with high FMR1 expression, functional FMRP may not be present or is present at very low amounts (FMRP protein levels were quantified for available samples (data not shown)).

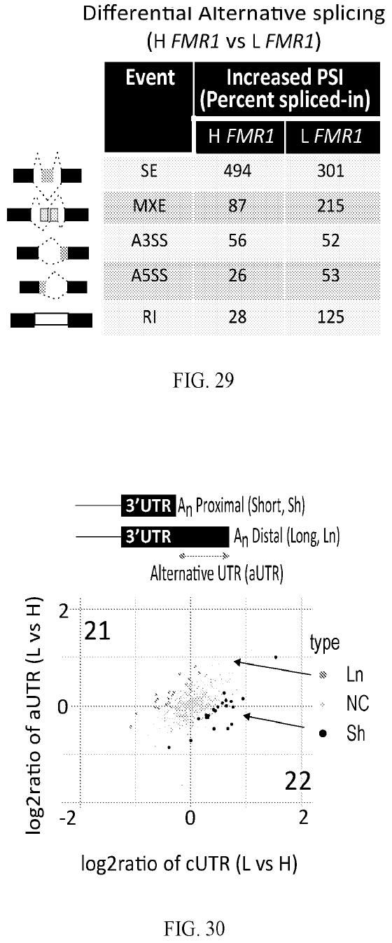

Whether stratification of FXS individuals, based on relatively high (H) or low (L) amounts of FMR1 (using a cutoff of 0.6 TPM), was reflected in transcriptome-wide RNA changes was examined. By reanalyzing FXS leukocyte RNA-seq data to compare significant RNA alterations between these two groups, hundreds of aberrant splicing events that tracked with the amount of this mis-spliced transcript were found (FIG. 29 and data not shown). Whether the parameters measured in WBCs correlated with IQ was investigated. Table 11 presents determinations of methylation status of the FMR1 gene (by PCR), FMRP levels (ng/μg protein), CGG repeat number, FMR1-217, full-length FMR1-205, all detected FMR1 isoforms, and IQ (Stanford-Binet test).

TABLE 11
Characterizing Leukocytes of Each FXS Individual

| | | | PBMC [ng | | | | |
|---|---|---|---|---|---|---|---|
| | CGG | | FMRP/μg | | | | |
| | repeat | Methylation | total | | FMR1 | FMR1-205 | FMR1-217 |
| Lab ID | number | (MPCR) | protein] | IQ | (TPM) | | |
| FXS01 | 140, | 140 100%, 175 | 6.56E−03 | 37.8 | 36.2 | 3.2 | 18.6 |
| | 175, >200 | 97%, >200 90% | | | | | |
| FXS02 | >200 | 81% | 2.07E−03 | 26.8 | 32.6 | 1.7 | 24.4 |
| FXS03 | 150, >200 | N/A | N/A | 52.0 | 28.5 | 2.0 | 10.6 |
| FXS04 | 102, >200 | N/A | N/A | 37.0 | 17.0 | 0.8 | 2.7 |
| FXS05 | >200 | >200 96% | 4.85E−04 | 35.1 | 10.9 | 0.0 | 10.5 |
| FXS06 | 65, >200 | 65 98%, >200 100% | 1.77E−02 | 55.0 | 8.4 | 0.5 | 0.2 |
| FXS07 | >200 | N/A | 2.28E−04 | 25.0 | 8.0 | 0.4 | 5.9 |
| FXS08 | 173, >200 | N/A | N/A | 39.9 | 4.9 | 0.0 | 2.9 |
| | (~710, ~613) | | | | | | |
| FXS09 | >200 | 100% | 4.40E−04 | 35.0 | 4.2 | 0.0 | 2.8 |
| FXS10 | >200 | 100% | 3.11E−04 | 56.0 | 3.8 | 0.0 | 2.7 |
| FXS11 | >200 | 100% | 6.50E−03 | 62.3 | 3.8 | 0.0 | 0.1 |
| FXS12 | >200 | 100% | 4.85E−04 | 26.9 | 2.9 | 0.1 | 2.3 |
| FXS13 | 102, | 102, 174 | 5.35E−04 | 27.6 | 2.9 | 0.0 | 0.6 |
| | 174, >200 | 100%, >200 100% | | | | | |
| FXS14 | >200 | N/A | N/A | 20.0 | 2.2 | 0.2 | 1.3 |
| FXS15 | >200 | 100% | 2.56E−04 | 45.9 | 2.1 | 0.1 | 1.5 |
| FXS16 | 63, >200 | 63.98%, 194 | 3.50E−03 | 53.5 | 2.0 | 0.0 | 0.9 |
| | | 36%, >200 100% | | | | | |
| FXS17 | >200 | 100% | 1.05E−04 | 44.0 | 1.6 | 0.0 | 1.1 |
| FXS18 | >200 | N/A | 1.34E−04 | 30.3 | 1.1 | 0.0 | 0.8 |
| FXS19 | >200 | N/A | N/A | 50.0 | 1.0 | 0.0 | 0.7 |
| FXS20 | >200 | 100% | 4.85E−04 | 29.6 | 0.6 | 0.0 | 0.4 |
| FXS21 | >200 | 100% | 2.00E−04 | 37.7 | 0.6 | 0.2 | 0.3 |
| FXS22 | >200 | N/A | 4.88E−04 | 35.8 | 0.0 | 0.0 | 0.0 |
| FXS23 | >200 | 94% | N/A | N/A | 0.0 | 0.0 | 0.0 |
| FXS24 | 28**, >200 | 100% | N/A | 37.6 | 0.0 | 0.0 | 0.0 |
| FXS25 | >200 | 100% | N/A | N/A | 0.0 | 0.0 | 0.0 |
| FXS26 | >200 | 100% | 4.85E−04 | 41.8 | 0.0 | 0.0 | 0.0 |
| FXS27 | >200 | >200 100% | 4.85E−04 | 20.2 | 0.0 | 0.0 | 0.0 |
| FXS28 | >200 | N/A | N/A | N/A | 0.0 | 0.0 | 0.0 |
| FXS29 | >200 | >200: 85% | N/A | 49 | 0 | 0 | 0 |

In Table 11, FMR1 gene methylation (MPCR): in percent as determined by PCR analysis; FMRP levels: ng/μg total protein; FMR1: all isoforms; IQ: Stanford-Binet; N/A: not available.

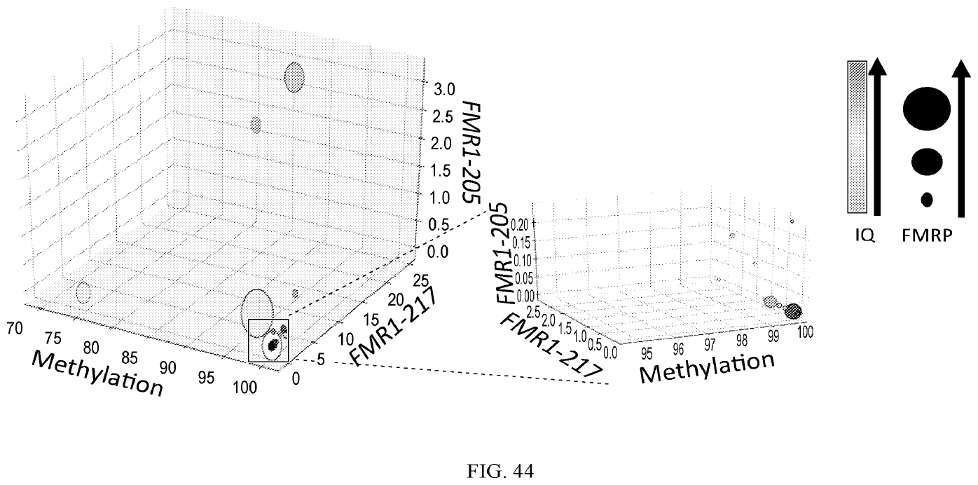

Table 12 presents correlation coefficients for pairwise comparisons of the measurements noted above. Methylation of the FMR1 gene is negatively correlated with FMR1-217 and FMR1-205 expression. More intriguing is the moderately positive correlation of IQ with FMRP protein levels. Somewhat surprisingly, FMR1-205, which encodes full-length FMRP, has no correlation with IQ. However, it is noted that while FMR1-205 encodes the complete 632-amino acid FMRP, other FMR1 isoforms, which vary in abundance, encode truncated FMRP proteins. Without presupposing functionality of truncated FMRP proteins, the canonical FMR1 isoform, FMR1-205, was used for further comparisons. FMR1-217 has a negative correlation with IQ, indicating a deleterious effect of this isoform. FIG. 44 displays a 3-dimensional comparison of all the parameters noted above. The inset shows that some FXS patients with a fully methylated FMR1 gene express FMR1 RNA and FMRP. Taken together, these results show several important findings. First, the FMR1 locus is frequently transcribed even when the FMR1 gene with a full CGG expansion is fully methylated. Second, FMRP levels in WBCs are positively correlated with IQ. Third, the negative correlation of FMR1-217 with IQ suggests that the process of mis-splicing, the 31-amino acid polypeptide derived from FMR1-217, and/or the FMR1-217 RNA itself (e.g., all three) might impart some toxic effect manifest in the brain (e.g., IQ). In any event, the levels of FMR1-217 expression, as well as additional transcriptome-wide changes in RNA processing events, may form the basis for molecular stratification of FXS individuals.

TABLE 12
Correlation coefficients for pairwise
comparisons for indicated parameters

| | | | | | FMR1- | FMR1- |
|---|---|---|---|---|---|---|
| | Methylation | FMRP | IQ | FMR1 | 205 | 217 |
| Methylation | 1.0 | −0.2 | 0.3 | −0.9 | −0.8 | −1.0 |
| FMRP | −0.2 | 1.0 | 0.5 | 0.3 | 0.3 | 0.0 |
| IQ | 0.3 | 0.5 | 1.0 | −0.2 | −0.1 | −0.3 |
| FMR1 | −0.9 | 0.3 | −0.2 | 1.0 | 0.9 | 1.0 |
| FMR1-205 | −0.8 | 0.3 | −0.1 | 0.9 | 1.0 | 0.8 |
| FMR1-217 | −1.0 | 0.0 | −0.3 | 1.0 | 0.8 | 1.0 |

In Table 12, +/− 0-0.1: no correlation; +/− 0.1-0.29: weak correlation; +/− 0.3-0.49: moderate correlation; +/− 0.5-1: strong correlation.

### Example 5. FMR1-217 is Expressed in Human FXS and Pre-Mutation Carrier Postmortem Brain

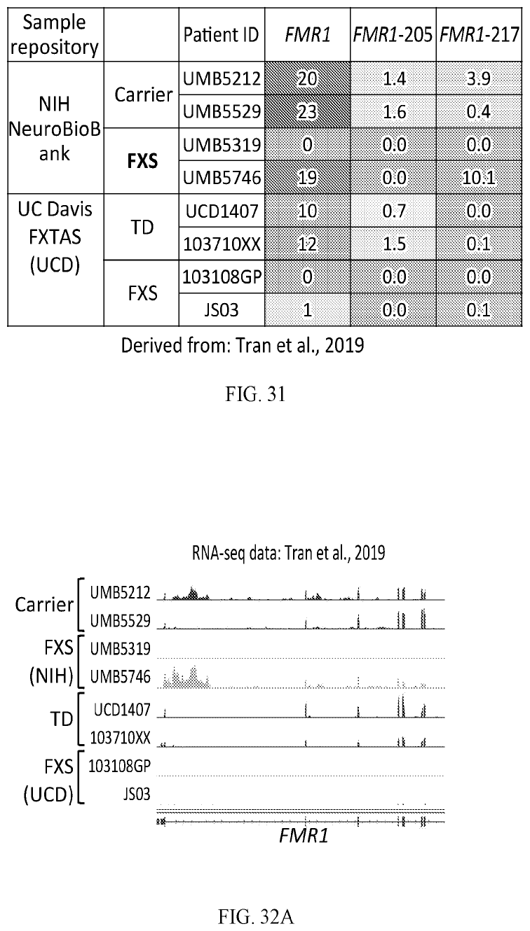

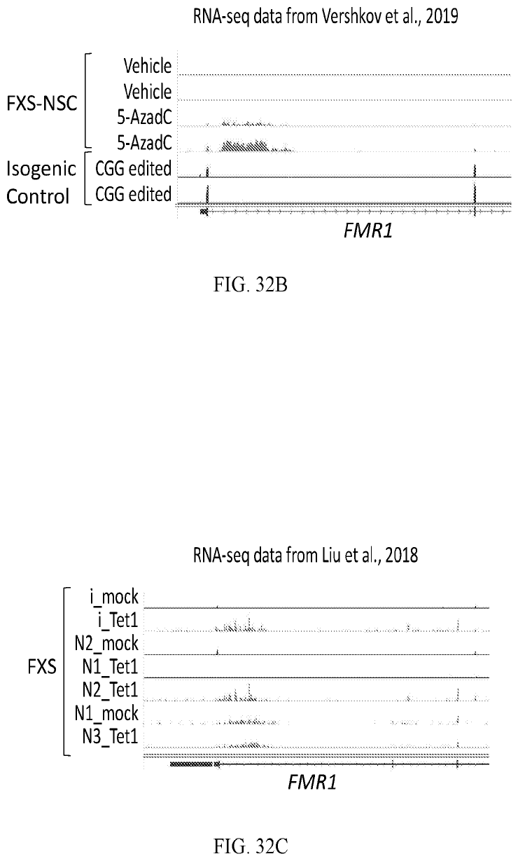

To investigate whether FMR1-217 is expressed in FXS brain, publicly available RNA-seq data of post-mortem frontal cortex tissues from FXS individuals (CGG repeats >200), FXS carriers (CGG repeats 55-200) and TD individuals (CGG repeats <55) (Tran et al., 2019) were analyzed. FMR1 RNA (TPM) levels are highest in pre-mutation carriers (FIG. 31). Interestingly, the FXS sample UMB5746 also displays high levels of FMR1 RNA (FIGS. 31 and 32A). This analysis shows that this individual expressed FMR1-217 as did FXS carrier UMB5212, who had Fragile X-associated tremor/ataxia syndrome (FXTAS) (FIGS. 31 and 32A). Neither TD individual had any RNA reads corresponding to FMR1-217 (FIGS. 31 and 32A). Thus, FMR1-217 RNA may be expressed in the brains of a subset of FXS individuals and premutation carriers.

A BLAST analysis showed that FMR1-217 aligned only with intron 1 of FMR1 and with no other region of the genome. Additional data showed unequivocally that FMR1-217 is derived from FMR1, and that its synthesis is dependent the CGG expansion in this gene. Vershkov et al. used CRISPR/Cas9 to delete the CGG expansion from FMR1 in FXS iPSC-derived neural stem cells (NSCs). Additional FXS NSCs were incubated with 5-AzadC, a nucleoside analogue that prevents DNA methylation. RNA sequencing from these samples, as well as from FXS NSCs incubated with vehicle, was then performed. The RNA-seq data from Vershkov et al. was reanalyzed, some of which is presented in FIG. 32B, and FMR1 transcript quantification (TPM) in Table 13. RNA-seq reads corresponding to FMR1-217 were clearly evident in the FXS-NSCs incubated with 5-AzadC, but not in the other samples. Moreover, the CGG edited cells, which were isogenic to the unedited FXS NSCs, had no FMR1-217 reads, but instead robust expression of full-length FMR1. Quantification of the RNA-seq reads (TPM) showed strong total FMR1 and FMR1-205 expression in the CGG-edited and 5-AzadC-treated cells but not in vehicle-treated cells. More importantly, strong FMR1-217 expression was observed only in the 5-AzadC-treated cells. Therefore, FMR1-217 is derived from the FMR1 locus and requires a CGG expansion.

TABLE 13
FMR1 (Total, −205 or −217) reads
(TPM) of the samples in FIG. 32B

| | FMR1 | FMR1-205 | FMR1-217 |
|---|---|---|---|
| Vehicle | 0.0 | 0.0 | 0.0 |
| Vehicle | 0.2 | 0.0 | 0.0 |
| 5-AzadC | 9.9 | 0.8 | 6.9 |
| 5-AzadC | 6.1 | 1.9 | 3.9 |
| CGG edited | 27.1 | 3.1 | 0.1 |
| CGG edited | 33.0 | 7.6 | 0.3 |

In a complementary study, Liu et al. performed a targeted FMR1 gene demethylation experiment by incubating FXS iPSC and FXS iPSC-derived neurons with a FMR1 small guide RNA and a catalytically inactive Cas9 fused to Tet1 demethylase sequences. Reanalysis of the subsequent RNA-seq data is shown in FIG. 32C, and FMR1 transcript quantification (TPM) in Table 8. Their experimental paradigm showed that FMR1-217 sequences were evident only when the gene was demethylated in the FXS cells. Quantification of the relevant transcripts in Table 14 showed that strong FMR1 and FMR1-205 expression was detected in the Tet1-treated samples (but inexplicably, no FMR1-205 in sample N1_Tet1), and FMR1-217 expression in all Tet1-treated samples. These data therefore show once again that FMR1-217 is derived from the FMR1 locus and requires a CGG expansion.

TABLE 14
FMR1 (Total, 205 or 217) reads (TPM)
of the samples in FIG. 32C.

| | FMR1 | FMR1-205 | FMR1-217 |
|---|---|---|---|
| i_mock | 0.1 | 0.0 | 0.0 |
| i_Tet1 | 69.9 | 0.0 | 7.3 |
| N1_mock | 0.1 | 0.0 | 0.0 |
| N2_mock | 0.1 | 0.0 | 0.0 |
| N1_Tet1 | 46.4 | 0.0 | 6.9 |
| N2_Tet1 | 81.3 | 22.7 | 13.3 |
| N3_Tet1 | 50.4 | 12.3 | 10.6 |

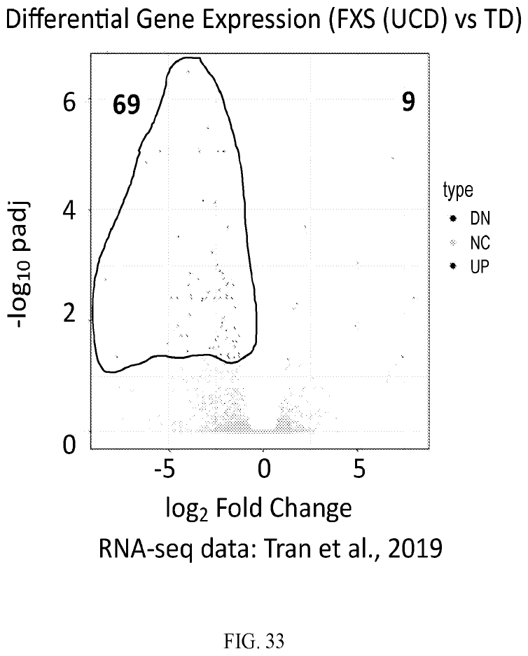

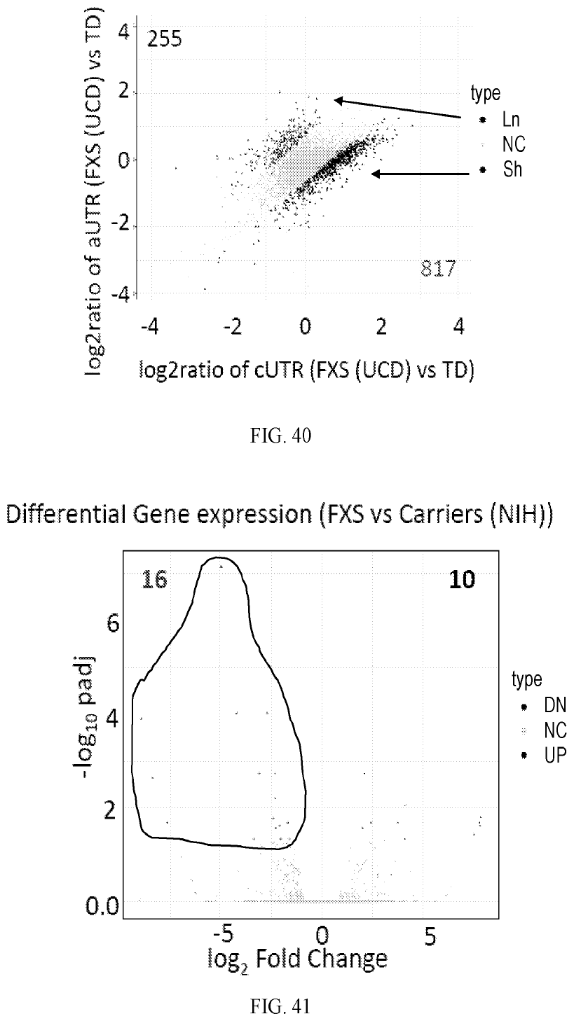

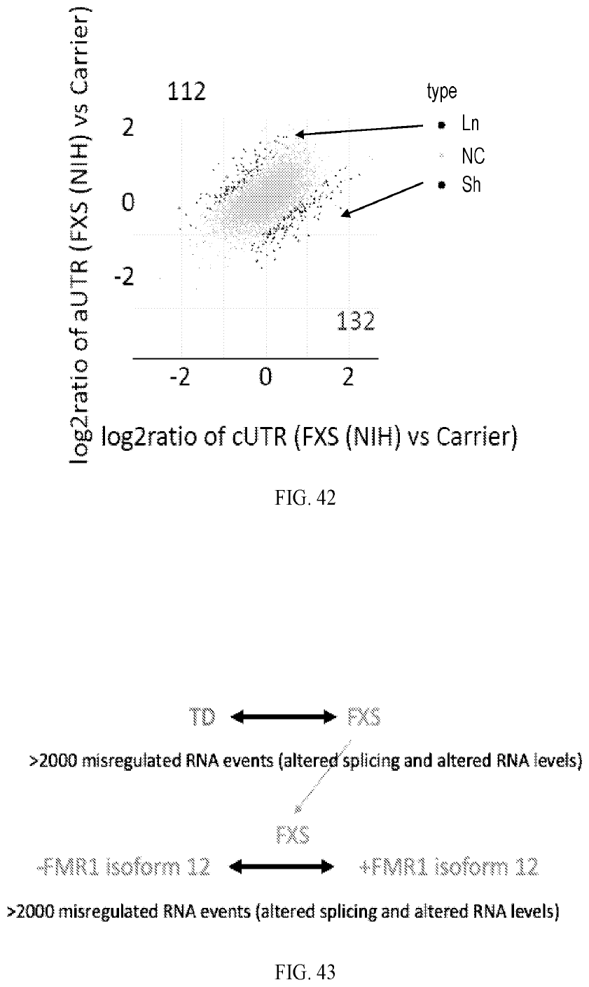

To determine whether transcriptome-wide changes in RNA expression could be detected in the frontal cortex RNA-seq data (Tran et al., 2019) from the FXS vs. TD or FXS vs. FXS carriers, DGE, DAS, and APA analysis was performed. Although the sample size is small, comparing FXS samples 103108GP and JS03 to TD samples UCD1407 and 103710XX (FIG. 31), changes in the levels of 78 RNAs (FIG. 33; 69 down-regulated and 9 up-regulated), 351 differential alternative splicing events (FIG. 34), and 1072 changes in 3′UTR length (FIG. 40; 255 long 3′UTR-distal poly(A) and 817 short 3′UTR-proximal poly(A)) were found. A comparison of FXS samples UMB5319 and UMB5746 to FXS carrier samples UMB5212 and UMB5529 (FIG. 31) shows that the levels of 26 RNAs (FIG. 41; 16 down-regulated and 10 up-regulated), 204 differential alternative splicing events (FIG. 34), and 244 changes in 3′UTR length (FIG. 42; 112 long 3′UTR-distal poly(A) and 132 3′UTR-proximal poly(A)) were altered in a statistically significant manner. Thus, in the available dataset, hundreds of transcriptomic changes are evident in brain tissue from FXS individuals and FXS carriers.

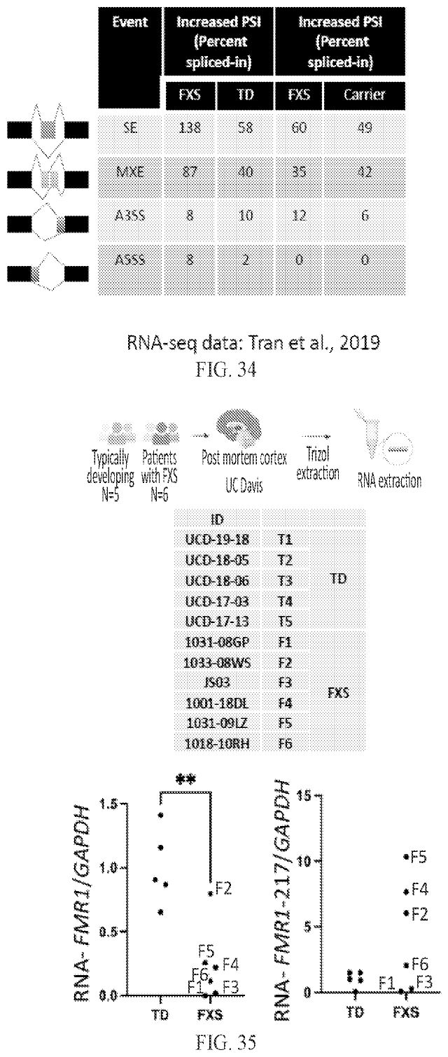

To confirm expression of FMR1-217 RNA in FXS brain tissue, frozen post-mortem cortex samples from six FXS males and five age-matched typically developing (TD) males (UC Davis Health) were obtained. Using RT-qPCR it was found that the FMR1 full length RNA was significantly reduced in the FXS individuals compared to that in the TD individuals. However, 3 or 4 of the 6 FXS individuals expressed varying levels of the FMR1 full-length RNA and also the FMR1-217 RNA (1031-09LZ, 1001-18DL and 1033-08WS) (FIG. 35). Expression of FMR1 RNA has been described previously for the sample 1031-09LZ (Esanov et al., 2016). The two FXS tissue samples (1031-08GP and JS03) studied in Tran et al, did not show FMR1 RNA expression as seen previously (FIGS. 31 and 35).

FMR1-217 RNA was detected in only one of the two premutation carrier samples. To gain greater insight into the relationship of FMR1-217 FXS carrier tissue (CGG repeats between 55-200), skin biopsies from 3 additional premutation carriers and 3 TD individuals (FIG. 36) were obtained. The skin samples were cultured in vitro to generate fibroblast cell lines for RNA analysis. Interestingly, using RT-qPCR cycle threshold (ct) traces from technical replicates, we detected FMR1-217 in one premutation carrier (C172) with 140 CGG repeats but not in samples with 77 or 98 CGG repeats (FIG. 36). There was no change in total FMR1 RNA levels among the samples (FIG. 36). Thus, generation of FMR1-217 may be linked to the number of CGG repeats in the FMR1 gene.

### Example 6. FMR1-217 RNA is Expressed in Lymphoblast Cell Cultures from FXS Individuals

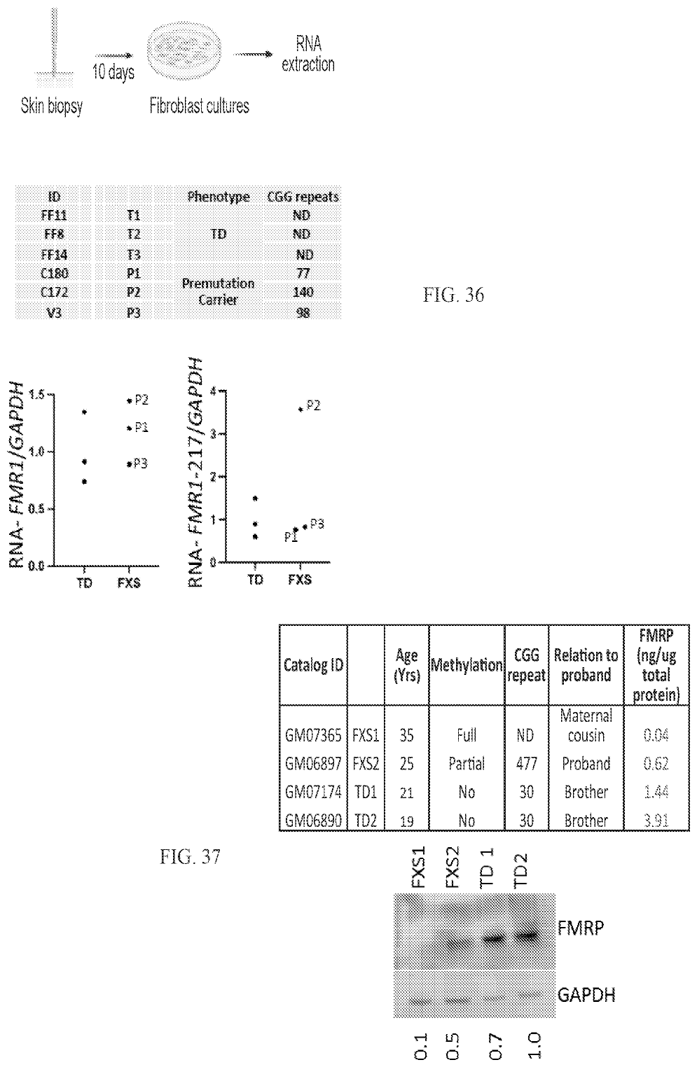

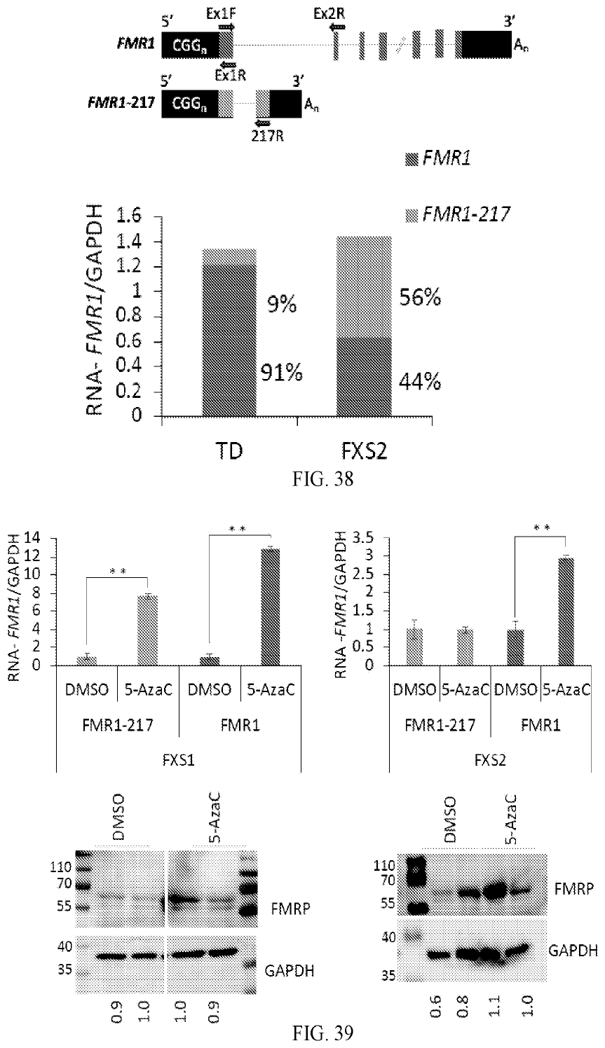

DNA methylation of the CpG island upstream of the FMR1 gene promoter in FXS individuals (MFM, Methylated full mutation) contributes to transcriptional silencing of the locus and loss of FMRP. FMR1 transcription can be reactivated by treatment with the nucleoside analogue 5-AzadC (5-aza-2′-deoxycytidine), which inhibits DNA methylation (Tabolacci et al., 2016b, 2016a). Consequently, whether re-activating FMR1 transcription in cells from FXS individuals with a presumably fully methylated and completely silenced FMR1 locus results in FMR1-217 expression was investigated. For these experiments, lymphoblast cell lines (LCLs) derived from a FXS individual with a fully methylated locus (MFM) that is transcriptionally inactive (FXS1, GM07365), a FXS individual with a presumably partially methylated locus (UFM) that expresses some FMR1 RNA (FXS2, GM06897), and two typically developing individuals (TD1, GM07174, and TD2, GM06890) (all samples from Corriell Institute, NJ, USA) (FIG. 37) were used. Western blot analysis shows that modest levels of FMRP are detected in FXS2, but not FXS1 cell lines. FMRP is strongly expressed in TD1 and TD2 cells (ratios of FMRP/GAPDH relative to TD2 are shown below the blot) (FIG. 37). Using RT-qPCR, it was found that FMR1-217 RNA is expressed in FXS2 LCLs and comprises 56% of the total FMR1 RNA compared to only 9% in TD cells (FIG. 38). It is noteworthy that although total FMR1 RNA levels in FXS2 cells are similar to those in TD cells, FMRP levels are much lower (FIGS. 37 and 38). Next, FXS1 and FXS2 cell lines were treated with the 5-AzadC and then measured FMR1 RNA and FMRP levels. In the FXS1 cell line, treatment with 5-AzadC for seven days resulted in significant increases of both full-length FMR1 and FMR1-217 RNAs relative to DMSO-treated cells (FIG. 39). However, in FXS2 cells, 5-AzadC treatment resulted in an increase of only full-length FMR1 RNA (FIG. 33). In neither cell line did 5-AzadC treatment induce an increase in FMRP (bottom panel of FIG. 39). Therefore, demethylation of fully methylated FMR1 induces FMR1-217 RNA expression. Most importantly, transcriptional activation of normally silenced FMR1 by demethylation alone does no commensurately induce FMRP expression.

TABLE 1
Non-Limiting Examples of RNA Markers with Increased Expression in FXS

| Gene symbol | ENSEMBL Gene ID | log2FoldChange | pvalue | padj |
|---|---|---|---|---|
| FAM3B | ENSG00000183844 | 5.94 | 0.000037 | 0.004264 |
| XKR3 | ENSG00000172967 | 2.80 | 0.000125 | 0.008997 |
| MAP3K15 | ENSG00000180815 | 2.08 | 0.000115 | 0.008550 |
| LEP | ENSG00000174697 | 2.03 | 0.000780 | 0.027134 |
| RP11-706O15.3 | nan | 1.97 | 0.001652 | 0.039687 |
| GCOM1 | ENSG00000137878 | 1.96 | 0.000285 | 0.015209 |
| CXCL6 | ENSG00000124875 | 1.47 | 0.000001 | 0.000440 |
| RGL3 | ENSG00000205517 | 1.43 | 0.000008 | 0.001802 |
| NECAB2 | ENSG00000103154 | 1.41 | 0.000030 | 0.003899 |
| TGM3 | ENSG00000125780 | 1.41 | 0.000176 | 0.011573 |
| LRRC6 | ENSG00000129295 | 1.31 | 0.000013 | 0.002488 |
| MAB21L3 | ENSG00000173212 | 1.31 | 0.000979 | 0.030144 |
| RP11-36B15.1 | nan | 1.29 | 0.001360 | 0.036019 |
| AC091878.1 | nan | 1.27 | 0.001202 | 0.033616 |
| RP11-154H23.3 | nan | 1.27 | 0.000120 | 0.008698 |
| NOV | ENSG00000136999 | 1.26 | 0.000020 | 0.003000 |
| AC093495.4 | nan | 1.23 | 0.000161 | 0.010925 |
| RP11-455F5.6 | nan | 1.22 | 0.000153 | 0.010637 |
| RGPD2 | ENSG00000185304 | 1.21 | 0.000081 | 0.006732 |
| COL9A3 | ENSG00000092758 | 1.21 | 0.000580 | 0.023121 |
| CLEC18A | ENSG00000157322 | 1.19 | 0.002008 | 0.045512 |
| RP11-256L6.2 | nan | 1.16 | 0.000829 | 0.027666 |
| LINC01127 | ENSG00000281162 | 1.15 | 0.000037 | 0.004264 |
| SLC38A11 | ENSG00000169507 | 1.14 | 0.000595 | 0.023337 |
| EFCAB12 | ENSG00000172771 | 1.12 | 0.000427 | 0.019671 |
| LA16c-380H5.5 | nan | 1.10 | 0.000155 | 0.010758 |
| CXCL1 | ENSG00000163739 | 1.10 | 0.001537 | 0.038485 |
| RP11-1334A24.5 | nan | 1.10 | 0.000012 | 0.002357 |
| AC100793.2 | nan | 1.07 | 0.000005 | 0.001405 |
| ANKDD1A | ENSG00000166839 | 1.07 | 0.000081 | 0.006732 |
| AVIL | ENSG00000135407 | 1.06 | 0.000089 | 0.007105 |
| RP11-44F14.8 | nan | 1.03 | 0.000021 | 0.003068 |
| RP11-290F20.1 | nan | 1.03 | 0.000426 | 0.019671 |
| AC116366.5 | nan | 1.03 | 0.000023 | 0.003198 |
| EPHB4 | ENSG00000196411 | 1.02 | 0.000000 | 0.000003 |
| ST6GALNAC3 | ENSG00000184005 | 1.02 | 0.001602 | 0.039149 |
| PANX2 | ENSG00000073150 | 1.01 | 0.000000 | 0.000081 |
| CREB5 | ENSG00000146592 | 1.01 | 0.000018 | 0.002853 |
| KIAA0319 | ENSG00000137261 | 1.00 | 0.000145 | 0.010196 |
| HECW2 | ENSG00000138411 | 0.99 | 0.000041 | 0.004422 |
| ADCY4 | ENSG00000129467 | 0.99 | 0.000001 | 0.000346 |
| LINC00173 | ENSG00000196668 | 0.99 | 0.000055 | 0.005277 |
| RP11-59D5_B.2 | nan | 0.99 | 0.000001 | 0.000408 |
| RP11-274B18.2 | nan | 0.97 | 0.001709 | 0.040781 |
| RP11-213H15.3 | nan | 0.97 | 0.000002 | 0.000670 |
| CORO7-PAM16 | ENSG00000103426 | 0.97 | 0.000004 | 0.001204 |
| HAL | ENSG00000084110 | 0.95 | 0.000013 | 0.002488 |
| DPEP3 | ENSG00000141096 | 0.95 | 0.000035 | 0.004173 |
| AC002467.7 | nan | 0.95 | 0.000859 | 0.028282 |
| MGAM | ENSG00000257335 | 0.95 | 0.000265 | 0.014805 |

TABLE 2
Non-Limiting Examples of RNA Markers with Reduced Expression in FXS

| Gene symbol | ENSEMBL Gene ID | log2FoldChange | pvalue | padj |
|---|---|---|---|---|
| S100B | ENSG00000160307 | −3.34 | 1.23E−07 | 0.00013926 |
| RP11-885N19.6 | nan | −3.11 | 8.50E−05 | 0.00695513 |
| RP11-545I5.3 | nan | −2.78 | 0.00171979 | 0.04085578 |
| AC091814.2 | nan | −2.52 | 0.00067871 | 0.02508099 |
| KLRC2 | ENSG00000205809 | −2.45 | 0.00063287 | 0.02384782 |
| L1TD1 | ENSG00000240563 | −2.41 | 1.70E−07 | 0.00015287 |
| PGBD5 | ENSG00000177614 | −2.27 | 0.00027758 | 0.01511272 |
| MXRA7 | ENSG00000182534 | −1.93 | 3.42E−05 | 0.0041732 |
| CROCC2 | ENSG00000226321 | −1.86 | 0.00076619 | 0.02700921 |
| SEMA5A | ENSG00000112902 | −1.84 | 1.60E−06 | 0.00063283 |
| PLA2G4C | ENSG00000105499 | −1.83 | 3.68E−06 | 0.00112744 |
| RP11-1008C21.1 | nan | −1.83 | 0.00077192 | 0.02708609 |
| TANC1 | ENSG00000115183 | −1.81 | 0.00062905 | 0.02384782 |
| C4orf50 | ENSG00000181215 | −1.79 | 7.02E−05 | 0.00610208 |
| NUAK1 | ENSG00000074590 | −1.76 | 0.00092774 | 0.02908497 |
| AC104809.4 | nan | −1.72 | 7.07E−05 | 0.00611524 |
| RGS17 | ENSG00000091844 | −1.71 | 0.00118851 | 0.03348616 |
| KCNS1 | ENSG00000124134 | −1.69 | 0.00032366 | 0.01655409 |
| DRAXIN | ENSG00000162490 | −1.69 | 0.00086416 | 0.02831001 |
| B3GAT1 | ENSG00000109956 | −1.68 | 0.00068513 | 0.02516046 |
| ARHGEF28 | ENSG00000214944 | −1.63 | 0.00213167 | 0.04671591 |
| KIF19 | ENSG00000196169 | −1.62 | 0.00115227 | 0.03276699 |
| APOL4 | ENSG00000100336 | −1.60 | 0.00133272 | 0.03567311 |
| GZMH | ENSG00000100450 | −1.58 | 0.00232882 | 0.04897115 |
| GAS1 | ENSG00000180447 | −1.52 | 1.80E−06 | 0.00067285 |
| SCD5 | ENSG00000145284 | −1.52 | 4.62E−07 | 0.00033415 |
| GLB1L2 | ENSG00000149328 | −1.50 | 0.00142343 | 0.03695763 |
| IGHA1 | ENSG00000211895 | −1.48 | 8.12E−10 | 3.11E−06 |
| KNDC1 | ENSG00000171798 | −1.45 | 0.0001015 | 0.00776551 |
| RP11-383H13.1 | nan | −1.44 | 0.00087252 | 0.02840134 |
| FGFR2 | ENSG00000066468 | −1.44 | 1.43E−05 | 0.00258244 |
| TFCP2L1 | ENSG00000115112 | −1.43 | 0.00033156 | 0.01674834 |
| PDGFRB | ENSG00000113721 | −1.41 | 0.0001128 | 0.00841832 |
| LAG3 | ENSG00000089692 | −1.38 | 1.35E−06 | 0.00058675 |
| GPR153 | ENSG00000158292 | −1.36 | 2.96E−05 | 0.00383882 |
| PODN | ENSG00000174348 | −1.36 | 6.61E−05 | 0.00586108 |
| CKB | ENSG00000166165 | −1.35 | 3.44E−05 | 0.0041732 |
| CERCAM | ENSG00000167123 | −1.31 | 3.11E−05 | 0.00396882 |
| ZNF365 | ENSG00000138311 | −1.30 | 0.00026806 | 0.01480521 |
| JUP | ENSG00000173801 | −1.28 | 8.13E−06 | 0.00182843 |
| TRNP1 | ENSG00000253368 | −1.28 | 0.00148083 | 0.03787471 |
| JAKMIP1 | ENSG00000152969 | −1.28 | 1.29E−05 | 0.00248842 |
| CPXM1 | ENSG00000088882 | −1.27 | 0.0011645 | 0.0330532 |
| SLC1A7 | ENSG00000162383 | −1.25 | 0.00025407 | 0.01434331 |
| LGR6 | ENSG00000133067 | −1.24 | 0.00046635 | 0.02056663 |
| FCRL6 | ENSG00000181036 | −1.23 | 0.00076397 | 0.02700921 |
| MORN4 | ENSG00000171160 | −1.23 | 9.68E−05 | 0.00748928 |
| TUBB2A | ENSG00000137267 | −1.21 | 0.00111934 | 0.03223865 |
| PRSS23 | ENSG00000150687 | −1.18 | 9.24E−08 | 0.00012854 |
| BFSP1 | ENSG00000125864 | −1.16 | 0.00018139 | 0.01165981 |

TABLE 3
Non-Limiting Examples of RNA Markers with Decreased Exon Skipping in FXS

| Gene symbol | ENSEMBL Gene ID | chr | strand | PValue | FDR | IncLevelDifference |
|---|---|---|---|---|---|---|
| NCALD | ENSG00000104490 | 8 | − | 4.96E−08 | 1.72E−05 | 0.505 |
| ZNF573 | ENSG00000189144 | 19 | − | 2.04E−05 | 0.00196032 | 0.339 |
| PAK1 | ENSG00000149269 | 11 | − | 0.00044025 | 0.02048598 | 0.31 |
| MIR4435-2HG | ENSG00000172965 | 2 | − | 8.29E−05 | 0.00579463 | 0.285 |
| CD8B | ENSG00000172116 | 2 | − | 1.81E−05 | 0.00177935 | 0.277 |
| PDGFC | ENSG00000145431 | 4 | − | 0.000346763 | 0.01709337 | 0.264 |
| TRAPPC2L | ENSG00000167515 | 16 | + | 0.000221258 | 0.01224535 | 0.257 |
| AC006504.5 | nan | 19 | + | 0.001123229 | 0.04142531 | 0.245 |
| ZNF512 | ENSG00000243943 | 2 | + | 0.000146908 | 0.00893035 | 0.232 |
| FAM228B | ENSG00000219626 | 2 | + | 0.001178813 | 0.04295611 | 0.226 |
| NEIL2 | ENSG00000154328 | 8 | + | 5.13E−05 | 0.00403721 | 0.215 |
| FAM78A | ENSG00000126882 | 9 | − | 0.000593929 | 0.02558632 | 0.21 |
| FYB1 | ENSG00000082074 | 5 | − | 3.81E−06 | 0.00053168 | 0.2 |
| RNF216P1 | ENSG00000196204 | 7 | + | 0.001005397 | 0.03818502 | 0.197 |
| ZCWPW1 | ENSG00000078487 | 7 | − | 0.000637671 | 0.02697481 | 0.194 |
| DTX2 | ENSG00000091073 | 7 | + | 0.000222932 | 0.01231721 | 0.187 |
| ATP5MD | ENSG00000173915 | 10 | − | 0.000189351 | 0.01083592 | 0.187 |
| MX2 | ENSG00000183486 | 21 | + | 0.000241142 | 0.01307969 | 0.18 |
| LYRM1 | ENSG00000102897 | 16 | + | 0.00024572 | 0.01325809 | 0.175 |
| GUF1 | ENSG00000151806 | 4 | + | 0.000727864 | 0.02995953 | 0.174 |
| DPH7 | ENSG00000148399 | 9 | − | 0.000264816 | 0.01399492 | 0.173 |
| NSFL1C | ENSG00000088833 | 20 | − | 3.62E−06 | 0.00051009 | 0.169 |
| MTMR1 | ENSG00000063601 | X | + | 0.00123067 | 0.0444019 | 0.169 |
| GTPBP10 | ENSG00000105793 | 7 | + | 0.001021594 | 0.03855642 | 0.165 |
| RGS3 | ENSG00000138835 | 9 | + | 8.35E−10 | 6.81E−07 | 0.165 |

| Gene symbol | exonStart_0base | exonEnd | upstreamES | upstreamEE | downstreamES | downstreamEE |
|---|---|---|---|---|---|---|
| NCALD | 101779923 | 101779979 | 101719385 | 101719648 | 101887140 | 101887227 |
| ZNF573 | 37759046 | 37759139 | 37738286 | 37740194 | 37770004 | 37770097 |
| PAK1 | 77397026 | 77397093 | 77392442 | 77392541 | 77408153 | 77408232 |
| MIR4435-2HG | 111247429 | 111247558 | 111239801 | 111239996 | 111248147 | 111248241 |
| CD8B | 86822320 | 86822374 | 86815346 | 86815718 | 86844921 | 86844958 |
| PDGFC | 156861428 | 156861489 | 156850220 | 156850416 | 156970785 | 156971315 |
| TRAPPC2L | 88859344 | 88859443 | 88858618 | 88858789 | 88859892 | 88859972 |
| AC006504.5 | 27797753 | 27798176 | 27793430 | 27793650 | 27805604 | 27805811 |
| ZNF512 | 27583657 | 27583716 | 27582968 | 27583142 | 27598069 | 27598254 |
| FAM228B | 24167626 | 24167683 | 24164197 | 24164335 | 24169355 | 24169496 |
| NEIL2 | 11770173 | 11770335 | 11769698 | 11769791 | 11771445 | 11771585 |
| FAM78A | 131263477 | 131263599 | 131258075 | 131261350 | 131270459 | 131270523 |
| FYB1 | 39212677 | 39213017 | 39202773 | 39202987 | 39274402 | 39274528 |
| RNF216P1 | 4995474 | 4995582 | 4984909 | 4985075 | 4996609 | 4996673 |
| ZCWPW1 | 100401888 | 100402041 | 100400852 | 100401336 | 100403693 | 100403785 |
| DTX2 | 76497336 | 76497477 | 76492152 | 76492253 | 76500440 | 76500520 |
| ATP5MD | 103394198 | 103394394 | 103392370 | 103392466 | 103395745 | 103396032 |
| MX2 | 41373897 | 41374127 | 41370212 | 41370645 | 41376835 | 41376949 |
| LYRM1 | 20902485 | 20902717 | 20900449 | 20900535 | 20920121 | 20920214 |
| GUF1 | 44688006 | 44688146 | 44686509 | 44686713 | 44689285 | 44689409 |
| DPH7 | 137577576 | 137577603 | 137576308 | 137576402 | 137578624 | 137578717 |
| NSFL1C | 1472714 | 1473012 | 1464328 | 1464426 | 1473791 | 1473842 |
| MTMR1 | 150751029 | 150751240 | 150750729 | 150750843 | 150762564 | 150762737 |
| GTPBP10 | 90354457 | 90354549 | 90352815 | 90353009 | 90372154 | 90372228 |
| RGS3 | 113565514 | 113565730 | 113536795 | 113536918 | 113583449 | 113584427 |

TABLE 4
Non-Limiting Examples of RNA Markers with Increased Exon Skipping in FXS

| Gene symbol | ENSEMBL Gene ID | chr | strand | PValue | FDR | IncLevelDifference |
|---|---|---|---|---|---|---|
| NCALD | ENSG00000104490 | 8 | − | 5.33E−15 | 2.91E−11 | −0.504 |
| DRAM2 | ENSG00000156171 | 1 | − | 2.71E−13 | 9.60E−10 | −0.441 |
| NCALD | ENSG00000104490 | 8 | − | 2.24E−07 | 5.54E−05 | −0.435 |
| RHOH | ENSG00000168421 | 4 | + | 0.000779534 | 0.031522709 | −0.374 |
| LAIR2 | ENSG00000167618 | 19 | + | 5.36E−06 | 0.000699899 | −0.363 |
| GBP3 | ENSG00000117226 | 1 | − | 7.09E−09 | 3.66E−06 | −0.359 |
| GTF2H1 | ENSG00000110768 | 11 | + | 0.000513829 | 0.022997385 | −0.353 |
| XPNPEP3 | ENSG00000196236 | 22 | + | 2.97E−09 | 1.81E−06 | −0.336 |
| DRAM2 | ENSG00000156171 | 1 | − | 3.11E−09 | 1.86E−06 | −0.308 |
| ZNF888 | ENSG00000213793 | 19 | − | 0.000197457 | 0.011192452 | −0.295 |
| TBC1D5 | ENSG00000131374 | 3 | − | 0.000208142 | 0.011652147 | −0.279 |
| AC060780.1 | nan | 17 | − | 0.000132965 | 0.008300559 | −0.276 |
| SDHAP2 | ENSG00000215837 | 3 | + | 8.95E−11 | 1.18E−07 | −0.254 |
| KMT2A | ENSG00000118058 | 11 | + | 2.66E−06 | 0.000398951 | −0.237 |
| SH3BP2 | ENSG00000087266 | 4 | + | 3.42E−09 | 2.00E−06 | −0.226 |
| TBC1D5 | ENSG00000131374 | 3 | − | 0.000917527 | 0.035663172 | −0.225 |
| CSNK1G2 | ENSG00000133275 | 19 | + | 4.33E−05 | 0.003529449 | −0.214 |
| ATP5MD | ENSG00000173915 | 10 | − | 0.000573727 | 0.025011619 | −0.214 |
| NSUN5P1 | ENSG00000223705 | 7 | + | 2.96E−06 | 0.000432046 | −0.204 |
| LINC01128 | ENSG00000228794 | 1 | + | 0.00014825 | 0.008981293 | −0.204 |
| RNF19A | ENSG00000034677 | 8 | − | 1.09E−05 | 0.001206693 | −0.194 |
| SNHG8 | ENSG00000269893 | 4 | + | 0.000256909 | 0.013715154 | −0.189 |
| SNHG8 | ENSG00000269893 | 4 | + | 0.000178693 | 0.010366148 | −0.182 |
| TOP1MT | ENSG00000184428 | 8 | − | 0.001185002 | 0.043130386 | −0.178 |
| AL135818.1 | nan | 14 | + | 0.000537267 | 0.023809757 | −0.174 |

| Gene symbol | exonStart_0base | exonEnd | upstreamES | upstreamEE | downstreamES | downstreamEE |
|---|---|---|---|---|---|---|
| NCALD | 101887140 | 101887227 | 101719251 | 101719648 | 101915808 | 101915858 |
| DRAM2 | 111139587 | 111139711 | 111137522 | 111137586 | 111140037 | 111140062 |
| NCALD | 101887140 | 101887227 | 101779923 | 101779979 | 101915808 | 101915858 |
| RHOH | 40235192 | 40235358 | 40193662 | 40193812 | 40242713 | 40242834 |
| LAIR2 | 54509034 | 54509085 | 54507890 | 54508184 | 54510525 | 54510687 |
| GBP3 | 89010903 | 89011116 | 89009391 | 89009494 | 89013184 | 89013427 |
| GTF2H1 | 18325946 | 18326153 | 18324235 | 18324296 | 18333059 | 18333183 |
| XPNPEP3 | 40860677 | 40860793 | 40857096 | 40857245 | 40861617 | 40862770 |
| DRAM2 | 111139500 | 111139666 | 111137522 | 111137586 | 111140037 | 111140090 |
| ZNF888 | 52918818 | 52918937 | 52917858 | 52917931 | 52923368 | 52923481 |
| TBC1D5 | 17622647 | 17622765 | 17562018 | 17562142 | 17623848 | 17623913 |
| AC060780.1 | 43166979 | 43167849 | 43148367 | 43148783 | 43170125 | 43170316 |
| SDHAP2 | 195663623 | 195663767 | 195662570 | 195662733 | 195664085 | 195664250 |
| KMT2A | 118439023 | 118439122 | 118436509 | 118436944 | 118468774 | 118468844 |
| SH3BP2 | 2794162 | 2794262 | 2793084 | 2793138 | 2796373 | 2796646 |
| TBC1D5 | 17518901 | 17518995 | 17508473 | 17508605 | 17562018 | 17562142 |
| CSNK1G2 | 1968790 | 1968905 | 1942458 | 1942613 | 1969507 | 1969959 |
| ATP5MD | 103395745 | 103396032 | 103394198 | 103394394 | 103396408 | 103396475 |
| NSUN5P1 | 75414639 | 75414798 | 75412788 | 75412932 | 75414884 | 75415024 |
| LINC01128 | 841199 | 841373 | 829002 | 829104 | 851926 | 852110 |
| RNF19A | 100300527 | 100300651 | 100287500 | 100288267 | 100303159 | 100303259 |
| SNHG8 | 118278943 | 118279137 | 118278754 | 118278792 | 118279388 | 118279431 |
| SNHG8 | 118278933 | 118279137 | 118278754 | 118278792 | 118279388 | 118279431 |
| TOP1MT | 143332486 | 143332657 | 143331223 | 143331339 | 143334107 | 143334285 |
| AL135818.1 | 91247489 | 91247659 | 91244354 | 91244499 | 91250764 | 91251228 |

TABLE 5
Non-Limiting Examples of RNA Markers with Decreased Mutually Exclusive Exon (MXE) Switching in FXS

| Gene symbol | ENSEMBL Gene ID | chr | strand | PValue | FDR | IncLevelDifference |
|---|---|---|---|---|---|---|
| CR1 | ENSG00000203710 | chr1 | + | 5.69E−06 | 0.000783263 | 0.395 |
| CRIM1 | ENSG00000150938 | chr2 | + | 8.09E−07 | 0.00015988 | 0.271 |
| ZCWPW1 | ENSG00000078487 | chr7 | − | 4.05E−07 | 8.88E−05 | 0.262 |
| NAP1L1 | ENSG00000187109 | chr12 | − | 0.000173204 | 0.01078021 | 0.238 |
| TBC1D5 | ENSG00000131374 | chr3 | − | 0.001307502 | 0.046275622 | 0.237 |
| MIR4435-2HG | ENSG00000172965 | chr2 | − | 0.00102711 | 0.038876331 | 0.236 |
| AC004593.2 | nan | chr7 | + | 5.31E−05 | 0.004472215 | 0.235 |
| GBP3 | ENSG00000117226 | chr1 | − | 0.000729866 | 0.030625267 | 0.222 |
| SEC61A2 | ENSG00000065665 | chr10 | + | 0.000855726 | 0.034366856 | 0.22 |
| PCNX2 | ENSG00000135749 | chr1 | − | 6.98E−05 | 0.0054603 | 0.215 |
| NAP1L1 | ENSG00000187109 | chr12 | − | 0.000695123 | 0.029500582 | 0.206 |
| TPT1-AS1 | ENSG00000170919 | chr13 | + | 8.19E−05 | 0.006166312 | 0.195 |
| HLA-A | ENSG00000206503 | chr6 | + | 9.84E−05 | 0.007006027 | 0.179 |
| HLA-A | ENSG00000206503 | chr6 | + | 9.64E−05 | 0.006902919 | 0.178 |
| LUCAT1 | ENSG00000248323 | chr5 | − | 0.000404613 | 0.020312094 | 0.176 |
| PTPN2 | ENSG00000175354 | chr18 | − | 0.000173086 | 0.01078021 | 0.174 |
| SEC31B | ENSG00000075826 | chr10 | − | 2.17E−05 | 0.002275108 | 0.168 |
| POLR2J3 | ENSG00000168255 | chr7 | − | 0.000306057 | 0.01649295 | 0.164 |
| POLR2J4 | ENSG00000214783 | chr7 | − | 5.42E−05 | 0.00453613 | 0.157 |
| CAST | ENSG00000153113 | chr5 | + | 5.09E−08 | 1.72E−05 | 0.148 |
| POLR2J4 | ENSG00000272655 | chr7 | − | 0.000252606 | 0.014256911 | 0.147 |
| NUMBL | ENSG00000105245 | chr19 | − | 0.000888058 | 0.035038326 | 0.144 |
| PRMT7 | ENSG00000132600 | chr16 | + | 0.000272932 | 0.015030406 | 0.143 |
| ATF7IP2 | ENSG00000166669 | chr16 | + | 0.001005564 | 0.038178258 | 0.14 |
| TIMM23B-AGAP6 | ENSG00000178440 | chr10 | + | 2.60E−08 | 9.71E−06 | 0.138 |

| Gene name | 1stExonStart_0base | 1stExonEnd | 2ndExonStart_0base | 2ndExonEnd | upstreamES | upstreamEE | downstreamES | downstreamEE |
|---|---|---|---|---|---|---|---|---|
| CR1 | 207526752 | 207526929 | 207545307 | 207545484 | 207523610 | 207524009 | 207545575 | 207545678 |
| CRIM1 | 36522091 | 36522313 | 36537351 | 36537546 | 36517326 | 36517542 | 36544375 | 36544498 |
| ZCWPW1 | 100403693 | 100403785 | 100404177 | 100404244 | 100400872 | 100401336 | 100405012 | 100405093 |
| NAP1L1 | 76060137 | 76060279 | 76074202 | 76074239 | 76056032 | 76056161 | 76084566 | 76084685 |
| TBC1D5 | 17518901 | 17518995 | 17562018 | 17562142 | 17508473 | 17508605 | 17586352 | 17586557 |
| MIR4435- | 111239801 | 111239996 | 111247429 | 111247558 | 111233627 | 111233739 | 111248147 | 111248241 |
| 2HG | | | | | | | | |
| AC004593.2 | 29146848 | 29146960 | 29354624 | 29354663 | 29145853 | 29146745 | 29367931 | 29367987 |
| GBP3 | 89011746 | 89012027 | 89013184 | 89013427 | 89010903 | 89011116 | 89014082 | 89014279 |
| SEC61A2 | 12149594 | 12149726 | 12149851 | 12149961 | 12143116 | 12143195 | 12156906 | 12157067 |
| PCNX2 | 233139713 | 233139855 | 233160282 | 233160433 | 233134783 | 233135190 | 233161270 | 233161363 |
| NAP1L1 | 76067370 | 76067473 | 76074202 | 76074239 | 76050530 | 76050653 | 76084566 | 76084735 |
| TPT1-AS1 | 45383090 | 45383235 | 45389734 | 45389820 | 45378529 | 45380001 | 45390713 | 45390902 |
| HLA-A | 29942756 | 29943543 | 29944121 | 29944397 | 29942551 | 29942626 | 29944499 | 29944616 |
| HLA-A | 29942756 | 29943026 | 29944121 | 29944397 | 29942244 | 29942626 | 29944499 | 29944634 |
| LUCAT1 | 91303872 | 91303971 | 91312294 | 91312495 | 91302730 | 91303161 | 91313077 | 91313229 |
| PTPN2 | 12819213 | 12819282 | 12821335 | 12821425 | 12814202 | 12814355 | 12825809 | 12825944 |
| SEC31B | 100490007 | 100490322 | 100495384 | 100495546 | 100489702 | 100489761 | 100496257 | 100496431 |
| POLR2J3 | 102544705 | 102544776 | 102566581 | 102566736 | 102541500 | 102541662 | 102566996 | 102567083 |
| POLR2J4 | 43986600 | 43986677 | 43987677 | 43987888 | 43973209 | 43973323 | 43988085 | 43988247 |
| CAST | 96740037 | 96740118 | 96740744 | 96740783 | 96737848 | 96737947 | 96741265 | 96741358 |
| POLR2J4 | 43986600 | 43986677 | 44013592 | 44013679 | 43973209 | 43973323 | 44014605 | 44014783 |
| NUMBL | 40673343 | 40673649 | 40677231 | 40677421 | 40669897 | 40670020 | 40680916 | 40681057 |
| PRMT7 | 68312041 | 68312176 | 68315896 | 68316074 | 68310973 | 68311099 | 68329065 | 68329174 |
| ATF7IP2 | 10473922 | 10473989 | 10480878 | 10480964 | 10473478 | 10473534 | 10481835 | 10483637 |
| TIMM23B- | 49987164 | 49987234 | 49988750 | 49988938 | 49973011 | 49973142 | 49989307 | 49989376 |
| AGAP6 | | | | | | | | |

TABLE 6
Non-Limiting Examples of RNA Markers with Increased Mutually Exclusive Exon (MXE) Switching in FXS

| Gene | | | | | | |
|---|---|---|---|---|---|---|
| symbol | ENSEMBL Gene ID | chr | strand | PValue | FDR | IncLevelDifference |
| HLA-A | ENSG00000206503 | chr6 | + | 0 | 0 | −0.569 |
| HLA-A | ENSG00000206503 | chr6 | + | 0 | 0 | −0.503 |
| HLA-A | ENSG00000206503 | chr6 | + | 0 | 0 | −0.482 |
| HLA-A | ENSG00000206503 | chr6 | + | 0 | 0 | −0.478 |
| HLA-A | ENSG00000206503 | chr6 | + | 0 | 0 | −0.469 |
| HLA-A | ENSG00000206503 | chr6 | + | 0 | 0 | −0.332 |
| ADGRE2 | ENSG00000127507 | chr19 | − | 0.000111359 | 0.007655312 | −0.298 |
| PAK1 | ENSG00000149269 | chr11 | − | 0.000566363 | 0.025601762 | −0.294 |
| TBC1D5 | ENSG00000131374 | chr3 | − | 6.39E−05 | 0.005090138 | −0.272 |
| GTF2H2B | ENSG00000226259 | chr5 | + | 0.001061621 | 0.039866163 | −0.26 |
| MICA | ENSG00000204520 | chr6 | + | 2.23E−06 | 0.000369146 | −0.24 |
| SLC29A2 | ENSG00000174669 | chr11 | − | 0.001359543 | 0.047413746 | −0.225 |
| HLA-A | ENSG00000206503 | chr6 | + | 1.60E−07 | 4.19E−05 | −0.209 |
| ZBTB10 | ENSG00000205189 | chr8 | + | 0.000213606 | 0.012757245 | −0.195 |
| NLGN3 | ENSG00000196338 | chrX | + | 0.001294693 | 0.045950152 | −0.194 |
| CAST | ENSG00000153113 | chr5 | + | 1.16E−08 | 4.98E−06 | −0.187 |
| METTL25 | ENSG00000127720 | chr12 | + | 1.66E−05 | 0.001861552 | −0.186 |
| MICA | ENSG00000204520 | chr6 | + | 0.000904573 | 0.0353971 | −0.181 |
| ADAM15 | ENSG00000143537 | chr1 | + | 0.000177579 | 0.011034472 | −0.179 |
| LUCAT1 | ENSG00000248323 | chr5 | − | 0.000129664 | 0.008649685 | −0.17 |
| TBC1D5 | ENSG00000131374 | chr3 | − | 6.26E−05 | 0.005018258 | −0.167 |
| SSH1 | ENSG00000084112 | chr12 | − | 5.43E−06 | 0.00075397 | −0.159 |
| ADAM15 | ENSG00000143537 | chr1 | + | 0.00025982 | 0.014540534 | −0.156 |
| SIRPB1 | ENSG00000101307 | chr20 | − | 6.72E−07 | 0.000137064 | −0.156 |
| GBP3 | ENSG00000117226 | chr1 | − | 9.61E−09 | 4.26E−06 | −0.155 |

| Gene | | | | | | | | |
|---|---|---|---|---|---|---|---|---|
| name | 1stExonStart_0base | 1stExonEnd | 2ndExonStart_0base | 2ndExonEnd | upstreamES | upstreamEE | downstreamES | downstreamEE |
| HLA-A | 29944121 | 29944397 | 29944499 | 29945091 | 29943284 | 29943543 | 29945233 | 29945281 |
| HLA-A | 29944121 | 29944397 | 29945058 | 29945091 | 29942756 | 29943543 | 29945233 | 29945281 |
| HLA-A | 29944499 | 29944616 | 29945233 | 29945281 | 29944121 | 29944397 | 29945450 | 29945870 |
| HLA-A | 29944121 | 29944397 | 29944499 | 29944616 | 29942756 | 29943543 | 29945058 | 29945091 |
| HLA-A | 29944499 | 29945091 | 29945233 | 29945281 | 29944121 | 29944397 | 29945450 | 29945884 |
| HLA-A | 29945058 | 29945091 | 29945233 | 29945281 | 29944499 | 29944616 | 29945450 | 29945870 |
| ADGRE2 | 14754953 | 14755127 | 14755653 | 14755844 | 14751435 | 14751671 | 14756237 | 14756345 |
| PAK1 | 77397026 | 77397158 | 77408153 | 77408232 | 77392330 | 77392541 | 77411773 | 77411933 |
| TBC1D5 | 17562018 | 17562142 | 17622647 | 17622765 | 17508473 | 17508605 | 17623848 | 17623913 |
| GTF2H2B | 70434914 | 70435022 | 70437530 | 70437594 | 70434178 | 70434266 | 70440755 | 70440859 |
| MICA | 31411071 | 31411359 | 31412324 | 31412460 | 31410542 | 31410797 | 31415011 | 31415313 |
| SLC29A2 | 66366125 | 66366231 | 66366430 | 66366564 | 66365935 | 66366021 | 66367463 | 66367548 |
| HLA-A | 29943267 | 29943543 | 29944499 | 29944616 | 29942756 | 29943026 | 29945058 | 29945091 |
| ZBTB10 | 80499493 | 80500382 | 80513909 | 80514008 | 80486212 | 80487782 | 80518402 | 80518438 |
| NLGN3 | 71164142 | 71164328 | 71167010 | 71167800 | 71155213 | 71155363 | 71169253 | 71171199 |
| CAST | 96740744 | 96740783 | 96741265 | 96741358 | 96740037 | 96740118 | 96741493 | 96741580 |
| METTL25 | 82456726 | 82456820 | 82476643 | 82476718 | 82438514 | 82438791 | 82477280 | 82477352 |
| MICA | 31411071 | 31411359 | 31411946 | 31412225 | 31410542 | 31410797 | 31412324 | 31412460 |
| ADAM15 | 155061417 | 155061489 | 155061903 | 155062117 | 155060204 | 155060343 | 155062244 | 155062369 |
| LUCAT1 | 91244819 | 91245010 | 91264662 | 91264713 | 91242940 | 91243002 | 91313637 | 91313777 |
| TBC1D5 | 17428449 | 17428519 | 17622647 | 17622765 | 17406417 | 17406526 | 17623848 | 17623913 |
| SSH1 | 108817037 | 108817159 | 108818248 | 108818313 | 108811259 | 108811328 | 108823257 | 108823361 |
| ADAM15 | 155061414 | 155061489 | 155061903 | 155062117 | 155060204 | 155060343 | 155062244 | 155062369 |
| SIRPB1 | 1566152 | 1566267 | 1578337 | 1578694 | 1564383 | 1565497 | 1588559 | 1588632 |
| GBP3 | 89009391 | 89009494 | 89011746 | 89012027 | 89008946 | 89009140 | 89013184 | 89013427 |

TABLE 7
Non-Limiting Examples of RNA Markers with Decreased Alternative 5′ Splice Site (A5SS) in FXS

| Gene symbol | ENSEMBL Gene ID | chr | strand | PValue | FDR | IncLevelDifference |
|---|---|---|---|---|---|---|
| PARP2 | ENSG00000129484 | chr14 | + | 1.47E−07 | 3.50E−05 | 0.307 |
| PACRGL | ENSG00000163138 | chr4 | + | 0.001882993 | 0.04083963 | 0.252 |
| ENTPD1-AS1 | ENSG00000226688 | chr10 | − | 0.000135031 | 0.005969913 | 0.217 |
| NEIL2 | ENSG00000154328 | chr8 | + | 0.000411623 | 0.01332848 | 0.203 |
| FUZ | ENSG00000010361 | chr19 | − | 0.000734977 | 0.020946846 | 0.184 |
| SDR39U1 | ENSG00000100445 | chr14 | − | 0.00031298 | 0.010957462 | 0.147 |
| ADAM15 | ENSG00000143537 | chr1 | + | 0.000445416 | 0.014027548 | 0.14 |
| SDR39U1 | ENSG00000100445 | chr14 | − | 0.000206025 | 0.00799062 | 0.136 |
| EPOR | ENSG00000187266 | chr19 | − | 0.002400735 | 0.048843263 | 0.118 |
| ZSCAN26 | ENSG00000197062 | chr6 | + | 0.001112686 | 0.027805061 | 0.113 |
| SNHG17 | ENSG00000196756 | chr20 | − | 0.001199516 | 0.029028276 | 0.113 |
| GPS2 | ENSG00000132522 | chr17 | − | 0.001308916 | 0.031129638 | 0.106 |
| NECAP1 | ENSG00000089818 | chr12 | + | 0.001379215 | 0.032576878 | 0.094 |
| MRPL11 | ENSG00000174547 | chr11 | − | 0.00088251 | 0.023776073 | 0.086 |
| DNAJC19 | ENSG00000205981 | chr3 | − | 0.000221009 | 0.008421537 | 0.085 |
| ANKZF1 | ENSG00000163516 | chr2 | + | 0.00119272 | 0.029005238 | 0.084 |
| C1orf162 | ENSG00000143110 | chr1 | + | 4.88E−06 | 0.000521701 | 0.077 |
| PIGT | ENSG00000124155 | chr20 | + | 0.000340165 | 0.011790904 | 0.073 |
| SLC25A37 | ENSG00000147454 | chr8 | + | 0.000418079 | 0.01347424 | 0.069 |
| AP1G1 | ENSG00000166747 | chr16 | − | 2.01E−05 | 0.001448127 | 0.061 |
| CIC | ENSG00000079432 | chr19 | + | 0.001042232 | 0.026722204 | 0.06 |
| ITGB7 | ENSG00000139626 | chr12 | − | 1.90E−05 | 0.001398195 | 0.059 |
| ATG16L2 | ENSG00000168010 | chr11 | + | 1.46E−06 | 0.000214648 | 0.058 |
| BECN1 | ENSG00000126581 | chr17 | − | 0.000617385 | 0.018137461 | 0.056 |
| ARHGEF40 | ENSG00000165801 | chr14 | + | 0.002236606 | 0.046323946 | 0.053 |

| Gene symbol | longExonStart_0base | longExonEnd | shortES | shortEE | flankingES | flankingEE |
|---|---|---|---|---|---|---|
| PARP2 | 20344931 | 20345126 | 20344931 | 20345087 | 20345393 | 20345464 |
| PACRGL | 20700457 | 20700787 | 20700457 | 20700611 | 20704465 | 20704533 |
| ENTPD1-AS1 | 95907662 | 95907903 | 95907777 | 95907903 | 95904829 | 95904938 |
| NEIL2 | 11769709 | 11770335 | 11769709 | 11769791 | 11771445 | 11771585 |
| FUZ | 49812446 | 49812736 | 49812637 | 49812736 | 49812250 | 49812335 |
| SDR39U1 | 24442108 | 24442260 | 24442177 | 24442260 | 24440782 | 24440926 |
| ADAM15 | 155061903 | 155062117 | 155061903 | 155061975 | 155062244 | 155062369 |
| SDR39U1 | 24442097 | 24442260 | 24442177 | 24442260 | 24440782 | 24440926 |
| EPOR | 11382846 | 11383232 | 11383096 | 11383232 | 11381929 | 11382105 |
| ZSCAN26 | 28271853 | 28272336 | 28271853 | 28271934 | 28272669 | 28272787 |
| SNHG17 | 38435026 | 38435352 | 38435196 | 38435352 | 38434503 | 38434626 |
| GPS2 | 7313567 | 7313988 | 7313905 | 7313988 | 7313211 | 7313291 |
| NECAP1 | 8090194 | 8091850 | 8090194 | 8090299 | 8092675 | 8092784 |
| MRPL11 | 66438631 | 66438848 | 66438709 | 66438848 | 66438163 | 66438259 |
| DNAJC19 | 180986947 | 180987022 | 180986997 | 180987022 | 180985925 | 180985996 |
| ANKZF1 | 219230227 | 219230405 | 219230227 | 219230350 | 219232259 | 219232362 |
| C1orf162 | 111476797 | 111477078 | 111476797 | 111476867 | 111477650 | 111477775 |
| PIGT | 45416516 | 45416694 | 45416516 | 45416654 | 45419294 | 45419395 |
| SLC25A37 | 23566107 | 23566813 | 23566107 | 23566336 | 23568321 | 23568378 |
| AP1G1 | 71808504 | 71808832 | 71808762 | 71808832 | 71789278 | 71789482 |
| CIC | 42294172 | 42294304 | 42294172 | 42294298 | 42294603 | 42294735 |
| ITGB7 | 53201117 | 53201194 | 53201146 | 53201194 | 53200242 | 53200446 |
| ATG16L2 | 72822477 | 72823797 | 72822477 | 72822543 | 72824059 | 72824122 |
| BECN1 | 42824132 | 42824282 | 42824154 | 42824282 | 42823747 | 42823879 |
| ARHGEF40 | 21075331 | 21075765 | 21075331 | 21075499 | 21076359 | 21076456 |

TABLE 8
Non-Limiting Examples of RNA Markers with Increased Alternative 5′ Splice Site (A5SS) in FXS

| Gene symbol | ENSEMBL Gene ID | chr | strand | PValue | FDR | IncLevelDifference |
|---|---|---|---|---|---|---|
| BANP | ENSG00000172530 | chr16 | + | 3.92E−05 | 0.002371624 | −0.251 |
| PIGA | ENSG00000165195 | chrX | − | 0.001051527 | 0.026841331 | −0.23 |
| SNHG8 | ENSG00000269893 | chr4 | + | 0.00023797 | 0.008824083 | −0.183 |
| PIGA | ENSG00000165195 | chrX | − | 0.001803685 | 0.039617882 | −0.172 |
| RAD52 | ENSG00000002016 | chr12 | − | 1.03E−05 | 0.000910652 | −0.152 |
| IRF3 | ENSG00000126456 | chr19 | − | 0.001145703 | 0.028226011 | −0.131 |
| CEP78 | ENSG00000148019 | chr9 | + | 1.28E−07 | 3.27E−05 | −0.115 |
| SPINT1 | ENSG00000166145 | chr15 | + | 0.001017573 | 0.026722204 | −0.103 |
| TMEM156 | ENSG00000121895 | chr4 | − | 8.68E−06 | 0.000801041 | −0.095 |
| NT5C3B | ENSG00000141698 | chr17 | − | 1.67E−09 | 1.28E−06 | −0.081 |
| PLD2 | ENSG00000129219 | chr17 | + | 0.001405759 | 0.032977951 | −0.079 |
| HLA-A | ENSG00000206503 | chr6 | + | 1.24E−06 | 0.000194652 | −0.078 |
| ANKRD12 | ENSG00000101745 | chr18 | + | 6.58E−08 | 1.89E−05 | −0.074 |
| CASP8 | ENSG00000064012 | chr2 | + | 0.000112282 | 0.005162722 | −0.071 |
| PACS2 | ENSG00000179364 | chr14 | + | 0.000167594 | 0.007048141 | −0.069 |
| HLA-DMA | ENSG00000204257 | chr6 | − | 0.00151269 | 0.034776744 | −0.066 |
| DHPS | ENSG00000095059 | chr19 | − | 7.10E−05 | 0.003816051 | −0.061 |
| PDCD6 | ENSG00000249915 | chr5 | + | 0.002223693 | 0.046323946 | −0.057 |

| Gene symbol | longExonStart_0base | longExonEnd | shortES | shortEE | flankingES | flankingEE |
|---|---|---|---|---|---|---|
| BANP | 87975047 | 87975207 | 87975047 | 87975203 | 87981035 | 87981127 |
| PIGA | 15331589 | 15331992 | 15331873 | 15331992 | 15325913 | 15326046 |
| SNHG8 | 118278708 | 118279137 | 118278708 | 118278792 | 118279388 | 118279431 |
| PIGA | 15331215 | 15331992 | 15331873 | 15331992 | 15325913 | 15326046 |
| RAD52 | 914410 | 914532 | 914430 | 914532 | 913893 | 914121 |
| IRF3 | 49664442 | 49664673 | 49664552 | 49664673 | 49663342 | 49663514 |
| CEP78 | 78236079 | 78236659 | 78236079 | 78236603 | 78240022 | 78240195 |
| SPINT1 | 40853710 | 40853929 | 40853710 | 40853881 | 40854059 | 40854086 |
| TMEM156 | 38998622 | 38998909 | 38998639 | 38998909 | 38993737 | 38993998 |
| NT5C3B | 41835587 | 41835957 | 41835858 | 41835957 | 41835202 | 41835272 |
| PLD2 | 4819428 | 4819582 | 4819428 | 4819549 | 4821792 | 4821907 |
| HLA-A | 29944499 | 29944634 | 29944499 | 29944616 | 29945058 | 29945091 |
| ANKRD12 | 9208656 | 9208803 | 9208656 | 9208797 | 9211583 | 9211784 |
| CASP8 | 201276826 | 201277104 | 201276826 | 201276968 | 201284815 | 201285317 |
| PACS2 | 105381913 | 105382058 | 105381913 | 105382046 | 105382476 | 105382581 |
| HLA-DMA | 32949270 | 32949399 | 32949273 | 32949399 | 32948612 | 32948868 |
| DHPS | 12680149 | 12680325 | 12680195 | 12680325 | 12679800 | 12679922 |
| PDCD6 | 304176 | 306754 | 304176 | 304221 | 311292 | 311402 |

TABLE 9
Non-Limiting Examples of RNA Markers with Decreased Alternative 3′ Splice Site (A3SS) in FXS

| Gene symbol | ENSEMBL Gene ID | chr | strand | PValue | FDR | IncLevelDifference |
|---|---|---|---|---|---|---|
| SNX5 | ENSG00000089006 | chr20 | − | 0.000194034 | 0.008919334 | 0.36 |
| POLR2J3 | ENSG00000168255 | chr7 | − | 0.000357592 | 0.013857389 | 0.274 |
| MPPE1 | ENSG00000154889 | chr18 | − | 0.00065011 | 0.021232751 | 0.225 |
| AC016394.2 | nan | chr10 | + | 0.000373496 | 0.014201229 | 0.224 |
| DPM1 | ENSG00000000419 | chr20 | − | 0.002112825 | 0.049154541 | 0.215 |
| E2F5 | ENSG00000133740 | chr8 | + | 0.000690702 | 0.022132884 | 0.193 |
| PTPN7 | ENSG00000143851 | chr1 | − | 0.000234758 | 0.010311143 | 0.192 |
| MTFP1 | ENSG00000242114 | chr22 | + | 0.001427494 | 0.036640206 | 0.189 |
| TOR1AIP1 | ENSG00000143337 | chr1 | + | 0.001713159 | 0.041763377 | 0.18 |
| POT1 | ENSG00000128513 | chr7 | − | 0.001333453 | 0.034930288 | 0.164 |
| JOSD2 | ENSG00000161677 | chr19 | − | 0.000481576 | 0.017268979 | 0.162 |
| NLRX1 | ENSG00000160703 | chr11 | + | 0.000579012 | 0.019601762 | 0.152 |
| FDXR | ENSG00000161513 | chr17 | − | 0.001067903 | 0.02935361 | 0.14 |
| DPM1 | ENSG00000000419 | chr20 | − | 6.02E−06 | 0.000681358 | 0.131 |
| ZDHHC16 | ENSG00000171307 | chr10 | + | 0.001216023 | 0.032437883 | 0.112 |
| ALKBH4 | ENSG00000160993 | chr7 | − | 0.000183349 | 0.00860979 | 0.109 |
| RPS9 | ENSG00000170889 | chr19 | + | 0.000143135 | 0.007114834 | 0.097 |
| POLR2J3 | ENSG00000168255 | chr7 | − | 0.00027444 | 0.011701012 | 0.097 |
| ZNF302 | ENSG00000089335 | chr19 | + | 6.14E−05 | 0.00398418 | 0.097 |
| TENT4B | ENSG00000121274 | chr16 | + | 0.001285955 | 0.033860147 | 0.093 |
| ADGRE2 | ENSG00000127507 | chr19 | − | 4.35E−08 | 1.58E−05 | 0.09 |
| TKT | ENSG00000163931 | chr3 | − | 3.23E−05 | 0.002352711 | 0.087 |
| CARD8 | ENSG00000105483 | chr19 | − | 3.04E−11 | 3.09E−08 | 0.085 |
| RBM26 | ENSG00000139746 | chr13 | − | 0.000290148 | 0.012018713 | 0.078 |
| WSB1 | ENSG00000109046 | chr17 | + | 0.000152823 | 0.007486868 | 0.077 |

| Gene symbol | longExonStart_0base | longExonEnd | shortES | shortEE | flankingES | flankingEE |
|---|---|---|---|---|---|---|
| SNX5 | 17953995 | 17954231 | 17953995 | 17954223 | 17955364 | 17955475 |
| POLR2J3 | 102543524 | 102543702 | 102543524 | 102543661 | 102544705 | 102544776 |
| MPPE1 | 11885675 | 11885912 | 11885675 | 11885816 | 11886916 | 11887025 |
| AC016394.2 | 73253800 | 73254349 | 73253892 | 73254349 | 73252790 | 73253095 |
| DPM1 | 50940864 | 50940955 | 50940864 | 50940933 | 50941104 | 50941209 |
| E2F5 | 85212156 | 85212204 | 85212159 | 85212204 | 85209141 | 85209409 |
| PTPN7 | 202158117 | 202158320 | 202158117 | 202158248 | 202159280 | 202159454 |
| MTFP1 | 30427121 | 30427403 | 30427239 | 30427403 | 30426716 | 30426844 |
| TOR1AIP1 | 179889309 | 179889369 | 179889312 | 179889369 | 179884691 | 179884769 |
| POT1 | 124928814 | 124928999 | 124928814 | 124928996 | 124929793 | 124929825 |
| JOSD2 | 50510285 | 50510448 | 50510285 | 50510445 | 50511074 | 50511159 |
| NLRX1 | 119183117 | 119184016 | 119183820 | 119184016 | 119182093 | 119182345 |
| FDXR | 74866783 | 74866879 | 74866783 | 74866876 | 74872035 | 74872107 |
| DPM1 | 50940864 | 50940955 | 50940864 | 50940933 | 50941128 | 50941209 |
| ZDHHC16 | 97455638 | 97455783 | 97455713 | 97455783 | 97454713 | 97454799 |
| ALKBH4 | 102459603 | 102459801 | 102459603 | 102459710 | 102464713 | 102464863 |
| RPS9 | 54206552 | 54207647 | 54207397 | 54207647 | 54201486 | 54201609 |
| POLR2J3 | 102566996 | 102568184 | 102566996 | 102567083 | 102569834 | 102569924 |
| ZNF302 | 34683151 | 34683238 | 34683154 | 34683238 | 34682776 | 34682897 |
| TENT4B | 50225097 | 50225285 | 50225238 | 50225285 | 50224890 | 50224994 |
| ADGRE2 | 14755653 | 14755877 | 14755653 | 14755844 | 14756237 | 14756345 |
| TKT | 53237796 | 53237921 | 53237796 | 53237820 | 53240250 | 53240348 |
| CARD8 | 48232452 | 48232789 | 48232452 | 48232493 | 48234402 | 48234543 |
| RBM26 | 79365577 | 79365733 | 79365577 | 79365718 | 79366054 | 79366195 |
| WSB1 | 27307743 | 27309272 | 27309099 | 27309272 | 27306781 | 27306882 |

TABLE 10
Non-Limiting Examples of RNA Markers with Increased Alternative 3′ Splice Site (A5SS) in FXS

| Gene symbol | ENSEMBL Gene ID | chr | strand | PValue | FDR | IncLevelDifference |
|---|---|---|---|---|---|---|
| DDX60L | ENSG00000181381 | chr4 | − | 4.07E−10 | 2.96E−07 | −0.305 |
| ATP11A | ENSG00000068650 | chr13 | + | 1.90E−06 | 0.000288722 | −0.234 |
| SRGAP2 | ENSG00000266028 | chr1 | + | 1.09E−06 | 0.000194134 | −0.201 |
| CEACAM21 | ENSG00000007129 | chr19 | + | 8.31E−06 | 0.000855104 | −0.122 |
| COX18 | ENSG00000163626 | chr4 | − | 0.00049716 | 0.017529627 | −0.106 |
| WDR47 | ENSG00000085433 | chr1 | − | 0.000157279 | 0.007668305 | −0.105 |
| PATZ1 | ENSG00000100105 | chr22 | − | 0.000630999 | 0.020753112 | −0.103 |
| POLM | ENSG00000122678 | chr7 | − | 0.000406598 | 0.015121295 | −0.097 |
| CC2D1B | ENSG00000154222 | chr1 | − | 0.000429204 | 0.015675962 | −0.07 |
| CLK4 | ENSG00000113240 | chr5 | − | 0.000414994 | 0.015321707 | −0.069 |
| MIB2 | ENSG00000197530 | chr1 | + | 0.000892878 | 0.026187361 | −0.059 |
| PHF1 | ENSG00000112511 | chr6 | + | 5.69E−06 | 0.000666824 | −0.058 |
| KANSL1 | ENSG00000120071 | chr17 | − | 2.89E−05 | 0.002165813 | −0.051 |
| TCF3 | ENSG00000071564 | chr19 | − | 7.88E−07 | 0.000157428 | −0.051 |

| Gene symbol | longExonStart_0base | longExonEnd | shortES | shortEE | flankingES | flankingEE |
|---|---|---|---|---|---|---|
| DDX60L | 168395958 | 168396127 | 168395958 | 168396124 | 168400825 | 168400978 |
| ATP11A | 112881815 | 112887168 | 112881875 | 112887168 | 112878216 | 112878303 |
| SRGAP2 | 206401420 | 206401645 | 206401423 | 206401645 | 206393544 | 206393673 |
| CEACAM21 | 41579269 | 41579628 | 41579352 | 41579628 | 41577199 | 41577559 |
| COX18 | 73064777 | 73064902 | 73064777 | 73064899 | 73065249 | 73065413 |
| WDR47 | 109004591 | 109004718 | 109004591 | 109004715 | 109010915 | 109011718 |
| PATZ1 | 31328786 | 31328924 | 31328786 | 31328859 | 31335691 | 31335863 |
| POLM | 44074130 | 44074233 | 44074130 | 44074220 | 44074397 | 44074530 |
| CC2D1B | 52359704 | 52359883 | 52359704 | 52359836 | 52360073 | 52360233 |
| CLK4 | 178619837 | 178620673 | 178619837 | 178619896 | 178623255 | 178623416 |
| MIB2 | 1629033 | 1629311 | 1629132 | 1629311 | 1628488 | 1628722 |
| PHF1 | 33415234 | 33415329 | 33415260 | 33415329 | 33414724 | 33414829 |
| KANSL1 | 46034160 | 46034285 | 46034160 | 46034282 | 46038537 | 46038686 |
| TCF3 | 1619315 | 1619474 | 1619315 | 1619471 | 1619779 | 1619853 |
| DDX60L | 168395958 | 168396127 | 168395958 | 168396124 | 168400825 | 168400978 |
| ATP11A | 112881815 | 112887168 | 112881875 | 112887168 | 112878216 | 112878303 |
| SRGAP2 | 206401420 | 206401645 | 206401423 | 206401645 | 206393544 | 206393673 |
| CEACAM21 | 41579269 | 41579628 | 41579352 | 41579628 | 41577199 | 41577559 |

TABLE 15
FMR1 transcript identification and corresponding predicted
amino acid number of encoded protein from ENSEMBL.

| Transcript ID | Name | bp | Protein | Biotype |
|---|---|---|---|---|
| ENST00000370475.9 | FMR1-205 | 4441 | 632aa | Protein coding |
| ENST00000690137.1 | FMR1-226 | 4166 | 615aa | Protein coding |
| ENST00000218200.12 | FMR1-201 | 4333 | 611aa | Protein coding |
| ENST00000691111.1 | FMR1-228 | 4154 | 599aa | Protein coding |
| ENST00000687593.1 | FMR1-223 | 4159 | 594aa | Protein coding |
| ENST00000439526.6 | FMR1-207 | 3699 | 592aa | Protein coding |
| ENST00000370470.5 | FMR1-203 | 1774 | 590aa | Protein coding |
| ENST00000690216.1 | FMR1-227 | 4008 | 587aa | Protein coding |
| ENST00000440235.6 | FMR1-208 | 4271 | 586aa | Protein coding |
| ENST00000370477.5 | FMR1-206 | 3437 | 582aa | Protein coding |
| ENST00000686086.1 | FMR1-222 | 3995 | 570aa | Protein coding |
| ENST00000691214.1 | FMR1-229 | 4067 | 569aa | Protein coding |
| ENST00000495717.6 | FMR1-212 | 2874 | 561aa | Protein coding |
| ENST00000685491.1 | FMR1-221 | 4109 | 559aa | Protein coding |
| ENST00000621453.5 | FMR1-218 | 1827 | 548aa | Protein coding |
| ENST00000370471.7 | FMR1-204 | 4125 | 537aa | Protein coding |
| ENST00000616382.5 | FMR1-214 | 2799 | 536aa | Protein coding |
| ENST00000692108.1 | FMR1-232 | 4252 | 509aa | Protein coding |
| ENST00000689517.1 | FMR1-224 | 4484 | 460aa | Protein coding |
| ENST00000693512.1 | FMR1-235 | 3402 | 398aa | Protein coding |
| ENST00000334557.10 | FMR1-202 | 1295 | 297aa | Protein coding |
| ENST00000621987.5 | FMR1-219 | 2440 | 297aa | NMD |
| ENST00000616614.4 | FMR1-215 | 1409 | 76aa | NMD |
| ENST00000693452.1 | FMR1-234 | 4093 | 49aa | NMD |
| ENST00000692091.1 | FMR1-231 | 3908 | 49aa | NMD |
| ENST00000475038.3 | FMR1-209 | 2747 | 49aa | NMD |
| ENST00000621447.1 | FMR1-217 | 1832 | 31aa | Protein coding |
| ENST00000691793.1 | FMR1-230 | 5731 | | RI |
| ENST00000492846.2 | FMR1-211 | 5650 | | RI |
| ENST00000689570.1 | FMR1-225 | 5650 | | RI |
| ENST00000620828.4 | FMR1-216 | 4830 | | RI |
| ENST00000693079.1 | FMR1-233 | 4647 | | RI |
| ENST00000643620.1 | FMR1-220 | 1439 | | RI |
| ENST00000611273.1 | FMR1-213 | 564 | | RI |
| ENST00000478848.1 | FMR1-210 | 541 | | RI |

TABLE 16
DE_FXSvsTD

| | baseMean | log2FoldChange | lfcSE | stat | pvalue | padj |
|---|---|---|---|---|---|---|
| ANAPC1P2 | 41.8 | 24.58 | 3.17 | 7.75 | 9.19E−15 | 1.70E−10 |
| FAM3B | 58.2 | 5.71 | 1.04 | 5.47 | 4.46E−08 | 2.91E−04 |
| HMGB1P5 | 82.4 | −1.99 | 0.36 | −5.46 | 4.73E−08 | 2.91E−04 |
| CYP4F22 | 127.3 | −1.16 | 0.23 | −5.11 | 3.15E−07 | 1.46E−03 |
| RHOC | 2690.6 | −0.68 | 0.13 | −5.04 | 4.69E−07 | 1.56E−03 |
| AGAP1 | 342.6 | −0.99 | 0.20 | −5.02 | 5.07E−07 | 1.56E−03 |
| CFAP70 | 140.9 | 0.65 | 0.14 | 4.61 | 3.97E−06 | 1.04E−02 |
| KNDC1 | 135.2 | −1.33 | 0.29 | −4.57 | 4.82E−06 | 1.04E−02 |
| PRR5L | 1483.6 | −0.59 | 0.13 | −4.56 | 5.05E−06 | 1.04E−02 |
| ZNF365 | 105.0 | −1.01 | 0.22 | −4.48 | 7.31E−06 | 1.35E−02 |
| DUSP5 | 776.8 | −0.62 | 0.14 | −4.29 | 1.80E−05 | 2.76E−02 |
| ARHGAP24 | 601.7 | 0.54 | 0.13 | 4.28 | 1.84E−05 | 2.76E−02 |
| EPOP | 37.3 | −0.92 | 0.22 | −4.27 | 1.94E−05 | 2.76E−02 |
| MXRA7 | 949.6 | −1.38 | 0.32 | −4.24 | 2.24E−05 | 2.95E−02 |
| TOMM5 | 1220.3 | −0.26 | 0.06 | −4.20 | 2.67E−05 | 3.09E−02 |
| TRBV2 | 183.2 | −0.76 | 0.18 | −4.20 | 2.68E−05 | 3.09E−02 |
| NKG7 | 13411.5 | −0.80 | 0.20 | −4.10 | 4.18E−05 | 4.22E−02 |
| CLEC5A | 417.1 | 0.72 | 0.18 | 4.09 | 4.28E−05 | 4.22E−02 |
| TKTL1 | 319.1 | −0.90 | 0.22 | −4.09 | 4.33E−05 | 4.22E−02 |
| RAB25 | 21.6 | 1.21 | 0.30 | 4.07 | 4.69E−05 | 4.34E−02 |
| COL13A1 | 61.1 | −1.39 | 0.34 | −4.04 | 5.35E−05 | 4.71E−02 |
| RBM11 | 82.6 | 0.68 | 0.17 | 3.96 | 7.55E−05 | 6.23E−02 |
| AC008764.4 | 9.4 | 3.06 | 0.78 | 3.94 | 8.20E−05 | 6.23E−02 |
| CKB | 231.1 | −0.87 | 0.22 | −3.93 | 8.33E−05 | 6.23E−02 |
| GNGT2 | 367.7 | −0.44 | 0.11 | −3.93 | 8.43E−05 | 6.23E−02 |
| LAMC3 | 33.6 | 1.19 | 0.30 | 3.91 | 9.19E−05 | 6.40E−02 |
| NEFL | 189.0 | 0.90 | 0.23 | 3.90 | 9.44E−05 | 6.40E−02 |
| ZNF154 | 506.7 | 0.69 | 0.18 | 3.90 | 9.69E−05 | 6.40E−02 |
| C12orf75 | 1265.5 | −0.43 | 0.11 | −3.88 | 1.03E−04 | 6.40E−02 |
| MSC-AS1 | 79.1 | −1.20 | 0.31 | −3.88 | 1.05E−04 | 6.40E−02 |
| RPL39L | 76.6 | −0.90 | 0.23 | −3.87 | 1.09E−04 | 6.40E−02 |
| PPFIBP1 | 129.1 | 0.65 | 0.17 | 3.87 | 1.11E−04 | 6.40E−02 |
| ACOT7 | 284.8 | −0.43 | 0.11 | −3.85 | 1.18E−04 | 6.51E−02 |
| CDKN1C | 1312.3 | −0.91 | 0.24 | −3.84 | 1.21E−04 | 6.51E−02 |
| CKS1B | 292.4 | −0.33 | 0.09 | −3.84 | 1.25E−04 | 6.51E−02 |
| LINC00174 | 418.7 | 0.45 | 0.12 | 3.83 | 1.28E−04 | 6.51E−02 |
| PALM | 9.6 | −1.60 | 0.42 | −3.83 | 1.30E−04 | 6.51E−02 |
| CABP4 | 57.2 | −0.81 | 0.21 | −3.81 | 1.36E−04 | 6.63E−02 |
| EFNA5 | 29.3 | −1.30 | 0.34 | −3.80 | 1.46E−04 | 6.92E−02 |
| LYPD2 | 140.9 | −1.25 | 0.33 | −3.77 | 1.64E−04 | 7.57E−02 |
| DRAXIN | 171.1 | −1.19 | 0.32 | −3.76 | 1.73E−04 | 7.78E−02 |
| B3GAT1 | 380.0 | −1.29 | 0.35 | −3.73 | 1.93E−04 | 8.17E−02 |
| TPST2 | 4420.9 | −0.30 | 0.08 | −3.72 | 1.96E−04 | 8.17E−02 |
| CROCC2 | 21.0 | −1.41 | 0.38 | −3.72 | 1.99E−04 | 8.17E−02 |
| FCRL6 | 1356.3 | −0.79 | 0.21 | −3.72 | 2.01E−04 | 8.17E−02 |
| AC026369.3 | 23.0 | −1.28 | 0.34 | −3.71 | 2.03E−04 | 8.17E−02 |
| C19orf12 | 1194.0 | −0.27 | 0.07 | −3.69 | 2.24E−04 | 8.72E−02 |
| S100B | 576.5 | −1.95 | 0.53 | −3.69 | 2.26E−04 | 8.72E−02 |
| GAS1 | 40.1 | −1.22 | 0.33 | −3.68 | 2.33E−04 | 8.80E−02 |
| JAKMIP1 | 389.4 | −0.81 | 0.22 | −3.67 | 2.40E−04 | 8.88E−02 |
| LINC02345 | 34.2 | −1.17 | 0.32 | −3.65 | 2.61E−04 | 9.45E−02 |
| GPR153 | 141.6 | −0.92 | 0.25 | −3.63 | 2.81E−04 | 9.86E−02 |
| S1PR5 | 2888.0 | −0.72 | 0.20 | −3.63 | 2.87E−04 | 9.86E−02 |
| MIR3150BHG | 23.3 | 1.37 | 0.38 | 3.63 | 2.88E−04 | 9.86E−02 |

TABLE 17
SE_WBC_FXSvSTD

| GeneID | chr | strand | exonStart_0base | exonEnd | PValue | FDR | IncLevelDifference |
|---|---|---|---|---|---|---|---|
| PARP6 | chr15 | − | 72253949 | 72254070 | 4.13E−05 | 0.0047705 | −0.261 |
| NCALD | chr8 | − | 101887140 | 101887227 | 3.53E−07 | 9.83E−05 | −0.259 |
| PACRGL | chr4 | + | 20709682 | 20709773 | 1.40E−11 | 1.94E−08 | −0.256 |
| TCF7 | chr5 | + | 134144796 | 134144869 | 0.0003658 | 0.02606201 | −0.233 |
| ADAM15 | chr1 | + | 155061903 | 155062117 | 0.0005209 | 0.03425292 | −0.214 |
| LAIR2 | chr19 | + | 54509034 | 54509085 | 3.42E−05 | 0.00409749 | −0.213 |
| XPNPEP3 | chr22 | + | 40860677 | 40860793 | 9.18E−05 | 0.00887286 | −0.212 |
| ADAM15 | chr1 | + | 155061903 | 155061975 | 0.00013706 | 0.01211011 | −0.209 |
| POLR2J3 | chr7 | − | 102543524 | 102543661 | 0.00010433 | 0.00977206 | −0.201 |
| POLR2J3 | chr7 | − | 102543524 | 102543697 | 0.00033231 | 0.02419548 | −0.191 |
| LINC00937 | chr12 | − | 8370212 | 8370429 | 0.00027255 | 0.02051526 | −0.189 |
| WARS1 | chr14 | − | 100374135 | 100374244 | 0.00017291 | 0.01455804 | −0.189 |
| ADAM15 | chr1 | + | 155061417 | 155061489 | 3.33E−05 | 0.00400639 | −0.188 |
| ADAM15 | chr1 | + | 155061414 | 155061489 | 3.65E−05 | 0.00430797 | −0.185 |
| AL135818.1 | chr14 | + | 91247489 | 91247659 | 8.11E−07 | 0.00019871 | −0.182 |
| AC092070.2 | chr19 | + | 53200623 | 53200703 | 5.18E−06 | 0.00087303 | −0.18 |
| TRPT1 | chr11 | − | 64224099 | 64224162 | 0.0001713 | 0.01445306 | −0.18 |
| DST | chr6 | − | 56468981 | 56468999 | 5.43E−05 | 0.00585469 | −0.177 |
| WARS1 | chr14 | − | 100374135 | 100374265 | 4.29E−05 | 0.00488853 | −0.177 |
| MIR4435-2HG | chr2 | − | 111239801 | 111239996 | 2.76E−08 | 1.26E−05 | −0.177 |
| LRRFIP1 | chr2 | + | 237739231 | 237739309 | 0.00012458 | 0.01126227 | −0.167 |
| ADCY10P1 | chr6 | + | 41132874 | 41133000 | 0.00040264 | 0.02818434 | −0.161 |
| RNF19A | chr8 | − | 100300527 | 100300651 | 0.00036871 | 0.02622929 | −0.159 |
| DRAM2 | chr1 | − | 111139587 | 111139711 | 4.04E−05 | 0.00467528 | −0.157 |
| TRPV2 | chr17 | + | 16433573 | 16433698 | 0.00020503 | 0.01646975 | −0.156 |
| CAST | chr5 | + | 96740744 | 96740783 | 5.98E−11 | 6.79E−08 | −0.154 |
| C11orf80 | chr11 | + | 66756352 | 66756505 | 0.00032101 | 0.0235863 | −0.154 |
| ZNF266 | chr19 | − | 9433667 | 9433786 | 4.78E−05 | 0.00534689 | −0.154 |
| HMOX2 | chr16 | + | 4483636 | 4483738 | 1.82E−05 | 0.00248133 | −0.152 |
| JPX | chrX | + | 73997040 | 73997166 | 2.91E−13 | 6.92E−10 | −0.152 |
| ZNF266 | chr19 | − | 9433667 | 9433783 | 0.00010286 | 0.00967682 | −0.15 |
| DPM1 | chr20 | − | 50941128 | 50941209 | 4.29E−08 | 1.78E−05 | −0.144 |
| FRG1 | chr4 | + | 189955036 | 189955151 | 9.86E−05 | 0.00935286 | −0.141 |
| FRG1 | chr4 | + | 189953067 | 189953125 | 0.00049781 | 0.03310633 | −0.14 |
| COPS3 | chr17 | − | 17261599 | 17261692 | 4.89E−05 | 0.0054405 | −0.136 |
| METTL8 | chr2 | − | 171330558 | 171330616 | 1.60E−08 | 7.78E−06 | −0.135 |
| FCRLA | chr1 | + | 161710474 | 161710492 | 0.00043161 | 0.02964294 | −0.134 |
| WARS1 | chr14 | − | 100374135 | 100374244 | 6.02E−09 | 3.43E−06 | −0.134 |
| WARS1 | chr14 | − | 100374135 | 100374265 | 3.15E−09 | 1.91E−06 | −0.131 |
| PMS2CL | chr7 | + | 6733398 | 6733527 | 0.00072483 | 0.04450076 | −0.128 |
| MUC20-OT1 | chr3 | + | 195663623 | 195663767 | 5.26E−05 | 0.00572117 | −0.128 |
| ZNF273 | chr7 | + | 64918196 | 64918292 | 1.51E−07 | 5.06E−05 | −0.127 |
| NQO2 | chr6 | + | 3003668 | 3003789 | 1.12E−05 | 0.00165072 | −0.127 |
| AC141586.1 | chr16 | + | 2613959 | 2614068 | 3.03E−07 | 8.65E−05 | −0.126 |
| PNPO | chr17 | + | 47945558 | 47945612 | 1.85E−05 | 0.0025123 | −0.125 |
| BFAR | chr16 | + | 14649803 | 14649973 | 2.33E−07 | 7.10E−05 | −0.12 |
| GBAP1 | chr1 | − | 155218387 | 155218479 | 1.00E−06 | 0.00023493 | −0.119 |
| RAB18 | chr10 | + | 27531528 | 27531615 | 1.38E−06 | 0.00030182 | −0.115 |
| GPS1 | chr17 | + | 82052255 | 82052459 | 0.00034465 | 0.02477726 | −0.114 |
| TAF5 | chr10 | + | 103378234 | 103378550 | 2.32E−05 | 0.00299072 | −0.11 |
| HMOX2 | chr16 | + | 4483636 | 4483754 | 3.00E−05 | 0.00368734 | −0.109 |
| PACRGL | chr4 | + | 20712787 | 20712922 | 7.13E−12 | 1.09E−08 | −0.109 |
| IZUMO4 | chr19 | + | 2097928 | 2098127 | 6.33E−10 | 4.73E−07 | −0.109 |
| ANKS3 | chr16 | − | 4726978 | 4727177 | 1.48E−06 | 0.0003182 | −0.106 |
| SEPTIN2 | chr2 | + | 241316421 | 241316540 | 3.83E−05 | 0.00448192 | −0.106 |
| YY1AP1 | chr1 | − | 155680381 | 155680456 | 3.89E−07 | 0.00010641 | −0.106 |
| OFD1 | chrX | + | 13752703 | 13753441 | 9.82E−06 | 0.00148129 | −0.104 |
| AC012184.3 | chr16 | − | 70341939 | 70342099 | 1.57E−05 | 0.00217971 | −0.102 |
| SEPTIN2 | chr2 | + | 241316421 | 241316575 | 0.00031701 | 0.02334237 | −0.1 |
| LAIR2 | chr19 | + | 54507890 | 54508184 | 8.07E−12 | 1.20E−08 | −0.099 |
| SEPTIN2 | chr2 | + | 241316421 | 241316555 | 0.00052281 | 0.03430146 | −0.099 |
| LDAH | chr2 | − | 20739970 | 20740205 | 4.09E−05 | 0.00472452 | −0.099 |
| CDC42BPG | chr11 | − | 64827685 | 64827783 | 3.34E−11 | 4.14E−08 | −0.099 |
| ZNF273 | chr7 | + | 64918196 | 64918292 | 1.40E−07 | 4.80E−05 | −0.098 |
| VMP1 | chr17 | + | 59834011 | 59834050 | 0.00046842 | 0.03154095 | −0.096 |
| ATP6AP1L | chr5 | + | 82304933 | 82305481 | 0.00046338 | 0.03133037 | −0.096 |
| COMMD2 | chr3 | − | 149750256 | 149750382 | 0.00060936 | 0.03880713 | −0.095 |
| PPRC1 | chr10 | + | 102138878 | 102138980 | 6.73E−05 | 0.00694378 | −0.095 |
| RHBDF2 | chr17 | − | 76487973 | 76488132 | 8.86E−06 | 0.0013551 | −0.095 |
| ZNF56 | chr19 | + | 19786608 | 19786691 | 0.00049007 | 0.03267808 | −0.095 |
| SRSF4 | chr1 | − | 29166728 | 29166912 | 9.13E−12 | 1.33E−08 | −0.094 |
| ZNF529-AS1 | chr19 | + | 36594155 | 36594299 | 4.85E−05 | 0.00540113 | −0.092 |
| TNK2 | chr3 | − | 195882444 | 195882527 | 0.00046703 | 0.03149965 | −0.092 |
| ANKS3 | chr16 | − | 4726658 | 4726776 | 1.18E−06 | 0.00026688 | −0.091 |
| SUCO | chr1 | + | 172585857 | 172585948 | 8.87E−05 | 0.00866587 | −0.09 |
| TBC1D19 | chr4 | + | 26717932 | 26718017 | 2.91E−08 | 1.31E−05 | −0.089 |
| WARS1 | chr14 | − | 100374135 | 100374179 | 3.29E−10 | 2.78E−07 | −0.089 |
| ITGB7 | chr12 | − | 53201146 | 53201194 | 8.02E−06 | 0.0012518 | −0.089 |
| CYB5RL | chr1 | − | 54190747 | 54190896 | 0.00010043 | 0.0094911 | −0.088 |
| WARS1 | chr14 | − | 100374135 | 100374244 | 9.70E−12 | 1.40E−08 | −0.086 |
| IFI44L | chr1 | + | 78627905 | 78628393 | 2.03E−05 | 0.00268837 | −0.086 |
| TGIF1 | chr18 | + | 3450321 | 3450505 | 7.31E−05 | 0.0074397 | −0.085 |
| ZBTB25 | chr14 | − | 64499440 | 64499563 | 2.44E−06 | 0.00047767 | −0.084 |
| FKRP | chr19 | + | 46748514 | 46748621 | 0.00019209 | 0.01575417 | −0.083 |
| NSUN5P1 | chr7 | + | 75414639 | 75414798 | 0.00080454 | 0.04836462 | −0.082 |
| ADAM15 | chr1 | + | 155060762 | 155060832 | 4.27E−07 | 0.00011535 | −0.082 |
| PRKCQ-AS1 | chr10 | + | 6615369 | 6615637 | 1.31E−12 | 2.48E−09 | −0.082 |
| TRPT1 | chr11 | − | 64224099 | 64224210 | 3.63E−11 | 4.46E−08 | −0.082 |
| NCAPG2 | chr7 | − | 158650831 | 158650972 | 4.67E−05 | 0.00523241 | −0.081 |
| IP6K2 | chr3 | − | 48694458 | 48694525 | 0.00031741 | 0.02336111 | −0.081 |
| ALS2CL | chr3 | − | 46683129 | 46683326 | 5.57E−06 | 0.00092703 | −0.081 |
| NFS1 | chr20 | − | 35675044 | 35675202 | 3.47E−05 | 0.00414001 | −0.08 |
| LINC00174 | chr7 | − | 66476258 | 66476327 | 1.41E−06 | 0.00030682 | −0.08 |
| CYRIB | chr8 | − | 129948965 | 129949067 | 1.23E−07 | 4.28E−05 | −0.079 |
| CDC27 | chr17 | − | 47171916 | 47172064 | 0.00075957 | 0.04618237 | −0.079 |
| ZNF202 | chr11 | − | 123727475 | 123727595 | 0.00013216 | 0.01174517 | −0.079 |
| GOLGA2P5 | chr12 | − | 100168451 | 100168633 | 1.97E−08 | 9.30E−06 | −0.079 |
| ZNF85 | chr19 | + | 20944251 | 20944296 | 0.00018604 | 0.0153503 | −0.079 |
| FBXW8 | chr12 | + | 116985205 | 116985402 | 1.98E−10 | 1.81E−07 | −0.078 |
| COA8 | chr14 | + | 103571622 | 103571820 | 0.00045145 | 0.03068469 | −0.078 |
| NSRP1 | chr17 | + | 30163066 | 30163162 | 7.97E−05 | 0.00795361 | −0.078 |
| KLRC4-KLRK1 | chr12 | − | 10391834 | 10391891 | 7.17E−05 | 0.00732472 | −0.077 |
| OFD1 | chrX | + | 13753367 | 13753441 | 3.23E−06 | 0.00059739 | −0.075 |
| FKRP | chr19 | + | 46748514 | 46748884 | 0.00011155 | 0.01033948 | −0.075 |
| BCLAF3 | chrX | − | 19929784 | 19929940 | 0.00060414 | 0.03851649 | −0.075 |
| SRPK2 | chr7 | − | 105268805 | 105268869 | 1.22E−06 | 0.00027299 | −0.073 |
| WARS1 | chr14 | − | 100374135 | 100374265 | 1.03E−13 | 2.84E−10 | −0.073 |
| HPCAL1 | chr2 | + | 10426219 | 10426495 | 1.27E−06 | 0.00028331 | −0.073 |
| RXYLT1 | chr12 | + | 63782540 | 63782704 | 0.00069902 | 0.04321791 | −0.073 |
| CARMIL1 | chr6 | + | 25604811 | 25604893 | 0.00024006 | 0.01852197 | −0.072 |
| RAD51C | chr17 | + | 58705877 | 58706022 | 9.33E−05 | 0.00897361 | −0.072 |
| HEATR6 | chr17 | − | 60046024 | 60046099 | 3.33E−07 | 9.41E−05 | −0.072 |
| CROCC | chr1 | + | 16950952 | 16951122 | 1.45E−08 | 7.20E−06 | −0.071 |
| AL732372.2 | chr1 | − | 502861 | 502955 | 4.19E−06 | 0.00073262 | −0.071 |
| DTNB | chr2 | − | 25607235 | 25607321 | 1.51E−05 | 0.00211344 | −0.071 |
| PLPP1 | chr5 | − | 55475298 | 55475450 | 2.31E−05 | 0.00298281 | −0.07 |
| CTNS | chr17 | + | 3647443 | 3647522 | 2.82E−06 | 0.00053717 | −0.07 |
| COP1 | chr1 | − | 176175909 | 176176007 | 6.87E−05 | 0.00706358 | −0.07 |
| NEK3 | chr13 | − | 52135728 | 52135863 | 2.07E−05 | 0.00273898 | −0.07 |
| POLA1 | chrX | + | 24745417 | 24745542 | 0.00041351 | 0.028716 | −0.068 |
| LSM14B | chr20 | + | 62126225 | 62126439 | 0.0001512 | 0.01303926 | −0.068 |
| CCDC18-AS1 | chr1 | − | 93269682 | 93269758 | 0.00018251 | 0.01513776 | −0.068 |
| LYPLAL1-DT | chr1 | − | 219156119 | 219156199 | 0.00057693 | 0.03713179 | −0.068 |
| SLC25A43 | chrX | + | 119410189 | 119410362 | 1.91E−06 | 0.00039254 | −0.067 |
| BRAF | chr7 | − | 140808236 | 140808316 | 2.75E−05 | 0.00342273 | −0.067 |
| STX3 | chr11 | + | 59795601 | 59795712 | 5.00E−05 | 0.00551868 | −0.067 |
| PPFIA1 | chr11 | + | 70350993 | 70351023 | 2.51E−05 | 0.00318434 | −0.067 |
| UBR2 | chr6 | + | 42592150 | 42592229 | 9.35E−05 | 0.00897617 | −0.066 |
| SRP14-AS1 | chr15 | + | 40060096 | 40060263 | 0.00014309 | 0.0124806 | −0.065 |
| ZBTB7B | chr1 | + | 155010915 | 155011015 | 4.05E−10 | 3.31E−07 | −0.065 |
| BCLAF3 | chrX | − | 19935808 | 19935898 | 0.00020359 | 0.01641398 | −0.064 |
| SFI1 | chr22 | + | 31603743 | 31603819 | 8.00E−12 | 1.20E−08 | −0.063 |
| FKRP | chr19 | + | 46748514 | 46748717 | 0.00019606 | 0.01598112 | −0.063 |
| STAG3L3 | chr7 | − | 72999054 | 72999162 | 1.40E−05 | 0.00199305 | −0.063 |
| IMMP1L | chr11 | − | 31473723 | 31473786 | 2.67E−11 | 3.43E−08 | −0.063 |
| PNPO | chr17 | + | 47945860 | 47945989 | 3.41E−06 | 0.00062323 | −0.063 |
| BBS2 | chr16 | − | 56500853 | 56500977 | 5.46E−05 | 0.0058751 | −0.063 |
| PIGT | chr20 | + | 45418771 | 45418979 | 0.0006181 | 0.03923712 | −0.062 |
| PAAF1 | chr11 | + | 73878782 | 73878819 | 1.82E−06 | 0.0003766 | −0.062 |
| NQO2 | chr6 | + | 3003668 | 3003789 | 5.29E−10 | 4.15E−07 | −0.062 |
| NT5DC2 | chr3 | − | 52524816 | 52524881 | 1.97E−05 | 0.00263448 | −0.062 |
| PRKCQ-AS1 | chr10 | + | 6608347 | 6608408 | 1.22E−12 | 2.38E−09 | −0.062 |
| OSBPL5 | chr11 | − | 3120543 | 3120624 | 0.00038382 | 0.0271477 | −0.062 |
| MAPKAPK5 | chr12 | + | 111868752 | 111868861 | 1.45E−05 | 0.00204087 | −0.062 |
| ITGAE | chr17 | − | 3723287 | 3723383 | 1.81E−07 | 5.85E−05 | −0.062 |
| PCBP1-AS1 | chr2 | − | 70081369 | 70081490 | 7.56E−06 | 0.00119125 | −0.062 |
| FAM229B | chr6 | + | 112097040 | 112097201 | 1.81E−05 | 0.00246972 | −0.061 |
| ZSCAN25 | chr7 | + | 99621372 | 99621574 | 9.90E−06 | 0.00149139 | −0.061 |
| OBSCN | chr1 | + | 228299881 | 228300145 | 2.61E−05 | 0.00329903 | −0.061 |
| HDAC10 | chr22 | − | 50247691 | 50247776 | 1.96E−05 | 0.00262346 | −0.061 |
| ACAD10 | chr12 | + | 111727961 | 111728143 | 7.99E−08 | 2.98E−05 | −0.061 |
| JPX | chrX | + | 73994839 | 73994961 | 2.10E−11 | 2.78E−08 | −0.061 |
| NUBP2 | chr16 | + | 1786756 | 1786955 | 0.00073545 | 0.04498019 | −0.061 |
| FRG1BP | chr20 | + | 30391196 | 30391308 | 0.00043121 | 0.02962953 | −0.06 |
| PDPR | chr16 | + | 70130422 | 70130544 | 0.0007527 | 0.04582763 | −0.06 |
| RAB3IP | chr12 | + | 69800208 | 69800332 | 2.98E−10 | 2.61E−07 | −0.06 |
| HEATR3 | chr16 | + | 50066366 | 50066539 | 4.11E−07 | 0.00011123 | −0.06 |
| LSM14B | chr20 | + | 62126225 | 62126439 | 2.59E−05 | 0.00327671 | −0.058 |
| POGLUT3 | chr11 | − | 108482005 | 108482222 | 2.48E−05 | 0.00315388 | −0.058 |
| TPP2 | chr13 | + | 102651338 | 102651397 | 2.08E−07 | 6.48E−05 | −0.058 |
| TRPM7 | chr15 | − | 50578558 | 50578664 | 2.19E−05 | 0.00287558 | −0.058 |
| NDUFAF5 | chr20 | + | 13794837 | 13794941 | 0.00013562 | 0.01199553 | −0.057 |
| WARS1 | chr14 | − | 100374135 | 100374220 | 1.07E−10 | 1.08E−07 | −0.057 |
| RAB3IP | chr12 | + | 69800208 | 69800337 | 3.88E−10 | 3.20E−07 | −0.057 |
| LARGE2 | chr11 | + | 45928176 | 45928372 | 1.94E−06 | 0.00039701 | −0.057 |
| DPAGT1 | chr11 | − | 119097854 | 119097925 | 0.00058182 | 0.0373643 | −0.057 |
| DUSP16 | chr12 | − | 12500518 | 12500682 | 8.11E−08 | 3.01E−05 | −0.057 |
| KDM3B | chr5 | + | 138386288 | 138386621 | 1.42E−05 | 0.00201007 | −0.056 |
| KMT2B | chr19 | + | 35719468 | 35719541 | 3.80E−06 | 0.00067495 | −0.056 |
| NQO2 | chr6 | + | 3004494 | 3004651 | 3.73E−07 | 0.00010259 | −0.056 |
| COA1 | chr7 | − | 43650611 | 43650712 | 1.57E−14 | 5.19E−11 | −0.056 |
| GARS1-DT | chr7 | − | 30551317 | 30551395 | 5.76E−06 | 0.00095019 | −0.055 |
| TPP2 | chr13 | + | 102651358 | 102651397 | 0.00039893 | 0.02799129 | −0.055 |
| NMRK1 | chr9 | − | 75069741 | 75069813 | 0 | 0 | −0.055 |
| MMEL1 | chr1 | − | 2591931 | 2592027 | 8.06E−06 | 0.00125569 | −0.055 |
| ABCA11P | chr4 | − | 437445 | 437511 | 0.00022197 | 0.01743278 | −0.055 |
| UPP1 | chr7 | + | 48103296 | 48103411 | 2.29E−07 | 7.03E−05 | −0.055 |
| PHF1 | chr6 | + | 33414954 | 33415081 | 4.31E−06 | 0.00075119 | −0.055 |
| TMEM218 | chr11 | − | 125110643 | 125110746 | 3.02E−05 | 0.00369849 | −0.055 |
| MAP7D3 | chrX | − | 136231543 | 136232097 | 3.35E−07 | 9.45E−05 | −0.054 |
| ZNF653 | chr19 | − | 11500677 | 11500815 | 0.00045619 | 0.0309349 | −0.054 |
| AC010175.1 | chr12 | + | 9247580 | 9247677 | 0.00035803 | 0.02558081 | −0.054 |
| DHODH | chr16 | + | 72022361 | 72022475 | 8.17E−07 | 0.00019983 | −0.054 |
| TUBGCP6 | chr22 | − | 50220191 | 50221874 | 6.63E−05 | 0.00686126 | −0.054 |
| TUBGCP6 | chr22 | − | 50221274 | 50221874 | 0.00012187 | 0.01108574 | −0.054 |
| TTLL3 | chr3 | + | 9817644 | 9817759 | 4.18E−06 | 0.00073262 | −0.053 |
| TEX10 | chr9 | − | 100308499 | 100308681 | 2.70E−07 | 7.88E−05 | −0.053 |
| SPATS2 | chr12 | + | 49486249 | 49486348 | 0.00012318 | 0.01117043 | −0.053 |
| ZNF76 | chr6 | + | 35293750 | 35293915 | 0.00082746 | 0.04947171 | −0.052 |
| FKRP | chr19 | + | 46748514 | 46748665 | 6.12E−06 | 0.0009992 | −0.052 |
| SAR1B | chr5 | − | 134632076 | 134632195 | 3.18E−05 | 0.0038478 | −0.052 |
| ZEB2 | chr2 | − | 144398300 | 144400270 | 3.78E−05 | 0.00444011 | −0.052 |
| COA1 | chr7 | − | 43657215 | 43657269 | 0.00051797 | 0.03412416 | −0.052 |
| SLC44A2 | chr19 | + | 10635430 | 10635515 | 0.00027587 | 0.02071182 | −0.052 |
| BCLAF3 | chrX | − | 19935808 | 19935898 | 0.00011236 | 0.01039656 | −0.052 |
| TRMT2B | chrX | − | 101041316 | 101041371 | 1.46E−06 | 0.00031309 | −0.052 |
| PITPNM2 | chr12 | − | 123013827 | 123014042 | 0.00064725 | 0.04070432 | −0.052 |
| IFT52 | chr20 | + | 43605001 | 43605216 | 0.00072713 | 0.04462656 | −0.051 |
| P4HA1 | chr10 | − | 73044980 | 73045051 | 0.0001243 | 0.01124914 | −0.051 |
| NUTM2A-AS1 | chr10 | − | 87241181 | 87245855 | 1.07E−06 | 0.00024572 | −0.051 |
| SIGIRR | chr11 | − | 406875 | 406993 | 9.72E−05 | 0.00925338 | −0.051 |
| ACAD10 | chr12 | + | 111702161 | 111702310 | 0.00018525 | 0.01530743 | −0.051 |
| ZMYM5 | chr13 | − | 19837655 | 19837821 | 1.92E−08 | 9.13E−06 | −0.051 |
| AC243960.1 | chr19 | + | 41551179 | 41551494 | 0.00041006 | 0.02852178 | −0.051 |
| ZMYM3 | chrX | − | 71251177 | 71251250 | 1.94E−07 | 6.19E−05 | −0.05 |
| VNN2 | chr6 | − | 132752672 | 132752749 | 0.00014537 | 0.01265431 | −0.05 |
| SERINC5 | chr5 | − | 80169334 | 80169546 | 0.00037287 | 0.02649055 | −0.05 |
| POGLUT3 | chr11 | − | 108479300 | 108479495 | 1.75E−06 | 0.00036475 | −0.05 |
| LAIR1 | chr19 | − | 54364294 | 54364486 | 0.00073871 | 0.04510092 | −0.05 |
| BAZ2A | chr12 | − | 56615013 | 56615128 | 0.00048423 | 0.03232551 | −0.05 |
| FCGRT | chr19 | + | 49525456 | 49525561 | 0.00035582 | 0.02549548 | −0.05 |
| GGA1 | chr22 | + | 37613118 | 37613191 | 8.63E−07 | 0.0002093 | 0.05 |
| TMEM161B- | chr5 | + | 88285743 | 88285851 | 0.00038272 | 0.02708038 | 0.05 |
| AS1 | | | | | | | |
| KRIT1 | chr7 | − | 92245426 | 92245595 | 2.69E−06 | 0.00051737 | 0.05 |
| SAMD4B | chr19 | + | 39351816 | 39352374 | 2.97E−05 | 0.00365176 | 0.05 |
| FRG1CP | chr20 | − | 28590556 | 28590614 | 2.46E−06 | 0.00048012 | 0.05 |
| ACADVL | chr17 | + | 7220463 | 7220529 | 0.00031328 | 0.02311622 | 0.05 |
| KIF27 | chr9 | − | 83880296 | 83880494 | 2.29E−05 | 0.00296755 | 0.05 |
| NBPF12 | chr1 | + | 146989573 | 146989746 | 1.43E−10 | 1.41E−07 | 0.05 |
| NUP62 | chr19 | − | 49927693 | 49927848 | 0.00014724 | 0.0127722 | 0.05 |
| CEP295 | chr11 | + | 93724253 | 93724375 | 1.70E−12 | 3.16E−09 | 0.05 |
| RHNO1 | chr12 | + | 2885282 | 2885534 | 0.00019107 | 0.01569934 | 0.05 |
| FANCI | chr15 | + | 89293832 | 89293997 | 0.00021701 | 0.01717253 | 0.05 |
| RMDN1 | chr8 | − | 86474819 | 86474914 | 5.68E−10 | 4.36E−07 | 0.05 |
| UBA52 | chr19 | + | 18573292 | 18573403 | 0.00068996 | 0.04276343 | 0.05 |
| ELF4 | chrX | − | 130081255 | 130081312 | 0.00056335 | 0.03639137 | 0.05 |
| MVK | chr12 | + | 109579801 | 109579946 | 9.67E−05 | 0.00923448 | 0.051 |
| WASHC2A | chr10 | + | 50127159 | 50127222 | 4.22E−05 | 0.00483907 | 0.051 |
| TMEM79 | chr1 | + | 156285183 | 156285267 | 4.51E−05 | 0.00510788 | 0.051 |
| BANP | chr16 | + | 88027482 | 88027650 | 1.03E−10 | 1.05E−07 | 0.051 |
| EBLN3P | chr9 | + | 37080341 | 37080536 | 5.97E−06 | 0.00098069 | 0.051 |
| ITGB3BP | chr1 | − | 63510064 | 63510181 | 6.66E−16 | 2.79E−12 | 0.051 |
| TMEM267 | chr5 | − | 43453657 | 43454043 | 0.00076186 | 0.04625754 | 0.051 |
| SLC25A37 | chr8 | + | 23543087 | 23543211 | 0.0003385 | 0.02448361 | 0.051 |
| SERPING1 | chr11 | + | 57599863 | 57600377 | 0.00046854 | 0.03154095 | 0.051 |
| AC087632.2 | chr15 | − | 64207508 | 64207594 | 0.00073729 | 0.0450565 | 0.051 |
| TGFB3 | chr14 | − | 75960268 | 75960386 | 3.21E−05 | 0.0038857 | 0.051 |
| TPT1-AS1 | chr13 | + | 45383759 | 45383838 | 9.55E−06 | 0.00144378 | 0.052 |
| LGMN | chr14 | − | 92706482 | 92706653 | 4.01E−06 | 0.0007065 | 0.052 |
| INO80C | chr18 | − | 35479299 | 35479411 | 1.46E−09 | 9.93E−07 | 0.052 |
| INO80C | chr18 | − | 35480452 | 35480563 | 1.92E−05 | 0.00258784 | 0.052 |
| TAF11 | chr6 | − | 34879966 | 34880063 | 0.00045678 | 0.03096276 | 0.052 |
| ATRIP | chr3 | + | 48461360 | 48461445 | 0.00076043 | 0.04619117 | 0.052 |
| NCOR2 | chr12 | − | 124457105 | 124457162 | 5.36E−05 | 0.00579242 | 0.052 |
| GTDC1 | chr2 | − | 143952772 | 143952843 | 0.00073332 | 0.04488078 | 0.052 |
| CPVL | chr7 | − | 29066022 | 29066121 | 1.65E−06 | 0.00034657 | 0.052 |
| PVT1 | chr8 | + | 128070159 | 128070272 | 2.50E−07 | 7.46E−05 | 0.052 |
| RNPS1 | chr16 | − | 2267174 | 2267218 | 3.71E−06 | 0.0006642 | 0.052 |
| A1BG-AS1 | chr19 | + | 58353320 | 58353474 | 1.29E−06 | 0.00028542 | 0.052 |
| PCBP1-AS1 | chr2 | − | 70053730 | 70053792 | 0.00013037 | 0.01163059 | 0.052 |
| CAMLG | chr5 | + | 134741062 | 134741523 | 1.57E−06 | 0.00033148 | 0.053 |
| TMBIM1 | chr2 | − | 218285762 | 218285938 | 0.00058267 | 0.03740563 | 0.053 |
| SLC15A2 | chr3 | + | 121940383 | 121940488 | 3.62E−07 | 0.00010052 | 0.053 |
| DENND4C | chr9 | + | 19371755 | 19371820 | 8.89E−07 | 0.00021467 | 0.053 |
| MAP3K20 | chr2 | + | 173090997 | 173091190 | 2.15E−14 | 6.53E−11 | 0.053 |
| RAB4A | chr1 | + | 229288728 | 229288843 | 0.00041343 | 0.028716 | 0.053 |
| NOD2 | chr16 | + | 50707854 | 50707960 | 8.66E−15 | 3.11E−11 | 0.053 |
| ERMARD | chr6 | + | 169753943 | 169754032 | 3.14E−10 | 2.68E−07 | 0.054 |
| ZNF354B | chr5 | + | 178885613 | 178885737 | 4.82E−11 | 5.72E−08 | 0.054 |
| NOTCH2 | chr1 | − | 119948413 | 119948566 | 2.89E−12 | 4.93E−09 | 0.054 |
| IP6K2 | chr3 | − | 48715314 | 48715527 | 0.00065065 | 0.04087433 | 0.054 |
| HLTF | chr3 | − | 149041489 | 149041668 | 4.24E−05 | 0.00485771 | 0.054 |
| TRAF3IP2-AS1 | chr6 | + | 111574620 | 111574802 | 0.00038608 | 0.02727479 | 0.054 |
| CCNL1 | chr3 | − | 157157010 | 157157083 | 0.00057255 | 0.03689057 | 0.054 |
| SYTL2 | chr11 | − | 85714412 | 85714507 | 4.65E−05 | 0.00522027 | 0.054 |
| DGUOK | chr2 | + | 73938909 | 73939022 | 0.00059549 | 0.0380615 | 0.054 |
| AGPAT5 | chr8 | + | 6747669 | 6747828 | 0.0001124 | 0.01039656 | 0.055 |
| VNN2 | chr6 | − | 132760631 | 132760803 | 0.00021126 | 0.01683401 | 0.055 |
| R3HCC1L | chr10 | + | 98162882 | 98162975 | 9.11E−07 | 0.00021858 | 0.055 |
| KIF27 | chr9 | − | 83859155 | 83859371 | 3.06E−06 | 0.00057281 | 0.055 |
| LINC00963 | chr9 | + | 129493415 | 129493595 | 5.11E−05 | 0.00560086 | 0.055 |
| JAK3 | chr19 | − | 17839179 | 17839293 | 0.00046688 | 0.03149965 | 0.055 |
| PPP4R1L | chr20 | − | 58236153 | 58236274 | 9.49E−07 | 0.00022605 | 0.055 |
| HM13 | chr20 | + | 31567639 | 31567740 | 3.04E−05 | 0.00371772 | 0.055 |
| HM13 | chr20 | + | 31567639 | 31567806 | 0.00024524 | 0.01881368 | 0.055 |
| GMFG | chr19 | − | 39335260 | 39335310 | 7.40E−07 | 0.0001842 | 0.055 |
| GGA1 | chr22 | + | 37613023 | 37613191 | 1.38E−08 | 6.96E−06 | 0.056 |
| CCM2 | chr7 | + | 45070093 | 45070168 | 0.0002009 | 0.0162487 | 0.056 |
| LY96 | chr8 | + | 74004795 | 74004885 | 2.58E−07 | 7.66E−05 | 0.056 |
| EIF4G3 | chr1 | − | 21002712 | 21002808 | 4.69E−13 | 1.03E−09 | 0.056 |
| SNX25 | chr4 | + | 185258847 | 185259064 | 2.92E−06 | 0.00055518 | 0.056 |
| METTL15 | chr11 | + | 28122141 | 28122180 | 2.46E−06 | 0.00048012 | 0.056 |
| CEP290 | chr12 | − | 88054339 | 88054413 | 0.00028011 | 0.02100352 | 0.056 |
| MAP4 | chr3 | − | 47867245 | 47867338 | 1.96E−05 | 0.00262346 | 0.056 |
| DPEP2 | chr16 | − | 67992949 | 67993257 | 1.30E−05 | 0.00187949 | 0.056 |
| TCEA3 | chr1 | − | 23408663 | 23408726 | 1.82E−09 | 1.21E−06 | 0.057 |
| ST6GALNAC4 | chr9 | − | 127909950 | 127910528 | 1.60E−08 | 7.79E−06 | 0.057 |
| CASP5 | chr11 | − | 105003273 | 105003383 | 0.00012589 | 0.01134592 | 0.057 |
| DLGAP4 | chr20 | + | 36465264 | 36465367 | 2.58E−08 | 1.18E−05 | 0.057 |
| RBIS | chr8 | − | 85219235 | 85219360 | 3.77E−12 | 6.29E−09 | 0.057 |
| LUCAT1 | chr5 | − | 91303872 | 91303971 | 4.81E−05 | 0.0053638 | 0.057 |
| PSTK | chr10 | + | 122982732 | 122983024 | 1.29E−10 | 1.27E−07 | 0.057 |
| GLT1D1 | chr12 | + | 128914932 | 128914989 | 3.25E−05 | 0.00392457 | 0.057 |
| GTF2I | chr7 | + | 74744138 | 74744250 | 7.66E−05 | 0.00771126 | 0.057 |
| NRDE2 | chr14 | − | 90317703 | 90317820 | 3.30E−06 | 0.00060518 | 0.057 |
| ST3GAL2 | chr16 | − | 70400695 | 70401094 | 1.71E−05 | 0.00235683 | 0.057 |
| SNAP23 | chr15 | + | 42529674 | 42529819 | 7.93E−05 | 0.00792063 | 0.057 |
| AC138894.1 | chr16 | − | 28491481 | 28491513 | 0.00033205 | 0.02418896 | 0.057 |
| LMAN2L | chr2 | − | 96733518 | 96733601 | 1.71E−05 | 0.00235722 | 0.057 |
| CERS4 | chr19 | + | 8257878 | 8257985 | 0.0001142 | 0.01051884 | 0.057 |
| PGAP2 | chr11 | + | 3822908 | 3822993 | 0.00024356 | 0.0187041 | 0.058 |
| SLC12A2 | chr5 | + | 128177104 | 128177152 | 1.89E−05 | 0.00255504 | 0.058 |
| CAMK4 | chr5 | + | 111376859 | 111376942 | 1.77E−05 | 0.0024254 | 0.059 |
| ABRAXAS1 | chr4 | − | 83470202 | 83470396 | 3.64E−05 | 0.00429266 | 0.059 |
| FCRL2 | chr1 | − | 157770408 | 157770666 | 3.04E−10 | 2.63E−07 | 0.059 |
| TANGO2 | chr22 | + | 20061529 | 20061683 | 0.00041414 | 0.02874869 | 0.059 |
| GGCT | chr7 | − | 30500535 | 30500681 | 0.00041456 | 0.02876643 | 0.059 |
| AP001781.2 | chr11 | − | 111876480 | 111876639 | 7.12E−08 | 2.70E−05 | 0.059 |
| NOD2 | chr16 | + | 50716890 | 50716974 | 9.32E−05 | 0.00897361 | 0.059 |
| FYN | chr6 | − | 111754433 | 111754677 | 2.00E−05 | 0.00265702 | 0.059 |
| CYBC1 | chr17 | − | 82449430 | 82449956 | 0.00083258 | 0.04972718 | 0.059 |
| CCDC191 | chr3 | − | 114046590 | 114046732 | 0.00033287 | 0.0242138 | 0.059 |
| ABHD12 | chr20 | − | 25314924 | 25314970 | 5.56E−13 | 1.15E−09 | 0.06 |
| FOPNL | chr16 | − | 15873490 | 15873627 | 0.00081018 | 0.04858225 | 0.06 |
| NEXN | chr1 | + | 77935822 | 77936044 | 6.34E−09 | 3.58E−06 | 0.06 |
| HFE | chr6 | + | 26090840 | 26091104 | 3.03E−09 | 1.85E−06 | 0.06 |
| TRMT2B | chrX | − | 101041316 | 101041371 | 2.02E−08 | 9.45E−06 | 0.06 |
| ABCD4 | chr14 | − | 74299547 | 74299675 | 0.00012991 | 0.0116016 | 0.06 |
| AC243960.1 | chr19 | + | 41553705 | 41553746 | 1.76E−07 | 5.74E−05 | 0.06 |
| PSMG4 | chr6 | + | 3264173 | 3264325 | 1.27E−13 | 3.44E−10 | 0.061 |
| IKBKG | chrX | + | 154560406 | 154560559 | 4.93E−08 | 2.01E−05 | 0.061 |
| TRMT61B | chr2 | − | 28852407 | 28852499 | 6.76E−09 | 3.78E−06 | 0.061 |
| IGHG3 | chr14 | − | 105770292 | 105770337 | 5.40E−11 | 6.16E−08 | 0.061 |
| ING3 | chr7 | + | 120964741 | 120964838 | 0.00069568 | 0.04302677 | 0.061 |
| RPL32P3 | chr3 | − | 129396216 | 129396420 | 0.00027031 | 0.02037313 | 0.061 |
| ABHD14A- | chr3 | + | 51988523 | 51988603 | 7.03E−07 | 0.00017643 | 0.061 |
| ACY1 | | | | | | | |
| TBCD | chr17 | + | 82909284 | 82909307 | 0.00017435 | 0.01463332 | 0.061 |
| RPAIN | chr17 | + | 5426235 | 5426299 | 0.00064222 | 0.04046014 | 0.061 |
| SULT1A1 | chr16 | − | 28609930 | 28610191 | 1.89E−09 | 1.24E−06 | 0.061 |
| TCFL5 | chr20 | − | 62857394 | 62857638 | 1.07E−05 | 0.0015881 | 0.062 |
| TCF25 | chr16 | + | 89893788 | 89893858 | 1.11E−16 | 5.28E−13 | 0.062 |
| COX20 | chr1 | + | 244841943 | 244842058 | 0.00042949 | 0.02952315 | 0.062 |
| SERGEF | chr11 | − | 18010074 | 18010139 | 1.95E−12 | 3.53E−09 | 0.062 |
| KIAA2026 | chr9 | − | 5910614 | 5910707 | 1.48E−07 | 5.01E−05 | 0.062 |
| CD40 | chr20 | + | 46121723 | 46121898 | 2.44E−10 | 2.19E−07 | 0.063 |
| KIR2DL1 | chr19 | + | 54780088 | 54780166 | 7.23E−06 | 0.00114802 | 0.063 |
| GPR141 | chr7 | + | 37709717 | 37709799 | 0.00012656 | 0.01138908 | 0.063 |
| LRRC37B | chr17 | + | 32027768 | 32027840 | 2.39E−07 | 7.24E−05 | 0.063 |
| TMEM44-AS1 | chr3 | + | 194589020 | 194589142 | 8.89E−05 | 0.00867794 | 0.063 |
| TYSND1 | chr10 | − | 70142667 | 70142853 | 1.98E−08 | 9.32E−06 | 0.063 |
| FGR | chr1 | − | 27621558 | 27621657 | 0.00027468 | 0.02064046 | 0.063 |
| ZNF133 | chr20 | + | 18305680 | 18305807 | 3.37E−08 | 1.47E−05 | 0.063 |
| BCL2L13 | chr22 | + | 17696140 | 17696210 | 3.41E−09 | 2.05E−06 | 0.064 |
| PMS2 | chr7 | − | 6005891 | 6006031 | 8.86E−06 | 0.0013551 | 0.064 |
| ARHGAP19 | chr10 | − | 97256317 | 97256404 | 4.64E−06 | 0.00079573 | 0.064 |
| TRIM34 | chr11 | + | 5642816 | 5642853 | 2.54E−07 | 7.57E−05 | 0.064 |
| ZBTB8OS | chr1 | − | 32634767 | 32634792 | 9.81E−07 | 0.00023146 | 0.064 |
| RESF1 | chr12 | + | 31980877 | 31985957 | 4.45E−09 | 2.59E−06 | 0.064 |
| CHD4 | chr12 | − | 6580581 | 6580679 | 1.97E−05 | 0.00263448 | 0.064 |
| ZNF133 | chr20 | + | 18305667 | 18305807 | 4.93E−08 | 2.01E−05 | 0.064 |
| RBM23 | chr14 | − | 22905605 | 22905659 | 1.87E−10 | 1.74E−07 | 0.064 |
| PCBP1-AS1 | chr2 | − | 70059642 | 70059680 | 0.00048609 | 0.03243737 | 0.064 |
| TMEM116 | chr12 | − | 111978735 | 111978787 | 0.00013785 | 0.0121498 | 0.065 |
| PI4KB | chr1 | − | 151324744 | 151324917 | 9.31E−07 | 0.00022272 | 0.065 |
| SNRPA1 | chr15 | − | 101291961 | 101292040 | 0.00015456 | 0.01326347 | 0.065 |
| PCBP1-AS1 | chr2 | − | 70053730 | 70053812 | 0.00067913 | 0.04228381 | 0.065 |
| HMOX2 | chr16 | + | 4483636 | 4483754 | 1.07E−07 | 3.79E−05 | 0.066 |
| GMDS-DT | chr6 | + | 2269697 | 2269799 | 2.75E−08 | 1.26E−05 | 0.066 |
| IL18R1 | chr2 | + | 102367824 | 102368068 | 0.00052261 | 0.03430141 | 0.066 |
| UPB1 | chr22 | + | 24523618 | 24523773 | 2.78E−06 | 0.00053081 | 0.067 |
| ZNF138 | chr7 | + | 64815575 | 64815653 | 6.47E−07 | 0.0001639 | 0.067 |
| GATC | chr12 | + | 120454932 | 120455030 | 1.41E−12 | 2.64E−09 | 0.067 |
| PIK3C2B | chr1 | − | 204433792 | 204433949 | 9.40E−05 | 0.00901632 | 0.067 |
| UPF3A | chr13 | + | 114286301 | 114286400 | 5.83E−07 | 0.00015145 | 0.067 |
| ANGEL2 | chr1 | − | 213013092 | 213013418 | 1.14E−06 | 0.00026008 | 0.067 |
| KRIT1 | chr7 | − | 92245426 | 92245574 | 5.06E−05 | 0.00556272 | 0.067 |
| CHD4 | chr12 | − | 6580581 | 6580699 | 4.43E−05 | 0.00502057 | 0.067 |
| SRPK2 | chr7 | − | 105117366 | 105117511 | 7.42E−07 | 0.00018426 | 0.068 |
| EVA1C | chr21 | + | 32457596 | 32457720 | 2.06E−06 | 0.00041493 | 0.068 |
| ACCS | chr11 | + | 44082064 | 44082192 | 3.48E−06 | 0.00063292 | 0.068 |
| DDX60L | chr4 | − | 168377727 | 168377843 | 0.00014501 | 0.01263489 | 0.069 |
| RBIS | chr8 | − | 85219235 | 85219363 | 3.62E−08 | 1.55E−05 | 0.069 |
| N4BP2L2 | chr13 | − | 32537005 | 32537027 | 0.00039412 | 0.02769757 | 0.069 |
| PCBP1-AS1 | chr2 | − | 70053730 | 70053815 | 0.00059604 | 0.03808323 | 0.07 |
| ERICH6-AS1 | chr3 | + | 150706926 | 150707171 | 9.04E−05 | 0.00878768 | 0.071 |
| ATP5MC2 | chr12 | − | 53669870 | 53670114 | 3.11E−05 | 0.00378606 | 0.071 |
| SRP54-AS1 | chr14 | − | 34963928 | 34964040 | 6.97E−09 | 3.85E−06 | 0.071 |
| ZBTB1 | chr14 | + | 64516599 | 64516751 | 7.89E−10 | 5.73E−07 | 0.071 |
| DTNBP1 | chr6 | − | 15651312 | 15651408 | 3.31E−05 | 0.00399223 | 0.072 |
| FAM228B | chr2 | + | 24139369 | 24139450 | 0.0002296 | 0.01792704 | 0.072 |
| METTL6 | chr3 | − | 15415497 | 15415583 | 2.01E−06 | 0.00040859 | 0.072 |
| KPTN | chr19 | − | 47483294 | 47483379 | 1.14E−12 | 2.25E−09 | 0.072 |
| POLK | chr5 | + | 75590343 | 75590440 | 1.54E−08 | 7.56E−06 | 0.072 |
| PGS1 | chr17 | + | 78392513 | 78392665 | 1.39E−05 | 0.00197384 | 0.072 |
| RPRD1B | chr20 | + | 38040434 | 38040564 | 6.21E−05 | 0.00651006 | 0.073 |
| LINC00426 | chr13 | − | 30367600 | 30367717 | 0.00015329 | 0.01317999 | 0.073 |
| CD160 | chr1 | + | 145730743 | 145731070 | 1.88E−06 | 0.00038822 | 0.073 |
| TAF2 | chr8 | − | 119801793 | 119802025 | 0.00021154 | 0.01683401 | 0.073 |
| IL15RA | chr10 | − | 5963742 | 5963841 | 0.00012622 | 0.01136434 | 0.073 |
| ANXA11 | chr10 | − | 80172806 | 80172869 | 5.72E−08 | 2.25E−05 | 0.073 |
| RALGAPB | chr20 | + | 38535085 | 38535207 | 1.10E−05 | 0.00162754 | 0.074 |
| FAM13B | chr5 | − | 138019880 | 138019977 | 0.00027015 | 0.02036945 | 0.074 |
| KIAA1191 | chr5 | − | 176359810 | 176359918 | 0.00024098 | 0.01856021 | 0.074 |
| DPEP2 | chr16 | − | 67991114 | 67991184 | 0.00021514 | 0.01706313 | 0.074 |
| CD226 | chr18 | − | 69873143 | 69873246 | 2.09E−09 | 1.36E−06 | 0.074 |
| PTPRA | chr20 | + | 2975214 | 2975241 | 0.00024877 | 0.01904312 | 0.075 |
| ZNF75D | chrX | − | 135291471 | 135291563 | 0.00037561 | 0.02663134 | 0.075 |
| NABP1 | chr2 | + | 191682491 | 191682595 | 2.46E−05 | 0.00313708 | 0.075 |
| EME2 | chr16 | + | 1774259 | 1774352 | 1.02E−07 | 3.66E−05 | 0.075 |
| SNRNP70 | chr19 | + | 49102113 | 49102185 | 0.00048055 | 0.0321037 | 0.075 |
| TRPV2 | chr17 | + | 16431783 | 16431850 | 0.00012972 | 0.01159338 | 0.076 |
| PLBD1-AS1 | chr12 | + | 14614521 | 14614570 | 0.00025283 | 0.01929505 | 0.076 |
| UBE3B | chr12 | + | 109529889 | 109530072 | 0.0004074 | 0.02842036 | 0.076 |
| CEP68 | chr2 | + | 65069398 | 65069801 | 4.26E−05 | 0.00486704 | 0.076 |
| ABRAXAS1 | chr4 | − | 83472221 | 83472288 | 0.0004238 | 0.02924008 | 0.077 |
| NLRP6 | chr11 | + | 280083 | 281839 | 0.00019803 | 0.01608321 | 0.077 |
| RIDA | chr8 | − | 98108645 | 98108768 | 0.00024359 | 0.0187041 | 0.077 |
| UBE3B | chr12 | + | 109481636 | 109481742 | 0.00033307 | 0.0242138 | 0.077 |
| AC243960.1 | chr19 | + | 41551179 | 41551494 | 1.83E−07 | 5.90E−05 | 0.077 |
| DPY19L1 | chr7 | − | 34957983 | 34958070 | 4.23E−05 | 0.00485388 | 0.078 |
| CLEC4C | chr12 | − | 7741420 | 7741531 | 0 | 0 | 0.078 |
| MADD | chr11 | + | 47273826 | 47273976 | 3.73E−08 | 1.59E−05 | 0.078 |
| CBWD3 | chr9 | + | 68266219 | 68266306 | 6.84E−05 | 0.00703795 | 0.078 |
| ANP32A | chr15 | − | 68784396 | 68784595 | 6.85E−08 | 2.64E−05 | 0.078 |
| ENDOV | chr17 | + | 80422205 | 80422245 | 1.11E−06 | 0.00025451 | 0.078 |
| FCGRT | chr19 | + | 49521694 | 49521813 | 3.73E−10 | 3.11E−07 | 0.078 |
| RNF8 | chr6 | + | 37374619 | 37374709 | 3.64E−06 | 0.00065582 | 0.079 |
| TFB1M | chr6 | − | 155298476 | 155298585 | 1.30E−08 | 6.59E−06 | 0.079 |
| LONP2 | chr16 | + | 48256609 | 48256741 | 9.13E−08 | 3.34E−05 | 0.079 |
| TMEM161B- | chr5 | + | 88283009 | 88283086 | 1.55E−11 | 2.10E−08 | 0.079 |
| AS1 | | | | | | | |
| TMEM161B- | chr5 | + | 88285743 | 88285851 | 4.76E−10 | 3.81E−07 | 0.079 |
| AS1 | | | | | | | |
| POLK | chr5 | + | 75547009 | 75547157 | 0.00014255 | 0.01245137 | 0.079 |
| PCBP1-AS1 | chr2 | − | 70053730 | 70053994 | 0.00034297 | 0.02468499 | 0.079 |
| RAB3A | chr19 | − | 18202512 | 18202855 | 1.38E−05 | 0.0019656 | 0.079 |
| SLC37A3 | chr7 | − | 140355667 | 140355764 | 4.06E−06 | 0.00071342 | 0.08 |
| MIR762HG | chr16 | + | 30888713 | 30888958 | 0.00074552 | 0.04546944 | 0.08 |
| MRS2 | chr6 | + | 24408407 | 24408444 | 1.21E−05 | 0.00176555 | 0.08 |
| TXNDC11 | chr16 | − | 11733981 | 11734079 | 9.01E−11 | 9.60E−08 | 0.08 |
| LUCAT1 | chr5 | − | 91313077 | 91313229 | 0.00013473 | 0.01192874 | 0.08 |
| CERS5 | chr12 | − | 50144768 | 50144969 | 4.45E−08 | 1.83E−05 | 0.08 |
| INO80E | chr16 | + | 30001211 | 30003013 | 0.00066614 | 0.04162435 | 0.081 |
| LUCAT1 | chr5 | − | 91312294 | 91313229 | 0.00012709 | 0.01141316 | 0.081 |
| AC096887.1 | chr3 | − | 53092368 | 53092618 | 5.55E−16 | 2.38E−12 | 0.081 |
| IRAK3 | chr12 | + | 66203710 | 66203893 | 0.00011632 | 0.01068068 | 0.081 |
| SGCB | chr4 | − | 52033430 | 52033640 | 1.65E−05 | 0.00228656 | 0.081 |
| CEP41 | chr7 | − | 130426645 | 130426759 | 0.00015201 | 0.0130958 | 0.082 |
| MPC1 | chr6 | − | 166366968 | 166367184 | 8.10E−05 | 0.00806148 | 0.082 |
| PPP2R2D | chr10 | + | 131940030 | 131940196 | 6.03E−10 | 4.54E−07 | 0.082 |
| TOGARAM2 | chr2 | + | 29022157 | 29022308 | 2.07E−06 | 0.0004182 | 0.082 |
| SLC25A37 | chr8 | + | 23543087 | 23543211 | 1.09E−07 | 3.84E−05 | 0.082 |
| ERLIN1 | chr10 | − | 100164003 | 100164095 | 1.49E−06 | 0.0003182 | 0.082 |
| COQ5 | chr12 | − | 120526456 | 120526546 | 2.19E−05 | 0.00287254 | 0.082 |
| TAF1C | chr16 | − | 84183071 | 84183149 | 0.00014808 | 0.01283276 | 0.082 |
| SNRNP70 | chr19 | + | 49102113 | 49103587 | 0.00039909 | 0.02799129 | 0.082 |
| INTS7 | chr1 | − | 211946606 | 211946705 | 5.71E−06 | 0.00094441 | 0.083 |
| DSTYK | chr1 | − | 205160113 | 205160270 | 8.50E−05 | 0.00838539 | 0.083 |
| CD44 | chr11 | + | 35196745 | 35196874 | 4.00E−15 | 1.53E−11 | 0.083 |
| RPL5 | chr1 | + | 92833388 | 92833458 | 0 | 0 | 0.084 |
| CCDC138 | chr2 | + | 108856793 | 108856970 | 0.00038746 | 0.02733931 | 0.084 |
| RMND5B | chr5 | + | 178131236 | 178131376 | 0.00051842 | 0.03414128 | 0.084 |
| SLC66A2 | chr18 | − | 79947266 | 79947341 | 0.0001841 | 0.01522639 | 0.084 |
| MAK | chr6 | − | 10830547 | 10830877 | 7.56E−09 | 4.13E−06 | 0.084 |
| ABITRAM | chr9 | + | 108939195 | 108939272 | 7.76E−05 | 0.00778989 | 0.084 |
| AC000120.4 | chr7 | − | 92244001 | 92244149 | 1.21E−07 | 4.24E−05 | 0.085 |
| TAMM41 | chr3 | − | 11830721 | 11830874 | 3.07E−05 | 0.00374074 | 0.085 |
| SP140 | chr2 | + | 230245862 | 230245940 | 0.00047899 | 0.03202374 | 0.085 |
| STAMBP | chr2 | + | 73870142 | 73870352 | 8.16E−08 | 3.02E−05 | 0.085 |
| UBXN8 | chr8 | + | 30754664 | 30754749 | 2.76E−09 | 1.71E−06 | 0.086 |
| CHROMR | chr2 | + | 178426365 | 178426505 | 0.00020583 | 0.0165108 | 0.086 |
| FANCG | chr9 | − | 35076970 | 35077101 | 3.72E−09 | 2.21E−06 | 0.086 |
| FGR | chr1 | − | 27623690 | 27623929 | 1.02E−06 | 0.00023778 | 0.086 |
| DPEP2 | chr16 | − | 67992063 | 67992193 | 4.27E−05 | 0.00487076 | 0.086 |
| RALGAPB | chr20 | + | 38535073 | 38535207 | 1.90E−05 | 0.00256257 | 0.087 |
| C1orf162 | chr1 | + | 111477333 | 111477428 | 5.66E−05 | 0.00604978 | 0.087 |
| GOLGA2P5 | chr12 | − | 100168451 | 100168633 | 8.10E−11 | 8.95E−08 | 0.087 |
| GMFG | chr19 | − | 39335434 | 39335554 | 0.00019532 | 0.01594451 | 0.087 |
| AC147651.1 | chr7 | + | 524079 | 524280 | 3.44E−05 | 0.00411258 | 0.088 |
| IQCB1 | chr3 | − | 121788283 | 121788432 | 1.08E−05 | 0.00160687 | 0.088 |
| ERLIN1 | chr10 | − | 100167347 | 100167406 | 7.06E−09 | 3.89E−06 | 0.088 |
| ATOX1 | chr5 | − | 151751703 | 151751779 | 7.38E−06 | 0.00116593 | 0.089 |
| RIDA | chr8 | − | 98108645 | 98108713 | 0.00041987 | 0.02908235 | 0.089 |
| TSPAN5 | chr4 | − | 98484398 | 98484598 | 2.50E−05 | 0.00316784 | 0.089 |
| SNHG17 | chr20 | − | 38422095 | 38422241 | 0.00082527 | 0.0493766 | 0.09 |
| MVK | chr12 | + | 109581394 | 109581550 | 0.00061365 | 0.03905218 | 0.09 |
| PSME3IP1 | chr16 | − | 57178501 | 57178852 | 2.40E−05 | 0.00308252 | 0.09 |
| POLK | chr5 | + | 75593877 | 75594049 | 3.06E−10 | 2.63E−07 | 0.091 |
| WDR20 | chr14 | + | 102208602 | 102209862 | 0.00010367 | 0.00973724 | 0.091 |
| DDX31 | chr9 | − | 132625663 | 132625745 | 2.64E−07 | 7.76E−05 | 0.092 |
| POLD1 | chr19 | + | 50395024 | 50395150 | 5.69E−06 | 0.00094098 | 0.093 |
| CAST | chr5 | + | 96726753 | 96726859 | 0.00042357 | 0.02924008 | 0.093 |
| TM7SF2 | chr11 | + | 65115313 | 65115390 | 0.00059335 | 0.03796218 | 0.093 |
| NSRP1 | chr17 | + | 30118079 | 30118173 | 0.00046935 | 0.03155949 | 0.093 |
| FAM210A | chr18 | − | 13671861 | 13671973 | 6.79E−05 | 0.00699038 | 0.093 |
| MYL5 | chr4 | + | 677921 | 678029 | 5.32E−07 | 0.00013988 | 0.094 |
| INO80C | chr18 | − | 35487385 | 35487823 | 2.87E−11 | 3.64E−08 | 0.094 |
| SIMC1 | chr5 | + | 176289653 | 176290955 | 3.16E−05 | 0.00383582 | 0.094 |
| ABHD16A | chr6 | − | 31702073 | 31702130 | 0.0005242 | 0.03436691 | 0.095 |
| DAP3 | chr1 | + | 155721516 | 155721618 | 2.29E−14 | 6.82E−11 | 0.095 |
| ANKRD26 | chr10 | − | 27005622 | 27005723 | 3.57E−05 | 0.00424836 | 0.096 |
| ADCY10P1 | chr6 | + | 41118128 | 41118184 | 0.00035189 | 0.02525519 | 0.096 |
| ADAP2 | chr17 | + | 30926826 | 30926918 | 0.00024331 | 0.0187041 | 0.096 |
| RPL5 | chr1 | + | 92833544 | 92833660 | 2.35E−12 | 4.14E−09 | 0.097 |
| ZCWPW1 | chr7 | − | 100408538 | 100408659 | 3.34E−12 | 5.64E−09 | 0.097 |
| SUSD3 | chr9 | + | 93077845 | 93077993 | 1.05E−05 | 0.00157203 | 0.097 |
| CHROMR | chr2 | + | 178426365 | 178426680 | 0.00040373 | 0.02824711 | 0.097 |
| TMEM126A | chr11 | + | 85650248 | 85650341 | 6.70E−05 | 0.00692502 | 0.097 |
| ZFP1 | chr16 | + | 75166769 | 75166913 | 8.78E−08 | 3.22E−05 | 0.097 |
| ZNF195 | chr11 | − | 3369356 | 3369502 | 0.00021825 | 0.01723225 | 0.099 |
| FPR2 | chr19 | + | 51763361 | 51763517 | 2.85E−07 | 8.23E−05 | 0.1 |
| DCLRE1C | chr10 | − | 14936537 | 14936593 | 1.62E−07 | 5.37E−05 | 0.1 |
| POU6F1 | chr12 | − | 51206788 | 51206883 | 0.00031273 | 0.02310465 | 0.1 |
| RPF2 | chr6 | + | 110988987 | 110989065 | 0.00017936 | 0.01491853 | 0.102 |
| MTSS1 | chr8 | − | 124557680 | 124557875 | 0.00019476 | 0.01590625 | 0.102 |
| TOP1MT | chr8 | − | 143334107 | 143334285 | 5.37E−11 | 6.16E−08 | 0.102 |
| CPVL | chr7 | − | 29071772 | 29071904 | 6.61E−07 | 0.00016671 | 0.102 |
| PTPA | chr9 | + | 129127963 | 129128068 | 7.41E−05 | 0.00751031 | 0.102 |
| PANK2 | chr20 | + | 3916926 | 3917050 | 0.00028416 | 0.02126905 | 0.104 |
| RGS18 | chr1 | + | 192161452 | 192161487 | 3.98E−12 | 6.55E−09 | 0.104 |
| CKLF | chr16 | + | 66563071 | 66563217 | 0.00060251 | 0.03842654 | 0.105 |
| PAK1 | chr11 | − | 77397026 | 77397093 | 2.40E−05 | 0.00308189 | 0.105 |
| HM13 | chr20 | + | 31548676 | 31548793 | 8.18E−08 | 3.02E−05 | 0.106 |
| LINC01934 | chr2 | + | 181354207 | 181354343 | 1.49E−06 | 0.00031858 | 0.107 |
| BBOF1 | chr14 | + | 74049701 | 74050195 | 1.99E−06 | 0.00040514 | 0.108 |
| BMPR2 | chr2 | + | 202555251 | 202556531 | 0 | 0 | 0.108 |
| AF117829.1 | chr8 | − | 89725517 | 89725641 | 6.29E−05 | 0.00658311 | 0.109 |
| THTPA | chr14 | + | 23556742 | 23557304 | 0.00023326 | 0.01811627 | 0.11 |
| C11orf80 | chr11 | + | 66762078 | 66762450 | 0.0006439 | 0.04054371 | 0.11 |
| UBL7 | chr15 | − | 74451435 | 74451520 | 4.13E−05 | 0.00476553 | 0.11 |
| TRIM5 | chr11 | − | 5665168 | 5665395 | 8.80E−05 | 0.00861499 | 0.11 |
| DICER1-AS1 | chr14 | + | 95158875 | 95159035 | 5.53E−08 | 2.20E−05 | 0.11 |
| AC009022.1 | chr16 | − | 69990596 | 69990683 | 5.88E−06 | 0.00096664 | 0.11 |
| POLR2J3 | chr7 | − | 102545302 | 102545420 | 4.56E−05 | 0.00515092 | 0.111 |
| MATR3 | chr5 | + | 139279934 | 139280058 | 1.83E−07 | 5.90E−05 | 0.111 |
| CBWD3 | chr9 | + | 68269599 | 68269722 | 2.16E−12 | 3.83E−09 | 0.112 |
| POU6F1 | chr12 | − | 51206788 | 51206947 | 2.80E−05 | 0.00347364 | 0.113 |
| TYSND1 | chr10 | − | 70143841 | 70143972 | 9.39E−06 | 0.00142387 | 0.113 |
| BBOF1 | chr14 | + | 74049701 | 74050195 | 8.37E−11 | 9.09E−08 | 0.114 |
| NBR2 | chr17 | + | 43132598 | 43132723 | 7.30E−07 | 0.00018223 | 0.114 |
| CCM2 | chr7 | + | 45071749 | 45071872 | 0.0005479 | 0.03553924 | 0.115 |
| MAP3K8 | chr10 | + | 30437175 | 30437406 | 4.96E−05 | 0.00548092 | 0.117 |
| AC008035.1 | chr12 | + | 46571255 | 46571412 | 5.89E−07 | 0.00015221 | 0.117 |
| LDLRAD4 | chr18 | + | 13484036 | 13484119 | 0.00071613 | 0.04404337 | 0.117 |
| PSMG4 | chr6 | + | 3264208 | 3264325 | 5.52E−13 | 1.15E−09 | 0.119 |
| ANAPC10 | chr4 | − | 144999256 | 144999294 | 0.00053917 | 0.03508711 | 0.12 |
| ZMYM1 | chr1 | + | 35095818 | 35095891 | 0.00016202 | 0.01381607 | 0.122 |
| ADCY10P1 | chr6 | + | 41138608 | 41138726 | 3.48E−05 | 0.00414421 | 0.124 |
| TCFL5 | chr20 | − | 62859363 | 62859526 | 2.67E−07 | 7.83E−05 | 0.126 |
| POLL | chr10 | − | 101585315 | 101585478 | 0.00015474 | 0.0132661 | 0.127 |
| DPH7 | chr9 | − | 137576308 | 137576402 | 0.00034008 | 0.02454786 | 0.127 |
| MOK | chr14 | − | 102231706 | 102231821 | 2.84E−08 | 1.28E−05 | 0.128 |
| MECP2 | chrX | − | 154056940 | 154057254 | 0.00057966 | 0.03725292 | 0.129 |
| HLA-DPA1 | chr6 | − | 33069018 | 33069300 | 8.76E−09 | 4.64E−06 | 0.131 |
| HM13 | chr20 | + | 31548379 | 31548793 | 5.44E−10 | 4.24E−07 | 0.131 |
| MVK | chr12 | + | 109579801 | 109579946 | 5.42E−05 | 0.0058498 | 0.133 |
| ZBTB8OS | chr1 | − | 32634767 | 32634792 | 0.00069151 | 0.04281367 | 0.133 |
| DOCK2 | chr5 | + | 170075946 | 170076084 | 1.42E−07 | 4.85E−05 | 0.136 |
| WBP1 | chr2 | + | 74458831 | 74459117 | 5.28E−05 | 0.00573478 | 0.137 |
| VMP1 | chr17 | + | 59764970 | 59765138 | 1.93E−06 | 0.00039672 | 0.138 |
| MYOM1 | chr18 | − | 3135546 | 3135730 | 2.96E−07 | 8.46E−05 | 0.139 |
| FYB1 | chr5 | − | 39212677 | 39213017 | 0.00024024 | 0.0185248 | 0.14 |
| EVA1C | chr21 | + | 32457596 | 32457720 | 0.00074596 | 0.04547061 | 0.146 |
| AC093157.1 | chr1 | + | 101072659 | 101072830 | 0.0003899 | 0.02748942 | 0.148 |
| TRAF3IP1 | chr2 | + | 238333959 | 238334035 | 1.10E−05 | 0.00162144 | 0.149 |
| SNHG17 | chr20 | − | 38422091 | 38422241 | 1.43E−05 | 0.00202187 | 0.151 |
| YWHAH | chr22 | + | 31945499 | 31945678 | 0.00019982 | 0.01618391 | 0.151 |
| ARMCX5- | chrX | + | 102639399 | 102639533 | 0 | 0 | 0.152 |
| GPRASP2 | | | | | | | |
| AC009022.1 | chr16 | − | 69992910 | 69993248 | 8.12E−05 | 0.00807253 | 0.152 |
| ZNF232 | chr17 | − | 5111075 | 5111219 | 0.00033549 | 0.02434619 | 0.154 |
| CENPN | chr16 | + | 81022596 | 81022652 | 0.0006022 | 0.03842064 | 0.155 |
| WBP1 | chr2 | + | 74458831 | 74458919 | 6.08E−05 | 0.00641584 | 0.157 |
| ITGB3BP | chr1 | − | 63510064 | 63510181 | 2.53E−10 | 2.26E−07 | 0.157 |
| MPV17 | chr2 | − | 27317077 | 27317234 | 2.53E−08 | 1.17E−05 | 0.157 |
| MTIF3 | chr13 | − | 27441016 | 27441077 | 2.66E−15 | 1.04E−11 | 0.16 |
| LRRC23 | chr12 | + | 6909889 | 6910026 | 0.00023432 | 0.01816614 | 0.16 |
| TMEM143 | chr19 | − | 48360071 | 48360176 | 1.29E−06 | 0.00028542 | 0.16 |
| NEIL2 | chr8 | + | 11770173 | 11770335 | 0.00071612 | 0.04404337 | 0.168 |
| TMEM161B- | chr5 | + | 88283009 | 88283086 | 7.14E−06 | 0.00113677 | 0.168 |
| AS1 | | | | | | | |
| INTS7 | chr1 | − | 211946606 | 211946645 | 0.0002666 | 0.02016249 | 0.17 |
| TRAF3IP2-AS1 | chr6 | + | 111574620 | 111574802 | 0.00055747 | 0.03605107 | 0.174 |
| GOLGA8A | chr15 | − | 34435382 | 34435471 | 0.00012208 | 0.01109905 | 0.174 |
| BBS9 | chr7 | + | 33352858 | 33352873 | 9.71E−06 | 0.00146679 | 0.175 |
| PPIEL | chr1 | − | 39523806 | 39523896 | 0.00050728 | 0.03362161 | 0.177 |
| TUBGCP4 | chr15 | + | 43399111 | 43399174 | 3.59E−05 | 0.00426133 | 0.177 |
| AC060780.1 | chr17 | − | 43159422 | 43159727 | 0.00051785 | 0.03412416 | 0.18 |
| ECHDC2 | chr1 | − | 52899173 | 52899224 | 1.41E−06 | 0.00030698 | 0.182 |
| UROS | chr10 | − | 125789282 | 125789363 | 0.00043473 | 0.02978484 | 0.182 |
| MAFG | chr17 | − | 81923352 | 81923416 | 6.25E−07 | 0.00015941 | 0.186 |
| GIPR | chr19 | + | 45671284 | 45671392 | 0.00031083 | 0.02297403 | 0.186 |
| HCLS1 | chr3 | − | 121636433 | 121636489 | 0 | 0 | 0.191 |
| AC016727.1 | chr2 | + | 61480299 | 61480583 | 0.00021804 | 0.01723082 | 0.192 |
| SNHG17 | chr20 | − | 38422091 | 38422241 | 0.00033397 | 0.02426582 | 0.195 |
| AC022137.3 | chr19 | + | 53446226 | 53446337 | 0.00020113 | 0.01625268 | 0.195 |
| CWF19L1 | chr10 | − | 100260217 | 100260319 | 0.00023318 | 0.01811627 | 0.201 |
| LRRC23 | chr12 | + | 6912729 | 6913027 | 7.97E−06 | 0.00124443 | 0.208 |
| TMEM143 | chr19 | − | 48360071 | 48360183 | 3.32E−05 | 0.0039934 | 0.209 |
| VNN2 | chr6 | − | 132760631 | 132760803 | 3.87E−05 | 0.0045206 | 0.214 |
| AAMDC | chr11 | + | 77841018 | 77841060 | 4.39E−07 | 0.00011773 | 0.22 |
| TPRKB | chr2 | − | 73734527 | 73734588 | 4.40E−05 | 0.00499846 | 0.228 |
| FCHSD1 | chr5 | − | 141642396 | 141642503 | 2.48E−06 | 0.00048344 | 0.246 |
| PYROXD2 | chr10 | − | 98388353 | 98388508 | 0.00010894 | 0.01012971 | 0.249 |
| STRA6LP | chr9 | + | 97347313 | 97347666 | 1.73E−08 | 8.32E−06 | 0.255 |
| SERPINB9P1 | chr6 | − | 2857910 | 2857991 | 0.00045903 | 0.03110341 | 0.266 |
| AC245100.4 | chr1 | + | 148424062 | 148424189 | 3.71E−07 | 0.0001022 | 0.268 |
| PGAP3 | chr17 | − | 39676613 | 39676755 | 2.22E−16 | 1.00E−12 | 0.319 |

TABLE 18
MXE_FXSvsTD

| GeneID | chr | strand | 1stExonStart_0base | 1stExonEnd | 2ndExonStart_0base | 2ndExonEnd |
|---|---|---|---|---|---|---|
| CR1 | chr1 | + | 207526752 | 207526929 | 207545307 | 207545484 |
| PIGL | chr17 | + | 16313546 | 16313614 | 16316680 | 16316712 |
| XPNPEP3 | chr22 | + | 40870047 | 40870139 | 40881769 | 40882177 |
| MAPKBP1 | chr15 | + | 41813620 | 41813781 | 41815258 | 41815405 |
| TBC1D12 | chr10 | + | 94522343 | 94522453 | 94531201 | 94531460 |
| AC004593.2 | chr7 | + | 29146848 | 29146960 | 29354624 | 29354663 |
| AC016831.6 | chr7 | − | 130912109 | 130912152 | 130945719 | 130945835 |
| WDR60 | chr7 | + | 158905988 | 158906091 | 158911549 | 158911679 |
| ZNF720 | chr16 | + | 31723257 | 31723353 | 31753765 | 31753900 |
| MYO15B | chr17 | + | 75603031 | 75603077 | 75605863 | 75606021 |
| HSD17B3 | chr9 | − | 96252802 | 96252910 | 96254867 | 96254943 |
| WHAMMP3 | chr15 | + | 22674420 | 22674586 | 22676804 | 22676934 |
| AL392172.1 | chr1 | + | 222835118 | 222835215 | 222836995 | 222837066 |
| ATXN2 | chr12 | − | 111456028 | 111456256 | 111464661 | 111464715 |
| AL356481.1 | chr9 | − | 128536826 | 128536955 | 128543378 | 128543684 |
| PDE8A | chr15 | + | 85109052 | 85109130 | 85113376 | 85113447 |
| ZNF266 | chr19 | − | 9433667 | 9433786 | 9434797 | 9434859 |
| AL162258.1 | chr1 | − | 153627482 | 153627598 | 153631010 | 153631078 |
| CAMKK1 | chr17 | − | 3882527 | 3882564 | 3883041 | 3883175 |
| PPFIA1 | chr11 | + | 70349922 | 70350039 | 70350993 | 70351023 |
| ZNF266 | chr19 | − | 9434074 | 9434235 | 9434797 | 9434859 |
| CFAP70 | chr10 | − | 73277239 | 73277361 | 73278178 | 73278337 |
| ACCS | chr11 | + | 44071255 | 44071315 | 44073446 | 44073517 |
| ATF7IP2 | chr16 | + | 10473922 | 10473989 | 10480878 | 10480964 |
| NEIL2 | chr8 | + | 11771445 | 11771590 | 11779597 | 11779950 |
| TAF4 | chr20 | − | 62006509 | 62006758 | 62007546 | 62007636 |
| PNPT1 | chr2 | − | 55666990 | 55667093 | 55671318 | 55671376 |
| HLA-DRB5 | chr6 | − | 32519369 | 32519651 | 32521904 | 32522174 |
| DDX60L | chr4 | − | 168391539 | 168391644 | 168395958 | 168396124 |
| YAF2 | chr12 | − | 42172139 | 42172209 | 42235613 | 42235755 |
| SP140 | chr2 | + | 230245862 | 230245940 | 230247915 | 230248065 |
| KIAA1841 | chr2 | + | 61073457 | 61073551 | 61076919 | 61077158 |
| TNNT3 | chr11 | + | 1926469 | 1926481 | 1929809 | 1929828 |
| ARFIP1 | chr4 | + | 152807203 | 152807248 | 152809672 | 152809733 |
| TUBD1 | chr17 | − | 59866608 | 59866749 | 59874538 | 59874703 |
| POMZP3 | chr7 | − | 76611453 | 76611591 | 76618182 | 76618300 |
| SH3TC1 | chr4 | + | 8225174 | 8225216 | 8226979 | 8228644 |
| POLR2J4 | chr7 | − | 43986600 | 43986677 | 44013592 | 44013679 |
| CDKL3 | chr5 | − | 134308573 | 134308727 | 134312291 | 134312380 |
| CD300LF | chr17 | − | 74696194 | 74696217 | 74698368 | 74698481 |
| ELP1 | chr9 | − | 108918810 | 108918901 | 108919252 | 108919349 |
| RPTOR | chr17 | + | 80707840 | 80707999 | 80730559 | 80730706 |
| ZNF266 | chr19 | − | 9434074 | 9434235 | 9434797 | 9434859 |
| POLR2J4 | chr7 | − | 43986600 | 43986677 | 43987677 | 43987888 |
| CBWD3 | chr9 | + | 68266219 | 68266306 | 68268933 | 68268978 |
| WDR12 | chr2 | − | 202894580 | 202894626 | 202897299 | 202897415 |
| PVT1 | chr8 | + | 128009589 | 128009718 | 128082752 | 128082848 |
| LLGL2 | chr17 | + | 75574212 | 75574260 | 75574609 | 75574668 |
| TCFL5 | chr20 | − | 62854015 | 62854157 | 62859363 | 62859526 |
| MCTP1 | chr5 | − | 94870416 | 94870491 | 94870871 | 94870973 |
| ST3GAL5 | chr2 | − | 85852859 | 85853099 | 85861180 | 85861292 |
| UBXN8 | chr8 | + | 30754664 | 30754787 | 30756764 | 30756887 |
| ATXN2 | chr12 | − | 111464661 | 111464715 | 111470107 | 111470240 |
| MICA | chr6 | + | 31410542 | 31410797 | 31411946 | 31412225 |
| CARD8 | chr19 | − | 48242539 | 48242644 | 48246074 | 48246381 |
| CARD8 | chr19 | − | 48242539 | 48242644 | 48246074 | 48246381 |
| AL121845.3 | chr20 | + | 63735158 | 63735564 | 63737532 | 63737647 |
| IL32 | chr16 | + | 3067983 | 3068010 | 3068179 | 3068239 |
| GMPPB | chr3 | − | 49722431 | 49722510 | 49722595 | 49722754 |
| ZEB2 | chr2 | − | 144403915 | 144404130 | 144424795 | 144424867 |
| TBC1D8 | chr2 | − | 101032267 | 101032385 | 101033543 | 101033758 |
| LEMD3 | chr12 | + | 65245668 | 65245774 | 65245860 | 65245939 |
| SLC38A1 | chr12 | − | 46201097 | 46201198 | 46203009 | 46203089 |
| JARID2 | chr6 | + | 15468541 | 15468718 | 15487306 | 15487542 |
| SLC38A9 | chr5 | − | 55633694 | 55635657 | 55645788 | 55645895 |
| AC119396.1 | chr19 | + | 7362780 | 7362905 | 7372811 | 7373071 |
| UBE3C | chr7 | + | 157216866 | 157216971 | 157220688 | 157220776 |
| MARCHF8 | chr10 | − | 45461230 | 45461411 | 45489366 | 45489417 |
| TUBGCP4 | chr15 | + | 43400043 | 43400221 | 43401715 | 43401850 |
| SLAMF1 | chr1 | − | 160624095 | 160624185 | 160634612 | 160634897 |
| GTF2H2 | chr5 | − | 71045443 | 71045507 | 71048005 | 71048113 |
| IRF8 | chr16 | + | 85913130 | 85913236 | 85918416 | 85918803 |
| POMZP3 | chr7 | − | 76611453 | 76611591 | 76611721 | 76611813 |
| MCM6 | chr2 | − | 135862606 | 135862748 | 135866131 | 135866277 |
| PVT1 | chr8 | + | 128070161 | 128070272 | 128082752 | 128082848 |
| NIBAN3 | chr19 | + | 17537375 | 17537543 | 17539149 | 17539265 |
| NFS1 | chr20 | − | 35675044 | 35675202 | 35680736 | 35680871 |
| KDM3B | chr5 | + | 138381515 | 138381590 | 138391012 | 138392261 |
| AC114490.2 | chr1 | − | 34983766 | 34983947 | 34992233 | 34992387 |
| ZC3H13 | chr13 | − | 46020448 | 46020557 | 46042163 | 46042275 |
| TGS1 | chr8 | + | 55786237 | 55787060 | 55790181 | 55790299 |
| CR1 | chr1 | + | 207609289 | 207609688 | 207611676 | 207611853 |
| POMZP3 | chr7 | − | 76618182 | 76618300 | 76625521 | 76625683 |
| POMZP3 | chr7 | − | 76618182 | 76618615 | 76625521 | 76625683 |
| DDX21 | chr10 | + | 68971890 | 68972052 | 68973544 | 68973664 |
| SYNE1 | chr6 | − | 152176393 | 152176560 | 152180135 | 152180294 |
| ZBTB17 | chr1 | − | 15945714 | 15945840 | 15946934 | 15947123 |
| MYOM1 | chr18 | − | 3075724 | 3075761 | 3079178 | 3079342 |
| TIMM23B-AGAP6 | chr10 | + | 49987164 | 49987234 | 49988750 | 49988938 |
| SBNO1 | chr12 | − | 123315547 | 123315660 | 123317220 | 123317356 |
| RAB11FIP3 | chr16 | + | 491133 | 491268 | 496823 | 496859 |
| CNOT1 | chr16 | − | 58558472 | 58558674 | 58560211 | 58560362 |
| NMRK1 | chr9 | − | 75069741 | 75069813 | 75069894 | 75070042 |
| IRF8 | chr16 | + | 85914472 | 85914520 | 85918416 | 85918803 |
| TSC2 | chr16 | + | 2079275 | 2079428 | 2079556 | 2079669 |
| RCHY1 | chr4 | − | 75491888 | 75491933 | 75494100 | 75494179 |
| PVT1 | chr8 | + | 128070159 | 128070361 | 128082752 | 128082848 |
| COG8 | chr16 | − | 69330812 | 69331095 | 69332713 | 69332882 |
| FKBP15 | chr9 | − | 113206508 | 113206578 | 113207211 | 113207296 |
| PLBD1-AS1 | chr12 | + | 14614521 | 14614570 | 14619151 | 14619373 |
| TOM1 | chr22 | + | 35345724 | 35345784 | 35346929 | 35346969 |
| SUGP1 | chr19 | − | 19296988 | 19297344 | 19310096 | 19310200 |
| MCTP1 | chr5 | − | 94708511 | 94708609 | 94710817 | 94710927 |
| ZNF185 | chrX | + | 152920706 | 152920748 | 152922172 | 152922256 |
| TAF1D | chr11 | − | 93733086 | 93733121 | 93733349 | 93733397 |
| CELF1 | chr11 | − | 47476845 | 47476959 | 47477296 | 47477425 |
| PIGG | chr4 | + | 515972 | 516185 | 523458 | 523913 |
| C11orf80 | chr11 | + | 66762078 | 66762450 | 66788159 | 66788269 |
| ZNF638 | chr2 | + | 71408121 | 71408247 | 71418601 | 71418639 |
| TRIM14 | chr9 | − | 98091908 | 98092001 | 98094866 | 98095029 |
| NEIL2 | chr8 | + | 11771445 | 11771585 | 11783202 | 11783399 |
| SNX9 | chr6 | + | 157896826 | 157896998 | 157901897 | 157902045 |
| NSUN6 | chr10 | − | 18585948 | 18586093 | 18596207 | 18596327 |
| MRTFA | chr22 | − | 40420846 | 40421100 | 40423535 | 40423685 |
| SLC3A2 | chr11 | + | 62884631 | 62884690 | 62885176 | 62885608 |
| SNAPC1 | chr14 | + | 61782246 | 61782397 | 61792806 | 61792902 |
| ING4 | chr12 | − | 6652944 | 6653050 | 6656726 | 6656798 |
| SERF2 | chr15 | + | 43801131 | 43801187 | 43801517 | 43801569 |
| FAM114A2 | chr5 | − | 154034277 | 154034377 | 154034743 | 154034967 |
| CELF1 | chr11 | − | 47476845 | 47476956 | 47477296 | 47477425 |
| NFKB2 | chr10 | + | 102398211 | 102398297 | 102398738 | 102398864 |
| VARS1 | chr6 | − | 31782529 | 31782633 | 31782720 | 31782845 |
| CLEC17A | chr19 | + | 14599716 | 14599812 | 14600030 | 14600182 |
| TMEM184B | chr22 | − | 38225423 | 38225593 | 38226778 | 38226870 |
| EML2 | chr19 | − | 45619059 | 45619191 | 45626704 | 45626839 |
| CCM2 | chr7 | + | 45038252 | 45038426 | 45064462 | 45064646 |
| WRNIP1 | chr6 | + | 2770119 | 2770361 | 2779262 | 2779492 |
| DENND4B | chr1 | − | 153932641 | 153932777 | 153932860 | 153933030 |
| AL392172.1 | chr1 | + | 222835118 | 222835215 | 222836995 | 222837066 |
| TMEM131 | chr2 | − | 97796843 | 97796986 | 97797364 | 97797516 |
| RYK | chr3 | − | 134191848 | 134191974 | 134195081 | 134195182 |
| TMCO3 | chr13 | + | 113534044 | 113534240 | 113547299 | 113547379 |
| FOXP1 | chr3 | − | 70965889 | 70966056 | 70970735 | 70970805 |
| DNMT1 | chr19 | − | 10139675 | 10139817 | 10140780 | 10140909 |
| ZNF254 | chr19 | + | 24105939 | 24106066 | 24106547 | 24106643 |
| TCFL5 | chr20 | − | 62854015 | 62854157 | 62859363 | 62859526 |
| PILRB | chr7 | + | 100355142 | 100355240 | 100356749 | 100356883 |
| IDH1 | chr2 | − | 208239070 | 208239233 | 208241993 | 208242145 |
| NUP98 | chr11 | − | 3699081 | 3699348 | 3702462 | 3702892 |
| DDB2 | chr11 | + | 47234756 | 47234934 | 47235269 | 47235412 |
| IGHD | chr14 | − | 105840900 | 105841224 | 105841960 | 105842032 |
| POLR2J3 | chr7 | − | 102543524 | 102543702 | 102566581 | 102566736 |
| UBAP2L | chr1 | + | 154266500 | 154266568 | 154268756 | 154268954 |
| TSEN2 | chr3 | + | 12505153 | 12505231 | 12516610 | 12516661 |
| COA1 | chr7 | − | 43650611 | 43650712 | 43656032 | 43656153 |
| MPHOSPH6 | chr16 | − | 82149308 | 82149403 | 82151423 | 82151514 |
| CERS4 | chr19 | + | 8257878 | 8257985 | 8261687 | 8261844 |
| AC069281.2 | chr7 | − | 100577272 | 100577389 | 100577496 | 100577558 |
| ATP2B4 | chr1 | + | 203722477 | 203722689 | 203723880 | 203723988 |
| EIF4G1 | chr3 | + | 184324877 | 184325114 | 184325268 | 184325373 |
| WDFY3 | chr4 | − | 84678918 | 84679242 | 84682373 | 84682470 |
| ECPAS | chr9 | − | 111378579 | 111378730 | 111383210 | 111383332 |
| SLC25A19 | chr17 | − | 75278151 | 75278335 | 75283422 | 75283593 |
| STAU2 | chr8 | − | 73697307 | 73697442 | 73709031 | 73709177 |
| RPS6KA3 | chrX | − | 20195064 | 20195145 | 20204021 | 20204103 |
| DUS2 | chr16 | + | 68075354 | 68075504 | 68076631 | 68076719 |
| MAN1B1 | chr9 | + | 137101483 | 137101672 | 137106124 | 137106315 |
| KMT2D | chr12 | − | 49022589 | 49022875 | 49024577 | 49024708 |
| RRP8 | chr11 | − | 6600668 | 6600775 | 6600925 | 6601602 |
| SPAG9 | chr17 | − | 50977107 | 50977221 | 50979745 | 50979917 |
| CPNE1 | chr20 | − | 35627279 | 35627413 | 35630740 | 35630795 |
| UBXN11 | chr1 | − | 26297426 | 26297481 | 26297961 | 26298062 |
| SLC44A2 | chr19 | + | 10635430 | 10635515 | 10636322 | 10636585 |
| KDM5A | chr12 | − | 356431 | 356537 | 362962 | 363097 |
| TNPO1 | chr5 | + | 72897055 | 72897151 | 72900005 | 72900081 |
| HEATR5B | chr2 | − | 36988645 | 36988859 | 36990647 | 36990799 |
| RNF170 | chr8 | − | 42865415 | 42865489 | 42870003 | 42870112 |
| RHOT1 | chr17 | + | 32176160 | 32176213 | 32183170 | 32183272 |
| TMEM50B | chr21 | − | 33439213 | 33439297 | 33448781 | 33448879 |
| SCLT1 | chr4 | − | 128888678 | 128888774 | 128891058 | 128891137 |
| MAN2B1 | chr19 | − | 12648174 | 12648402 | 12649135 | 12649216 |
| PCBP1-AS1 | chr2 | − | 69988741 | 69988838 | 69996685 | 69996888 |
| AC069281.2 | chr7 | − | 100577496 | 100577558 | 100577663 | 100577740 |
| DTNBP1 | chr6 | − | 15637743 | 15637804 | 15651312 | 15651363 |
| PINK1 | chr1 | + | 20644489 | 20644672 | 20645559 | 20645723 |
| HDAC7 | chr12 | − | 47795905 | 47796016 | 47796214 | 47796298 |
| CMTM1 | chr16 | + | 66571136 | 66571270 | 66577103 | 66577202 |
| CDC23 | chr5 | − | 138191861 | 138191937 | 138192268 | 138192388 |
| PCBP1-AS1 | chr2 | − | 70055713 | 70055910 | 70083506 | 70083687 |
| PCBP1-AS1 | chr2 | − | 70055713 | 70055910 | 70083537 | 70083687 |
| EHBP1L1 | chr11 | + | 65579935 | 65579989 | 65580080 | 65580259 |
| ANKZF1 | chr2 | + | 219234132 | 219234288 | 219234825 | 219235312 |
| AC118553.2 | chr1 | + | 100049908 | 100050004 | 100058665 | 100058728 |
| NAE1 | chr16 | − | 66816580 | 66816672 | 66816964 | 66817028 |
| GIT1 | chr17 | − | 29575629 | 29575703 | 29575811 | 29575898 |
| CREBBP | chr16 | − | 3770569 | 3770986 | 3773750 | 3773930 |
| RALY | chr20 | + | 34073818 | 34073866 | 34075873 | 34076040 |
| XAF1 | chr17 | + | 6759255 | 6759718 | 6760405 | 6760601 |
| CCM2 | chr7 | + | 45038252 | 45038426 | 45063917 | 45064001 |
| CPSF7 | chr11 | − | 61416104 | 61416162 | 61416363 | 61416519 |
| CAST | chr5 | + | 96740037 | 96740118 | 96740744 | 96740783 |
| SMG5 | chr1 | − | 156259004 | 156259163 | 156260450 | 156260626 |
| PVT1 | chr8 | + | 128070159 | 128070272 | 128082752 | 128082848 |
| RGS6 | chr14 | + | 72518350 | 72518537 | 72536185 | 72536275 |
| SUMF2 | chr7 | + | 56074173 | 56074218 | 56074585 | 56074736 |
| RALY | chr20 | + | 34073818 | 34073866 | 34075873 | 34076040 |
| RALY | chr20 | + | 34073818 | 34073866 | 34075873 | 34076040 |
| MLKL | chr16 | − | 74678898 | 74678980 | 74682650 | 74682786 |
| ITPA | chr20 | + | 3218516 | 3218632 | 3221840 | 3221917 |
| GATAD2A | chr19 | + | 19494293 | 19494383 | 19495753 | 19495885 |
| MKNK1 | chr1 | − | 46568442 | 46568498 | 46572062 | 46572167 |
| MIR4435-2HG | chr2 | − | 111248147 | 111248241 | 111344060 | 111344237 |
| SSBP4 | chr19 | + | 18427897 | 18427982 | 18430840 | 18430930 |
| CCM2 | chr7 | + | 45038252 | 45038426 | 45063917 | 45064001 |
| TNFRSF1A | chr12 | − | 6329777 | 6330066 | 6330852 | 6330926 |
| TLE3 | chr15 | − | 70056297 | 70056374 | 70057458 | 70057658 |
| ARHGAP25 | chr2 | + | 68775220 | 68775420 | 68782232 | 68782320 |
| TRAK1 | chr3 | + | 42200817 | 42201054 | 42202435 | 42202752 |
| LRCH3 | chr3 | + | 197858833 | 197858905 | 197865422 | 197865471 |
| TLE3 | chr15 | − | 70056297 | 70056374 | 70057473 | 70057658 |
| PLD3 | chr19 | + | 40366772 | 40366915 | 40367695 | 40367879 |
| ADA | chr20 | − | 44623006 | 44623078 | 44624201 | 44624329 |
| SHISA5 | chr3 | − | 48469360 | 48469573 | 48469727 | 48469843 |
| NCF1 | chr7 | + | 74787983 | 74788088 | 74788558 | 74788704 |
| NUP88 | chr17 | − | 5387778 | 5387904 | 5388801 | 5388960 |
| USB1 | chr16 | + | 58014272 | 58014326 | 58017333 | 58017439 |
| FMNL1 | chr17 | + | 45240475 | 45240625 | 45241128 | 45241230 |
| CYB5R4 | chr6 | + | 83914415 | 83914448 | 83922437 | 83922470 |
| NFKB2 | chr10 | + | 102398384 | 102398523 | 102399287 | 102399497 |
| NUP88 | chr17 | − | 5387778 | 5387952 | 5388801 | 5388960 |
| RCBTB2 | chr13 | − | 48511769 | 48511877 | 48512015 | 48512174 |
| STRADA | chr17 | − | 63713405 | 63713527 | 63714005 | 63714108 |
| CRTC2 | chr1 | − | 153951259 | 153951666 | 153952017 | 153952262 |
| UNC13D | chr17 | − | 75839838 | 75839942 | 75840017 | 75840110 |
| ICE1 | chr5 | + | 5460435 | 5465226 | 5466333 | 5466502 |
| TMBIM1 | chr2 | − | 218278514 | 218278565 | 218279037 | 218279091 |
| SH2D3C | chr9 | − | 127747146 | 127747271 | 127749210 | 127749665 |
| SLC44A2 | chr19 | + | 10635162 | 10635255 | 10635430 | 10635515 |
| SORL1 | chr11 | + | 121488031 | 121488193 | 121490042 | 121490110 |
| LTBR | chr12 | + | 6386346 | 6386444 | 6388397 | 6388505 |
| CHP1 | chr15 | + | 41262755 | 41262883 | 41270556 | 41270618 |
| CTDNEP1 | chr17 | − | 7244550 | 7244635 | 7246025 | 7246137 |
| RPS12 | chr6 | + | 132816460 | 132816563 | 132816959 | 132817061 |
| WDFY4 | chr10 | + | 48963841 | 48964054 | 48966525 | 48966673 |
| FANCA | chr16 | − | 89748658 | 89748767 | 89749729 | 89749902 |
| CPNE1 | chr20 | − | 35627279 | 35627413 | 35630438 | 35631034 |
| CPNE1 | chr20 | − | 35627279 | 35627413 | 35630900 | 35631034 |
| OSBPL11 | chr3 | − | 125563697 | 125563843 | 125567393 | 125567595 |
| RHOT1 | chr17 | + | 32176160 | 32176213 | 32182756 | 32182865 |
| ITGAL | chr16 | + | 30513770 | 30513846 | 30516972 | 30517086 |
| INTS8 | chr8 | + | 94831991 | 94832174 | 94836523 | 94836631 |
| DBNL | chr7 | + | 44058431 | 44058480 | 44058901 | 44058983 |
| GSN | chr9 | + | 121321267 | 121321401 | 121324553 | 121324644 |
| CNOT3 | chr19 | + | 54146600 | 54146657 | 54148147 | 54148535 |
| DAGLB | chr7 | − | 6421726 | 6421804 | 6425987 | 6426114 |
| DBNL | chr7 | + | 44058431 | 44058480 | 44058877 | 44058983 |
| STXBP2 | chr19 | + | 7645196 | 7645306 | 7646248 | 7646344 |
| SNHG29 | chr17 | + | 16439527 | 16439703 | 16440184 | 16440253 |
| UNC13D | chr17 | − | 75831097 | 75831169 | 75831242 | 75831348 |
| FCHO1 | chr19 | + | 17766668 | 17766810 | 17770424 | 17770577 |
| SLC35C2 | chr20 | − | 46350760 | 46350890 | 46352050 | 46352226 |
| WDR47 | chr1 | − | 108981732 | 108981864 | 108982608 | 108982785 |
| MKNK2 | chr19 | − | 2041039 | 2041204 | 2041839 | 2042034 |
| INPP5D | chr2 | + | 233125747 | 233125919 | 233130507 | 233130648 |
| LRCH4 | chr7 | − | 100576901 | 100577005 | 100577496 | 100577558 |
| TTC7A | chr2 | + | 47011330 | 47011435 | 47021861 | 47021979 |
| UNC93B1 | chr11 | − | 67997674 | 67997799 | 67998358 | 67998452 |
| PDE4B | chr1 | + | 66363406 | 66363571 | 66365666 | 66365766 |
| HLA-DRB1 | chr6 | − | 32581556 | 32581838 | 32584108 | 32584378 |
| TNFRSF1A | chr12 | − | 6329777 | 6330066 | 6330266 | 6330295 |
| AC118553.2 | chr1 | + | 100017681 | 100017815 | 100049908 | 100050004 |
| FGR | chr1 | − | 27615688 | 27615844 | 27616856 | 27617006 |
| COG4 | chr16 | − | 70481358 | 70481487 | 70481763 | 70481865 |
| FLOT1 | chr6 | − | 30740678 | 30740798 | 30741613 | 30741704 |
| CAMK1D | chr10 | + | 12791157 | 12791233 | 12814194 | 12814307 |
| LMBR1 | chr7 | − | 156724111 | 156724178 | 156734176 | 156734257 |
| TSC2 | chr16 | + | 2058746 | 2058873 | 2060669 | 2060813 |
| FGR | chr1 | − | 27621558 | 27621657 | 27625088 | 27625151 |
| NAA60 | chr16 | + | 3448470 | 3448540 | 3479470 | 3479600 |
| INTS6 | chr13 | − | 51395299 | 51395483 | 51423029 | 51423090 |
| TTC16 | chr9 | + | 127724755 | 127724897 | 127726238 | 127726404 |
| H2AZ2 | chr7 | − | 44835528 | 44835658 | 44843276 | 44843354 |
| AC004997.1 | chr22 | − | 30288705 | 30288776 | 30292772 | 30292851 |
| SPTLC1 | chr9 | − | 92076797 | 92077035 | 92079433 | 92079566 |
| BAG6 | chr6 | − | 31640384 | 31640528 | 31640644 | 31640704 |
| TTC17 | chr11 | + | 43414589 | 43414776 | 43436163 | 43436334 |
| CDK16 | chrX | + | 47227378 | 47227469 | 47228566 | 47228644 |
| NAXD | chr13 | + | 110622215 | 110622366 | 110624233 | 110624279 |
| IGFLR1 | chr19 | − | 35739709 | 35740086 | 35740379 | 35740564 |
| GOLGA7 | chr8 | + | 41490832 | 41490965 | 41497508 | 41497661 |
| YY1AP1 | chr1 | − | 155679408 | 155679512 | 155680415 | 155680456 |
| YY1AP1 | chr1 | − | 155679408 | 155679512 | 155680415 | 155680456 |
| CASC4 | chr15 | + | 44328684 | 44328787 | 44338236 | 44338317 |
| PTDSS1 | chr8 | + | 96284108 | 96284153 | 96287021 | 96287146 |
| CEP290 | chr12 | − | 88059897 | 88060020 | 88060829 | 88060994 |
| TIMM23B-AGAP6 | chr10 | + | 49973011 | 49973142 | 49987164 | 49987234 |
| IL21R | chr16 | + | 27430055 | 27430120 | 27434346 | 27434449 |
| DNMT1 | chr19 | − | 10180777 | 10180885 | 10182040 | 10182077 |
| PDIA4 | chr7 | − | 149005140 | 149005374 | 149005896 | 149006053 |
| SIPA1 | chr11 | + | 65647383 | 65647658 | 65649261 | 65649480 |
| POLR2F | chr22 | + | 37956772 | 37956842 | 37959345 | 37959476 |
| POLL | chr10 | − | 101582762 | 101582891 | 101583507 | 101583681 |
| PSMA5 | chr1 | − | 109415236 | 109415363 | 109421859 | 109421926 |
| PAFAH2 | chr1 | − | 25989447 | 25989601 | 25990726 | 25990921 |
| MOV10 | chr1 | + | 112674847 | 112675049 | 112688934 | 112689138 |
| HMGB2 | chr4 | − | 173333068 | 173333214 | 173333499 | 173333669 |
| PYCR2 | chr1 | − | 225922203 | 225922383 | 225923700 | 225923771 |
| B4GALT3 | chr1 | − | 161173858 | 161174049 | 161176433 | 161176579 |
| RHOT2 | chr16 | + | 672488 | 672566 | 672702 | 672825 |
| DDX17 | chr22 | − | 38494629 | 38494802 | 38495795 | 38495937 |
| NQO2 | chr6 | + | 3004494 | 3004651 | 3006467 | 3006559 |
| WDR70 | chr5 | + | 37437921 | 37437981 | 37443238 | 37443372 |
| INTS8 | chr8 | + | 94841490 | 94841591 | 94842346 | 94842488 |
| EXOSC1 | chr10 | − | 97441170 | 97441259 | 97445731 | 97445847 |
| RABEPK | chr9 | + | 125203007 | 125203066 | 125213369 | 125213522 |
| UTP6 | chr17 | − | 31884423 | 31884505 | 31885979 | 31886061 |
| TRPC4AP | chr20 | − | 35021189 | 35021356 | 35035122 | 35035308 |
| XAF1 | chr17 | + | 6759661 | 6759718 | 6762154 | 6762240 |
| ZDHHC4 | chr7 | + | 6578565 | 6578720 | 6580554 | 6580678 |
| PHYKPL | chr5 | − | 178224447 | 178224564 | 178231404 | 178231523 |
| CAST | chr5 | + | 96675538 | 96675601 | 96695835 | 96695907 |
| PPA2 | chr4 | − | 105437949 | 105438036 | 105453597 | 105453642 |
| RBM6 | chr3 | + | 49972058 | 49972148 | 49975322 | 49975392 |
| DDX21 | chr10 | + | 68974669 | 68974743 | 68977528 | 68977688 |
| RCBTB2 | chr13 | − | 48502723 | 48502914 | 48510628 | 48510771 |
| ABCA7 | chr19 | + | 1050920 | 1051052 | 1051154 | 1051294 |
| LMF2 | chr22 | − | 50506284 | 50506502 | 50506637 | 50506666 |
| PHC2 | chr1 | − | 33332274 | 33332404 | 33334184 | 33334292 |
| WDSUB1 | chr2 | − | 159248371 | 159248512 | 159259809 | 159259843 |
| JAK1 | chr1 | − | 64883276 | 64883475 | 64886258 | 64886341 |
| IFTAP | chr11 | + | 36610080 | 36610239 | 36633283 | 36633438 |
| RABEPK | chr9 | + | 125207563 | 125207721 | 125213369 | 125213522 |
| SNX10 | chr7 | + | 26346419 | 26346466 | 26360974 | 26361061 |
| DAGLB | chr7 | − | 6432836 | 6432959 | 6445952 | 6446104 |
| DEK | chr6 | − | 18257952 | 18258062 | 18258303 | 18258405 |
| WDSUB1 | chr2 | − | 159248371 | 159248512 | 159271701 | 159271795 |
| LAIR1 | chr19 | − | 54360915 | 54361209 | 54364294 | 54364330 |
| VDAC2 | chr10 | + | 75212229 | 75212298 | 75214020 | 75214070 |
| PPP4R3B | chr2 | − | 55598415 | 55599039 | 55603977 | 55604076 |
| DLEU2 | chr13 | − | 50044639 | 50044768 | 50049583 | 50049662 |
| STK38 | chr6 | − | 36525590 | 36525642 | 36540071 | 36540207 |
| STAT5B | chr17 | − | 42227528 | 42227685 | 42231999 | 42232137 |
| DESI1 | chr22 | − | 41604043 | 41604153 | 41607261 | 41607331 |
| CCM2 | chr7 | + | 45038252 | 45038426 | 45069825 | 45069961 |
| PREX1 | chr20 | − | 48708259 | 48708421 | 48747808 | 48747880 |
| TMEM87A | chr15 | − | 42233212 | 42233306 | 42236319 | 42236419 |
| NEPRO | chr3 | − | 113011044 | 113011406 | 113012733 | 113013403 |
| WDR37 | chr10 | + | 1072115 | 1072293 | 1077906 | 1078003 |
| LAIR1 | chr19 | − | 54360915 | 54361206 | 54364294 | 54364330 |
| LAIR1 | chr19 | − | 54360915 | 54361243 | 54364294 | 54364330 |
| SUPT20H | chr13 | − | 37047534 | 37047601 | 37047877 | 37047936 |
| CDC16 | chr13 | + | 114236644 | 114236699 | 114236798 | 114236896 |
| JPX | chrX | + | 73946998 | 73947374 | 73994863 | 73994961 |
| RBM23 | chr14 | − | 22911785 | 22911903 | 22913723 | 22913845 |
| RPL18A | chr19 | + | 17861292 | 17861472 | 17862093 | 17862223 |
| LINC00893 | chrX | − | 149529512 | 149529593 | 149532881 | 149533055 |
| ZDHHC4 | chr7 | + | 6578565 | 6578746 | 6580554 | 6580678 |
| SND1 | chr7 | + | 127686612 | 127686762 | 127694827 | 127694948 |
| SLC3A2 | chr11 | + | 62881892 | 62882066 | 62882907 | 62882999 |
| RBM33 | chr7 | + | 155700772 | 155700944 | 155706859 | 155707068 |
| TMEM63A | chr1 | − | 225867887 | 225868030 | 225871075 | 225871113 |
| MEF2D | chr1 | − | 156477011 | 156477202 | 156479289 | 156479346 |
| NLRC5 | chr16 | + | 57028071 | 57028385 | 57029772 | 57029856 |
| POLD2 | chr7 | − | 44117942 | 44118064 | 44121833 | 44122109 |
| TNFSF13 | chr17 | + | 7559623 | 7559702 | 7560048 | 7560167 |
| NTAN1 | chr16 | − | 15044333 | 15044407 | 15047417 | 15047550 |
| VPS53 | chr17 | − | 661808 | 661895 | 697417 | 697484 |
| TANGO2 | chr22 | + | 20036759 | 20037094 | 20043354 | 20043443 |
| ARHGEF40 | chr14 | + | 21073044 | 21073242 | 21073931 | 21075180 |
| ZDHHC24 | chr11 | − | 66526935 | 66527006 | 66543703 | 66543981 |
| ABCA7 | chr19 | + | 1055906 | 1055939 | 1056065 | 1056243 |
| SAMD9L | chr7 | − | 93144731 | 93144833 | 93145384 | 93145532 |
| EPSTI1 | chr13 | − | 42953947 | 42954021 | 42969093 | 42970670 |
| POLR3C | chr1 | + | 145833489 | 145833582 | 145838055 | 145838206 |
| ECHDC1 | chr6 | − | 127316449 | 127316502 | 127330808 | 127331030 |
| GMDS-DT | chr6 | + | 2269697 | 2269799 | 2271815 | 2273417 |
| WDSUB1 | chr2 | − | 159256195 | 159256375 | 159271701 | 159271795 |
| TYROBP | chr19 | − | 35907445 | 35907580 | 35907729 | 35907762 |
| TTC13 | chr1 | − | 230931297 | 230931472 | 230931735 | 230931877 |
| PHF20L1 | chr8 | + | 132814636 | 132814889 | 132816887 | 132817076 |
| CORO1C | chr12 | − | 108662028 | 108662158 | 108701123 | 108701323 |
| ECHDC1 | chr6 | − | 127316449 | 127316502 | 127326997 | 127327144 |
| SMIM8 | chr6 | + | 87337008 | 87337166 | 87371592 | 87371712 |
| NEPRO | chr3 | − | 113011044 | 113011165 | 113012733 | 113013403 |
| HLA-C | chr6 | − | 31269337 | 31269385 | 31269492 | 31269525 |
| BBS9 | chr7 | + | 33357854 | 33357995 | 33367766 | 33367862 |
| AMD1 | chr6 | + | 110887504 | 110887591 | 110888856 | 110888983 |
| ERCC1 | chr19 | − | 45413962 | 45414034 | 45414860 | 45414960 |
| DLG1 | chr3 | − | 197194424 | 197194589 | 197282678 | 197282845 |
| ZFAND1 | chr8 | − | 81713917 | 81714039 | 81714803 | 81715114 |
| MAPKAPK5 | chr12 | + | 111865249 | 111865323 | 111867571 | 111867669 |
| CELF1 | chr11 | − | 47473087 | 47473231 | 47475335 | 47475521 |
| PVT1 | chr8 | + | 127795893 | 127796008 | 128070159 | 128070272 |
| WASH3P | chr15 | + | 101966069 | 101966223 | 101971822 | 101972049 |
| CNOT2 | chr12 | + | 70278131 | 70278274 | 70310894 | 70311017 |
| RFFL | chr17 | − | 35021370 | 35021781 | 35026373 | 35026561 |
| LONP2 | chr16 | + | 48258617 | 48258740 | 48261423 | 48261587 |
| ARIH2 | chr3 | + | 48961611 | 48961679 | 48967124 | 48967275 |
| SNX22 | chr15 | + | 64152637 | 64152742 | 64153651 | 64153684 |
| SCAP | chr3 | − | 47417121 | 47417207 | 47417303 | 47417823 |
| VASP | chr19 | + | 45522717 | 45522818 | 45523840 | 45523877 |
| FKBP15 | chr9 | − | 113199813 | 113199963 | 113202530 | 113202629 |
| PSD4 | chr2 | + | 113184956 | 113185073 | 113185364 | 113185440 |
| INTS8 | chr8 | + | 94838462 | 94838618 | 94842346 | 94842488 |
| TANGO2 | chr22 | + | 20036759 | 20036854 | 20043354 | 20043443 |
| TMEM127 | chr2 | − | 96254832 | 96254997 | 96265137 | 96265512 |
| LRCH3 | chr3 | + | 197854391 | 197854445 | 197858833 | 197858905 |
| KDM5C | chrX | − | 53218275 | 53218398 | 53220838 | 53220916 |
| BBS1 | chr11 | + | 66519616 | 66519748 | 66521269 | 66521376 |
| SRP14-AS1 | chr15 | + | 40056452 | 40056641 | 40064295 | 40064601 |
| SOS1 | chr2 | − | 38987472 | 38987591 | 38995122 | 38995387 |
| RNF121 | chr11 | + | 71994718 | 71994852 | 71995439 | 71995551 |
| PDCD6 | chr5 | + | 272710 | 272772 | 311292 | 311402 |
| SEC16A | chr9 | − | 136451255 | 136451408 | 136453427 | 136453510 |
| ADAP2 | chr17 | + | 30931888 | 30931968 | 30934184 | 30934297 |
| DNAJC2 | chr7 | − | 103337735 | 103337811 | 103341763 | 103341865 |
| RPS6KB1 | chr17 | + | 59914634 | 59914703 | 59926434 | 59926582 |
| RNF121 | chr11 | + | 71994718 | 71994852 | 71995449 | 71995551 |
| CEP290 | chr12 | − | 88059897 | 88060020 | 88060829 | 88060994 |
| PVT1 | chr8 | + | 127795893 | 127796071 | 128070159 | 128070272 |
| EXOSC9 | chr4 | + | 121802914 | 121803017 | 121804621 | 121804759 |
| CLPTM1 | chr19 | + | 44961962 | 44962075 | 44973086 | 44973210 |
| PREX1 | chr20 | − | 48734545 | 48734650 | 48747808 | 48747880 |
| IL15 | chr4 | + | 141688821 | 141689035 | 141720468 | 141720566 |
| MTA1 | chr14 | + | 105445417 | 105445511 | 105450057 | 105450184 |
| EFCAB13 | chr17 | + | 47361377 | 47361521 | 47391436 | 47391580 |
| OCEL1 | chr19 | + | 17226993 | 17227199 | 17228255 | 17228309 |
| ZC3H7A | chr16 | − | 11763477 | 11763659 | 11765052 | 11765153 |
| DAGLB | chr7 | − | 6416258 | 6416754 | 6416840 | 6416921 |
| EIF2B4 | chr2 | − | 27368371 | 27368463 | 27369005 | 27369212 |
| EIF2D | chr1 | − | 206599002 | 206599092 | 206599462 | 206599612 |
| GUSBP11 | chr22 | − | 23705428 | 23705541 | 23709129 | 23709241 |
| FBXO3 | chr11 | − | 33768850 | 33769014 | 33770740 | 33770830 |
| VASP | chr19 | + | 45522717 | 45522818 | 45523643 | 45523695 |
| GRK3 | chr22 | + | 25703509 | 25703576 | 25704108 | 25704209 |
| PIK3AP1 | chr10 | − | 96656797 | 96656934 | 96709566 | 96709983 |
| CDC16 | chr13 | + | 114236644 | 114236699 | 114236801 | 114236896 |
| RALGAPB | chr20 | + | 38565358 | 38565478 | 38570768 | 38570847 |
| PAPSS1 | chr4 | − | 107693770 | 107694006 | 107701170 | 107701285 |
| RABGAP1L | chr1 | + | 174370978 | 174371072 | 174393994 | 174394145 |
| C1orf21 | chr1 | + | 184477385 | 184477603 | 184507587 | 184507682 |
| LAIR1 | chr19 | − | 54360021 | 54360072 | 54361009 | 54361209 |
| ST6GAL1 | chr3 | + | 187038741 | 187038873 | 187042653 | 187043310 |
| OGFOD1 | chr16 | + | 56466151 | 56466268 | 56466875 | 56466967 |
| ARAP2 | chr4 | − | 36046709 | 36046849 | 36052005 | 36052053 |
| FDX1 | chr11 | + | 110435833 | 110435958 | 110456917 | 110457047 |
| TTC39B | chr9 | − | 15225916 | 15226012 | 15267913 | 15267948 |
| CD320 | chr19 | − | 8302776 | 8302980 | 8303854 | 8304088 |
| RUNX3 | chr1 | − | 24919239 | 24919344 | 24927573 | 24927730 |
| COP1 | chr1 | − | 176163814 | 176163891 | 176175909 | 176176007 |
| ANKRD27 | chr19 | − | 32644324 | 32644479 | 32646458 | 32646615 |
| TXLNA | chr1 | + | 32180313 | 32180514 | 32184524 | 32184616 |
| TPGS2 | chr18 | − | 36807846 | 36807934 | 36818893 | 36818973 |
| MCTP1 | chr5 | − | 94942347 | 94942427 | 94953218 | 94953361 |
| AD000671.1 | chr19 | + | 35752803 | 35752833 | 35752913 | 35752999 |
| COQ7 | chr16 | + | 19075720 | 19075860 | 19077305 | 19077374 |
| NRF1 | chr7 | + | 129657342 | 129657574 | 129671428 | 129671543 |
| IFTAP | chr11 | + | 36610080 | 36610239 | 36633283 | 36633438 |
| SCFD1 | chr14 | + | 30633946 | 30634037 | 30638124 | 30638247 |
| LY96 | chr8 | + | 74010000 | 74010129 | 74026788 | 74026841 |
| TMCO4 | chr1 | − | 19770541 | 19770569 | 19780579 | 19780766 |
| ANKZF1 | chr2 | + | 219231927 | 219232040 | 219232259 | 219232362 |
| SCYL1 | chr11 | + | 65537966 | 65538182 | 65538269 | 65538324 |
| PTPRA | chr20 | + | 2923206 | 2923285 | 2986764 | 2986849 |
| DRAM1 | chr12 | + | 101897862 | 101897930 | 101908185 | 101908363 |
| USP48 | chr1 | − | 21747066 | 21747149 | 21748137 | 21748271 |
| ARHGEF40 | chr14 | + | 21076562 | 21076643 | 21076773 | 21076890 |
| CHFR | chr12 | − | 132877554 | 132877654 | 132887195 | 132887340 |
| GYS1 | chr19 | − | 48978097 | 48978157 | 48981529 | 48981636 |
| RORC | chr1 | − | 151813238 | 151813346 | 151813487 | 151813702 |
| UVRAG | chr11 | + | 76004004 | 76004089 | 76007533 | 76007621 |
| SIRPB1 | chr20 | − | 1566152 | 1566267 | 1578337 | 1578694 |
| TTI2 | chr8 | − | 33509745 | 33509932 | 33511966 | 33512712 |
| COP1 | chr1 | − | 176149005 | 176149074 | 176184632 | 176184692 |
| TMEM8B | chr9 | + | 35829876 | 35829955 | 35834460 | 35834650 |
| CAPN12 | chr19 | − | 38734318 | 38734389 | 38735369 | 38735429 |
| HIPK3 | chr11 | + | 33286412 | 33287511 | 33328509 | 33328633 |
| SRC | chr20 | + | 37401601 | 37401678 | 37402434 | 37402588 |
| HERPUD2 | chr7 | − | 35667433 | 35667588 | 35694183 | 35694627 |
| KLRG1 | chr12 | + | 8992205 | 8992310 | 9008974 | 9009075 |
| COP1 | chr1 | − | 176149005 | 176149074 | 176175909 | 176176007 |
| WDSUB1 | chr2 | − | 159257757 | 159257864 | 159271701 | 159271795 |
| SPG7 | chr16 | + | 89526328 | 89526468 | 89529476 | 89529579 |
| P4HA1 | chr10 | − | 73043886 | 73043957 | 73044980 | 73045051 |
| IKBKG | chrX | + | 154558531 | 154558650 | 154560406 | 154560559 |
| METTL26 | chr16 | − | 634888 | 634956 | 635280 | 635340 |
| DNAJC1 | chr10 | − | 21920797 | 21920963 | 21929039 | 21929141 |
| NACC2 | chr9 | − | 136016264 | 136016429 | 136049635 | 136050580 |
| THOC6 | chr16 | + | 3025923 | 3025988 | 3026062 | 3026166 |
| ALG13 | chrX | + | 111682131 | 111682235 | 111682235 | 111682294 |
| RB1 | chr13 | + | 48376917 | 48377034 | 48380052 | 48380084 |
| LRP8 | chr1 | − | 53266472 | 53266647 | 53275630 | 53275753 |
| BCAS3 | chr17 | + | 60947218 | 60947352 | 60989970 | 60990235 |
| RNF170 | chr8 | − | 42870003 | 42870112 | 42873930 | 42874006 |
| MDM4 | chr1 | + | 204530683 | 204530817 | 204532190 | 204532246 |
| METTL26 | chr16 | − | 634888 | 635063 | 635280 | 635340 |
| FANCA | chr16 | − | 89758576 | 89758705 | 89761948 | 89762022 |
| CWF19L1 | chr10 | − | 100260217 | 100260319 | 100261978 | 100262063 |
| LRRK2 | chr12 | + | 40251464 | 40251544 | 40252909 | 40253016 |
| PRMT9 | chr4 | − | 147642786 | 147642940 | 147653851 | 147654566 |
| NUP54 | chr4 | − | 76134174 | 76134362 | 76136185 | 76136412 |
| ANKZF1 | chr2 | + | 219230227 | 219230405 | 219232259 | 219232362 |
| NAE1 | chr16 | − | 66821449 | 66821559 | 66823226 | 66823306 |
| PPP2R2D | chr10 | + | 131901237 | 131901330 | 131934457 | 131934555 |
| TSEN2 | chr3 | + | 12516610 | 12516661 | 12519058 | 12519197 |
| IKBKG | chrX | + | 154558531 | 154558650 | 154560406 | 154560559 |
| THEM4 | chr1 | − | 151889213 | 151889373 | 151895007 | 151895194 |
| COP1 | chr1 | − | 176149005 | 176149074 | 176175909 | 176176007 |
| CAST | chr5 | + | 96740744 | 96740783 | 96741265 | 96741358 |
| AGPAT3 | chr21 | + | 43903956 | 43904019 | 43959633 | 43959859 |
| TRPM7 | chr15 | − | 50586391 | 50586488 | 50589591 | 50589656 |
| RAD1 | chr5 | − | 34911553 | 34911812 | 34914694 | 34914961 |
| RNPC3 | chr1 | + | 103527694 | 103527742 | 103533738 | 103533857 |
| WDSUB1 | chr2 | − | 159257757 | 159257864 | 159259809 | 159259843 |
| SLBP | chr4 | − | 1700010 | 1700070 | 1703595 | 1703700 |
| KIF1C | chr17 | + | 4999849 | 4999970 | 5000219 | 5000352 |
| FOXRED1 | chr11 | + | 126274926 | 126275021 | 126275326 | 126275428 |
| DICER1-AS1 | chr14 | + | 95158192 | 95158234 | 95158875 | 95159035 |
| CRBN | chr3 | − | 3154746 | 3154831 | 3156218 | 3156281 |
| PACC1 | chr1 | − | 212375192 | 212375300 | 212379894 | 212380037 |
| SPATA13 | chr13 | + | 24284134 | 24284271 | 24286213 | 24286393 |
| ABCB7 | chrX | − | 75076521 | 75076654 | 75098941 | 75099061 |
| DENND4B | chr1 | − | 153933196 | 153933319 | 153933482 | 153933871 |
| JAK2 | chr9 | + | 4985939 | 4986022 | 5021962 | 5022213 |
| N4BP2L1 | chr13 | − | 32404320 | 32404397 | 32407249 | 32407338 |
| IMMP1L | chr11 | − | 31460625 | 31460714 | 31473723 | 31473786 |
| TBC1D2B | chr15 | − | 78044899 | 78045068 | 78054033 | 78054187 |
| ATF7IP | chr12 | + | 14423908 | 14425473 | 14434339 | 14434423 |
| SLBP | chr4 | − | 1700010 | 1700091 | 1703595 | 1703700 |
| ARHGAP19 | chr10 | − | 97256317 | 97256404 | 97259401 | 97259628 |
| POLL | chr10 | − | 101582762 | 101582891 | 101587245 | 101587406 |
| IL15 | chr4 | + | 141656185 | 141656307 | 141719365 | 141719476 |
| RIN3 | chr14 | + | 92555750 | 92555955 | 92615406 | 92615479 |
| PXN | chr12 | − | 120216272 | 120216581 | 120219206 | 120220091 |
| PLXDC1 | chr17 | − | 39109247 | 39109391 | 39139653 | 39139832 |
| N4BP2L1 | chr13 | − | 32404320 | 32404397 | 32407644 | 32407772 |
| PI4KAP2 | chr22 | − | 21487871 | 21488023 | 21491993 | 21492094 |
| SUZ12P1 | chr17 | + | 30734896 | 30734943 | 30735032 | 30735097 |
| SCAF8 | chr6 | + | 154778000 | 154778045 | 154787860 | 154788022 |
| GTF2IRD2B | chr7 | + | 75112396 | 75112535 | 75120890 | 75121010 |
| SENP6 | chr6 | + | 75623899 | 75623960 | 75634706 | 75634811 |
| DNMT3A | chr2 | − | 25246619 | 25246776 | 25247590 | 25247749 |
| DCP1B | chr12 | − | 1993263 | 1993391 | 1997934 | 1997975 |
| TMEM39A | chr3 | − | 119452446 | 119452530 | 119458017 | 119458240 |
| CBWD3 | chr9 | + | 68269599 | 68269722 | 68270301 | 68270518 |
| RETSAT | chr2 | − | 85345974 | 85346094 | 85350779 | 85351021 |
| DENND6A | chr3 | − | 57633264 | 57633354 | 57634557 | 57634622 |
| ZSWIM7 | chr17 | − | 15977578 | 15977700 | 15977799 | 15977913 |
| AK5 | chr1 | + | 77286940 | 77287127 | 77293792 | 77293960 |
| MDM1 | chr12 | − | 68326656 | 68327021 | 68331106 | 68331221 |
| ITGA6 | chr2 | + | 172467480 | 172467560 | 172470973 | 172471105 |
| HDAC4 | chr2 | − | 239163802 | 239163923 | 239176412 | 239176563 |
| SIRPB1 | chr20 | − | 1571719 | 1572037 | 1578337 | 1578694 |
| ELMO2 | chr20 | − | 46380252 | 46380303 | 46383415 | 46383494 |
| ABCA7 | chr19 | + | 1055096 | 1055351 | 1055906 | 1055939 |
| HLA-B | chr6 | − | 31355106 | 31355223 | 31356687 | 31356957 |
| KIAA0753 | chr17 | − | 6622881 | 6623097 | 6623508 | 6623571 |
| MRNIP | chr5 | − | 179847977 | 179848066 | 179853196 | 179853276 |
| RIOK1 | chr6 | + | 7395052 | 7395143 | 7396702 | 7396772 |
| HLA-B | chr6 | − | 31355316 | 31355592 | 31356687 | 31356957 |
| RNF144B | chr6 | + | 18399498 | 18399699 | 18427580 | 18427685 |
| RAB35 | chr12 | − | 120099029 | 120099154 | 120108416 | 120108467 |
| DOCK10 | chr2 | − | 224916694 | 224916784 | 224931548 | 224931668 |
| FBXL4 | chr6 | − | 98917373 | 98917719 | 98934761 | 98934879 |
| TMEM189-UBE2V1 | chr20 | − | 50130855 | 50130947 | 50143501 | 50143621 |
| SEPTIN11 | chr4 | + | 76996424 | 76996539 | 77005600 | 77005796 |
| RBM22 | chr5 | − | 150699241 | 150699271 | 150700443 | 150700497 |
| BSCL2 | chr11 | − | 62702467 | 62702549 | 62705467 | 62705617 |
| SNHG17 | chr20 | − | 38422091 | 38422241 | 38426418 | 38426503 |
| SORBS3 | chr8 | + | 22566344 | 22566484 | 22566660 | 22566868 |
| SPOPL | chr2 | + | 138550156 | 138550294 | 138550482 | 138550604 |
| SLC7A6 | chr16 | + | 68274690 | 68275249 | 68287745 | 68287871 |
| NDRG1 | chr8 | − | 133280231 | 133280267 | 133284248 | 133284329 |
| LAIR1 | chr19 | − | 54361009 | 54361209 | 54364294 | 54364330 |
| LAIR1 | chr19 | − | 54361009 | 54361243 | 54364294 | 54364330 |
| ATL2 | chr2 | − | 38309378 | 38309506 | 38343267 | 38343512 |
| SCAF8 | chr6 | + | 154773988 | 154774072 | 154787860 | 154788022 |
| AC009061.2 | chr16 | + | 67484109 | 67484328 | 67492024 | 67492121 |
| TMCO4 | chr1 | − | 19770541 | 19770569 | 19771307 | 19771482 |
| BSCL2 | chr11 | − | 62702467 | 62702549 | 62705300 | 62705617 |
| MAPK6 | chr15 | + | 52045829 | 52047015 | 52049992 | 52050137 |
| PLSCR3 | chr17 | − | 7393466 | 7393509 | 7394103 | 7394267 |
| FOXJ3 | chr1 | − | 42188736 | 42188928 | 42189302 | 42189404 |
| SLC39A11 | chr17 | − | 72947730 | 72947875 | 73031555 | 73031714 |
| PYROXD1 | chr12 | + | 21440367 | 21440448 | 21445346 | 21445466 |
| RNF144B | chr6 | + | 18399498 | 18399699 | 18459606 | 18459751 |
| TMEM220 | chr17 | − | 10725010 | 10725134 | 10726203 | 10726264 |
| TADA2A | chr17 | + | 37442563 | 37442652 | 37444695 | 37444768 |
| CYRIB | chr8 | − | 129912469 | 129912515 | 129948965 | 129949067 |
| PEX1 | chr7 | − | 92518994 | 92519078 | 92522101 | 92522245 |
| TSPAN2 | chr1 | − | 115058882 | 115058981 | 115072904 | 115073007 |
| TTLL12 | chr22 | − | 43180741 | 43180940 | 43182979 | 43183149 |
| TMEM234 | chr1 | − | 32216383 | 32216517 | 32216888 | 32216947 |
| BCL9L | chr11 | − | 118909913 | 118910015 | 118918825 | 118918879 |
| AC008073.3 | chr2 | + | 24164197 | 24164335 | 24177801 | 24177870 |
| GNB4 | chr3 | − | 179419398 | 179419505 | 179420888 | 179420927 |
| TSPAN2 | chr1 | − | 115060463 | 115060538 | 115072904 | 115073007 |
| TMEM234 | chr1 | − | 32215135 | 32215216 | 32216888 | 32216947 |
| ERAP2 | chr5 | + | 96896731 | 96896863 | 96900120 | 96900245 |
| ANKRD6 | chr6 | + | 89606006 | 89606105 | 89612271 | 89612370 |
| DDX60L | chr4 | − | 168394460 | 168394617 | 168395958 | 168396127 |
| GTF2H2B | chr5 | + | 70434178 | 70434266 | 70434914 | 70435022 |
| SLC35F2 | chr11 | − | 107804717 | 107804770 | 107805358 | 107805515 |
| TBCD | chr17 | + | 82763964 | 82764062 | 82766266 | 82766368 |
| ZFR | chr5 | − | 32417647 | 32417792 | 32419820 | 32420103 |
| PXYLP1 | chr3 | + | 141260122 | 141260254 | 141278341 | 141278500 |
| SOS1 | chr2 | − | 39058672 | 39058804 | 39067627 | 39067753 |
| SNHG17 | chr20 | − | 38422086 | 38422241 | 38425934 | 38426052 |
| CA5B | chrX | + | 15721496 | 15721592 | 15745630 | 15745710 |
| SNHG17 | chr20 | − | 38422091 | 38422241 | 38425934 | 38426052 |
| ERC1 | chr12 | + | 1263033 | 1263165 | 1289851 | 1290012 |
| CYRIB | chr8 | − | 129912469 | 129912515 | 129948965 | 129949067 |
| RANBP10 | chr16 | − | 67772033 | 67772086 | 67805427 | 67805539 |
| AC092070.2 | chr19 | + | 53200623 | 53200703 | 53204015 | 53204142 |
| SETD6 | chr16 | + | 58516477 | 58516672 | 58516807 | 58516928 |
| ITGB2 | chr21 | − | 44910283 | 44910372 | 44910724 | 44910785 |
| CEP170 | chr1 | − | 243142366 | 243142463 | 243156220 | 243156347 |
| SNHG17 | chr20 | − | 38422086 | 38422241 | 38422290 | 38422368 |
| LRP8 | chr1 | − | 53262067 | 53262207 | 53264168 | 53264396 |
| SNHG17 | chr20 | − | 38422091 | 38422241 | 38422290 | 38422368 |
| ANKRD36B | chr2 | − | 97515731 | 97515945 | 97523325 | 97523467 |

| GeneID | IncFormLen | SkipFormLen | PValue | FDR | IncLevelDifference |
|---|---|---|---|---|---|
| CR1 | 298 | 298 | 5.48E−12 | 7.24E−09 | 0.438 |
| PIGL | 217 | 181 | 9.48E−08 | 2.53E−05 | 0.239 |
| XPNPEP3 | 241 | 298 | 0.00048788 | 0.0195785 | 0.238 |
| MAPKBP1 | 298 | 296 | 4.39E−05 | 0.0032844 | 0.231 |
| TBC1D12 | 259 | 298 | 0.00020981 | 0.01073042 | 0.188 |
| AC004593.2 | 261 | 188 | 0.00070623 | 0.02506553 | 0.184 |
| AC016831.6 | 265 | 192 | 4.24E−06 | 0.00053836 | 0.184 |
| WDR60 | 252 | 279 | 0.00102682 | 0.03186336 | 0.179 |
| ZNF720 | 245 | 284 | 0.0002187 | 0.01104665 | 0.171 |
| MYO15B | 195 | 298 | 0.00016888 | 0.00909469 | 0.167 |
| HSD17B3 | 225 | 257 | 0.0015585 | 0.04254252 | 0.165 |
| WHAMMP3 | 298 | 279 | 0.00094552 | 0.03016446 | 0.164 |
| AL392172.1 | 246 | 220 | 3.30E−05 | 0.00261998 | 0.163 |
| ATXN2 | 203 | 298 | 0.00025889 | 0.01240124 | 0.152 |
| AL356481.1 | 298 | 278 | 0.00055035 | 0.02126769 | 0.149 |
| PDE8A | 227 | 220 | 0.00048117 | 0.01943914 | 0.148 |
| ZNF266 | 211 | 268 | 0.00177726 | 0.04646003 | 0.148 |
| AL162258.1 | 217 | 265 | 3.47E−05 | 0.00273279 | 0.146 |
| CAMKK1 | 283 | 186 | 0.00012409 | 0.00717412 | 0.145 |
| PPFIA1 | 266 | 179 | 0.0014832 | 0.04131168 | 0.143 |
| ZNF266 | 211 | 298 | 0.00055393 | 0.02130244 | 0.143 |
| CFAP70 | 298 | 271 | 0.00026034 | 0.01245667 | 0.139 |
| ACCS | 209 | 220 | 0.00058237 | 0.02197879 | 0.138 |
| ATF7IP2 | 216 | 235 | 7.24E−05 | 0.0047584 | 0.137 |
| NEIL2 | 294 | 298 | 0.00065277 | 0.02401196 | 0.136 |
| TAF4 | 239 | 298 | 4.42E−08 | 1.38E−05 | 0.129 |
| PNPT1 | 207 | 252 | 0.00025212 | 0.01216099 | 0.128 |
| HLA-DRB5 | 298 | 298 | 0.0009505 | 0.0302426 | 0.128 |
| DDX60L | 298 | 254 | 0.00155 | 0.04252019 | 0.126 |
| YAF2 | 291 | 219 | 0.00039101 | 0.01661163 | 0.126 |
| SP140 | 227 | 298 | 0.0011168 | 0.03387702 | 0.125 |
| KIAA1841 | 243 | 298 | 0.00125417 | 0.03671553 | 0.125 |
| TNNT3 | 161 | 168 | 1.22E−05 | 0.00123514 | 0.125 |
| ARFIP1 | 194 | 210 | 0.00171986 | 0.04549068 | 0.12 |
| TUBD1 | 298 | 290 | 0.0015442 | 0.04244113 | 0.12 |
| POMZP3 | 267 | 287 | 0.00102346 | 0.03182082 | 0.117 |
| SH3TC1 | 191 | 298 | 1.21E−05 | 0.00122804 | 0.117 |
| POLR2J4 | 236 | 226 | 0.00010481 | 0.00636332 | 0.116 |
| CDKL3 | 238 | 298 | 0.00089596 | 0.02913041 | 0.112 |
| CD300LF | 262 | 172 | 0.00169595 | 0.04510458 | 0.112 |
| ELP1 | 246 | 240 | 0.00119097 | 0.0355404 | 0.111 |
| RPTOR | 298 | 296 | 9.05E−05 | 0.00569354 | 0.111 |
| ZNF266 | 211 | 298 | 0.0011912 | 0.0355404 | 0.111 |
| POLR2J4 | 298 | 226 | 0.000146 | 0.00815921 | 0.11 |
| CBWD3 | 236 | 194 | 0.0001464 | 0.00817119 | 0.11 |
| WDR12 | 265 | 195 | 8.91E−06 | 0.00098839 | 0.109 |
| PVT1 | 278 | 245 | 5.56E−05 | 0.00395755 | 0.109 |
| LLGL2 | 197 | 208 | 8.52E−05 | 0.00546071 | 0.107 |
| TCFL5 | 298 | 291 | 0.00019662 | 0.01021553 | 0.104 |
| MCTP1 | 251 | 224 | 9.96E−06 | 0.00108492 | 0.104 |
| ST3GAL5 | 261 | 298 | 0.00151188 | 0.04186972 | 0.103 |
| UBXN8 | 272 | 272 | 0.00048321 | 0.01948491 | 0.102 |
| ATXN2 | 282 | 203 | 3.72E−06 | 0.0004904 | 0.102 |
| MICA | 298 | 298 | 5.91E−06 | 0.0007001 | 0.101 |
| CARD8 | 298 | 254 | 0.00105347 | 0.03233151 | 0.101 |
| CARD8 | 298 | 254 | 0.00171755 | 0.04545715 | 0.101 |
| AL121845.3 | 298 | 264 | 3.17E−05 | 0.00255419 | 0.099 |
| IL32 | 176 | 209 | 0.00097148 | 0.03059748 | 0.099 |
| GMPPB | 298 | 228 | 8.07E−05 | 0.00521996 | 0.098 |
| ZEB2 | 221 | 298 | 0.00044122 | 0.01814406 | 0.098 |
| TBC1D8 | 298 | 267 | 4.63E−07 | 9.26E−05 | 0.098 |
| LEMD3 | 255 | 228 | 4.39E−05 | 0.00328553 | 0.098 |
| SLC38A1 | 229 | 250 | 0.00082857 | 0.02784684 | 0.098 |
| JARID2 | 298 | 298 | 0.000533 | 0.02081864 | 0.097 |
| SLC38A9 | 256 | 298 | 1.03E−05 | 0.00111287 | 0.096 |
| AC119396.1 | 274 | 298 | 0.00027994 | 0.0130508 | 0.096 |
| UBE3C | 254 | 237 | 1.32E−07 | 3.29E−05 | 0.095 |
| MARCHF8 | 200 | 298 | 1.37E−05 | 0.00132994 | 0.095 |
| TUBGCP4 | 298 | 284 | 0.00073663 | 0.02574939 | 0.095 |
| SLAMF1 | 298 | 239 | 0.00023608 | 0.01166772 | 0.092 |
| GTF2H2 | 257 | 213 | 0.00105913 | 0.03246645 | 0.091 |
| IRF8 | 255 | 298 | 0.00017435 | 0.00929061 | 0.091 |
| POMZP3 | 241 | 287 | 0.00176872 | 0.04633317 | 0.09 |
| MCM6 | 295 | 291 | 0.00111899 | 0.03391976 | 0.09 |
| PVT1 | 260 | 245 | 0.00025768 | 0.01237056 | 0.09 |
| NIBAN3 | 298 | 265 | 0.00144908 | 0.04067329 | 0.09 |
| NFS1 | 284 | 298 | 0.00055428 | 0.02130244 | 0.088 |
| KDM3B | 224 | 298 | 0.00105281 | 0.03233151 | 0.088 |
| AC114490.2 | 298 | 298 | 0.0002489 | 0.01206848 | 0.088 |
| ZC3H13 | 261 | 258 | 0.00157672 | 0.04279528 | 0.088 |
| TGS1 | 298 | 267 | 0.00094666 | 0.03016446 | 0.087 |
| CR1 | 298 | 298 | 4.31E−05 | 0.0032423 | 0.087 |
| POMZP3 | 298 | 267 | 0.00123126 | 0.03638696 | 0.087 |
| POMZP3 | 298 | 298 | 0.00093008 | 0.02983237 | 0.087 |
| DDX21 | 298 | 269 | 0.00092869 | 0.02980962 | 0.087 |
| SYNE1 | 298 | 298 | 2.82E−06 | 0.00039618 | 0.086 |
| ZBTB17 | 298 | 275 | 0.00017027 | 0.00914048 | 0.086 |
| MYOM1 | 298 | 186 | 0.00175913 | 0.04613745 | 0.085 |
| TIMM23B-AGAP6 | 219 | 298 | 4.87E−06 | 0.00060876 | 0.085 |
| SBNO1 | 285 | 262 | 0.00123301 | 0.03641393 | 0.084 |
| RAB11FIP3 | 284 | 185 | 8.48E−05 | 0.00544454 | 0.084 |
| CNOT1 | 298 | 298 | 0.00118129 | 0.0353477 | 0.084 |
| NMRK1 | 297 | 221 | 5.01E−05 | 0.00365048 | 0.083 |
| IRF8 | 197 | 298 | 0.00022063 | 0.01111798 | 0.083 |
| TSC2 | 298 | 262 | 0.00192466 | 0.04877674 | 0.083 |
| RCHY1 | 228 | 194 | 0.00039417 | 0.01667546 | 0.083 |
| PVT1 | 298 | 245 | 0.0004551 | 0.01857509 | 0.082 |
| COG8 | 298 | 298 | 0.00011458 | 0.00677684 | 0.082 |
| FKBP15 | 234 | 219 | 6.52E−06 | 0.00076584 | 0.081 |
| PLBD1-AS1 | 198 | 298 | 0.00184072 | 0.04752409 | 0.081 |
| TOM1 | 209 | 189 | 0.00020697 | 0.01062645 | 0.08 |
| SUGP1 | 253 | 298 | 0.00036801 | 0.01585242 | 0.08 |
| MCTP1 | 259 | 247 | 0.0001488 | 0.00827339 | 0.08 |
| ZNF185 | 191 | 233 | 0.00028197 | 0.01310331 | 0.08 |
| TAF1D | 197 | 184 | 0.00013431 | 0.00759342 | 0.08 |
| CELF1 | 278 | 263 | 0.00078092 | 0.02680339 | 0.08 |
| PIGG | 298 | 298 | 2.92E−05 | 0.00240459 | 0.08 |
| C11orf80 | 298 | 259 | 0.00133562 | 0.03847959 | 0.079 |
| ZNF638 | 275 | 187 | 0.00013735 | 0.00772555 | 0.079 |
| TRIM14 | 298 | 242 | 0.0004024 | 0.01695619 | 0.079 |
| NEIL2 | 289 | 298 | 0.0013671 | 0.03905099 | 0.078 |
| SNX9 | 298 | 297 | 0.00156255 | 0.04254252 | 0.078 |
| NSUN6 | 269 | 294 | 0.00107771 | 0.03285145 | 0.078 |
| MRTFA | 298 | 298 | 0.00083327 | 0.02794029 | 0.078 |
| SLC3A2 | 208 | 298 | 2.27E−05 | 0.00196658 | 0.077 |
| SNAPC1 | 298 | 245 | 0.00038758 | 0.01651474 | 0.077 |
| ING4 | 221 | 255 | 0.00083585 | 0.02796217 | 0.077 |
| SERF2 | 205 | 201 | 0.00050539 | 0.0200273 | 0.077 |
| FAM114A2 | 298 | 249 | 1.69E−05 | 0.00155636 | 0.076 |
| CELF1 | 278 | 260 | 0.0012026 | 0.03575811 | 0.076 |
| NFKB2 | 235 | 275 | 0.00089617 | 0.02913041 | 0.076 |
| VARS1 | 274 | 253 | 0.00078159 | 0.02680525 | 0.074 |
| CLEC17A | 245 | 298 | 1.59E−08 | 6.01E−06 | 0.074 |
| TMEM184B | 241 | 298 | 0.00103551 | 0.03206607 | 0.074 |
| EML2 | 284 | 281 | 0.000151 | 0.00838483 | 0.074 |
| CCM2 | 298 | 298 | 0.00022421 | 0.01125968 | 0.073 |
| WRNIP1 | 298 | 298 | 0.00108961 | 0.03312142 | 0.073 |
| DENND4B | 298 | 285 | 0.00043357 | 0.01791425 | 0.073 |
| AL392172.1 | 246 | 220 | 2.53E−06 | 0.00036164 | 0.073 |
| TMEM131 | 298 | 292 | 0.00054011 | 0.02094966 | 0.073 |
| RYK | 250 | 275 | 0.00084547 | 0.02819757 | 0.073 |
| TMCO3 | 298 | 229 | 1.54E−05 | 0.00144515 | 0.073 |
| FOXP1 | 219 | 298 | 1.92E−05 | 0.00171489 | 0.072 |
| DNMT1 | 278 | 291 | 5.58E−05 | 0.00395965 | 0.072 |
| ZNF254 | 276 | 245 | 0.00018757 | 0.00986269 | 0.072 |
| TCFL5 | 298 | 291 | 0.00147091 | 0.04110058 | 0.071 |
| PILRB | 247 | 283 | 0.00170619 | 0.04532157 | 0.071 |
| IDH1 | 298 | 298 | 5.73E−05 | 0.00403193 | 0.071 |
| NUP98 | 298 | 298 | 0.00124158 | 0.03651864 | 0.071 |
| DDB2 | 298 | 292 | 0.00048574 | 0.01953283 | 0.071 |
| IGHD | 221 | 298 | 0.00034604 | 0.01522089 | 0.071 |
| POLR2J3 | 298 | 298 | 3.00E−05 | 0.00243688 | 0.07 |
| UBAP2L | 217 | 298 | 0.00020992 | 0.01073042 | 0.07 |
| TSEN2 | 227 | 200 | 2.20E−10 | 1.85E−07 | 0.07 |
| COA1 | 270 | 250 | 8.68E−11 | 8.23E−08 | 0.07 |
| MPHOSPH6 | 240 | 244 | 0.00193992 | 0.04896416 | 0.07 |
| CERS4 | 256 | 298 | 0.00074575 | 0.02594337 | 0.07 |
| AC069281.2 | 211 | 266 | 0.00028772 | 0.01328564 | 0.069 |
| ATP2B4 | 298 | 257 | 0.00064377 | 0.02384251 | 0.069 |
| EIF4G1 | 298 | 254 | 0.00083901 | 0.02804646 | 0.069 |
| WDFY3 | 246 | 298 | 0.0007332 | 0.02565006 | 0.069 |
| ECPAS | 271 | 298 | 0.00089734 | 0.02913442 | 0.069 |
| SLC25A19 | 298 | 298 | 0.00154334 | 0.04244113 | 0.069 |
| STAU2 | 295 | 284 | 0.00021038 | 0.01073818 | 0.068 |
| RPS6KA3 | 231 | 230 | 0.00182483 | 0.04717926 | 0.068 |
| DUS2 | 298 | 237 | 0.00023172 | 0.01151709 | 0.068 |
| MAN1B1 | 298 | 298 | 4.34E−05 | 0.00325834 | 0.068 |
| KMT2D | 280 | 298 | 0.00030157 | 0.01363622 | 0.068 |
| RRP8 | 298 | 256 | 0.00142702 | 0.0403396 | 0.068 |
| SPAG9 | 298 | 263 | 0.00029058 | 0.01334711 | 0.068 |
| CPNE1 | 204 | 283 | 0.00121605 | 0.03605966 | 0.067 |
| UBXN11 | 250 | 204 | 0.00015983 | 0.0087416 | 0.067 |
| SLC44A2 | 234 | 298 | 0.0001527 | 0.00844861 | 0.067 |
| KDM5A | 284 | 255 | 0.0017128 | 0.04543138 | 0.067 |
| TNPO1 | 245 | 225 | 0.00057925 | 0.0218988 | 0.067 |
| HEATR5B | 298 | 298 | 1.12E−05 | 0.00115495 | 0.066 |
| RNF170 | 258 | 223 | 0.00135517 | 0.03880519 | 0.066 |
| RHOT1 | 202 | 251 | 8.29E−05 | 0.0053315 | 0.066 |
| TMEM50B | 247 | 233 | 0.00034375 | 0.01515092 | 0.065 |
| SCLT1 | 228 | 245 | 0.00127582 | 0.03719944 | 0.065 |
| MAN2B1 | 230 | 298 | 0.00062591 | 0.0233192 | 0.065 |
| PCBP1-AS1 | 298 | 246 | 9.80E−05 | 0.00602325 | 0.065 |
| AC069281.2 | 226 | 211 | 0.0001373 | 0.00772555 | 0.064 |
| DTNBP1 | 200 | 210 | 0.00131318 | 0.03800884 | 0.064 |
| PINK1 | 298 | 298 | 0.00059704 | 0.02243514 | 0.064 |
| HDAC7 | 233 | 260 | 7.99E−07 | 0.00014692 | 0.064 |
| CMTM1 | 283 | 248 | 0.00050909 | 0.0201369 | 0.064 |
| CDC23 | 269 | 225 | 0.00174379 | 0.04592855 | 0.064 |
| PCBP1-AS1 | 298 | 298 | 0.0006505 | 0.02396901 | 0.064 |
| PCBP1-AS1 | 298 | 298 | 0.00070862 | 0.02506553 | 0.064 |
| EHBP1L1 | 203 | 298 | 0.0009658 | 0.03051008 | 0.063 |
| ANKZF1 | 298 | 298 | 0.00121385 | 0.03604339 | 0.063 |
| AC118553.2 | 245 | 212 | 1.04E−06 | 0.00018014 | 0.063 |
| NAE1 | 213 | 241 | 3.00E−05 | 0.00243688 | 0.063 |
| GIT1 | 236 | 223 | 0.00037497 | 0.01607167 | 0.063 |
| CREBBP | 298 | 298 | 0.00103022 | 0.03193966 | 0.063 |
| RALY | 197 | 298 | 1.24E−05 | 0.00124332 | 0.062 |
| XAF1 | 298 | 298 | 1.26E−08 | 4.95E−06 | 0.062 |
| CCM2 | 298 | 233 | 0.00057549 | 0.02182961 | 0.062 |
| CPSF7 | 298 | 207 | 6.88E−05 | 0.00458061 | 0.062 |
| CAST | 230 | 188 | 0.00165527 | 0.04422018 | 0.062 |
| SMG5 | 298 | 298 | 4.67E−05 | 0.00344756 | 0.062 |
| PVT1 | 262 | 245 | 7.86E−05 | 0.00511519 | 0.062 |
| RGS6 | 298 | 239 | 0.00044255 | 0.01818155 | 0.062 |
| SUMF2 | 194 | 298 | 0.00095616 | 0.03033397 | 0.062 |
| RALY | 197 | 298 | 1.26E−05 | 0.00125274 | 0.061 |
| RALY | 197 | 298 | 1.29E−05 | 0.00127168 | 0.061 |
| MLKL | 285 | 231 | 0.00048689 | 0.0195609 | 0.061 |
| ITPA | 265 | 226 | 6.31E−05 | 0.00430417 | 0.061 |
| GATAD2A | 239 | 281 | 0.00077975 | 0.0268032 | 0.061 |
| MKNK1 | 254 | 205 | 0.0010365 | 0.03206607 | 0.061 |
| MIR4435-2HG | 298 | 243 | 0.00118027 | 0.0353477 | 0.061 |
| SSBP4 | 234 | 239 | 2.11E−05 | 0.00184807 | 0.061 |
| CCM2 | 298 | 233 | 0.00096661 | 0.03051008 | 0.06 |
| TNFRSF1A | 223 | 298 | 0.00065811 | 0.02414738 | 0.06 |
| TLE3 | 298 | 226 | 0.0001088 | 0.00655037 | 0.06 |
| ARHGAP25 | 298 | 237 | 0.00089763 | 0.02913442 | 0.059 |
| TRAK1 | 298 | 298 | 0.00181709 | 0.04709083 | 0.059 |
| LRCH3 | 221 | 198 | 0.00169016 | 0.04503314 | 0.059 |
| TLE3 | 298 | 226 | 0.00012445 | 0.0071754 | 0.059 |
| PLD3 | 292 | 298 | 0.00182639 | 0.0471916 | 0.059 |
| ADA | 277 | 221 | 0.00043265 | 0.01789317 | 0.058 |
| SHISA5 | 265 | 298 | 0.00024278 | 0.01189061 | 0.058 |
| NCF1 | 254 | 295 | 0.00029681 | 0.013463 | 0.058 |
| NUP88 | 298 | 275 | 0.00051554 | 0.02027376 | 0.058 |
| USB1 | 203 | 255 | 0.00032957 | 0.01463688 | 0.058 |
| FMNL1 | 298 | 251 | 0.0006872 | 0.02482549 | 0.058 |
| CYB5R4 | 182 | 182 | 7.92E−05 | 0.00514256 | 0.057 |
| NFKB2 | 288 | 298 | 0.00105104 | 0.03230934 | 0.057 |
| NUP88 | 298 | 298 | 0.0010677 | 0.03263743 | 0.057 |
| RCBTB2 | 298 | 257 | 0.0014398 | 0.04060637 | 0.057 |
| STRADA | 252 | 271 | 0.00042276 | 0.01758391 | 0.057 |
| CRTC2 | 298 | 298 | 0.00089401 | 0.02910375 | 0.057 |
| UNC13D | 242 | 253 | 0.00027138 | 0.01280955 | 0.057 |
| ICE1 | 298 | 298 | 0.00178031 | 0.04649707 | 0.056 |
| TMBIM1 | 203 | 200 | 1.05E−05 | 0.00112187 | 0.056 |
| SH2D3C | 298 | 274 | 0.00020997 | 0.01073042 | 0.056 |
| SLC44A2 | 242 | 234 | 0.00041184 | 0.0172616 | 0.056 |
| SORL1 | 298 | 217 | 8.62E−05 | 0.00549336 | 0.056 |
| LTBR | 247 | 257 | 0.00153637 | 0.04227925 | 0.056 |
| CHP1 | 277 | 21 | 6.70E−05 | 0.00448333 | 0.056 |
| CTDNEP1 | 261 | 234 | 0.0005603 | 0.02140532 | 0.056 |
| RPS12 | 252 | 251 | 1.94E−05 | 0.00172366 | 0.055 |
| WDFY4 | 298 | 297 | 0.0017349 | 0.04579958 | 0.055 |
| FANCA | 298 | 258 | 4.75E−05 | 0.00349735 | 0.055 |
| CPNE1 | 298 | 283 | 0.00087971 | 0.0289142 | 0.054 |
| CPNE1 | 283 | 283 | 0.00073825 | 0.0257833 | 0.054 |
| OSBPL11 | 298 | 295 | 0.00071835 | 0.02533312 | 0.054 |
| RHOT1 | 202 | 258 | 0.00064741 | 0.02392665 | 0.054 |
| ITGAL | 225 | 263 | 0.00087182 | 0.02877966 | 0.054 |
| INTS8 | 298 | 257 | 0.00141944 | 0.04017762 | 0.053 |
| DBNL | 198 | 231 | 0.00064505 | 0.02386895 | 0.053 |
| GSN | 283 | 240 | 0.00058209 | 0.02197879 | 0.053 |
| CNOT3 | 206 | 298 | 0.00029295 | 0.01339947 | 0.052 |
| DAGLB | 276 | 227 | 0.00013979 | 0.00783195 | 0.052 |
| DBNL | 198 | 255 | 0.0006283 | 0.02338822 | 0.052 |
| STXBP2 | 259 | 245 | 0.0003417 | 0.01507544 | 0.052 |
| SNHG29 | 298 | 218 | 1.38E−06 | 0.00022001 | 0.052 |
| UNC13D | 255 | 221 | 0.0007848 | 0.02685201 | 0.052 |
| FCHO1 | 291 | 298 | 0.00067804 | 0.02459404 | 0.052 |
| SLC35C2 | 298 | 279 | 0.00151146 | 0.04186972 | 0.051 |
| WDR47 | 298 | 28 | 2.15E−07 | 4.90E−05 | 0.051 |
| MKNK2 | 298 | 298 | 0.00108304 | 0.03299074 | 0.051 |
| INPP5D | 298 | 290 | 0.00177622 | 0.04646003 | 0.051 |
| LRCH4 | 211 | 253 | 0.00093846 | 0.03005708 | 0.051 |
| TTC7A | 254 | 267 | 0.00035511 | 0.01547905 | 0.051 |
| UNC93B1 | 243 | 274 | 0.00132996 | 0.0383674 | 0.051 |
| PDE4B | 298 | 249 | 0.00184489 | 0.04755705 | 0.051 |
| HLA-DRB1 | 298 | 298 | 3.00E−07 | 6.47E−05 | 0.05 |
| TNFRSF1A | 178 | 298 | 0.00070901 | 0.02506553 | 0.05 |
| AC118553.2 | 283 | 245 | 0.0008742 | 0.02880289 | 0.05 |
| FGR | 298 | 298 | 0.00055303 | 0.02130244 | 0.05 |
| COG4 | 251 | 278 | 0.00197944 | 0.04970273 | 0.05 |
| FLOT1 | 240 | 269 | 0.00107116 | 0.0326975 | −0.05 |
| CAMK1D | 225 | 262 | 0.00038266 | 0.01636919 | −0.05 |
| LMBR1 | 230 | 216 | 0.00053683 | 0.02093045 | −0.05 |
| TSC2 | 276 | 293 | 6.70E−11 | 6.49E−08 | −0.05 |
| FGR | 212 | 248 | 1.94E−05 | 0.00172366 | −0.05 |
| NAA60 | 219 | 279 | 4.67E−11 | 4.73E−08 | −0.05 |
| INTS6 | 210 | 298 | 5.78E−07 | 0.00011104 | −0.05 |
| TTC16 | 291 | 298 | 0.00022158 | 0.01115312 | −0.051 |
| H2AZ2 | 227 | 279 | 0.00036803 | 0.01585242 | −0.051 |
| AC004997.1 | 228 | 220 | 1.39E−08 | 5.32E−06 | −0.051 |
| SPTLC1 | 282 | 298 | 0.00072102 | 0.02536662 | −0.051 |
| BAG6 | 209 | 29 | 0.00024165 | 0.01186215 | −0.051 |
| TTC17 | 298 | 298 | 0.00073059 | 0.02560087 | −0.051 |
| CDK16 | 240 | 227 | 0.00150986 | 0.04186697 | −0.051 |
| NAXD | 298 | 195 | 1.48E−05 | 0.00140621 | −0.051 |
| IGFLR1 | 298 | 298 | 0.00060981 | 0.02280057 | −0.051 |
| GOLGA7 | 282 | 298 | 1.17E−06 | 0.00019558 | −0.052 |
| YY1AP1 | 190 | 253 | 4.95E−08 | 1.49E−05 | −0.052 |
| YY1AP1 | 190 | 253 | 2.74E−08 | 9.63E−06 | −0.052 |
| CASC4 | 252 | 230 | 0.00188468 | 0.04827725 | −0.052 |
| PTDSS1 | 194 | 274 | 0.00061092 | 0.02281929 | −0.052 |
| CEP290 | 298 | 272 | 0.00041501 | 0.01732787 | −0.052 |
| TIMM23B-AGAP6 | 280 | 219 | 3.22E−05 | 0.00258438 | −0.052 |
| IL21R | 214 | 252 | 0.00013945 | 0.00782313 | −0.052 |
| DNMT1 | 186 | 257 | 0.00010149 | 0.0061957 | −0.052 |
| PDIA4 | 298 | 298 | 0.00012638 | 0.00723902 | −0.053 |
| SIPA1 | 298 | 298 | 0.00126219 | 0.03690062 | −0.053 |
| POLR2F | 219 | 280 | 0.00181023 | 0.04698247 | −0.053 |
| POLL | 298 | 278 | 0.00174752 | 0.04597122 | −0.053 |
| PSMA5 | 216 | 276 | 0.00019112 | 0.01001319 | −0.053 |
| PAFAH2 | 298 | 298 | 0.00041279 | 0.01727535 | −0.053 |
| MOV10 | 298 | 298 | 0.00032378 | 0.01447397 | −0.053 |
| HMGB2 | 298 | 295 | 0.00137005 | 0.03910973 | −0.053 |
| PYCR2 | 220 | 298 | 0.00130572 | 0.03786846 | −0.053 |
| B4GALT3 | 295 | 298 | 0.0009858 | 0.03098116 | −0.053 |
| RHOT2 | 227 | 272 | 0.00156139 | 0.04254252 | −0.053 |
| DDX17 | 29 | 298 | 0.00160304 | 0.04332089 | −0.054 |
| NQO2 | 298 | 241 | 0 | 0 | −0.054 |
| WDR70 | 209 | 283 | 0.00085794 | 0.0284391 | −0.054 |
| INTS8 | 250 | 291 | 0.00195612 | 0.04931592 | −0.054 |
| EXOSC1 | 265 | 238 | 0.00055147 | 0.02129198 | −0.054 |
| RABEPK | 208 | 298 | 7.05E−08 | 1.97E−05 | −0.054 |
| UTP6 | 231 | 231 | 0.00040601 | 0.01708287 | −0.054 |
| TRPC4AP | 298 | 298 | 0.00020351 | 0.0104982 | −0.055 |
| XAF1 | 206 | 235 | 0.00144628 | 0.04064685 | −0.055 |
| ZDHHC4 | 298 | 273 | 0.00064379 | 0.02384251 | −0.055 |
| PHYKPL | 268 | 266 | 0.00114423 | 0.03454075 | −0.055 |
| CAST | 212 | 22 | 0.00133554 | 0.03847959 | −0.055 |
| PPA2 | 194 | 236 | 0.0015332 | 0.04221873 | −0.055 |
| RBM6 | 239 | 219 | 0.00066847 | 0.02442454 | −0.055 |
| DDX21 | 223 | 298 | 0.00066557 | 0.02434895 | −0.055 |
| RCBTB2 | 292 | 298 | 0.00151538 | 0.04192845 | −0.055 |
| ABCA7 | 281 | 289 | 0.0002251 | 0.0112652 | −0.055 |
| LMF2 | 178 | 298 | 8.30E−06 | 0.00092973 | −0.056 |
| PHC2 | 257 | 279 | 0.00111505 | 0.03384752 | −0.056 |
| WDSUB1 | 183 | 290 | 4.05E−06 | 0.00052126 | −0.056 |
| JAK1 | 232 | 298 | 0.00082215 | 0.02767365 | −0.056 |
| IFTAP | 298 | 298 | 0.00134759 | 0.03864545 | −0.056 |
| RABEPK | 298 | 298 | 3.56E−10 | 2.72E−07 | −0.056 |
| SNX10 | 196 | 236 | 0.00049787 | 0.01981726 | −0.057 |
| DAGLB | 298 | 272 | 0.00012302 | 0.00712155 | −0.057 |
| DEK | 251 | 259 | 0.00187567 | 0.04817936 | −0.057 |
| WDSUB1 | 243 | 290 | 0.00010243 | 0.00624453 | −0.057 |
| LAIR1 | 185 | 298 | 0.00032322 | 0.0144653 | −0.057 |
| VDAC2 | 218 | 199 | 5.50E−06 | 0.00067116 | −0.057 |
| PPP4R3B | 248 | 298 | 0.00184323 | 0.04754237 | −0.057 |
| DLEU2 | 228 | 278 | 0.0001427 | 0.00798477 | −0.057 |
| STK38 | 285 | 201 | 6.60E−05 | 0.00444473 | −0.057 |
| STAT5B | 287 | 298 | 0.00157672 | 0.04279528 | −0.057 |
| DESI1 | 219 | 259 | 0.00040756 | 0.01713186 | −0.058 |
| CCM2 | 298 | 285 | 1.11E−05 | 0.00115158 | −0.058 |
| PREX1 | 221 | 298 | 0.00028737 | 0.01328362 | −0.058 |
| TMEM87A | 249 | 243 | 0.00126932 | 0.03705946 | −0.058 |
| NEPRO | 298 | 298 | 0.00053956 | 0.02094966 | −0.058 |
| WDR37 | 298 | 246 | 3.99E−07 | 8.20E−05 | −0.058 |
| LAIR1 | 185 | 298 | 0.00029592 | 0.01345083 | −0.058 |
| LAIR1 | 185 | 298 | 0.00031595 | 0.01419835 | −0.058 |
| SUPT20H | 208 | 216 | 0.00158899 | 0.04302106 | −0.058 |
| CDC16 | 204 | 247 | 0.00104674 | 0.03224559 | −0.058 |
| JPX | 298 | 247 | 0.00119803 | 0.03569523 | −0.058 |
| RBM23 | 27 | 267 | 3.25E−06 | 0.00044033 | −0.058 |
| RPL18A | 298 | 279 | 0.00066958 | 0.0244326 | −0.058 |
| LINC00893 | 298 | 230 | 5.96E−10 | 4.26E−07 | −0.059 |
| ZDHHC4 | 298 | 273 | 0.00011885 | 0.00697279 | −0.059 |
| SND1 | 298 | 270 | 0.00179492 | 0.04671089 | −0.059 |
| SLC3A2 | 298 | 241 | 0.00010972 | 0.00657882 | −0.059 |
| RBM33 | 298 | 298 | 4.11E−05 | 0.0031397 | −0.059 |
| TMEM63A | 187 | 292 | 0.00099706 | 0.03120015 | −0.059 |
| MEF2D | 206 | 298 | 0.00174503 | 0.04593336 | −0.059 |
| NLRC5 | 298 | 233 | 0.00142567 | 0.04032756 | −0.059 |
| POLD2 | 298 | 271 | 0.00122113 | 0.03618564 | −0.059 |
| TNFSF13 | 228 | 268 | 0.0006477 | 0.02392665 | −0.06 |
| NTAN1 | 282 | 223 | 0.00189266 | 0.04833099 | −0.06 |
| VPS53 | 216 | 236 | 0.00027857 | 0.01304269 | −0.06 |
| TANGO2 | 298 | 238 | 1.92E−06 | 0.00028707 | −0.06 |
| ARHGEF40 | 298 | 298 | 0.00070675 | 0.02506553 | −0.06 |
| ZDHHC24 | 298 | 220 | 0.00184863 | 0.04758306 | −0.06 |
| ABCA7 | 182 | 298 | 0.00094423 | 0.03016446 | −0.06 |
| SAMD9L | 297 | 251 | 7.31E−06 | 0.00084266 | −0.061 |
| EPSTI1 | 298 | 223 | 0.00039413 | 0.01667546 | −0.061 |
| POLR3C | 242 | 298 | 0.00022449 | 0.01126053 | −0.061 |
| ECHDC1 | 298 | 202 | 0.00189734 | 0.04838041 | −0.061 |
| GMDS-DT | 251 | 298 | 5.82E−08 | 1.74E−05 | −0.061 |
| WDSUB1 | 243 | 298 | 9.35E−05 | 0.00583102 | −0.061 |
| TYROBP | 182 | 284 | 0.00025084 | 0.01213547 | −0.061 |
| TTC13 | 291 | 298 | 0.0010256 | 0.03186336 | −0.061 |
| PHF20L1 | 298 | 298 | 0.00124025 | 0.03650415 | −0.061 |
| CORO1C | 298 | 279 | 0.0014424 | 0.04061638 | −0.061 |
| ECHDC1 | 296 | 202 | 7.33E−05 | 0.00479702 | −0.062 |
| SMIM8 | 298 | 269 | 1.06E−07 | 2.80E−05 | −0.063 |
| NEPRO | 298 | 270 | 0.0003125 | 0.01405763 | −0.063 |
| HLA-C | 182 | 197 | 0.00038413 | 0.01641601 | −0.063 |
| BBS9 | 290 | 245 | 2.87E−05 | 0.00237292 | −0.063 |
| AMD1 | 236 | 276 | 0.00132144 | 0.03817193 | −0.063 |
| ERCC1 | 249 | 221 | 8.87E−05 | 0.00562885 | −0.063 |
| DLG1 | 298 | 298 | 0.00023498 | 0.01162618 | −0.064 |
| ZFAND1 | 298 | 27 | 1.54E−06 | 0.00024138 | −0.064 |
| MAPKAPK5 | 223 | 247 | 0.00151934 | 0.04194292 | −0.064 |
| CELF1 | 298 | 293 | 0.00114354 | 0.03454075 | −0.065 |
| PVT1 | 264 | 262 | 0.0019744 | 0.04963339 | −0.065 |
| WASH3P | 298 | 298 | 1.18E−05 | 0.0012005 | −0.065 |
| CNOT2 | 292 | 272 | 1.91E−06 | 0.00028707 | −0.065 |
| RFFL | 298 | 298 | 5.51E−09 | 2.40E−06 | −0.065 |
| LONP2 | 272 | 298 | 2.97E−05 | 0.00243429 | −0.066 |
| ARIH2 | 217 | 298 | 0.00035947 | 0.01560666 | −0.066 |
| SNX22 | 254 | 182 | 0.00018453 | 0.00973803 | −0.067 |
| SCAP | 298 | 235 | 0.00094253 | 0.03016446 | −0.067 |
| VASP | 250 | 186 | 0.00124461 | 0.03658295 | −0.067 |
| FKBP15 | 248 | 298 | 0.0007852 | 0.02685201 | −0.067 |
| PSD4 | 266 | 225 | 0.00161595 | 0.04361577 | −0.068 |
| INTS8 | 298 | 291 | 0.000363 | 0.01572898 | −0.068 |
| TANGO2 | 244 | 238 | 4.69E−06 | 0.00058734 | −0.068 |
| TMEM127 | 298 | 298 | 0.00069557 | 0.02499364 | −0.068 |
| LRCH3 | 203 | 221 | 0.00022765 | 0.01136697 | −0.068 |
| KDM5C | 227 | 272 | 0.0007861 | 0.02685427 | −0.068 |
| BBS1 | 281 | 256 | 4.89E−06 | 0.00060876 | −0.068 |
| SRP14-AS1 | 298 | 298 | 0.00177782 | 0.04646003 | −0.069 |
| SOS1 | 298 | 268 | 0.00127929 | 0.03724715 | −0.069 |
| RNF121 | 283 | 261 | 0.00090561 | 0.02926942 | −0.069 |
| PDCD6 | 211 | 259 | 0.00087283 | 0.02877966 | −0.069 |
| SEC16A | 232 | 298 | 5.17E−06 | 0.00063693 | −0.069 |
| ADAP2 | 229 | 262 | 3.24E−10 | 2.54E−07 | −0.069 |
| DNAJC2 | 251 | 225 | 0.00180778 | 0.04698247 | −0.07 |
| RPS6KB1 | 218 | 297 | 0.00011991 | 0.00699306 | −0.07 |
| RNF121 | 283 | 251 | 0.0006571 | 0.02413051 | −0.07 |
| CEP290 | 298 | 272 | 0.00187361 | 0.04815481 | −0.07 |
| PVT1 | 298 | 262 | 0.00027267 | 0.01283561 | −0.07 |
| EXOSC9 | 252 | 287 | 0.00077921 | 0.0268032 | −0.07 |
| CLPTM1 | 262 | 273 | 0.00025327 | 0.01219911 | −0.07 |
| PREX1 | 221 | 254 | 0.00088261 | 0.02893181 | −0.071 |
| IL15 | 298 | 247 | 2.01E−06 | 0.00029751 | −0.071 |
| MTA1 | 243 | 276 | 0.00035329 | 0.01544943 | −0.071 |
| EFCAB13 | 293 | 293 | 0.00015383 | 0.00849893 | −0.071 |
| OCEL1 | 298 | 203 | 1.47E−07 | 3.56E−05 | −0.071 |
| ZC3H7A | 250 | 298 | 3.00E−05 | 0.00243688 | −0.072 |
| DAGLB | 230 | 298 | 1.11E−16 | 8.07E−13 | −0.072 |
| EIF2B4 | 298 | 241 | 0.00074311 | 0.0258929 | −0.072 |
| EIF2D | 298 | 239 | 0.00071125 | 0.02512434 | −0.072 |
| GUSBP11 | 261 | 262 | 1.21E−05 | 0.00122804 | −0.073 |
| FBXO3 | 239 | 298 | 0.00069608 | 0.02499364 | −0.073 |
| VASP | 250 | 201 | 0.00069033 | 0.02488956 | −0.073 |
| GRK3 | 216 | 250 | 0.00080576 | 0.02729079 | −0.073 |
| PIK3AP1 | 298 | 286 | 0.00084136 | 0.02809443 | −0.073 |
| CDC16 | 204 | 244 | 0.00028081 | 0.01306337 | −0.073 |
| RALGAPB | 269 | 228 | 0.00165704 | 0.04423183 | −0.074 |
| PAPSS1 | 264 | 298 | 0.00011063 | 0.00660603 | −0.074 |
| RABGAP1L | 243 | 298 | 0.00020998 | 0.01073042 | −0.074 |
| C1orf21 | 298 | 244 | 0.0004357 | 0.01796803 | −0.074 |
| LAIR1 | 298 | 200 | 0.00089099 | 0.02907056 | −0.074 |
| ST6GAL1 | 281 | 298 | 0.00029172 | 0.01337047 | −0.074 |
| OGFOD1 | 266 | 241 | 0.00190074 | 0.04838041 | −0.075 |
| ARAP2 | 197 | 289 | 8.93E−10 | 5.64E−07 | −0.075 |
| FDX1 | 274 | 279 | 0.00193158 | 0.04883842 | −0.075 |
| TTC39B | 184 | 245 | 0.00133992 | 0.03857788 | −0.075 |
| CD320 | 298 | 298 | 0.00131934 | 0.03813661 | −0.075 |
| RUNX3 | 298 | 254 | 0.00175825 | 0.04613745 | −0.076 |
| COP1 | 247 | 226 | 0.00120909 | 0.03592659 | −0.076 |
| ANKRD27 | 298 | 298 | 0.00067269 | 0.02447613 | −0.076 |
| TXLNA | 298 | 241 | 0.00021137 | 0.01077625 | −0.076 |
| TPGS2 | 229 | 237 | 0.00027989 | 0.0130508 | −0.076 |
| MCTP1 | 292 | 229 | 0.00173859 | 0.04584711 | −0.077 |
| AD000671.1 | 179 | 235 | 0.00034451 | 0.01516883 | −0.077 |
| COQ7 | 289 | 218 | 3.53E−05 | 0.00277052 | −0.077 |
| NRF1 | 298 | 264 | 0.00037239 | 0.01597691 | −0.077 |
| IFTAP | 298 | 298 | 1.55E−05 | 0.00145463 | −0.077 |
| SCFD1 | 240 | 272 | 0.00161509 | 0.04361577 | −0.077 |
| LY96 | 278 | 202 | 0.0011815 | 0.0353477 | −0.078 |
| TMCO4 | 298 | 177 | 0.0015068 | 0.04180873 | −0.078 |
| ANKZF1 | 262 | 252 | 0.00020283 | 0.01047538 | −0.078 |
| SCYL1 | 298 | 204 | 0.00101661 | 0.03167554 | −0.078 |
| PTPRA | 228 | 234 | 0.0006988 | 0.02501772 | −0.079 |
| DRAM1 | 217 | 298 | 0.00042518 | 0.0176592 | −0.079 |
| USP48 | 283 | 232 | 3.92E−06 | 0.00050756 | −0.079 |
| ARHGEF40 | 230 | 266 | 0.00047117 | 0.01910525 | −0.079 |
| CHFR | 294 | 249 | 4.18E−05 | 0.00318095 | −0.079 |
| GYS1 | 256 | 209 | 0.00050246 | 0.01992941 | −0.079 |
| RORC | 298 | 257 | 4.60E−08 | 1.41E−05 | −0.08 |
| UVRAG | 234 | 237 | 0.00116826 | 0.03513824 | −0.08 |
| SIRPB1 | 298 | 264 | 4.88E−07 | 9.67E−05 | −0.08 |
| TTI2 | 298 | 298 | 0.00013362 | 0.00756404 | −0.08 |
| COP1 | 209 | 218 | 0.00134847 | 0.03864545 | −0.081 |
| TMEM8B | 228 | 298 | 2.02E−07 | 4.64E−05 | −0.081 |
| CAPN12 | 209 | 220 | 0.00018348 | 0.00970603 | −0.081 |
| HIPK3 | 298 | 273 | 0.00189156 | 0.04833099 | −0.081 |
| SRC | 226 | 298 | 0.00074393 | 0.02590081 | −0.082 |
| HERPUD2 | 298 | 298 | 3.50E−06 | 0.00046474 | −0.082 |
| KLRG1 | 254 | 250 | 0.00030383 | 0.01372427 | −0.082 |
| COP1 | 247 | 218 | 0.00022269 | 0.01119627 | −0.082 |
| WDSUB1 | 243 | 256 | 0.00153258 | 0.04221873 | −0.082 |
| SPG7 | 289 | 252 | 0.00027005 | 0.01278465 | −0.082 |
| P4HA1 | 220 | 220 | 0.00184918 | 0.04758306 | −0.083 |
| IKBKG | 268 | 298 | 1.24E−09 | 7.49E−07 | −0.083 |
| METTL26 | 209 | 217 | 0.00019291 | 0.01009501 | −0.083 |
| DNAJC1 | 251 | 298 | 0.00166863 | 0.04451387 | −0.083 |
| NACC2 | 298 | 298 | 0.00148703 | 0.04136536 | −0.083 |
| THOC6 | 214 | 253 | 0.0002775 | 0.01300654 | −0.083 |
| ALG13 | 253 | 208 | 5.04E−05 | 0.00366823 | −0.084 |
| RB1 | 266 | 181 | 0.0001852 | 0.00976185 | −0.084 |
| LRP8 | 272 | 298 | 0.00180895 | 0.04698247 | −0.084 |
| BCAS3 | 283 | 298 | 0.00106496 | 0.03259934 | −0.084 |
| RNF170 | 225 | 258 | 0.00089894 | 0.0291555 | −0.085 |
| MDM4 | 283 | 205 | 0.00198866 | 0.04990405 | −0.085 |
| METTL26 | 209 | 298 | 9.09E−05 | 0.00570301 | −0.085 |
| FANCA | 223 | 278 | 1.53E−05 | 0.00144217 | −0.085 |
| CWF19L1 | 234 | 251 | 0.00020514 | 0.01055723 | −0.085 |
| LRRK2 | 229 | 256 | 8.92E−05 | 0.00562885 | −0.085 |
| PRMT9 | 298 | 298 | 0.00090271 | 0.02923408 | −0.086 |
| NUP54 | 298 | 298 | 0.00035264 | 0.01544943 | −0.086 |
| ANKZF1 | 298 | 252 | 0.00140909 | 0.04001439 | −0.086 |
| NAE1 | 229 | 259 | 1.68E−05 | 0.00155321 | −0.086 |
| PPP2R2D | 242 | 247 | 0.00124751 | 0.03661993 | −0.087 |
| TSEN2 | 200 | 288 | 1.51E−12 | 2.19E−09 | −0.087 |
| IKBKG | 268 | 298 | 0.00147604 | 0.04121753 | −0.088 |
| THEM4 | 298 | 298 | 0.00090918 | 0.02933466 | −0.088 |
| COP1 | 247 | 218 | 0.00016503 | 0.00894743 | −0.088 |
| CAST | 188 | 242 | 0.00097954 | 0.03082905 | −0.089 |
| AGPAT3 | 212 | 298 | 0.00028944 | 0.01332258 | −0.089 |
| TRPM7 | 214 | 246 | 0.0002457 | 0.01196632 | −0.089 |
| RAD1 | 298 | 298 | 0.00069471 | 0.02498552 | −0.09 |
| RNPC3 | 197 | 268 | 0.00131201 | 0.03800026 | −0.09 |
| WDSUB1 | 183 | 256 | 2.72E−05 | 0.00227432 | −0.09 |
| SLBP | 254 | 209 | 0.00024552 | 0.01196632 | −0.09 |
| KIF1C | 270 | 282 | 1.46E−05 | 0.00139182 | −0.091 |
| FOXRED1 | 244 | 251 | 2.62E−05 | 0.00220967 | −0.092 |
| DICER1-AS1 | 191 | 298 | 1.17E−10 | 1.06E−07 | −0.092 |
| CRBN | 212 | 234 | 0.00018808 | 0.00987763 | −0.092 |
| PACC1 | 292 | 257 | 0.00035769 | 0.01555 | −0.093 |
| SPATA13 | 286 | 298 | 0.00021538 | 0.01091448 | −0.093 |
| ABCB7 | 269 | 282 | 0.00191844 | 0.0487041 | −0.094 |
| DENND4B | 298 | 272 | 0.00049381 | 0.01972988 | −0.094 |
| JAK2 | 232 | 298 | 6.86E−05 | 0.00458061 | −0.095 |
| N4BP2L1 | 238 | 226 | 0.00038747 | 0.01651474 | −0.095 |
| IMMP1L | 212 | 238 | 2.98E−06 | 0.0004115 | −0.096 |
| TBC1D2B | 298 | 298 | 0.0016479 | 0.0442044 | −0.096 |
| ATF7IP | 298 | 233 | 0.00096862 | 0.03052935 | −0.097 |
| SLBP | 254 | 230 | 9.08E−05 | 0.0057021 | −0.098 |
| ARHGAP19 | 298 | 236 | 0.00012221 | 0.00709339 | −0.098 |
| POLL | 298 | 278 | 0.00058748 | 0.02211403 | −0.099 |
| IL15 | 271 | 260 | 2.34E−07 | 5.28E−05 | −0.099 |
| RIN3 | 298 | 222 | 0.00053977 | 0.02094966 | −0.099 |
| PXN | 298 | 298 | 2.77E−05 | 0.00230546 | −0.099 |
| PLXDC1 | 298 | 293 | 0.00022564 | 0.01127917 | −0.099 |
| N4BP2L1 | 277 | 226 | 0.00163752 | 0.04403428 | −0.099 |
| PI4KAP2 | 250 | 298 | 3.39E−06 | 0.00045189 | −0.099 |
| SUZ12P1 | 196 | 214 | 3.26E−14 | 7.90E−11 | −0.099 |
| SCAF8 | 194 | 298 | 0.00012491 | 0.00718321 | −0.1 |
| GTF2IRD2B | 288 | 269 | 0.0005915 | 0.02224625 | −0.1 |
| SENP6 | 210 | 254 | 0.0016522 | 0.04422018 | −0.101 |
| DNMT3A | 298 | 298 | 0.00012862 | 0.00730948 | −0.101 |
| DCP1B | 190 | 277 | 0.00099676 | 0.03120015 | −0.101 |
| TMEM39A | 298 | 233 | 0.00066584 | 0.02434895 | −0.102 |
| CBWD3 | 272 | 298 | 0.0003886 | 0.01653813 | −0.102 |
| RETSAT | 298 | 269 | 0.00012069 | 0.00701945 | −0.102 |
| DENND6A | 214 | 239 | 0.00039441 | 0.01667546 | −0.103 |
| ZSWIM7 | 263 | 271 | 0.00088488 | 0.02894276 | −0.104 |
| AK5 | 298 | 298 | 0.00033047 | 0.01465444 | −0.105 |
| MDM1 | 264 | 298 | 0.00150232 | 0.04171098 | −0.105 |
| ITGA6 | 229 | 281 | 0.00100373 | 0.03136399 | −0.106 |
| HDAC4 | 298 | 270 | 0.00043444 | 0.01793299 | −0.108 |
| SIRPB1 | 298 | 298 | 8.21E−08 | 2.24E−05 | −0.108 |
| ELMO2 | 228 | 200 | 0.00091362 | 0.02943425 | −0.109 |
| ABCA7 | 298 | 182 | 0.00063596 | 0.02361074 | −0.109 |
| HLA-B | 298 | 266 | 3.86E−06 | 0.00050187 | −0.111 |
| KIAA0753 | 212 | 298 | 0.00068798 | 0.02482549 | −0.111 |
| MRNIP | 229 | 238 | 0.00057184 | 0.02176982 | −0.111 |
| RIOK1 | 240 | 219 | 0.00081991 | 0.02761981 | −0.111 |
| HLA-B | 298 | 298 | 4.68E−08 | 1.42E−05 | −0.112 |
| RNF144B | 298 | 254 | 0.00187716 | 0.04818926 | −0.112 |
| RAB35 | 200 | 274 | 4.90E−05 | 0.00358265 | −0.112 |
| DOCK10 | 269 | 239 | 0.00063491 | 0.02359385 | −0.112 |
| FBXL4 | 267 | 298 | 0.0009662 | 0.03051008 | −0.113 |
| TMEM189-UBE2V1 | 269 | 241 | 0.00134654 | 0.03864097 | −0.114 |
| SEPTIN11 | 264 | 298 | 0.00051596 | 0.02027376 | −0.115 |
| RBM22 | 203 | 179 | 1.13E−05 | 0.00115495 | −0.116 |
| BSCL2 | 298 | 231 | 0.00026261 | 0.01252435 | −0.116 |
| SNHG17 | 234 | 298 | 1.00E−12 | 1.61E−09 | −0.117 |
| SORBS3 | 289 | 298 | 0.00044716 | 0.01831936 | −0.117 |
| SPOPL | 287 | 271 | 0.00179263 | 0.04667914 | −0.117 |
| SLC7A6 | 298 | 275 | 0.00041005 | 0.01721963 | −0.117 |
| NDRG1 | 230 | 185 | 0.00031194 | 0.01405763 | −0.118 |
| LAIR1 | 185 | 298 | 0.00017114 | 0.00917576 | −0.118 |
| LAIR1 | 185 | 298 | 0.00015273 | 0.00844861 | −0.119 |
| ATL2 | 298 | 277 | 0.00128019 | 0.03724715 | −0.119 |
| SCAF8 | 233 | 298 | 0.00030632 | 0.01382264 | −0.12 |
| AC009061.2 | 298 | 246 | 0.00033755 | 0.01492256 | −0.12 |
| TMCO4 | 298 | 177 | 0.00094567 | 0.03016446 | −0.121 |
| BSCL2 | 298 | 231 | 0.00011789 | 0.00693877 | −0.121 |
| MAPK6 | 298 | 294 | 0.00036117 | 0.01566509 | −0.121 |
| PLSCR3 | 298 | 192 | 0.00049483 | 0.01974139 | −0.122 |
| FOXJ3 | 251 | 298 | 8.50E−08 | 2.29E−05 | −0.124 |
| SLC39A11 | 298 | 294 | 0.0004892 | 0.01959948 | −0.124 |
| PYROXD1 | 230 | 269 | 0.00161977 | 0.04369174 | −0.125 |
| RNF144B | 298 | 294 | 0.00126505 | 0.03695957 | −0.125 |
| TMEM220 | 210 | 273 | 0.00021375 | 0.01085943 | −0.126 |
| TADA2A | 238 | 222 | 0.00134462 | 0.03861135 | −0.126 |
| CYRIB | 251 | 195 | 2.83E−06 | 0.00039618 | −0.127 |
| PEX1 | 293 | 233 | 0.00165522 | 0.04422018 | −0.129 |
| TSPAN2 | 252 | 248 | 0.00032321 | 0.0144653 | −0.129 |
| TTLL12 | 298 | 298 | 7.17E−05 | 0.00474121 | −0.132 |
| TMEM234 | 208 | 283 | 9.00E−07 | 0.00016147 | −0.136 |
| BCL9L | 203 | 25 | 0.001622 | 0.04369782 | −0.136 |
| AC008073.3 | 287 | 218 | 2.45E−13 | 5.08E−10 | −0.137 |
| GNB4 | 188 | 256 | 0.00086786 | 0.02868069 | −0.138 |
| TSPAN2 | 252 | 224 | 0.00088015 | 0.0289142 | −0.139 |
| TMEM234 | 208 | 230 | 0.00016122 | 0.00879489 | −0.142 |
| ERAP2 | 281 | 274 | 0.00013662 | 0.00770413 | −0.143 |
| ANKRD6 | 248 | 248 | 0.0018827 | 0.04827458 | −0.144 |
| DDX60L | 298 | 298 | 2.02E−08 | 7.45E−06 | −0.147 |
| GTF2H2B | 237 | 257 | 3.38E−07 | 7.18E−05 | −0.148 |
| SLC35F2 | 298 | 202 | 0.00104997 | 0.03230339 | −0.148 |
| TBCD | 247 | 251 | 5.79E−05 | 0.00407092 | −0.149 |
| ZFR | 298 | 294 | 0.00189196 | 0.04833099 | −0.156 |
| PXYLP1 | 281 | 298 | 6.15E−05 | 0.00424162 | −0.158 |
| SOS1 | 275 | 281 | 8.02E−06 | 0.00090772 | −0.16 |
| SNHG17 | 267 | 298 | 0.00088276 | 0.02893181 | −0.164 |
| CA5B | 245 | 229 | 0.00028838 | 0.01330213 | −0.168 |
| SNHG17 | 267 | 298 | 6.29E−05 | 0.00429436 | −0.171 |
| ERC1 | 281 | 298 | 0.00148698 | 0.04136536 | −0.175 |
| CYRIB | 251 | 195 | 5.35E−05 | 0.00385796 | −0.176 |
| RANBP10 | 261 | 202 | 1.39E−05 | 0.00134185 | −0.183 |
| AC092070.2 | 229 | 276 | 0.00081014 | 0.02737517 | −0.187 |
| SETD6 | 298 | 270 | 0.00048118 | 0.01943914 | −0.2 |
| ITGB2 | 210 | 238 | 1.28E−08 | 4.97E−06 | −0.209 |
| CEP170 | 276 | 246 | 0.00175625 | 0.0461252 | −0.21 |
| SNHG17 | 227 | 298 | 0.00023949 | 0.01180907 | −0.221 |
| LRP8 | 298 | 289 | 4.05E−05 | 0.0031146 | −0.225 |
| SNHG17 | 227 | 298 | 9.64E−05 | 0.00595458 | −0.233 |
| ANKRD36B | 291 | 298 | 0.00016386 | 0.00889504 | −0.254 |

TABLE 19
A3SS_FXSvsTD

| GeneID | chr | strand | longExonStart_0base | longExonEnd | PValue | FDR | IncLevelDifference |
|---|---|---|---|---|---|---|---|
| PATZ1 | chr22 | − | 31328786 | 31328924 | 0.00025746 | 0.00975747 | −0.093 |
| TPTEP1 | chr22 | + | 16647604 | 16647785 | 0.00139658 | 0.03495258 | −0.149 |
| PABPC1L | chr20 | + | 44933056 | 44933185 | 1.94E−07 | 3.31E−05 | 0.079 |
| SNHG17 | chr20 | − | 38421005 | 38422368 | 0.00023295 | 0.00885665 | −0.17 |
| CPNE1 | chr20 | − | 35626566 | 35626803 | 3.72E−07 | 5.77E−05 | −0.086 |
| CPNE1 | chr20 | − | 35626566 | 35626826 | 1.32E−06 | 0.00015625 | −0.12 |
| ZNF160 | chr19 | − | 53091412 | 53091529 | 1.67E−08 | 4.89E−06 | −0.052 |
| XAF1 | chr17 | + | 6759255 | 6759718 | 2.15E−08 | 5.85E−06 | 0.084 |
| LSM7 | chr19 | − | 2324124 | 2324196 | 0.0004093 | 0.01372545 | −0.055 |
| POLR2J3 | chr7 | − | 102543524 | 102543702 | 6.03E−05 | 0.00325599 | 0.252 |
| TSNARE1 | chr8 | − | 142315002 | 142315092 | 0.00127149 | 0.03292635 | 0.089 |
| THADA | chr2 | − | 43551788 | 43551925 | 6.89E−14 | 1.37E−10 | −0.065 |
| TRIM73 | chr7 | + | 75405181 | 75405605 | 6.33E−09 | 2.52E−06 | −0.149 |
| AEBP1 | chr7 | + | 44109656 | 44110124 | 3.25E−08 | 7.61E−06 | 0.068 |
| TBC1D7 | chr6 | − | 13326786 | 13326963 | 0.00161522 | 0.03864227 | 0.117 |
| DDX60L | chr4 | − | 168395958 | 168396127 | 2.77E−08 | 6.74E−06 | −0.204 |
| ASNS | chr7 | − | 97854579 | 97854754 | 9.99E−05 | 0.00451674 | 0.062 |
| MTSS1 | chr8 | − | 124556231 | 124556405 | 0.00060128 | 0.01859606 | 0.1 |
| MZB1 | chr5 | − | 139388460 | 139388667 | 3.46E−05 | 0.00205267 | 0.052 |
| SEPTIN8 | chr5 | − | 132763705 | 132763892 | 7.91E−06 | 0.00061701 | 0.134 |
| PPWD1 | chr5 | + | 65569631 | 65569732 | 0.00141214 | 0.03512102 | 0.062 |
| TMEM116 | chr12 | − | 111932585 | 111932731 | 3.98E−05 | 0.00229594 | 0.097 |
| TMEM116 | chr12 | − | 111933885 | 111936830 | 8.69E−06 | 0.00065975 | 0.118 |
| BRD9 | chr5 | − | 889586 | 889766 | 8.61E−06 | 0.00065881 | 0.062 |
| SDHA | chr5 | + | 254357 | 254506 | 0.00213689 | 0.04777197 | −0.156 |
| IKBKG | chrX | + | 154558531 | 154558650 | 8.24E−05 | 0.00393314 | −0.118 |
| ZNF707 | chr8 | + | 143691596 | 143691713 | 0.00019848 | 0.00779409 | 0.104 |
| CEP131 | chr17 | − | 81191192 | 81191350 | 0.00028481 | 0.01042946 | 0.103 |
| DXO | chr6 | − | 31970342 | 31970478 | 3.22E−07 | 5.27E−05 | −0.054 |
| TAFA2 | chr12 | − | 62258762 | 62258910 | 0.00132443 | 0.03378436 | −0.102 |
| TPRG1 | chr3 | + | 189150655 | 189151136 | 8.85E−06 | 0.00066422 | 0.07 |
| DLG1 | chr3 | − | 197076585 | 197076685 | 0.00145615 | 0.03592374 | −0.092 |
| GGT1 | chr22 | + | 24627409 | 24627619 | 0.00063066 | 0.01930477 | 0.059 |
| SRGAP2 | chr1 | + | 206401420 | 206401645 | 3.54E−05 | 0.00207054 | −0.203 |
| TMEM161B- | chr5 | + | 88287435 | 88287622 | 2.70E−07 | 4.47E−05 | 0.062 |
| AS1 | | | | | | | |
| HLA-C | chr6 | − | 31269492 | 31269543 | 3.39E−08 | 7.78E−06 | 0.056 |
| CCDC163 | chr1 | − | 45495417 | 45495545 | 0.00136754 | 0.03458837 | 0.105 |
| NABP1 | chr2 | + | 191683289 | 191683804 | 0.00173822 | 0.04076781 | 0.093 |
| RNF181 | chr2 | + | 85596952 | 85597178 | 2.72E−05 | 0.00169037 | −0.101 |
| CENPT | chr16 | − | 67828473 | 67828605 | 0.00025869 | 0.00977295 | 0.061 |
| KHDC4 | chr1 | − | 155916624 | 155916737 | 0.00171796 | 0.040372 | −0.066 |
| ZNF7 | chr8 | + | 144837390 | 144837507 | 2.62E−08 | 6.74E−06 | 0.068 |
| DCAF11 | chr14 | + | 24115514 | 24115726 | 0.00153675 | 0.03706217 | 0.106 |
| AK5 | chr1 | + | 77293792 | 77293960 | 0.00155273 | 0.03737185 | −0.074 |
| ARRB2 | chr17 | + | 4715150 | 4715336 | 0.00120351 | 0.03130181 | 0.091 |
| ARHGAP25 | chr2 | + | 68807272 | 68807480 | 0.00100499 | 0.02745428 | 0.081 |
| AHSA2P | chr2 | + | 61178980 | 61179171 | 1.21E−05 | 0.00086042 | −0.056 |
| AZIN1 | chr8 | − | 102849998 | 102850165 | 5.32E−06 | 0.0004736 | 0.056 |
| ZNF540 | chr19 | + | 37601006 | 37601105 | 0.00117877 | 0.03072519 | −0.095 |
| MFSD9 | chr2 | − | 102723699 | 102723909 | 2.27E−06 | 0.0002421 | −0.061 |
| JKAMP | chr14 | + | 59486712 | 59486804 | 0.00199134 | 0.04528113 | 0.058 |
| HARS2 | chr5 | + | 140693490 | 140693665 | 0.00183476 | 0.04253071 | 0.065 |
| RNF32 | chr7 | + | 156676294 | 156677130 | 2.11E−06 | 0.00022679 | 0.054 |
| BPHL | chr6 | + | 3138040 | 3140509 | 0.00093256 | 0.02612624 | 0.068 |
| PPP6R3 | chr11 | + | 68587797 | 68588024 | 1.58E−11 | 1.45E−08 | −0.052 |
| PPP6R3 | chr11 | + | 68587944 | 68588024 | 1.38E−11 | 1.37E−08 | −0.066 |
| HELB | chr12 | + | 66324906 | 66325126 | 6.89E−06 | 0.00055934 | −0.058 |
| GALT | chr9 | + | 34648047 | 34648171 | 1.40E−05 | 0.00097725 | 0.131 |
| ALAD | chr9 | − | 113393446 | 113393634 | 0.00135144 | 0.03432669 | 0.053 |
| LAIR1 | chr19 | − | 54364294 | 54364486 | 0.00015155 | 0.00628201 | 0.053 |
| LAIR1 | chr19 | − | 54364294 | 54364486 | 0.00058777 | 0.01832059 | 0.058 |
| AC016394.2 | chr10 | + | 73253800 | 73254349 | 5.90E−05 | 0.00321776 | 0.187 |
| CTSB | chr8 | − | 11867253 | 11867526 | 1.02E−08 | 3.59E−06 | 0.105 |
| NAP1L4 | chr11 | − | 2989128 | 2989271 | 5.70E−07 | 7.73E−05 | 0.067 |
| ZNF195 | chr11 | − | 3361742 | 3368855 | 1.89E−05 | 0.00124681 | 0.147 |
| TCTN1 | chr12 | + | 110628766 | 110628918 | 0.00016797 | 0.00679743 | −0.067 |
| PHB2 | chr12 | − | 6969955 | 6970068 | 3.96E−06 | 0.00038479 | −0.154 |
| PHF1 | chr6 | + | 33415234 | 33415329 | 4.42E−06 | 0.00041542 | −0.101 |
| ATG16L2 | chr11 | + | 72821198 | 72821741 | 0.00066749 | 0.02012249 | 0.07 |
| TMEM25 | chr11 | + | 118532149 | 118532461 | 0.00073253 | 0.02138132 | −0.056 |
| SPSB2 | chr12 | − | 6872237 | 6872997 | 0.00076683 | 0.02221956 | 0.097 |
| YAF2 | chr12 | − | 42210547 | 42210680 | 9.65E−05 | 0.0044638 | −0.067 |
| MAP3K12 | chr12 | − | 53486946 | 53487155 | 6.24E−10 | 3.10E−07 | 0.149 |
| ATXN2 | chr12 | − | 111510384 | 111510582 | 0.00010536 | 0.00469336 | −0.059 |
| ARL6IP4 | chr12 | + | 122981570 | 122981879 | 2.03E−07 | 3.41E−05 | 0.059 |
| TMEM273 | chr10 | − | 49165204 | 49165361 | 0.00146171 | 0.03597907 | −0.064 |
| MPP5 | chr14 | + | 67279017 | 67279537 | 3.14E−10 | 1.63E−07 | 0.078 |
| ALDH6A1 | chr14 | − | 74071194 | 74071660 | 0.00067898 | 0.02040466 | 0.087 |
| LTK | chr15 | − | 41508068 | 41508236 | 2.79E−06 | 0.0002818 | −0.065 |
| FAM219B | chr15 | − | 74904981 | 74905059 | 5.18E−06 | 0.00047073 | 0.134 |
| MAN2A2 | chr15 | + | 90911366 | 90911550 | 0.00069732 | 0.0206644 | −0.053 |
| PSTPIP1 | chr15 | + | 77027799 | 77027914 | 0.00080743 | 0.02322665 | 0.081 |
| ZNF23 | chr16 | − | 71447596 | 71449885 | 1.15E−05 | 0.00082499 | 0.054 |
| SPG7 | chr16 | + | 89548979 | 89549336 | 3.10E−08 | 7.39E−06 | 0.108 |
| GABBR1 | chr6 | − | 29629086 | 29629247 | 0.00059447 | 0.01846376 | 0.072 |
| MLX | chr17 | + | 42569203 | 42569606 | 1.89E−15 | 4.51E−12 | 0.064 |
| BRCA1 | chr17 | − | 43076487 | 43076614 | 2.10E−05 | 0.0013478 | 0.233 |
| TSPOAP1- | chr17 | + | 58351734 | 58351914 | 6.16E−05 | 0.00330253 | −0.108 |
| AS1 | | | | | | | |
| DPH2 | chr1 | + | 43970905 | 43971189 | 0.00114495 | 0.03010664 | 0.08 |
| MPPE1 | chr18 | − | 11885675 | 11885912 | 0.00047539 | 0.01559111 | 0.073 |
| CCDC191 | chr3 | − | 114042702 | 114042846 | 6.39E−08 | 1.39E−05 | −0.065 |
| PCBP1-AS1 | chr2 | − | 70048777 | 70050252 | 0.00149611 | 0.03637583 | 0.083 |
| PCBP1-AS1 | chr2 | − | 70053730 | 70053812 | 0.000231 | 0.00885665 | 0.067 |
| PCBP1-AS1 | chr2 | − | 70053730 | 70053815 | 0.00045916 | 0.01518421 | 0.061 |
| VPS33B | chr15 | − | 91016962 | 91017064 | 3.16E−06 | 0.00031467 | −0.053 |
| UNC50 | chr2 | + | 98618145 | 98618515 | 5.88E−05 | 0.00321776 | −0.083 |
| ZNF345 | chr19 | + | 36851727 | 36851904 | 4.12E−05 | 0.00235209 | 0.105 |

TABLE 20
A5SS_FXSvsTD

| GeneID | chr | strand | longExonStart_0base | longExonEnd | PValue | FDR | IncLevelDifference |
|---|---|---|---|---|---|---|---|
| PQBP1 | chrX | + | 48897911 | 48898093 | 1.81E−06 | 0.00019968 | −0.203 |
| PIGT | chr20 | + | 45416516 | 45418979 | 0.00059641 | 0.02023817 | −0.057 |
| TTPAL | chr20 | + | 44484336 | 44484530 | 0.00052122 | 0.01860505 | −0.05 |
| FNTA | chr8 | + | 43077215 | 43080441 | 2.52E−05 | 0.00166816 | −0.075 |
| NEIL2 | chr8 | + | 11769709 | 11770335 | 0.00039292 | 0.01516544 | 0.181 |
| CEP152 | chr15 | − | 48741600 | 48741704 | 0 | 0 | 0.063 |
| DEPDC5 | chr22 | + | 31797599 | 31797703 | 1.88E−09 | 9.00E−07 | −0.076 |
| HLA-DMA | chr6 | − | 32949270 | 32949399 | 3.71E−06 | 0.00034707 | −0.055 |
| MICA | chr6 | + | 31399783 | 31400783 | 0.00057334 | 0.01978835 | 0.103 |
| MSTO1 | chr1 | + | 155611215 | 155611355 | 4.27E−07 | 6.33E−05 | −0.068 |
| MSTO1 | chr1 | + | 155611215 | 155611355 | 5.95E−05 | 0.00350918 | 0.056 |
| MSTO1 | chr1 | + | 155613656 | 155613766 | 0.00143232 | 0.04097644 | −0.153 |
| ERAP2 | chr5 | + | 96909579 | 96909809 | 1.80E−05 | 0.00136676 | 0.094 |
| PARP2 | chr14 | + | 20344931 | 20345126 | 2.07E−05 | 0.00149022 | 0.161 |
| ZSCAN25 | chr7 | + | 99619560 | 99621574 | 4.73E−07 | 6.88E−05 | −0.089 |
| HAGHL | chr16 | + | 728115 | 728424 | 0.00107855 | 0.03289787 | −0.072 |
| BUD31 | chr7 | + | 99408968 | 99409245 | 0.00031267 | 0.0126686 | 0.095 |
| FCRL1 | chr1 | − | 157797680 | 157797820 | 0.0005073 | 0.01836214 | 0.139 |
| AL392172.1 | chr1 | + | 222836995 | 222837090 | 0.00036831 | 0.01442067 | 0.166 |
| NDUFV2 | chr18 | + | 9124873 | 9124996 | 2.27E−12 | 3.08E−09 | 0.063 |
| ERVK13-1 | chr16 | − | 2671023 | 2672570 | 1.53E−07 | 3.06E−05 | 0.061 |
| ERVK13-1 | chr16 | − | 2671344 | 2672570 | 3.84E−07 | 5.79E−05 | 0.061 |
| APTR | chr7 | − | 77695895 | 77696255 | 0.00076843 | 0.02493262 | −0.057 |
| KLRD1 | chr12 | + | 10311463 | 10311619 | 1.49E−05 | 0.00116929 | −0.057 |
| IRF3 | chr19 | − | 49665483 | 49665857 | 0.00024168 | 0.01060085 | 0.102 |
| CUL7 | chr6 | − | 43046510 | 43047107 | 0.00164534 | 0.04549124 | −0.104 |
| ZDHHC3 | chr3 | − | 44959126 | 44959460 | 6.47E−07 | 9.09E−05 | −0.198 |
| ZDHHC3 | chr3 | − | 44959126 | 44959460 | 0.00129914 | 0.03792196 | 0.204 |
| RBIS | chr8 | − | 85219235 | 85219363 | 0.00120913 | 0.03607004 | 0.181 |
| PCBP4 | chr3 | − | 51961748 | 51962088 | 0.00127596 | 0.0374347 | 0.113 |
| ATP5F1A | chr18 | − | 46094979 | 46095131 | 9.62E−08 | 2.30E−05 | 0.063 |
| LUCAT1 | chr5 | − | 91313961 | 91314404 | 0.00047925 | 0.01758106 | 0.073 |
| CCDC14 | chr3 | − | 123946802 | 123947319 | 0.00045948 | 0.01700894 | 0.052 |
| UBE2I | chr16 | + | 1312346 | 1312603 | 2.00E−05 | 0.00145726 | −0.155 |
| ELOA-AS1 | chr1 | − | 23772267 | 23772426 | 0.00160251 | 0.04469475 | 0.177 |
| SLC25A37 | chr8 | + | 23566107 | 23566813 | 8.74E−05 | 0.00484387 | 0.058 |
| AGA | chr4 | − | 177436275 | 177436351 | 0.00063198 | 0.02118043 | −0.062 |
| CNBP | chr3 | − | 129171651 | 129171771 | 6.98E−08 | 1.78E−05 | −0.059 |
| PPIEL | chr1 | − | 39529015 | 39529164 | 1.31E−05 | 0.0010346 | 0.066 |
| AC022400.7 | chr10 | − | 73801339 | 73802776 | 0.00106853 | 0.03271456 | 0.073 |
| MRPL43 | chr10 | − | 100986686 | 100986975 | 1.19E−09 | 6.58E−07 | −0.142 |
| MRPL43 | chr10 | − | 100986686 | 100986975 | 2.63E−06 | 0.00026084 | −0.108 |
| SERGEF | chr11 | − | 18010070 | 18010139 | 9.45E−05 | 0.00520023 | −0.159 |
| STAT2 | chr12 | − | 56349344 | 56349509 | 7.15E−05 | 0.0041026 | 0.053 |
| ULK3 | chr15 | − | 74837750 | 74837798 | 1.85E−07 | 3.42E−05 | −0.216 |
| SLC6A12 | chr12 | − | 209772 | 210043 | 3.17E−05 | 0.00205171 | 0.131 |
| ANXA2 | chr15 | − | 60396150 | 60396427 | 0.00021158 | 0.00976282 | 0.125 |
| ITGB7 | chr12 | − | 53201117 | 53201194 | 3.57E−12 | 3.68E−09 | 0.07 |
| TM6SF1 | chr15 | + | 83119577 | 83119734 | 0.00024211 | 0.01060085 | 0.059 |
| ARL6IP4 | chr12 | + | 122981128 | 122981299 | 2.53E−06 | 0.00025747 | −0.052 |
| DDX51 | chr12 | − | 132141102 | 132141420 | 1.26E−06 | 0.0001579 | 0.081 |
| UBAC2- | chr13 | − | 99200365 | 99200710 | 1.22E−05 | 0.00098372 | −0.1 |
| AS1 | | | | | | | |
| PXN-AS1 | chr12 | + | 120206544 | 120206712 | 0.00017269 | 0.0083217 | 0.058 |
| APEX1 | chr14 | + | 20455577 | 20455773 | 0.0001489 | 0.00748522 | 0.074 |
| NEK3 | chr13 | − | 52143914 | 52143987 | 8.80E−09 | 3.26E−06 | 0.277 |
| NEK3 | chr13 | − | 52143914 | 52143987 | 1.92E−05 | 0.00142168 | 0.251 |
| TPM1 | chr15 | + | 63060868 | 63061273 | 3.76E−06 | 0.00034791 | 0.066 |
| SMPD1 | chr11 | + | 6391383 | 6392196 | 3.02E−07 | 5.03E−05 | 0.06 |
| UBC | chr12 | − | 124913320 | 124913774 | 1.50E−07 | 3.06E−05 | −0.053 |
| DVL2 | chr17 | − | 7230045 | 7230155 | 1.45E−06 | 0.00017071 | 0.119 |
| DPH2 | chr1 | + | 43970595 | 43970817 | 0.00138759 | 0.04007282 | 0.083 |
| AC243960.1 | chr19 | + | 41551179 | 41551494 | 3.62E−12 | 3.68E−09 | 0.065 |

TABLE 21
RI_FXSvsTD

| GeneID | chr | strand | riExonStart_0base | riExonEnd | PValue | FDR | IncLevelDifference |
|---|---|---|---|---|---|---|---|
| GPATCH4 | chr1 | − | 156596080 | 156596478 | 0.0019027 | 0.01963858 | 0.193 |
| CAPN3 | chr15 | + | 42409930 | 42410496 | 0.00478108 | 0.03931402 | 0.177 |
| SUN1 | chr7 | + | 852608 | 852952 | 8.12E−05 | 0.00164027 | 0.174 |
| ZNF7 | chr8 | + | 144829042 | 144829604 | 0.00314975 | 0.0295129 | 0.166 |
| ATAT1 | chr6 | + | 30645894 | 30646109 | 5.97E−05 | 0.00129679 | 0.163 |
| PER1 | chr17 | − | 8146896 | 8147381 | 0.00140278 | 0.01570375 | 0.162 |
| MSTO1 | chr1 | + | 155611215 | 155611603 | 0.0001029 | 0.00195911 | 0.158 |
| DHRS4L2 | chr14 | + | 24000862 | 24001084 | 0.00022375 | 0.00366117 | 0.158 |
| RELA-DT | chr11 | + | 65663426 | 65665543 | 0.00238179 | 0.02353446 | 0.154 |
| TM7SF2 | chr11 | + | 65112810 | 65113414 | 3.92E−06 | 0.00012959 | 0.154 |
| DERL3 | chr22 | − | 23834502 | 23837154 | 0.00111055 | 0.01306862 | 0.148 |
| ASB16-AS1 | chr17 | − | 44181622 | 44183093 | 0.00548944 | 0.04382915 | 0.148 |
| FAN1 | chr15 | + | 30925788 | 30928656 | 0.00248416 | 0.02434479 | 0.147 |
| ZFC3H1 | chr12 | − | 71656342 | 71657301 | 3.74E−08 | 2.98E−06 | 0.139 |
| NRBP2 | chr8 | − | 143839508 | 143839825 | 0.00367966 | 0.03306421 | 0.137 |
| OGFOD2 | chr12 | + | 122975810 | 122976767 | 6.82E−05 | 0.00142008 | 0.137 |
| SUN1 | chr7 | + | 852608 | 852952 | 2.27E−05 | 0.00056537 | 0.133 |
| AAAS | chr12 | − | 53315093 | 53315782 | 0.00404198 | 0.03496806 | 0.127 |
| LILRA1 | chr19 | + | 54594196 | 54594476 | 0.0033528 | 0.03102633 | 0.127 |
| BPHL | chr6 | + | 3138040 | 3140509 | 9.52E−05 | 0.00185296 | 0.125 |
| DDX51 | chr12 | − | 132140830 | 132141420 | 6.79E−08 | 4.78E−06 | 0.123 |
| ZNF266 | chr19 | − | 9418761 | 9419299 | 1.61E−09 | 2.29E−07 | 0.118 |
| WDR54 | chr2 | + | 74423318 | 74423982 | 5.76E−08 | 4.30E−06 | 0.117 |
| BCS1L | chr2 | + | 218662196 | 218662679 | 0.00293925 | 0.02793454 | 0.116 |
| FCSK | chr16 | + | 70466881 | 70467471 | 5.73E−05 | 0.00125974 | 0.115 |
| SEC31B | chr10 | − | 100505360 | 100506201 | 1.22E−05 | 0.00034557 | 0.114 |
| POLG | chr15 | − | 89317938 | 89318749 | 2.16E−05 | 0.0005451 | 0.114 |
| CD3E | chr11 | + | 118312152 | 118313874 | 6.25E−09 | 7.17E−07 | 0.113 |
| CD19 | chr16 | + | 28932345 | 28933114 | 0.00108799 | 0.01282845 | 0.113 |
| TUBGCP4 | chr15 | + | 43401715 | 43403799 | 0.00575984 | 0.04548526 | 0.112 |
| AVIL | chr12 | − | 57807330 | 57807727 | 0.00021838 | 0.00359632 | 0.111 |
| ZNF160 | chr19 | − | 53091412 | 53091720 | 0.00300071 | 0.02833848 | 0.108 |
| MINK1 | chr17 | + | 4889646 | 4890735 | 0.00204261 | 0.02078327 | 0.108 |
| TTC16 | chr9 | + | 127724119 | 127724897 | 0.00531007 | 0.04271109 | 0.105 |
| ING4 | chr12 | − | 6652661 | 6653050 | 0.0001088 | 0.00204534 | 0.105 |
| FCGR2B | chr1 | + | 161673959 | 161677365 | 3.97E−05 | 0.00091656 | 0.102 |
| GINS4 | chr8 | + | 41541808 | 41545030 | 0.00049979 | 0.00677488 | 0.098 |
| C1R | chr12 | − | 7091451 | 7092441 | 1.81E−12 | 9.01E−10 | 0.097 |
| TRIM27 | chr6 | − | 28903001 | 28908840 | 1.44E−06 | 5.66E−05 | 0.097 |
| RABEPK | chr9 | + | 125200541 | 125200906 | 0.00202431 | 0.02066921 | 0.096 |
| CTC1 | chr17 | − | 8229141 | 8229446 | 2.68E−05 | 0.00065908 | 0.093 |
| MAN2C1 | chr15 | − | 75359058 | 75359425 | 0.00034973 | 0.0050993 | 0.091 |
| RASGRP4 | chr19 | − | 38419859 | 38420262 | 0.00279479 | 0.02686051 | 0.09 |
| WDR24 | chr16 | − | 685868 | 686186 | 0.00536989 | 0.04299383 | 0.088 |
| RINL | chr19 | − | 38876330 | 38876781 | 0.00332881 | 0.03085216 | 0.087 |
| ZSCAN25 | chr7 | + | 99619560 | 99621574 | 0.00151166 | 0.01661155 | 0.086 |
| OGFOD2 | chr12 | + | 122975810 | 122976767 | 1.95E−06 | 7.14E−05 | 0.086 |
| C1orf174 | chr1 | − | 3890568 | 3892996 | 0.00276344 | 0.02668211 | 0.085 |
| PAXX | chr9 | + | 136992639 | 136992974 | 0.003216 | 0.02999259 | 0.081 |
| LINC01128 | chr1 | + | 851926 | 852766 | 1.72E−06 | 6.51E−05 | 0.08 |
| PRICKLE3 | chrX | − | 49177992 | 49178475 | 0.00019694 | 0.00331628 | 0.078 |
| IRF3 | chr19 | − | 49664673 | 49665857 | 3.28E−06 | 0.00011384 | 0.078 |
| MUTYH | chr1 | − | 45332762 | 45333324 | 2.15E−10 | 4.94E−08 | 0.077 |
| FAHD2A | chr2 | + | 95410526 | 95411026 | 1.55E−05 | 0.00042512 | 0.075 |
| RIOK1 | chr6 | + | 7402602 | 7402897 | 0.00164758 | 0.0177605 | 0.074 |
| ZC3H14 | chr14 | + | 88609266 | 88609803 | 0.00024537 | 0.00388056 | 0.074 |
| TMEM208 | chr16 | + | 67228796 | 67229278 | 7.49E−10 | 1.18E−07 | 0.074 |
| HARS2 | chr5 | + | 140693490 | 140693665 | 6.43E−07 | 3.03E−05 | 0.073 |
| MAP4K2 | chr11 | − | 64791908 | 64792422 | 0.00106376 | 0.01267351 | 0.072 |
| WDR6 | chr3 | + | 49011634 | 49014293 | 0.00060222 | 0.00794777 | 0.071 |
| ZNF649 | chr19 | − | 51899797 | 51900294 | 2.34E−08 | 2.06E−06 | 0.07 |
| NEPRO | chr3 | − | 113012733 | 113013403 | 5.38E−07 | 2.60E−05 | 0.07 |
| BEST1 | chr11 | + | 61959891 | 61962893 | 4.53E−06 | 0.00014591 | 0.07 |
| THOC6 | chr16 | + | 3025923 | 3026288 | 0 | 0 | 0.069 |
| PFKM | chr12 | + | 48134942 | 48135383 | 0.00071197 | 0.00915305 | 0.069 |
| ADA | chr20 | − | 44622828 | 44623078 | 4.54E−05 | 0.00103647 | 0.068 |
| IFT43 | chr14 | + | 76082294 | 76082692 | 1.97E−05 | 0.00050797 | 0.067 |
| PPIE | chr1 | + | 39741894 | 39743297 | 0.00287993 | 0.02745813 | 0.065 |
| BTN3A3 | chr6 | + | 26443956 | 26445985 | 0.00184582 | 0.01925713 | 0.064 |
| SAMD9L | chr7 | − | 93145384 | 93146040 | 0.00556426 | 0.04423294 | 0.062 |
| MPPE1 | chr18 | − | 11886498 | 11886778 | 0.00210294 | 0.02124996 | 0.062 |
| IFFO1 | chr12 | − | 6548064 | 6548545 | 0.00345324 | 0.03171044 | 0.061 |
| NAGK | chr2 | + | 71068649 | 71070586 | 0.00204834 | 0.02078939 | 0.06 |
| CCDC159 | chr19 | + | 11353450 | 11353874 | 0.00085438 | 0.01055261 | 0.06 |
| SAP30BP | chr17 | + | 75705610 | 75706092 | 0.00126274 | 0.01451666 | 0.059 |
| MRNIP | chr5 | − | 179837275 | 179840959 | 0.00332081 | 0.03082575 | 0.059 |
| ARHGAP9 | chr12 | − | 57476589 | 57476963 | 1.26E−07 | 8.02E−06 | 0.059 |
| XPOT | chr12 | + | 64425037 | 64425457 | 0.00082895 | 0.01033549 | 0.058 |
| P3H1 | chr1 | − | 42746374 | 42747412 | 0.00012633 | 0.00230957 | 0.058 |
| REC8 | chr14 | + | 24172898 | 24173198 | 0.00028464 | 0.00440822 | 0.057 |
| SCML4 | chr6 | − | 107744948 | 107746889 | 0.00017617 | 0.00301757 | 0.056 |
| APEX1 | chr14 | + | 20455225 | 20455703 | 0.00023697 | 0.00381833 | 0.056 |
| ENDOV | chr17 | + | 80415452 | 80415821 | 0.00366074 | 0.03305725 | 0.056 |
| HMBS | chr11 | + | 119092124 | 119092523 | 5.26E−05 | 0.00117316 | 0.055 |
| BRPF1 | chr3 | + | 9745572 | 9745930 | 0.00157081 | 0.01710441 | 0.055 |
| DHRS1 | chr14 | − | 24292183 | 24292784 | 2.94E−05 | 0.00071464 | 0.054 |
| NPHP3 | chr3 | − | 132684553 | 132686387 | 0.00128041 | 0.0146074 | 0.054 |
| KLF4 | chr9 | − | 107487027 | 107488267 | 7.52E−14 | 6.42E−11 | 0.054 |
| CALCOCO1 | chr12 | − | 53713700 | 53714241 | 0.00180502 | 0.01893054 | 0.054 |
| RPAIN | chr17 | + | 5425970 | 5428211 | 3.43E−07 | 1.79E−05 | 0.054 |
| CIRBP | chr19 | + | 1271980 | 1274440 | 0.00092856 | 0.01130533 | 0.053 |
| KRIT1 | chr7 | − | 92235402 | 92236542 | 0.0031679 | 0.0296365 | 0.053 |
| BSDC1 | chr1 | − | 32394079 | 32394425 | 9.98E−05 | 0.0019249 | 0.053 |
| GALT | chr9 | + | 34648333 | 34648894 | 0.00071067 | 0.00915305 | 0.053 |
| TMEM150A | chr2 | − | 85601020 | 85601482 | 0.0017761 | 0.01869283 | 0.053 |
| ZSWIM8 | chr10 | + | 73794146 | 73794639 | 0.00220143 | 0.02211789 | 0.052 |
| DPH1 | chr17 | + | 2040217 | 2040605 | 0.00490181 | 0.03997685 | 0.052 |
| TMEM205 | chr19 | − | 11345519 | 11346268 | 1.23E−05 | 0.00034559 | 0.052 |
| CARD8 | chr19 | − | 48232452 | 48234543 | 0.00069379 | 0.00897719 | 0.051 |
| LFNG | chr7 | + | 2525218 | 2525567 | 5.96E−08 | 4.35E−06 | 0.051 |
| NDUFAF7 | chr2 | + | 37241577 | 37242693 | 0.00032606 | 0.00484879 | 0.051 |
| ARGLU1 | chr13 | − | 106557047 | 106559657 | 4.99E−06 | 0.00015788 | 0.051 |
| RNF44 | chr5 | − | 176529730 | 176530206 | 3.42E−05 | 0.00081234 | 0.05 |
| NRBP2 | chr8 | − | 143835819 | 143836030 | 1.42E−06 | 5.66E−05 | 0.05 |
| ERVK13-1 | chr16 | − | 2669410 | 2672570 | 2.29E−06 | 8.31E−05 | 0.05 |
| ZNF7 | chr8 | + | 144829042 | 144829604 | 1.14E−07 | 7.47E−06 | −0.05 |
| MSTO1 | chr1 | + | 155611215 | 155611603 | 4.87E−05 | 0.00110649 | −0.052 |
| MIB2 | chr1 | + | 1628272 | 1628722 | 1.27E−05 | 0.00035643 | −0.055 |
| TMEM147 | chr19 | + | 35546671 | 35547240 | 0.0001275 | 0.00232369 | −0.055 |
| BBS1 | chr11 | + | 66515539 | 66515731 | 0.00033646 | 0.00494192 | −0.057 |
| ZNF160 | chr19 | − | 53091412 | 53091720 | 4.30E−11 | 1.11E−08 | −0.058 |
| ZNF577 | chr19 | − | 51886820 | 51887936 | 2.03E−05 | 0.00052052 | −0.059 |
| MFSD2A | chr1 | + | 39967627 | 39967916 | 0.00034008 | 0.00498285 | −0.064 |
| VPS11 | chr11 | + | 119078172 | 119078997 | 2.56E−08 | 2.21E−06 | −0.064 |
| PPOX | chr1 | + | 161170906 | 161171181 | 0.0063269 | 0.04924767 | −0.065 |
| CDK16 | chrX | + | 47225964 | 47226703 | 0.00034662 | 0.00506624 | −0.069 |
| MIB2 | chr1 | + | 1625545 | 1626754 | 0.00490882 | 0.03997944 | −0.074 |
| PRPF40B | chr12 | + | 49642234 | 49642675 | 0.00182504 | 0.01909004 | −0.079 |
| STARD5 | chr15 | − | 81322407 | 81324141 | 1.83E−06 | 6.84E−05 | −0.083 |
| MIB2 | chr1 | + | 1626836 | 1627207 | 1.48E−07 | 9.09E−06 | −0.085 |
| CDK16 | chrX | + | 47226993 | 47227223 | 0.001357 | 0.01521984 | −0.085 |
| ALG9 | chr11 | − | 111782194 | 111786520 | 7.34E−05 | 0.00150848 | −0.089 |
| PPIEL | chr1 | − | 39547578 | 39548951 | 0.00030855 | 0.00466963 | −0.099 |
| HTRA2 | chr2 | + | 74531338 | 74531702 | 0.00282866 | 0.02709895 | −0.113 |
| MAN2C1 | chr15 | − | 75358461 | 75358808 | 0.00237556 | 0.02353446 | −0.124 |
| ZNF528- | chr19 | − | 52396609 | 52397267 | 1.36E−05 | 0.00038005 | −0.131 |
| AS1 | | | | | | | |
| MYO1G | chr7 | − | 44972114 | 44975227 | 0.00166505 | 0.01780618 | −0.16 |
| PHYHD1 | chr9 | + | 128941444 | 128942038 | 0.00127981 | 0.0146074 | −0.162 |
| SNHG17 | chr20 | − | 38421005 | 38422241 | 6.79E−05 | 0.00141902 | −0.165 |
| TCL6 | chr14 | + | 95668125 | 95669617 | 2.48E−05 | 0.0006141 | −0.167 |
| NMRK1 | chr9 | − | 75068995 | 75070042 | 3.94E−10 | 6.90E−08 | −0.264 |

TABLE 22
CARRIER_VS_TD_SE

| geneSymbol | chr | strand | exonStart_0base | exonEnd | PValue | FDR | type |
|---|---|---|---|---|---|---|---|
| EXOC7 | chr17 | − | 76091142 | 76091235 | 9.34E−09 | 8.57E−06 | UP |
| LRRC23 | chr12 | + | 6909889 | 6910026 | 0.00032136 | 0.01394753 | DN |
| TPD52L1 | chr6 | + | 125252021 | 125252036 | 5.23E−05 | 0.00421507 | UP |
| NRBP1 | chr2 | + | 27435722 | 27435746 | 0.00064854 | 0.02084914 | DN |
| COX5A | chr15 | − | 74926765 | 74926887 | 0.00096182 | 0.02686428 | NC |
| UBE2Q2 | chr15 | + | 75859877 | 75859982 | 0.00052428 | 0.01842707 | UP |
| SAT2 | chr17 | − | 7626942 | 7627044 | 1.38E−09 | 2.26E−06 | DN |
| ABLIM1 | chr10 | − | 114447879 | 114448026 | 0.00118326 | 0.03071357 | DN |
| TTC3 | chr21 | + | 37150819 | 37150884 | 0.00080955 | 0.02389588 | NC |
| SZRD1 | chr1 | + | 16391374 | 16391424 | 0.00013927 | 0.00836857 | UP |
| KIF21A | chr12 | − | 39346465 | 39346504 | 0.00050149 | 0.01792614 | UP |
| CDC16 | chr13 | − | 114261886 | 114261948 | 0.00069299 | 0.02167629 | UP |
| PRPF38B | chr1 | + | 108693589 | 108693642 | 0.00224369 | 0.04603797 | UP |
| MOBP | chr3 | + | 39499466 | 39499532 | 0.00115894 | 0.03026943 | UP |
| ZNF148 | chr3 | − | 125314936 | 125315053 | 0.00137804 | 0.0337425 | UP |
| GAB1 | chr4 | + | 143457679 | 143457769 | 0.00064841 | 0.02084914 | DN |
| PCDHGC3 | chr5 | + | 141478393 | 141478546 | 0.00022554 | 0.01127053 | DN |
| ZFAND5 | chr9 | − | 72363469 | 72363606 | 0.00042095 | 0.0161939 | UP |
| SGIP1 | chr1 | + | 66695433 | 66695493 | 0.00098237 | 0.02725673 | UP |
| LRRC75A- | chr17 | + | 16439527 | 16439703 | 8.35E−09 | 7.91E−06 | DN |
| AS1 | | | | | | | |
| LRRC75A- | chr17 | + | 16439580 | 16439703 | 9.84E−10 | 1.81E−06 | DN |
| AS1 | | | | | | | |
| LRRC75A- | chr17 | + | 16439659 | 16439703 | 6.77E−07 | 0.00019304 | NC |
| AS1 | | | | | | | |
| PCBP4 | chr3 | − | 51962809 | 51962892 | 1.87E−07 | 8.09E−05 | UP |
| CAMK2A | chr5 | − | 150239703 | 150239736 | 8.02E−05 | 0.00557276 | DN |
| CWC27 | chr5 | + | 64977134 | 64977238 | 0.00191753 | 0.04167371 | DN |
| ARPP21 | chr3 | + | 35717297 | 35717357 | 4.58E−06 | 0.00087943 | UP |
| LSM14A | chr19 | + | 34226407 | 34226464 | 2.33E−05 | 0.0025537 | UP |
| PCBP2 | chr12 | + | 53467804 | 53467843 | 0.00033827 | 0.01426026 | UP |
| ARHGAP26 | chr5 | + | 143207197 | 143207473 | 0.0024596 | 0.04885996 | DN |
| IDH3B | chr20 | − | 2658758 | 2658837 | 0.00056708 | 0.01932662 | UP |
| NOVA1 | chr14 | − | 26472319 | 26472391 | 0.00174589 | 0.03927977 | DN |
| LINC-PINT | chr7 | − | 130959868 | 130959970 | 0.00130581 | 0.03270973 | UP |
| ZNF207 | chr17 | + | 32366664 | 32366757 | 1.22E−06 | 0.00030003 | DN |
| GKAP1 | chr9 | − | 83768817 | 83768970 | 0.00202314 | 0.043108 | DN |
| CDC42BPA | chr1 | − | 227112670 | 227112913 | 0.00072056 | 0.02216975 | UP |
| MAP7D1 | chr1 | + | 36174897 | 36175008 | 0.00042274 | 0.01621597 | DN |
| PAQR6 | chr1 | − | 156245534 | 156245661 | 0.00037187 | 0.01523943 | UP |
| MADD | chr11 | + | 47288967 | 47289027 | 0.00155148 | 0.0363534 | DN |
| MADD | chr11 | + | 47308979 | 47309042 | 0.00143511 | 0.03453551 | UP |
| KIF13A | chr6 | − | 17789871 | 17789910 | 0.00014729 | 0.00865225 | UP |
| UNC79 | chr14 | + | 93634520 | 93634637 | 0.00035917 | 0.01484301 | DN |
| NCAM1 | chr11 | + | 113236300 | 113236315 | 2.97E−05 | 0.00293482 | DN |
| NCAM1 | chr11 | + | 113246367 | 113246370 | 0.00119711 | 0.03080089 | DN |
| CAMK2D | chr4 | − | 113502935 | 113502977 | 0.00178425 | 0.03972543 | DN |
| TRIM33 | chr1 | − | 114397939 | 114397990 | 0.00215217 | 0.04488091 | UP |
| CCAR1 | chr10 | + | 68756272 | 68756483 | 8.10E−05 | 0.0056135 | DN |
| RP5- | chr22 | + | 40364879 | 40365056 | 0.00198136 | 0.04248854 | DN |
| 1042K10.14 | | | | | | | |
| CAPN3 | chr15 | + | 42408210 | 42408324 | 0.00022642 | 0.01127607 | UP |
| HSD11B1L | chr19 | + | 5686415 | 5686527 | 0.00179896 | 0.03992359 | DN |
| UQCRH | chr1 | + | 46309100 | 46309127 | 2.30E−11 | 2.25E−07 | UP |
| MARK2 | chr11 | + | 63908259 | 63908304 | 0.0001404 | 0.00840188 | UP |
| BIN1 | chr2 | − | 127051153 | 127051243 | 0.00149082 | 0.03549823 | NC |
| AIFM3 | chr22 | + | 20980746 | 20980767 | 3.67E−06 | 0.00077479 | DN |
| COG1 | chr17 | + | 73207701 | 73207739 | 2.82E−07 | 0.00011036 | UP |
| SORBS1 | chr10 | − | 95414493 | 95414862 | 0.00180639 | 0.04002812 | DN |
| BAZ2B | chr2 | − | 159400598 | 159400664 | 0.00109098 | 0.0290259 | NC |
| BRSK2 | chr11 | + | 1459191 | 1459239 | 0.0005302 | 0.01852433 | DN |
| ARMC10 | chr7 | + | 103092476 | 103092653 | 0.00039015 | 0.01563491 | DN |
| AC005154.6 | chr7 | − | 30552099 | 30552179 | 0.00057621 | 0.01942803 | UP |
| LCMT1 | chr16 | + | 25161101 | 25161204 | 0.00013305 | 0.00807714 | UP |
| USMG5 | chr10 | − | 103394198 | 103394394 | 2.73E−06 | 0.00059877 | UP |
| USMG5 | chr10 | − | 103394198 | 103394394 | 1.35E−08 | 1.05E−05 | NC |
| CLSTN1 | chr1 | − | 9737497 | 9737554 | 0.00093918 | 0.02646864 | NC |
| ZMIZ2 | chr7 | + | 44760150 | 44760228 | 0.00028376 | 0.01304811 | UP |
| UQCRB | chr8 | − | 96231378 | 96231515 | 4.33E−08 | 2.60E−05 | NC |
| SH3GLB2 | chr9 | − | 129021090 | 129021219 | 0.00242444 | 0.04832932 | DN |
| CAMK2G | chr10 | − | 73825278 | 73825347 | 0.00199683 | 0.04266565 | UP |
| CAMK2G | chr10 | − | 73828088 | 73828121 | 0.00067016 | 0.0212065 | DN |
| CAMK2G | chr10 | − | 73828088 | 73828121 | 0.00212556 | 0.04445219 | DN |
| ACBD4 | chr17 | + | 45137018 | 45137139 | 0.00069627 | 0.0216951 | DN |
| GPM6B | chrX | − | 13807649 | 13807769 | 9.76E−06 | 0.00139273 | UP |
| MTDH | chr8 | + | 97699753 | 97699852 | 0.00028272 | 0.01303802 | DN |
| MTDH | chr8 | + | 97719048 | 97719189 | 1.45E−05 | 0.00182274 | NC |
| MAP4K4 | chr2 | + | 101860824 | 101860986 | 0.00207288 | 0.04372388 | UP |
| MAP4K4 | chr2 | + | 101863820 | 101864051 | 2.52E−10 | 6.73E−07 | DN |
| BCAS1 | chr20 | − | 53957431 | 53957497 | 0.00014617 | 0.00862437 | UP |
| TRAPPC12 | chr2 | + | 3477694 | 3477795 | 0.00185734 | 0.04087953 | UP |
| PHYHIP | chr8 | − | 22228787 | 22228909 | 1.61E−05 | 0.00196115 | UP |
| SYNE1 | chr6 | − | 152130719 | 152130778 | 0.00033978 | 0.01429316 | UP |
| KCNC3 | chr19 | − | 50316032 | 50316091 | 0.00011194 | 0.0070884 | UP |
| ACAA1 | chr3 | − | 38126161 | 38126341 | 0.00057348 | 0.01939066 | DN |
| RNF146 | chr6 | + | 127285207 | 127285348 | 0.00027653 | 0.01288046 | UP |
| GSTO1 | chr10 | + | 104262978 | 104263077 | 2.59E−07 | 0.00010575 | DN |
| ARHGAP12 | chr10 | − | 31839636 | 31839711 | 0.00092024 | 0.02613029 | UP |
| AP1G2 | chr14 | − | 23565815 | 23565892 | 4.26E−05 | 0.00368385 | DN |
| PHLDB1 | chr11 | + | 118655859 | 118655892 | 9.45E−05 | 0.00623718 | UP |
| SH3YL1 | chr2 | − | 224863 | 224920 | 0.00048615 | 0.01763532 | UP |
| NRCAM | chr7 | − | 108237751 | 108237769 | 0.00029057 | 0.01323689 | UP |
| ASPH | chr8 | − | 61646749 | 61646878 | 0.00104222 | 0.02827661 | UP |
| ASPDH | chr19 | − | 50512926 | 50513011 | 0.00108324 | 0.02890907 | DN |
| ZIM2 | chr19 | − | 56826387 | 56826463 | 0.00058246 | 0.01955921 | UP |
| CHL1 | chr3 | + | 342983 | 343031 | 0.0006118 | 0.02015719 | DN |
| COL16A1 | chr1 | − | 31679803 | 31679851 | 0.00221949 | 0.04572039 | DN |
| PRKACB | chr1 | + | 84175798 | 84175807 | 1.79E−05 | 0.00209292 | UP |
| GANAB | chr11 | − | 62634309 | 62634375 | 2.51E−06 | 0.00055786 | DN |
| RHBDD2 | chr7 | + | 75881364 | 75881486 | 0.00130743 | 0.03270973 | UP |
| TJP1 | chr15 | − | 29719776 | 29720016 | 3.72E−06 | 0.00078017 | DN |
| KIF3A | chr5 | − | 132708906 | 132708974 | 9.44E−06 | 0.00137048 | DN |
| GOLIM4 | chr3 | − | 168040785 | 168040869 | 0.00117628 | 0.0306136 | UP |
| CBWD1 | chr9 | − | 177722 | 177820 | 0.00042182 | 0.01620172 | DN |
| ITSN2 | chr2 | − | 24284762 | 24284843 | 7.09E−06 | 0.00115732 | UP |
| DAP3 | chr1 | + | 155729041 | 155729122 | 0.00082594 | 0.02426863 | UP |
| CCDC136 | chr7 | + | 128806236 | 128806395 | 0.00165475 | 0.03789665 | DN |
| CLASP2 | chr3 | − | 33577201 | 33577264 | 0.00090364 | 0.02587886 | DN |
| NCOA1 | chr2 | + | 24768078 | 24768135 | 2.16E−05 | 0.00242146 | UP |
| GNAS | chr20 | + | 58898940 | 58898985 | 0.00164511 | 0.03776438 | NC |
| RBFOX1 | chr16 | + | 7693314 | 7693367 | 0.00099681 | 0.02736915 | UP |
| CEP290 | chr12 | − | 88054339 | 88054413 | 9.27E−05 | 0.00618541 | UP |
| PUF60 | chr8 | − | 143820665 | 143820716 | 6.52E−05 | 0.00489236 | UP |
| SLC25A23 | chr19 | − | 6453980 | 6454088 | 0.00112685 | 0.02964206 | NC |
| MEG3 | chr14 | + | 100834631 | 100834751 | 6.17E−05 | 0.0047638 | NC |
| MEG3 | chr14 | + | 100835738 | 100835879 | 0.00141988 | 0.03434764 | NC |
| FN1 | chr2 | − | 215372108 | 215372300 | 3.99E−06 | 0.00081892 | UP |
| FN1 | chr2 | − | 215372108 | 215372375 | 7.23E−07 | 0.00020244 | UP |
| DTNA | chr18 | + | 34866110 | 34866131 | 0.00228755 | 0.04652658 | DN |
| GPM6A | chr4 | − | 175812190 | 175812249 | 1.24E−08 | 1.02E−05 | UP |
| OLA1 | chr2 | − | 174246714 | 174246815 | 0.00057014 | 0.01936578 | DN |
| YWHAZ | chr8 | − | 100951928 | 100952125 | 6.18E−05 | 0.0047638 | UP |
| RNASE1 | chr14 | − | 20802248 | 20802319 | 9.31E−05 | 0.00618541 | DN |
| RNASE1 | chr14 | − | 20802248 | 20802331 | 6.42E−05 | 0.00487467 | DN |
| CALU | chr7 | + | 128754528 | 128754722 | 0.00187284 | 0.04109756 | UP |
| ALG8 | chr11 | − | 78102873 | 78102948 | 3.69E−07 | 0.00012922 | UP |

TABLE 23
CARRIER_VS_TD_MXE

| geneSymbol | chr | strand | 1stExonStart_0base | 1stExonEnd | 2ndExonStart_0base | 2ndExonEnd | PValue | FDR | type |
|---|---|---|---|---|---|---|---|---|---|
| FYN | chr6 | − | 111699514 | 111699670 | 111700103 | 111700268 | 0.0001885 | 0.0096127 | UP |
| PIBF1 | chr13 | + | 72965273 | 72965404 | 72973590 | 72973675 | 0.00116618 | 0.02927732 | DN |
| TBC1D5 | chr3 | − | 17238162 | 17238419 | 17258505 | 17258591 | 0.00019191 | 0.0096127 | UP |
| TNRC6A | chr16 | + | 24758338 | 24758360 | 24776932 | 24777358 | 0.0001441 | 0.00827948 | UP |
| SEZ6L | chr22 | + | 26365371 | 26365566 | 26375574 | 26375689 | 1.72E−05 | 0.00172028 | DN |
| SEZ6L | chr22 | + | 26365371 | 26365566 | 26375577 | 26375689 | 1.05E−05 | 0.0011766 | DN |
| ZMAT4 | chr8 | − | 40581164 | 40581261 | 40674703 | 40674931 | 0.00022382 | 0.01053576 | UP |
| MOBP | chr3 | + | 39480039 | 39480123 | 39499466 | 39499532 | 0.0007262 | 0.02234068 | NC |
| PDXDC1 | chr16 | + | 15001775 | 15001856 | 15004186 | 15004333 | 0.0011669 | 0.02927732 | UP |
| TPM3 | chr1 | − | 154170648 | 154170711 | 154172028 | 154172104 | 0.00213151 | 0.04360117 | DN |
| ANKRD24 | chr19 | + | 4199874 | 4200005 | 4200082 | 4200171 | 0.00012217 | 0.00723224 | DN |
| WIPF2 | chr17 | + | 40262524 | 40262641 | 40264489 | 40265146 | 0.00096758 | 0.02662204 | UP |
| ATRNL1 | chr10 | + | 115315517 | 115315736 | 115334281 | 115334419 | 3.92E−05 | 0.00312596 | UP |
| FXYD7 | chr19 | + | 35151253 | 35151328 | 35151439 | 35151473 | 0.00212545 | 0.04360117 | DN |
| TSSC1 | chr2 | − | 3257298 | 3257455 | 3338016 | 3338149 | 2.65E−05 | 0.00229997 | DN |
| RP11- | chr18 | + | 9124873 | 9124983 | 9126830 | 9126907 | 2.08E−08 | 9.02E−06 | DN |
| 21J18.1 | | | | | | | | | |
| PAQR6 | chr1 | − | 156245534 | 156245661 | 156245781 | 156245987 | 0.00210179 | 0.04360117 | NC |
| MGEA5 | chr10 | − | 101800241 | 101800400 | 101803734 | 101804019 | 0.0005927 | 0.01945942 | DN |
| ANK3 | chr10 | − | 60200128 | 60200227 | 60203001 | 60203100 | 0.00116899 | 0.02927732 | DN |
| PITPNC1 | chr17 | + | 67532801 | 67532950 | 67552256 | 67552345 | 0.00090702 | 0.02552935 | UP |
| MAPT | chr17 | + | 45962320 | 45962470 | 45971858 | 45971945 | 0.0003531 | 0.01385376 | UP |
| VCAN | chr5 | + | 83519348 | 83522309 | 83537006 | 83542268 | 8.51E−05 | 0.00563641 | DN |
| DAB2IP | chr9 | + | 121759884 | 121760439 | 121763504 | 121763649 | 0.00077255 | 0.02321801 | DN |
| PRKAG2 | chr7 | − | 151595344 | 151595454 | 151632068 | 151632138 | 0.0018563 | 0.04029196 | UP |
| SETD4 | chr21 | − | 36048307 | 36048396 | 36053582 | 36053620 | 0.00204734 | 0.04330727 | UP |
| SMARCA4 | chr19 | + | 11007901 | 11008023 | 11010380 | 11010531 | 0.00043714 | 0.01611235 | UP |
| BAZ2B | chr2 | − | 159448241 | 159448403 | 159453612 | 159453801 | 0.00088355 | 0.02552935 | DN |
| BAZ2B | chr2 | − | 159448241 | 159448409 | 159453612 | 159453801 | 0.00207445 | 0.04334168 | DN |
| RBFA | chr18 | + | 80038504 | 80038617 | 80042134 | 80042219 | 2.78E−05 | 0.00236311 | UP |
| LUC7L | chr16 | − | 220648 | 220747 | 227241 | 227336 | 0.00146784 | 0.03454721 | DN |
| AIF1 | chr6 | + | 31615669 | 31615736 | 31616103 | 31616145 | 0.00177591 | 0.0396485 | DN |
| PDE2A | chr11 | − | 72631077 | 72631139 | 72642253 | 72642326 | 0.00250474 | 0.0491759 | DN |
| CAMK2G | chr10 | − | 73825278 | 73825347 | 73828088 | 73828121 | 0.00035499 | 0.01385376 | DN |
| CAMK2G | chr10 | − | 73825278 | 73825347 | 73828088 | 73828121 | 0.00047163 | 0.01675142 | DN |
| CCDC7 | chr10 | + | 32567669 | 32567891 | 32583033 | 32583307 | 0.00205141 | 0.04330727 | UP |
| PMS1 | chr2 | + | 189863742 | 189863974 | 189867798 | 189867929 | 0.00166957 | 0.037989 | UP |
| PMS1 | chr2 | + | 189863742 | 189864228 | 189867798 | 189867929 | 0.00115616 | 0.02927732 | UP |
| ADCK4 | chr19 | − | 40705324 | 40705447 | 40710058 | 40710136 | 0.00139712 | 0.03328376 | UP |
| CRELD2 | chr22 | + | 49923233 | 49923317 | 49924359 | 49924455 | 0.00031569 | 0.01326252 | DN |
| ARID4A | chr14 | + | 58360900 | 58361042 | 58364169 | 58365300 | 6.83E−05 | 0.00503336 | UP |
| MTDH | chr8 | + | 97699753 | 97699852 | 97706625 | 97706750 | 0.00032343 | 0.01330165 | DN |
| MAP4K4 | chr2 | + | 101860824 | 101860986 | 101863820 | 101864051 | 4.74E−14 | 1.85E−10 | UP |
| ACTR1B | chr2 | − | 97660570 | 97660646 | 97661881 | 97661946 | 0.000288 | 0.01250239 | DN |
| BCAS3 | chr17 | + | 61078332 | 61078529 | 61084466 | 61084564 | 9.91E−05 | 0.00604969 | DN |
| GTF2A2 | chr15 | − | 59650668 | 59650773 | 59652205 | 59652326 | 0.00074271 | 0.02266993 | UP |
| C11orf80 | chr11 | + | 66788159 | 66788269 | 66796280 | 66796364 | 8.12E−06 | 0.00103464 | UP |
| ZNF106 | chr15 | − | 42442072 | 42442414 | 42444201 | 42444262 | 0.00031208 | 0.01325331 | UP |
| ZNF106 | chr15 | − | 42448071 | 42448705 | 42466052 | 42466114 | 0.00107974 | 0.02850378 | DN |
| SESN1 | chr6 | − | 108990644 | 108990835 | 108992786 | 108992899 | 1.37E−08 | 8.90E−06 | UP |
| PHYHD1 | chr9 | + | 128940368 | 128940497 | 128940598 | 128940715 | 0.00056706 | 0.01893578 | DN |
| GRID1 | chr10 | − | 85869009 | 85869180 | 85916185 | 85916239 | 0.00253 | 0.0494236 | UP |
| MAP9 | chr4 | − | 155355715 | 155355884 | 155357448 | 155357519 | 0.00091464 | 0.02552935 | DN |
| NRCAM | chr7 | − | 108166920 | 108167073 | 108168276 | 108168402 | 0.00206172 | 0.04330727 | UP |
| ASPDH | chr19 | − | 50512135 | 50512290 | 50512926 | 50513011 | 0.00042917 | 0.01596935 | UP |
| VIPR1 | chr3 | + | 42532241 | 42532333 | 42534974 | 42535104 | 8.20E−05 | 0.00562087 | DN |
| PLEKHA6 | chr1 | − | 204250545 | 204250614 | 204251530 | 204251611 | 9.64E−05 | 0.00597536 | DN |
| ADAL | chr15 | + | 43334944 | 43335170 | 43335695 | 43335826 | 2.97E−06 | 0.00048297 | UP |
| C11orf74 | chr11 | + | 36636050 | 36636117 | 36648015 | 36648155 | 0.00195189 | 0.04167242 | DN |
| SYMPK | chr19 | − | 45838460 | 45838615 | 45842249 | 45842489 | 0.00033373 | 0.01358205 | UP |
| RANGAP1 | chr22 | − | 41274599 | 41274727 | 41280932 | 41281082 | 0.00168609 | 0.03807833 | DN |
| PON2 | chr7 | − | 95412311 | 95412477 | 95416241 | 95416297 | 0.00249155 | 0.04916413 | UP |
| ITSN2 | chr2 | − | 24275712 | 24275849 | 24286211 | 24286351 | 0.00018966 | 0.0096127 | DN |
| SNW1 | chr14 | − | 77720710 | 77720828 | 77723180 | 77723277 | 0.00015567 | 0.00856649 | UP |
| SPG11 | chr15 | − | 44585635 | 44585850 | 44589251 | 44589414 | 0.00149353 | 0.03479665 | UP |
| RGL1 | chr1 | + | 183806374 | 183806485 | 183847565 | 183847774 | 0.00015307 | 0.00854367 | UP |
| CCDC136 | chr7 | + | 128807359 | 128807545 | 128817757 | 128817864 | 0.0019377 | 0.0415967 | NC |
| HACE1 | chr6 | − | 104833041 | 104833173 | 104843222 | 104843298 | 0.00239699 | 0.04778084 | UP |
| SLC1A3 | chr5 | + | 36608328 | 36608604 | 36629449 | 36629587 | 0.00182552 | 0.04006917 | DN |
| BRWD1 | chr21 | − | 39238478 | 39238573 | 39247700 | 39247832 | 0.00104763 | 0.02803498 | DN |
| PSMB1 | chr6 | − | 170537233 | 170537340 | 170543600 | 170543730 | 0.00042318 | 0.01589761 | DN |
| CUX2 | chr12 | + | 111291417 | 111291552 | 111293445 | 111293569 | 0.00020506 | 0.01001475 | DN |
| VPS26A | chr10 | + | 69166041 | 69166110 | 69168488 | 69168631 | 2.22E−05 | 0.00201507 | DN |
| EGFL7 | chr9 | + | 136664691 | 136664785 | 136668240 | 136668362 | 0.00049378 | 0.01738029 | DN |
| ADCK2 | chr7 | + | 140681041 | 140681137 | 140686989 | 140687241 | 0.00035813 | 0.01385376 | DN |
| ZNF415 | chr19 | − | 53115209 | 53115307 | 53115703 | 53115811 | 0.00104564 | 0.02803498 | DN |
| ANAPC16 | chr10 | + | 72223887 | 72224056 | 72230365 | 72230440 | 0.00044252 | 0.01615836 | UP |
| CCM2 | chr7 | + | 45038252 | 45038426 | 45072725 | 45072783 | 0.00064624 | 0.02046533 | DN |
| CCM2 | chr7 | + | 45069825 | 45069961 | 45072725 | 45072783 | 0.00072127 | 0.02234068 | NC |
| ARL3 | chr10 | − | 102699372 | 102699489 | 102705345 | 102705489 | 0.00103662 | 0.02803498 | UP |
| ARCN1 | chr11 | + | 118593589 | 118593698 | 118597706 | 118597911 | 2.49E−06 | 0.00046342 | DN |
| CALU | chr7 | + | 128754261 | 128754455 | 128754528 | 128754722 | 5.21E−05 | 0.00407347 | DN |

TABLE 24
CARRIER_VS_TD_DE

| gene_symbol | FXS_patient_vs_healthy_type |
|---|---|
| ADGRL4 | DN |
| APOLD1 | DN |
| BLOC1S5-TXNDC5 | UP |
| C10orf10 | DN |
| CD93 | DN |
| ELOVL7 | DN |
| F8A3 | DN |
| FCGR3A | DN |
| FER1L6 | UP |
| ITGA6 | DN |
| KIF25 | UP |
| MIR143HG | UP |
| NUTM2E | UP |
| OR7D2 | DN |
| PECAM1 | DN |
| POC1B-GALNT4 | UP |
| RP11-17M24.3 | UP |
| RP11-73M18.2 | DN |
| RP11-986E7.7 | DN |
| RPL17-C18orf32 | DN |
| S100A9 | DN |
| SST | UP |
| TBC1D3L | UP |
| TVP23C-CDRT4 | DN |
| VSIG4 | DN |
| XXbac-BPG246D15.9 | UP |

### Legends for Tables 3-10 and 16-24:

SE: Skipped exon
MXE: Mutually exclusive exons
A3SS: Alternative 3′ splice site
A5SS: Alternative 5′ splice site
ENSEMBL Gene ID
Chr: chromosome
Strand: Strand
exonStart_0base: start position of the skipped exon
exonEnd: end position of the skipped exon
1 stExonStart_0base: start position of the 1st exon in MXE
1 stExonEnd: end position of the 1st exon in MXE
2ndExonStart_0base: start position of the 2nd exon in MXE
2nd ExonEnd: end position of the 2nd exon in MXE
longExonStart_0base: start position of the long exon in A3SS or A5SS
longExonEnd: end position of the long exon in A3SS or A5SS
shortES: start position of the short exon in A3SS or A5SS
shortEE: end position of the short exon in A3SS or A5SS
flankingES: start position of the closest flanking exon in A3SS or A5SS
flankingEE: end position of the closest flanking exon in A3SS or A5SS
upstreamES: start position of the closest upstream exon
upstreamEE: end position of the closest upstream exon
downstreamES: start position of the closest downstream exon
downstreamEE: end position of the closest downstream exon
IncLevelDifference: IncLevelDifference: average (IncLevel FXS)—average (IncLevel Control)
Type: Up, significantly increased splicing in FXS; DN, significantly increased splicing in Control; NC, no change.
P-Value: Significance of splicing difference between two groups.
FDR: False Discovery Rate calculated from p-value.

### EMBODIMENTS

1. A method of diagnosing a subject as having, or having a propensity to develop, fragile X syndrome (FXS), comprising assaying at least one RNA biomarker in a biological sample from the subject, wherein the level and/or splicing of the at least one RNA biomarker in the biological sample is indicative of the subject as having, or having a propensity to develop, FXS, and wherein the biological sample is a non-neural biological sample.

2. A method of prognosing fragile X syndrome (FXS) in a subject, comprising assaying at least one RNA biomarker in a biological sample from the subject, wherein the level and/or splicing of the at least one RNA biomarker in the biological sample is indicative of the subject as having a propensity to have a poorer prognosis of FXS, and wherein the biological sample is a non-neural biological sample.

3. A method of predicting a treatment outcome of fragile X syndrome (FXS) in a subject, comprising assaying at least one RNA biomarker in a biological sample from the subject, wherein the level and/or splicing of the at least one RNA biomarker in the biological sample is indicative of the subject as having a propensity to have a poorer treatment outcome, and wherein the biological sample is a non-neural biological sample.

4. A method of stratifying a set of subjects having fragile X syndrome (FXS), comprising assaying at least one RNA biomarker in a biological sample from the subject, and stratifying the set of subjects for treatment based on the level or splicing of the at least one RNA biomarker in the biological sample, wherein the biological sample is a non-neural biological sample.

5. The method of any one of Embodiments 1-4, wherein the biological sample is a bodily fluid sample, a hair sample, buccal swab sample or a skin sample.

6. The method of Embodiment 5, wherein the bodily fluid sample comprises blood, saliva, tears, urine or semen.

7. The method of Embodiment 5, wherein the bodily fluid sample comprises white blood cells.

8. The method of any one of Embodiments 1-7, wherein the at least one RNA biomarker is selected from the group consisting of AGAP1, RAB25, FAM3B, XKR3, MAP3K15, LEP, RP11-706O15.3, GCOM1, CXCL6, RGL3, NECAB2, TGM3, LRRC6, MAB21L3, RP11-36B15.1, AC091878.1, RP11-154H23.3, NOV, AC093495.4, RP11-455F5.6, RGPD2, COL9A3, CLEC18A, RP11-256L6.2, LINC01127, SLC38A11, EFCAB12, LA16c-380H5.5, CXCL1, RP11-1334A24.5, AC100793.2, ANKDD1A, AVIL, RP11-44F14.8, RP11-290F20.1, AC116366.5, EPHB4, ST6GALNAC3, PANX2, CREB5, KIAA0319, HECW2, ADCY4, LINC000173, RP11-59D5_B.2, RP11-274B18.2, RP11-213H15.3, CORO7-PAM16, HAL, DPEP3, AC002467.7, MGAM, PNMA8A, FMR1, S100B, RP11-885N19.6, RP11-545I5.3, AC091814.2, KLRC2, L1TD1, PGBD5, MXRA7, CROCC2, SEMA5A, PLA2G4C, RP11-1008C21.1, TANC1, C4orf50, NUAK1, AC104809.4, RGS17, KCNS1, DRAXIN, B3GAT1, ARHGEF28, KIF19, APOL4, GZMH, GAS1, SCD5, GLB1L2, IGHA1, KNDC1, RP11-383H13.1, FGFR2, TFCP2L1, PDGFRB, LAG3, GPR153, PODN, CKB, CERCAM, ZNF365, JUP, TRNP1, JAKMIP1, CPXM1, SLC1A7, LGR6, FCRL6, MORN4, TUBB2A, PRSS23, BFSP1, NCALD, ZNF573, PAK1, MIR4435-2HG, CD8B, PDGFC, TRAPPC2L, AC006504.5, ZNF512, FAM228B, NEIL2, FAM78A, FYB1, RNF216P1, ZCWPW1, DTX2, ATP5MD, MX2, LYRM1, GUF1, DPH7, NSFL1C, MTMR1, GTPBP10, RGS3, DRAM2, RHOH, LAIR2, GBP3, GTF2H1, XPNPEP3, ZNF888, TBC1D5, AC060780.1, SDHAP2, KMT2A, SH3BP2, CSNK1G2, NSUN5P1, LINC01128, RNF19A, SNHG8, TOP1MT, AL135818.1, CR1, CRIM1, NAP1L1, AC004593.2, SEC61A2, PCNX2, TPT1-AS1, HLA-A, LUCAT1, PTPN2, SEC31B, POLR2J3, POLR2J4, CAST, POLR2J4, NUMBL, PRMT7, ATF7IP2, TIMM23B-AGAP6, ADGRE2, GTF2H2B, MICA, SLC29A2, ZBTB10, NLGN3, METTL25, ADAM15, SSH1, SIRPB1, PARP2, PACRGL, ENTPD1-AS1, FUZ, SDR39U1, EPOR, ZSCAN26, SNHG17, GPS2, NECAP1, MRPLI1, DNAJC19, ANKZF1, C1orf162, PIGT, SLC25A37, AP1G1, CIC, ITGB7, ATG16L2, BECN1, ARHGEF40, BANP, PIGA, RAD52, IRF3, CEP78, SPINT1, TMEM156, NT5C3B, PLD2, ANKRD12, CASP8, PACS2, HLA-DMA, DHPS, PDCD6, SNX5, MPPE1, AC016394.2, DPM1, E2F5, PTPN7, MTFP1, TOR1AIP1, POT1, JOSD2, NLRX1, FDXR, ZDHHC16, ALKBH4, RPS9, ZNF302, TENT4B, TKT, CARD8, RBM26, WSB1, DDX60L, ATP11A, SRGAP2, CEACAM21, COX18, WDR47, PATZ1, POLM, CC2D1B, CLK4, MIB2, PHF1, KANSL1, TCF3, and combinations thereof.

9. The method of Embodiment 8, wherein the at least one RNA biomarker has an increased expression in the biological sample, relative to a control sample.

10. The method of Embodiment 9, wherein the expression of at least one RNA biomarker has a log 2 fold increase of ≥0.80 in the biological sample, relative to a control sample, optionally, wherein the log 2 fold increase is ≥0.95.

11. The method of Embodiment 9 or 10, wherein the at least one RNA biomarker is selected from the group consisting of AGAP1, RAB25, FAM3B, XKR3, MAP3K15, LEP, RP11-706O15.3, GCOM1, CXCL6, RGL3, NECAB2, TGM3, LRRC6, MAB21L3, RP11-36B15.1, AC091878.1, RP11-154H23.3, NOV, AC093495.4, RP11-455F5.6, RGPD2, COL9A3, CLEC18A, RP11-256L6.2, LINC01127, SLC38A11, EFCAB12, LA16c-380H5.5, CXCL1, RP11-1334A24.5, AC100793.2, ANKDD1A, AVIL, RP11-44F14.8, RP11-290F20.1, AC116366.5, EPHB4, ST6GALNAC3, PANX2, CREB5, KIAA0319, HECW2, ADCY4, LINC00173, RP11-59D5_B.2, RP11-274B18.2, RP11-213H15.3, CORO7-PAM16, HAL, DPEP3, AC002467.7, MGAM, PNMA8A, and combinations thereof.

12. The method of Embodiment 8, wherein the at least one RNA biomarker has a reduced expression in the biological sample, relative to a control sample.

13. The method of Embodiment 12, wherein the expression of the at least one RNA biomarker has a log 2 fold reduction of ≥1.00 in the biological sample, relative to a control sample, optionally, the log 2 fold reduction is ≥1.16.

14. The method of Embodiment 12 or 13, wherein the at least one RNA biomarker is selected from the group consisting of FMR1, S100B, RP11-885N19.6, RP11-545I5.3, AC091814.2, KLRC2, L1TD1, PGBD5, MXRA7, CROCC2, SEMA5A, PLA2G4C, RP11-1008C21.1, TANC1, C4orf50, NUAK1, AC104809.4, RGS17, KCNS1, DR AXIN, B3GAT1, ARHGEF28, KIF19, APOL4, GZMH, GAS1, SCD5, GLB1L2, IGHA1, KNDC1, RP11-383H13.1, FGFR2, TFCP2L1, PDGFRB, LAG3, GPR153, PODN, CKB, CERCAM, ZNF365, JUP, TRNP1, JAKMIP1, CPXM1, SLC1A7, LGR6, FCRL6, MORN4, TUBB2A, PRSS23, BFSP1, and combinations thereof.

15. The method of Embodiment 8, wherein the at least one RNA biomarker has a reduced exon skipping in the biological sample, relative to a control sample.

16. The method of Embodiment 15, wherein the reduction is ≥13.0%, optionally, the reduction is ≥16.0%.

17. The method of Embodiment 15 or 16, wherein the at least one RNA biomarker is selected from the group consisting of NCALD, ZNF573, PAK1, MIR4435-2HG, CD8B, PDGFC, TRAPPC2L, AC006504.5, ZNF512, FAM228B, NEIL2, FAM78A, FYB1, RNF216P1, ZCWPW1, DTX2, ATP5MD, MX2, LYRM1, GUF1, DPH7, NSFL1C, MTMR1, GTPBP10, RGS3, and combinations thereof.

18. The method of Embodiment 17, wherein the skipped exon is selected from the group consisting of the skipped exons listed in Table 3.

19. The method of Embodiment 8, wherein the at least one RNA biomarker has an increased exon skipping in the biological sample, relative to a control sample.

20. The method of Embodiment 19, wherein the increase is ≥13.0%, optionally, wherein the increase is ≥17.0%.

21. The method of Embodiment 19 or 20, wherein the at least one RNA biomarker is selected from the group consisting of NCALD, DRAM2, RHOH, LAIR2, GBP3, GTF2H1, XPNPEP3, ZNF888, TBC1D5, AC060780.1, SDHAP2, KMT2A, SH3BP2, CSNK1G2, ATP5MD, NSUN5P1, LINC01128, RNF19A, SNHG8, TOP1MT, AL135818.1, and combinations thereof.

22. The method of Embodiment 21, wherein the skipped exon is selected from the group consisting of the skipped exons listed in Table 4.

23. The method of Embodiment 8, wherein the at least one RNA biomarker has a reduced mutually exclusive exon switching in the biological sample, relative to a control sample.

24. The method of Embodiment 23, wherein the reduction is ≥10.0%, optionally, the reduction is ≥13.0%.

25. The method of Embodiment 23 or 24, wherein the at least one RNA biomarker is selected from the group consisting of CR1, CRIM1, ZCWPW1, NAPIL1, TBC1D5, MIR4435-2HG, AC004593.2, GBP3, SEC61A2, PCNX2, TPT1-AS1, HLA-A, LUCAT1, PTPN2, SEC31B, POLR2J3, POLR2J4, CAST, NUMBL, PRMT7, ATF7IP2, TIMM23B-AGAP6, and combinations thereof.

26. The method of Embodiment 25, wherein the mutually exclusive exon is selected from the group consisting of the mutually exclusive exons listed in Table 5.

27. The method of Embodiment 8, wherein the at least one RNA biomarker has an increased mutually exclusive exon switching in the biological sample, relative to a control sample.

28. The method of Embodiment 23, wherein the increase is ≥12.0%, optionally, the increase is ≥15.0%.

29. The method of Embodiment 27 or 28, wherein the at least one RNA biomarker is selected from the group consisting of HLA-A, ADGRE2, PAK1, TBC1D5, GTF2H2B, MICA, SLC29A2, ZBTB10, NLGN3, CAST, METTL25, ADAM15, LUCAT1, SSH1, SIRPB1, GBP3, and combinations thereof.

30. The method of Embodiment 29, wherein the mutually exclusive exon is selected from the group consisting of the mutually exclusive exons listed in Table 6.

31. The method of Embodiment 8, wherein the at least one RNA biomarker has a reduced alternative 5′ splicing in the biological sample, relative to a control sample.

32. The method of Embodiment 31, wherein the reduction is ≥4.5%, optionally, the reduction is ≥5.0%.

33. The method of Embodiment 31 or 32, wherein the at least one RNA biomarker is selected from the group consisting of PARP2, PACRGL, ENTPD1-AS1, NEIL2, FUZ, SDR39U, ADAM15, EPOR, ZSCAN26, SNHG17, GPS2, NECAP1, MRPLI1, DNAJC9, ANKZF1, C1orf162, PIGT, SLC25A37, AP1G1, CIC, ITGB7, ATG16L2, BECN1, ARHGEF40, and combinations thereof.

34. The method of Embodiment 33, wherein the alternative 5′ splicing site is selected from the alternative 5′ splicing sites listed in Table 7.

35. The method of Embodiment 8, wherein the at least one RNA biomarker has an increased alternative 5′ splicing in the biological sample, relative to a control sample.

36. The method of Embodiment 35, wherein the increase is 4.5%, optionally, the increase is ≥5.5%.

37. The method of Embodiment 35 or 36, wherein the at least one RNA biomarker is selected from the group consisting of BANP, PIGA, SNHG8, RAD52, IRF3, CEP78, SPINT1, TMEM156, NT5C3B, PLD2, HLA-A, ANKRD12, CASP8, PACS2, HLA-DMA, DHPS, PDCD6, and combinations thereof.

38. The method of Embodiment 37, wherein the alternative 5′ splicing site is selected from the alternative 5′ splicing sites listed in Table 8.

39. The method of Embodiment 8, wherein the at least one RNA biomarker has a reduced alternative 3′ splicing in the biological sample, relative to a control sample.

40. The method of Embodiment 39, wherein the reduction is 6.5%, optionally, the reduction is ≥7.5%.

41. The method of Embodiment 39 or 40, wherein the at least one RNA biomarker is selected from the group consisting of SNX5, POLR2J3, MPPE1, AC016394.2, DPM1, E2F5, PTPN7, MTFP1, TOR1AIP1, POT1, JOSD2, NLRX1, FDXR, ZDHHC16, ALKBH4, RPS9, ZNF302, TENT4B, ADGRE2, TKT, CARD8, RBM26, WSB1, and combinations thereof.

42. The method of Embodiment 41, wherein the alternative 3′ splicing site is selected from the alternative 3′ splicing sites listed in Table 9.

43. The method of Embodiment 8, wherein the at least one RNA biomarker has an increased alternative 3′ splicing in the biological sample, relative to a control sample.

44. The method of Embodiment 43, wherein the increase is ≥4.5%, optionally, the increase is ≥5.0%.

45. The method of Embodiment 43 or 44, wherein the at least one RNA biomarker is selected from the group consisting of DDX60L, ATP11A, SRGAP2, CEACAM21, COX18, WDR47, PATZ1, POLM, CC2DIB, CLK4, MIB2, PHF1, KANSL1, TCF3, and combinations thereof.

46. The method of Embodiment 45, wherein the alternative 3′ splicing site is selected from the alternative 3′ splicing sites listed in Table 10.

47. The method of any one of Embodiments 1-46, comprising assaying at least 2, 3, 4, 5, 10, 15, 20, 25, 30, 35, 40 or 45 RNA markers in the biological sample from the subject.

48. The method of any one of Embodiments 1-47, wherein assaying the at least one RNA biomarker comprises performing quantitative RT-PCR, microarray, cDNA sequencing (RNA-Seq), or a combination thereof.

49. The method of any one of Embodiments 1-48, wherein the subject is a human male.

50. The method of any one of Embodiments 1-48, wherein the subject is a human female.

51. The method of any one of Embodiments 1-50, wherein the at least one RNA biomarker comprises fragile X mental retardation 1 (FMR1).

52. The method of Embodiment 51, wherein isoform 12 of FMR1 RNA has an increased expression in the biological sample, relative to a control sample.

53. The method of any one of Embodiments 1-52, wherein the control sample is from an age-matched sample from a typically developing subject.

54. The method of any one of Embodiments 1-52, wherein the control sample is a theoretical value calculated from the general population.

55. The method of any one of Embodiments 1-52, wherein the control sample is a baseline sample of the subject.

56. The method of any one of Embodiments 1-55, further comprising treating the subject.

57. A system, comprising one or more polynucleotide probes and/or one or more polynucleotide primers configured to detect, in a biological sample, the level and/or splicing of the at least one RNA biomarker associated with fragile X syndrome (FXS).

58. The system of Embodiment 57, wherein the at least one RNA biomarker is selected from the group consisting of AGAP1, RAB25, FAM3B, XKR3, MAP3K15, LEP, RP11-706O15.3, GCOM1, CXCL6, RGL3, NECAB2, TGM3, LRRC6, MAB21L3, RP11-36B15.1, AC091878.1, RP11-154H23.3, NOV, AC093495.4, RP11-455F5.6, RGPD2, COL9A3, CLEC18A, RP11-256L6.2, LINC01127, SLC38A11, EFCAB12, LA16c-380H5.5, CXCL1, RP11-1334A24.5, AC100793.2, ANKDD1A, AVIL, RP11-44F14.8, RP11-290F20.1, AC116366.5, EPHB4, ST6GALNAC3, PANX2, CREB5, KIAA0319, HECW2, ADCY4, LINC00173, RP11-59D5_B.2, RP11-274B18.2, RP11-213H15.3, CORO7-PAM16, HAL, DPEP3, AC002467.7, MGAM, PNMA8A, FMR1, S100B, RP11-885N19.6, RP11-545I5.3, AC091814.2, KLRC2, L1TD1, PGBD5, MXRA7, CROCC2, SEMA5A, PLA2G4C, RP11-1008C21.1, TANC1, C4orf50, NUAK1, AC104809.4, RGS17, KCNS1, DRAXIN, B3GAT1, ARHGEF28, KIF19, APOL4, GZMH, GAS1, SCD5, GLB1L2, IGHA1, KNDC1, RP11-383H13.1, FGFR2, TFCP2L1, PDGFRB, LAG3, GPR153, PODN, CKB, CERCAM, ZNF365, JUP, TRNP1, JAKMIP1, CPXM1, SLC1A7, LGR6, FCRL6, MORN4, TUBB2A, PRSS23, BFSP1, NCALD, ZNF573, PAK1, MIR4435-2HG, CD8B, PDGFC, TRAPPC2L, AC006504.5, ZNF512, FAM228B, NEIL2, FAM78A, FYB1, RNF216P1, ZCWPW1, DTX2, ATP5MD, MX2, LYRM1, GUF1, DPH7, NSFL1C, MTMR1, GTPBP10, RGS3, DRAM2, RHOH, LAIR2, GBP3, GTF2H1, XPNPEP3, ZNF888, TBC1D5, AC060780.1, SDHAP2, KMT2A, SH3BP2, CSNK1G2, NSUN5P1, LINC01128, RNF19A, SNHG8, TOP1MT, AL135818.1, CR1, CRIM1, NAP1L1, AC004593.2, SEC61A2, PCNX2, TPT1-AS1, HLA-A, LUCAT1, PTPN2, SEC31B, POLR2J3, POLR2J4, CAST, POLR2J4, NUMBL, PRMT7, ATF7IP2, TIMM23B-AGAP6, ADGRE2, GTF2H2B, MICA, SLC29A2, ZBTB10, NLGN3, METTL25, ADAM15, SSH1, SIRPB1, PARP2, PACRGL, ENTPD1-AS1, FUZ, SDR39U1, EPOR, ZSCAN26, SNHG17, GPS2, NECAP1, MRPL11, DNAJC19, ANKZF1, C1orf162, PIGT, SLC25A37, AP1G1, CIC, ITGB7, ATG16L2, BECN1, ARHGEF40, BANP, PIGA, RAD52, IRF3, CEP78, SPINT1, TMEM156, NT5C3B, PLD2, ANKRD12, CASP8, PACS2, HLA-DMA, DHPS, PDCD6, SNX5, MPPE1, AC016394.2, DPM1, E2F5, PTPN7, MTFP1, TOR1AIP1, POT1, JOSD2, NLRX1, FDXR, ZDHHC16, ALKBH4, RPS9, ZNF302, TENT4B, TKT, CARD8, RBM26, WSB1, DDX60L, ATP11A, SRGAP2, CEACAM21, COX18, WDR47, PATZ1, POLM, CC2D1B, CLK4, MIB2, PHF1, KANSL1, TCF3, and combinations thereof.

59. The system of Embodiment 58, wherein one or more polynucleotide probes are immobilized on a solid substrate.

60. The system of Embodiment 59, wherein the system is a microarray.

61. A method of stratifying a population of subjects having, or having a propensity to develop, fragile X syndrome (FXS), comprising assaying biological samples from the subjects for the presence of fragile X mental retardation 1 (FMR1) RNA isoform 12.

62. A method for assessing the efficacy of a drug for treatment of fragile X syndrome (FXS), comprising stratifying a population of subjects by the method of Embodiment 61 to create a stratified population comprising a subpopulation who has the FMR1 RNA isoform 12 and a subpopulation who does not have the FMR1 RNA isoform 12, and administering the drug to the subpopulation who has FMR1 RNA isoform 12, or to both subpopulations.

63. A method of stratifying a set of subjects having fragile X syndrome (FXS), comprising assaying fragile X mental retardation 1 (FMR1) RNA in a biological sample from the subject, and stratifying the set of subjects for treatment based on the presence and/or level of the FMR1 RNA isoform 12 in the biological sample.

64. The method of any one of Embodiments 61-63, wherein the biological sample is a non-neural biological sample.

65. The method of Embodiment 1, further comprising treating the subject if the subject is diagnosed to have, or has a propensity to develop, fragile X syndrome (FXS).

66. The method of any one of Embodiments 1-56, wherein the at least one RNA biomarker is selected from the group consisting of ANAPC1P2, FAM3B, HMGB1P5, CYP4F22, RHOC, AGAP1, CFAP70, KNDC1, PRR5L, ZNF365, DUSP5, ARHGAP24, EPOP, MXRA7, TOMM5, TRBV2, NKG7, CLEC5A, TKTL1, RAB25, COL13A1, RBM11, AC008764.4, CKB, GNGT2, LAMC3, NEFL, ZNF154, C12orf75, MSC-AS1, RPL39L, PPFIBP1, ACOT7, CDKN1C, CKS1B, LINC00174, PALM, CABP4, EFNA5, LYPD2, DRAXIN, B3GAT1, TPST2, CROCC2, FCRL6, AC026369.3, C19orf12, S100B, GAS1, JAKMIP1, LINC02345, GPR153, S1PR5, MIR3150BHG, and combinations thereof.

67. The method of any one of Embodiments 1-56, wherein the skipped exon is selected from the group consisting of PARP6, NCALD, PACRGL, TCF7, ADAM15, LAIR2, XPNPEP3, ADAM15, POLR2J3, POLR2J3, LINC00937, WARS1, ADAM15, ADAM15, AL135818.1, AC092070.2, TRPT1, DST, WARS1, MIR4435-2HG, LRRFIP1, ADCY10P1, RNF19A, DRAM2, TRPV2, CAST, C11orf80, ZNF266, HMOX2, JPX, ZNF266, DPM1, FRG1, FRG1, COPS3, METTL8, FCRLA, WARS1, WARS1, PMS2CL, MUC20-OT1, ZNF273, NQO2, AC141586.1, PNPO, BFAR, GBAP1, RAB18, GPS1, TAF5, HMOX2, PACRGL, IZUMO4, ANKS3, SEPTIN2, YY1AP1, OFD1, AC012184.3, SEPTIN2, LAIR2, SEPTIN2, LDAH, CDC42BPG, ZNF273, VMP1, ATP6AP1L, COMMD2, PPRC1, RHBDF2, ZNF56, SRSF4, ZNF529-AS1, TNK2, ANKS3, SUCO, TBC1D19, WARS1, ITGB7, CYB5RL, WARS1, IFI44L, TGIF1, ZBTB25, FKRP, NSUN5P1, ADAM15, PRKCQ-AS1, TRPT1, NCAPG2, IP6K2, ALS2CL, NFS1, LINC00174, CYRIB, CDC27, ZNF202, GOLGA2P5, ZNF85, FBXW8, COA8, NSRP1, KLRC4-KLRK1, OFD1, FKRP, BCLAF3, SRPK2, WARS1, HPCAL1, RXYLT1, CARMIL1, RAD51C, HEATR6, CROCC, AL732372.2, DTNB, PLPP1, CTNS, COP1, NEK3, POLA1, LSM14B, CCDC18-AS1, LYPLAL1-DT, SLC25A43, BRAF, STX3, PPFIA1, UBR2, SRP14-AS1, ZBTB7B, BCLAF3, SF11, FKRP, STAG3L3, IMMP1L, PNPO, BBS2, PIGT, PAAF1, NQO2, NT5DC2, PRKCQ-AS1, OSBPL5, MAPKAPK5, ITGAE, PCBP1-AS1, FAM229B, ZSCAN25, OBSCN, HDAC10, ACAD10, JPX, NUBP2, FRG1BP, PDPR, RAB3IP, HEATR3, LSM14B, POGLUT3, TPP2, TRPM7, NDUFAF5, WARS1, RAB3IP, LARGE2, DPAGT1, DUSP16, KDM3B, KMT2B, NQO2, COA1, GARS1-DT, TPP2, NMRK1, MMEL1, ABCA11P, UPP1, PHF1, TMEM218, MAP7D3, ZNF653, AC010175.1, DHODH, TUBGCP6, TUBGCP6, TTLL3, TEX10, SPATS2, ZNF76, FKRP, SAR1B, ZEB2, COA1, SLC44A2, BCLAF3, TRMT2B, PITPNM2, IFT52, P4HA1, NUTM2A-AS1, SIGIRR, ACAD10, ZMYM5, AC243960.1, ZMYM3, VNN2, SERINC5, POGLUT3, LAIR1, BAZ2A, FCGRT, GGA1, TMEM161B-AS1, KRIT1, SAMD4B, FRG1CP, ACADVL, KIF27, NBPF12, NUP62, CEP295, RHNO1, FANCI, RMDN1, UBA52, ELF4, MVK, WASHC2A, TMEM79, BANP, EBLN3P, ITGB3BP, TMEM267, SLC25A37, SERPING1, AC087632.2, TGFB3, TPT1-AS1, LGMN, INO80C, INO80C, TAF11, ATRIP, NCOR2, GTDC1, CPVL, PVT1, RNPS1, A1BG-AS1, PCBP1-AS1, CAMLG, TMBIM1, SLC15A2, DENND4C, MAP3K20, RAB4A, NOD2, ERMARD, ZNF354B, NOTCH2, IP6K2, HLTF, TRAF3IP2-AS1, CCNL1, SYTL2, DGUOK, AGPAT5, VNN2, R3HCC1L, KIF27, LINC00963, JAK3, PPP4R1L, HM13, HM13, GMFG, GGA1, CCM2, LY96, EIF4G3, SNX25, METTL15, CEP290, MAP4, DPEP2, TCEA3, ST6GALNAC4, CASP5, DLGAP4, RBIS, LUCAT1, PSTK, GLT1D1, GTF2I, NRDE2, ST3GAL2, SNAP23, AC138894.1, LMAN2L, CERS4, PGAP2, SLC12A2, CAMK4, ABRAXAS1, FCRL2, TANGO2, GGCT, AP001781.2, NOD2, FYN, CYBC1, CCDC191, ABHD12, FOPNL, NEXN, HFE, TRMT2B, ABCD4, AC243960.1, PSMG4, IKBKG, TRMT61B, IGHG3, ING3, RPL32P3, ABHD14A-ACY1, TBCD, RPAIN, SULT1A1, TCFL5, TCF25, COX20, SERGEF, KIAA2026, CD40, KIR2DL1, GPR141, LRRC37B, TMEM44-AS1, TYSND1, FGR, ZNF133, BCL2L13, PMS2, ARHGAP19, TRIM34, ZBTB8OS, RESF1, CHD4, ZNF133, RBM23, PCBP1-AS1, TMEM116, PI4KB, SNRPA1, PCBP1-AS1, HMOX2, GMDS-DT, IL18R1, UPB1, ZNF138, GATC, PIK3C2B, UPF3A, ANGEL2, KRIT1, CHD4, SRPK2, EVA1C, ACCS, DDX60L, RBIS, N4BP2L2, PCBP1-AS1, ERICH6-AS1, ATP5MC2, SRP54-AS1, ZBTB1, DTNBP1, FAM228B, METTL6, KPTN, POLK, PGS1, RPRD1B, LINC00426, CD160, TAF2, IL15RA, ANXA11, RALGAPB, FAM13B, KIAA1191, DPEP2, CD226, PTPRA, ZNF75D, NABP1, EME2, SNRNP70, TRPV2, PLBD1-AS1, UBE3B, CEP68, ABRAXAS1, NLRP6, RIDA, UBE3B, AC243960.1, DPY19L1, CLEC4C, MADD, CBWD3, ANP32A, ENDOV, FCGRT, RNF8, TFB1M, LONP2, TMEM161B-AS1, TMEM161B-AS1, POLK, PCBP1-AS1, RAB3A, SLC37A3, MIR762HG, MRS2, TXNDC11, LUCAT1, CERS5, INO80E, LUCAT1, AC096887.1, IRAK3, SGCB, CEP41, MPC1, PPP2R2D, TOGARAM2, SLC25A37, ERLIN1, COQ5, TAF1C, SNRNP70, INTS7, DSTYK, CD44, RPL5, CCDC138, RMND5B, SLC66A2, MAK, ABITRAM, AC000120.4, TAMM41, SP140, STAMBP, UBXN8, CHROMR, FANCG, FGR, DPEP2, RALGAPB, C1orf162, GOLGA2P5, GMFG, AC147651.1, IQCB1, ERLIN1, ATOX1, RIDA, TSPAN5, SNHG17, MVK, PSME3IP1, POLK, WDR20, DDX31, POLD1, CAST, TM7SF2, NSRP1, FAM210A, MYL5, INO80C, SIMC1, ABHD16A, DAP3, ANKRD26, ADCY10P1, ADAP2, RPL5, ZCWPW1, SUSD3, CHROMR, TMEM126A, ZFP1, ZNF195, FPR2, DCLRE1C, POU6F1, RPF2, MTSS1, TOP1MT, CPVL, PTPA, PANK2, RGS18, CKLF, PAK1, HM13, LINC01934, BBOF1, BMPR2, AF117829.1, THTPA, C11orf80, UBL7, TRIM5, DICER1-AS1, AC009022.1, POLR2J3, MATR3, CBWD3, POU6F1, TYSND1, BBOF1, NBR2, CCM2, MAP3K8, AC008035.1, LDLRAD4, PSMG4, ANAPC10, ZMYM1, ADCY10P1, TCFL5, POLL, DPH7, MOK, MECP2, HLA-DPA1, HM13, MVK, ZBTB8OS, DOCK2, WBP1, VMP1, MYOM1, FYB1, EVA1C, AC093157.1, TRAF3IP1, SNHG17, YWHAH, ARMCX5-GPRASP2, AC009022.1, ZNF232, CENPN, WBP1, ITGB3BP, MPV17, MTIF3, LRRC23, TMEM143, NEIL2, TMEM161B-AS1, INTS7, TRAF3IP2-AS1, GOLGA8A, BBS9, PPIEL, TUBGCP4, AC060780.1, ECHDC2, UROS, MAFG, GIPR, HCLS1, AC016727.1, SNHG17, AC022137.3, CWF19L1, LRRC23, TMEM143, VNN2, AAMDC, TPRKB, FCHSD1, PYROXD2, STRA6LP, SERPINB9P1, AC245100.4, PGAP3, and combinations thereof.

68. The method of any one of Embodiments 1-56, wherein the mutually exclusive exon is selected from the group consisting of CR1, PIGL, XPNPEP3, MAPKBP1, TBC1D12, AC004593.2, AC016831.6, WDR60, ZNF720, MYO15B, HSD17B3, WHAMMP3, AL392172.1, ATXN2, AL356481.1, PDE8A, ZNF266, AL162258.1, CAMKK1, PPFIA1, ZNF266, CFAP70, ACCS, ATF7IP2, NEIL2, TAF4, PNPT1, HLA-DRB5, DDX60L, YAF2, SP140, KIAA1841, TNNT3, ARFIP1, TUBD1, POMZP3, SH3TC1, POLR2J4, CDKL3, CD300LF, ELP1, RPTOR, ZNF266, POLR2J4, CBWD3, WDR12, PVT1, LLGL2, TCFL5, MCTP1, ST3GAL5, UBXN8, ATXN2, MICA, CARD8, CARD8, AL121845.3, IL32, GMPPB, ZEB2, TBC1D8, LEMD3, SLC38A1, JARID2, SLC38A9, AC119396.1, UBE3C, MARCHF8, TUBGCP4, SLAMF1, GTF2H2, IRF8, POMZP3, MCM6, PVT1, NIBAN3, NFS1, KDM3B, AC114490.2, ZC3H13, TGS1, CR1, POMZP3, POMZP3, DDX21, SYNE1, ZBTB17, MYOM1, TIMM23B-AGAP6, SBNO1, RAB11FIP3, CNOT1, NMRK1, IRF8, TSC2, RCHY1, PVT1, COG8, FKBP15, PLBD1-AS1, TOM1, SUGP1, MCTP1, ZNF185, TAF1D, CELF1, PIGG, C11orf80, ZNF638, TRIM14, NEIL2, SNX9, NSUN6, MRTFA, SLC3A2, SNAPC1, ING4, SERF2, FAM114A2, CELF1, NFKB2, VARS1, CLEC17A, TMEM184B, EML2, CCM2, WRNIP1, DENND4B, AL392172.1, TMEM131, RYK, TMCO3, FOXP1, DNMT1, ZNF254, TCFL5, PILRB, IDH1, NUP98, DDB2, IGHD, POLR2J3, UBAP2L, TSEN2, COA1, MPHOSPH6, CERS4, AC069281.2, ATP2B4, EIF4G1, WDFY3, ECPAS, SLC25A19, STAU2, RPS6KA3, DUS2, MAN1B1, KMT2D, RRP8, SPAG9, CPNE1, UBXN11, SLC44A2, KDM5A, TNPO1, HEATR5B, RNF170, RHOT1, TMEM50B, SCLT1, MAN2B1, PCBP1-AS1, AC069281.2, DTNBP1, PINK1, HDAC7, CMTM1, CDC23, PCBP1-AS1, PCBP1-AS1, EHBP1L1, ANKZF1, AC118553.2, NAE1, GIT1, CREBBP, RALY, XAF1, CCM2, CPSF7, CAST, SMG5, PVT1, RGS6, SUMF2, RALY, RALY, MLKL, ITPA, GATAD2A, MKNK1, MIR4435-2HG, SSBP4, CCM2, TNFRSF1A, TLE3, ARHGAP25, TRAK1, LRCH3, TLE3, PLD3, ADA, SHISA5, NCF1, NUP88, USB1, FMNL1, CYB5R4, NFKB2, NUP88, RCBTB2, STRADA, CRTC2, UNC13D, ICE1, TMBIM1, SH2D3C, SLC44A2, SORL1, LTBR, CHP1, CTDNEP1, RPS12, WDFY4, FANCA, CPNE1, CPNE1, OSBPL11, RHOT1, ITGAL, INTS8, DBNL, GSN, CNOT3, DAGLB, DBNL, STXBP2, SNHG29, UNC13D, FCHO1, SLC35C2, WDR47, MKNK2, INPP5D, LRCH4, TTC7A, UNC93B1, PDE4B, HLA-DRB1, TNFRSF1A, AC118553.2, FGR, COG4, FLOT1, CAMK1D, LMBR1, TSC2, FGR, NAA60, INTS6, TTC16, H2AZ2, AC004997.1, SPTLC1, BAG6, TTC17, CDK16, NAXD, IGFLR1, GOLGA7, YY1AP1, YY1AP1, CASC4, PTDSS1, CEP290, TIMM23B-AGAP6, IL21R, DNMT1, PDIA4, SIPA1, POLR2F, POLL, PSMA5, PAFAH2, MOV10, HMGB2, PYCR2, B4GALT3, RHOT2, DDX17, NQO2, WDR70, INTS8, EXOSC1, RABEPK, UTP6, TRPC4AP, XAF1, ZDHHC4, PHYKPL, CAST PPA2, RBM6, DDX21, RCBTB2, ABCA7, LMF2, PHC2, WDSUB1, JAK1, IFTAP, RABEPK, SNX10, DAGLB, DEK, WDSUB1, LAIR1, VDAC2, PPP4R3B, DLEU2, STK38, STAT5B, DES11, CCM2, PREX1, TMEM87A, NEPRO, WDR37, LAIR1, LAIR1, SUPT20H, CDC16, JPX, RBM23, RPL18A, LINC00893, ZDHHC4, SND1, SLC3A2, RBM33, TMEM63A, MEF2D, NLRC5, POLD2, TNFSF13, NTAN1, VPS53, TANGO2, ARHGEF40, ZDHHC24, ABCA7, SAMD9L, EPSTI1, POLR3C, ECHDC1, GMDS-DT, WDSUB1, TYROBP, TTC13, PHF20L1, CORO1C, ECHDC1, SMIM8, NEPRO, HLA-C, BBS9, AMD1, ERCC1, DLG1, ZFAND1, MAPKAPK5, CELF1, PVT1, WASH3P, CNOT2, RFFL, LONP2, ARIH2, SNX22, SCAP, VASP, FKBP15, PSD4, INTS8, TANGO2, TMEM127, LRCH3, KDM5C, BBS1, SRP14-AS1, SOS1, RNF121, PDCD6, SEC16A, ADAP2, DNAJC2, RPS6 KB1, RNF121, CEP290, PVT1, EXOSC9, CLPTM1, PREX1, IL15, MTA1, EFCAB13, OCEL1, ZC3H7A, DAGLB, EIF2B4, EIF2D, GUSBP11, FBXO3, VASP, GRK3, PIK3AP1, CDC16, RALGAPB, PAPSS1, RABGAP1L, C1orf21, LAIR1, ST6GAL1, OGFOD1, ARAP2, FDX1, TTC39B, CD320, RUNX3, COP1, ANKRD27, TXLNA, TPGS2, MCTP1, AD000671.1, COQ7, NRF1, IFTAP, SCFD1, LY96, TMCO4, ANKZF1, SCYL1, PTPRA, DRAM1, USP48, ARHGEF40, CHFR, GYS1, RORC, UVRAG, SIRPB1, TTI2, COP1, TMEM8B, CAPN12, HIPK3, SRC, HERPUD2, KLRG1, COP1, WDSUB1, SPG7, P4HA1, IKBKG, METTL26, DNAJC1, NACC2, THOC6, ALG13, RB1, LRP8, BCAS3, RNF170, MDM4, METTL26, FANCA, CWF19L1, LRRK2, PRMT9, NUP54, ANKZF1, NAE1, PPP2R2D, TSEN2, IKBKG, THEM4, COP1, CAST, AGPAT3, TRPM7, RAD1, RNPC3, WDSUB1, SLBP, KIF1C, FOXRED1, DICER1-AS1, CRBN, PACC1, SPATA13, ABCB7, DENND4B, JAK2, N4BP2L1, IMMP1L, TBC1D2B, ATF7IP, SLBP, ARHGAP19, POLL, IL15, RIN3, PXN, PLXDC1, N4BP2L1, PI4KAP2, SUZ12P1, SCAF8, GTF2IRD2B, SENP6, DNMT3A, DCP1B, TMEM39A, CBWD3, RETSAT, DENND6A, ZSWIM7, AK5, MDM1, ITGA6, HDAC4, SIRPB1, ELMO2, ABCA7, HLA-B, KIAA0753, MRNIP, RIOK1, HLA-B, RNF144B, RAB35, DOCK10, FBXL4, TMEM189-UBE2V1, SEPTIN11, RBM22, BSCL2, SNHG17, SORBS3, SPOPL, SLC7A6, NDRG1, LAIR1, LAIR1, ATL2, SCAF8, AC009061.2, TMCO4, BSCL2, MAPK6, PLSCR3, FOXJ3, SLC39A11, PYROXD1, RNF144B, TMEM220, TADA2A, CYRIB, PEX1, TSPAN2, TTLL12, TMEM234, BCL9L, AC0008073.3, GNB4, TSPAN2, TMEM234, ERAP2, ANKRD6, DDX60L, GTF2H2B, SLC35F2, TBCD, ZFR, PXYLP1, SOS1, SNHG17, CA5B, SNHG17, ERC1, CYRIB, RANBP10, AC092070.2, SETD6, ITGB2, CEP170, SNHG17, LRP8, SNHG17, ANKRD36B, and combinations thereof.

69. The method of any one of Embodiments 1-56, wherein the alternative 3′ splicing site is selected from the group consisting of PATZ1, TPTEP1, PABPC1L, SNHG17, CPNE1, CPNE1, ZNF160, XAF1, LSM7, POLR2J3, TSNARE1, THADA, TRIM73, AEBP1, TBC1D7, DDX60L, ASNS, MTSS1, MZB1, SEPTIN8, PPWD1, TMEM116, TMEM116, BRD9, SDHA, IKBKG, ZNF707, CEP131, DXO, TAFA2, TPRG1, DLG1, GGT1, SRGAP2, TMEM161B-AS1, HLA-C, CCDC163, NABP1, RNF181, CENPT, KHDC4, ZNF7, DCAF11, AK5, ARRB2, ARHGAP25, AHSA2P, AZIN1, ZNF540, MFSD9, JKAMP, HARS2, RNF32, BPHL, PPP6R3, PPP6R3, HELB, GALT, ALAD, LAIR1, LAIR1, AC016394.2, CTSB, NAPIL4, ZNF195, TCTN1, PHB2, PHF1, ATG16L2, TMEM25, SPSB2, YAF2, MAP3K12, ATXN2, ARL6IP4, TMEM273, MPP5, ALDH6A1, LTK, FAM219B, MAN2A2, PSTPIP1, ZNF23, SPG7, GABBR1, MLX, BRCA1, TSPOAP1-AS1, DPH2, MPPE1, CCDC191, PCBP1-AS1, PCBP1-AS1, PCBP1-AS1, VPS33B, UNC50, ZNF345, and combinations thereof.

70. The method of any one of Embodiments 1-56, wherein the alternative 5′ splicing site is selected from the group consisting of PQBP1, PIGT, TTPAL, FNTA, NEIL2, CEP152, DEPDC5, HLA-DMA, MICA, MSTO1, MSTO1, MSTO1, ERAP2, PARP2, ZSCAN25, HAGHL, BUD31, FCRL1, AL392172.1, NDUFV2, ERVK13-1, ERVK13-1, APTR, KLRD1, IRF3, CUL7, ZDHHC3, ZDHHC3, RBIS, PCBP4, ATP5F1A, LUCAT1, CCDC14, UBE2I, ELOA-AS1, SLC25A37, AGA, CNBP, PPIEL, AC022400.7, MRPL43, MRPL43, SERGEF, STAT2, ULK3, SLC6A12, ANXA2, ITGB7, TM6SF1, ARL6IP4, DDX51, UBAC2-AS1, PXN-AS1, APEX1, NEK3, NEK3, TPM1, SMPD1, UBC, DVL2, DPH2, AC243960.1, and combinations thereof.

71. The method of any one Embodiments 1-56, wherein the gene is selected from the group consisting of GPATCH4, CAPN3, SUN1, ZNF7, ATAT1, PER1, MSTO1, DHRS4L2, RELA-DT, TM7SF2, DERL3, ASB16-AS1, FAN1, ZFC3H1, NRBP2, OGFOD2, SUN1, AAAS, LILRA1, BPHL, DDX51, ZNF266, WDR54, BCS1L, FCSK, SEC31B, POLG, CD3E, CD19, TUBGCP4, AVIL, ZNF160, MINK1, TTC16, ING4, FCGR2B, GINS4, C1R, TRIM27, RABEPK, CTC1, MAN2C1, RASGRP4, WDR24, RINL, ZSCAN25, OGFOD2, C1orf174, PAXX LINC01128, PRICKLE3, IRF3, MUTYH, FAHD2A, RIOK1, ZC3H14, TMEM208, HARS2, MAP4K2, WDR6, ZNF649, NEPRO, BEST1, THOC6, PFKM, ADA, IFT43, PPIE, BTN3A3, SAMD9L, MPPE1, IFFO1, NAGK, CCDC159, SAP30BP, MRNIP, ARHGAP9, XPOT, P3H1, REC8, SCML4, APEX1, ENDOV, HMBS, BRPF1, DHRS1, NPHP3, KLF4, CALCOCO1, RPAIN, CIRBP, KRIT1, BSDCI, GALT, TMEM150A, ZSWIM8, DPH1, TMEM205, CARD8, LFNG, NDUFAF7, ARGLU1, RNF44, NRBP2, ERVK13-1, ZNF7, MSTO1, MIB2, TMEM147, BBS1, ZNF160, ZNF577, MFSD2A, VPS11, PPOX, CDK16, MIB2, PRPF40B, STARD5, MIB2, CDK16, ALG9, PPIEL, HTRA2, MAN2C1, ZNF528-AS1, MYO1G, PHYHD1, SNHG17, TCL6, NMRK1, and combinations thereof.

### REFERENCES

1. Shah S, Molinaro G, Liu B, Wang R, Huber K M, Richter J D. FMRP Control of Ribosome Translocation Promotes Chromatin Modifications and Alternative Splicing of Neuronal Genes Linked to Autism. Cell Rep. 2020 Mar. 31; 30(13):4459-4472.e6.
2. Shen S, Park J W, Lu Z X, Lin L, Henry M D, Wu Y N, Zhou Q, Xing Y. rMATS: robust and flexible detection of differential alternative splicing from replicate RNA-Seq data. Proc Natl Acad Sci USA. 2014 Dec. 23; 111(51):E5593-601.
3. Yukselen O, Turkyilmaz O, Ozturk A R, Garber M, Kucukural A. DolphinNext: a distributed data processing platform for high throughput genomics. BMC Genomics. 2020 Apr. 19; 21(1):310.
4. Schena M, Shalon D, Davis R W, Brown P O. Quantitative monitoring of gene expression patterns with a complementary DNA microarray. Science. 1995 Oct. 20; 270(5235):467-70.
5. Barbulovic-Nad I, Lucente M, Sun Y, Zhang M, Wheeler A R, Bussmann M. Bio-microarray fabrication techniques—a review. Crit Rev Biotechnol. 2006 October-December; 26(4):237-59.
6. Hatje K, Rahman R U, Vidal R O, Simm D, Hammesfahr B, Bansal V, Rajput A, Mickael M E, Sun T, Bonn S, Kollmar M. The landscape of human mutually exclusive splicing. Mol Syst Biol. 2017 Dec. 14; 13(12):959.
7. Letunic I, Copley R R, Bork P. Common exon duplication in animals and its role in alternative splicing. Hum Mol Genet. 2002 Jun. 15; 11(13):1561-7.
8. Meijers R, Puettmann-Holgado R, Skiniotis G, Liu J H, Walz T, Wang J H, Schmucker D. Structural basis of Dscam isoform specificity. Nature. 2007 Sep. 27; 449(7161):487-91.
9. Pohl M, Bortfeldt R H, Grützmann K, Schuster S. Alternative splicing of mutually exclusive exons—a review. Biosystems. 2013 October; 114(1):31-8.
10. Tress M L, Abascal F, Valencia A. Alternative Splicing May Not Be the Key to Proteome Complexity. Trends Biochem Sci. 2017 February; 42(2):98-110.
11. Bennett C F, Krainer A R, Cleveland D W. Antisense Oligonucleotide Therapies for Neurodegenerative Diseases. Annu Rev Neurosci. 2019 Jul. 8; 42:385-406.
12. Graef J D, Wu H, Ng C, Sun C, Villegas V, Qadir D, Jesseman K, Warren S T, Jaenisch R, Cacace A, Wallace O. Partial FMRP expression is sufficient to normalize neuronal hyperactivity in Fragile X neurons. Eur J Neurosci. 2020 May; 51(10):2143-2157.
13. Hagerman P J, Hagerman R. Fragile X syndrome. Curr Biol. 2021 Mar. 22; 31(6):R273-R275.
14. Hooper A W M, Wong H, Niibori Y, Abdoli R, Karumuthil-Melethil S, Qiao C, Danos O, Bruder J T, Hampson D R. Gene therapy using an ortholog of human fragile X mental retardation protein partially rescues behavioral abnormalities and EEG activity. Mol Ther Methods Clin Dev. 2021 Jul. 16; 22:196-209.
15. Jewett K A, Lee K Y, Eagleman D E, Soriano S, Tsai N P. Dysregulation and restoration of homeostatic network plasticity in fragile X syndrome mice. Neuropharmacology. 2018 August; 138:182-192.
16. Jonak C R, Lovelace J W, Ethell I M, Razak K A, Binder D K. Multielectrode array analysis of EEG biomarkers in a mouse model of Fragile X Syndrome. Neurobiol Dis. 2020 May; 138:104794.
17. Khoo B, Krainer A R. Splicing therapeutics in SMN2 and APOB. Curr Opin Mol Ther. 2009 April; 11(2):108-15.
18. Malik I, Tseng Y J, Wright S E, Zheng K, Ramaiyer P, Green K M, Todd P K SRSF protein kinase 1 modulates RAN translation and suppresses CGG repeat toxicity. EMBO Mol Med. 2021 Sep. 20:e14163.
19. Park C Y, Halevy T, Lee D R, Sung J J, Lee J S, Yanuka O, Benvenisty N, Kim D W. Reversion of FMR1 Methylation and Silencing by Editing the Triplet Repeats in Fragile X iPSC-Derived Neurons. Cell Rep. 2015 Oct. 13; 13(2):234-41
20. Protic D, Salcedo-Arellano M J, Dy J B, Potter L A, Hagerman R J. New Targeted Treatments for Fragile X Syndrome. Curr Pediatr Rev. 2019; 15(4):251-258.
21. Richter J D, Bassell G J, Klann E. Dysregulation and restoration of translational homeostasis in fragile X syndrome. Nat Rev Neurosci. 2015 October; 16(10):595-605.
22. Richter J D, Zhao X. The molecular biology of FMRP: new insights into fragile X syndrome. Nat Rev Neurosci. 2021 April; 22(4):209-222.
23. Rodriguez C M, Wright S E, Kearse M G, Haenfler J M, Flores B N, Liu Y, Ifrim M F, Glineburg M R, Krans A, Jafar-Nejad P, Sutton M A, Bassell G J, Parent J M, Rigo F, Barmada S J, Todd P K. A native function for RAN translation and CGG repeats in regulating fragile X protein synthesis. Nat Neurosci. 2020 March; 23(3):386-397.
24. Sellier C, Buijsen RAM, He F, Natla S, Jung L, Tropel P, Gaucherot A, Jacobs H, Meziane H, Vincent A, Champy M F, Sorg T, Pavlovic G, Wattenhofer-Donze M, Birling M C, Oulad-Abdelghani M, Eberling P, Ruffenach F, Joint M, Anheim M, Martinez-Cerdeno V, Tassone F, Willemsen R, Hukema R K, Viville S, Martinat C, Todd P K, Charlet-Berguerand N. Translation of Expanded CGG Repeats into FMRpolyG Is Pathogenic and May Contribute to Fragile X Tremor Ataxia Syndrome. Neuron. 2017 Jan. 18; 93(2):331-347.
25. Utami K H, Skotte N H, Colaço A R, Yusof NABM, Sim B, Yeo X Y, Bae H G, Garcia-Miralles M, Radulescu C I, Chen Q, Chaldaiopoulou G, Liany H, Nama S, Peteri U A, Sampath P, Castrén M L, Jung S, Mann M, Pouladi M A. Integrative Analysis Identifies Key Molecular Signatures Underlying Neurodevelopmental Deficits in Fragile X Syndrome. Biol Psychiatry. 2020 Sep. 15; 88(6):500-511.
26. Xie N, Gong H, Suhl J A, Chopra P, Wang T, Warren S T. Reactivation of FMR1 by CRISPR/Cas9-Mediated Deletion of the Expanded CGG-Repeat of the Fragile X Chromosome. PLoS One. 2016 Oct. 21; 11(10):e0165499.
27. Macosko E Z, Basu A, Satija R, Nemesh J, Shekhar K, Goldman M, Tirosh I, Bialas A R, Kamitaki N, Martersteck E M, Trombetta J J, Weitz D A, Sanes J R, Shalek A K, Regev A, McCarroll S A. Highly Parallel Genome-wide Expression Profiling of Individual Cells Using Nanoliter Droplets. Cell. 2015 May 21; 161(5):1202-1214.
28. Ziegenhain C, Vieth B, Parekh S, Reinius B, Guillaumet-Adkins A, Smets M, Leonhardt H, Heyn H, Hellmann I, Enard W. Comparative Analysis of Single-Cell RNA Sequencing Methods. Mol Cell. 2017 Feb. 16; 65(4):631-643.
29. Esanov R, Andrade N S, Bennison S, Wahlestedt C, Zeier Z (2016) The FMR1 promoter is selectively hydroxymethylated in primary neurons of fragile X syndrome patients.
30. Jr M A, Mukherjee N, Bandaru P, Miller J B, Corcoran D L, Langlois C, Munschauer M, Dewell S, Hafner M, Williams Z, Ohler U, Tuschl T (2013) Protein Expression. 492:382-386.
31. Moazami M P, Rembetsy-Brown J M, Wang F, Krishnamurthy P M, Weiss A, Marosfoi M, King R M, Motwani M, Gray-Edwards H, Fitzgerald K A, Brown R H, Watts J K (n.d.) Quantifying and Mitigating Motor Phenotypes Induced by Antisense Oligonucleotides in the Central 1 Nervous System 2.
32. Parikshak N N, Swarup V, Belgard T G, Irimia M, Ramaswami G, Gandal M J, Hartl C, Leppa V, Ubieta LDLT, Huang J, Lowe J K, Blencowe B J, Horvath S, Geschwind D H (2016) Genome-wide changes in lncRNA, splicing, and regional gene expression patterns in autism. Nature.
33. Quesnel-Vallières M, Weatheritt R J, Cordes S P, Blencowe B J (2019) Autism spectrum disorder: insights into convergent mechanisms from transcriptomics. Nat Rev Genet.
34. Roid G H, Pomplun M (2012) The Stanford-Binet Intelligence Scales, Fifth Edition. In: Contemporary intellectual assessment: Theories, tests, and issues, 3rd ed., pp 249-268. New York, N Y, U S: The Guilford Press.
35. Shah S, Molinaro G, Liu B, Wang R, Huber K M, Richter J D (2020) FMRP Control of Ribosome Translocation Promotes Chromatin Modifications and Alternative Splicing of Neuronal Genes Linked to Autism. Cell Rep.
36. Shen S, Park J W, Lu Z, Lin L, Henry M D, Wu Y N, Zhou Q, Xing Y (2014) rMATS: Robust and flexible detection of differential alternative splicing from replicate RNA-Seq data. Proc Natl Acad Sci.
37. Stamova B S, Tian Y, Nordahl C W, Shen M D, Rogers S, Amaral D G, Sharp F R (2013) Evidence for differential alternative splicing in blood of young boys with autism spectrum disorders. Mol Autism 4:1-16.
38. Tabolacci E, Mancano G, Lanni S, Palumbo F, Goracci M, Ferrè F, Helmer-Citterich M, Neri G (2016a) Genome-wide methylation analysis demonstrates that 5-aza-2-deoxycytidine treatment does not cause random DNA demethylation in fragile X syndrome cells. Epigenetics and Chromatin 9:1-16.
39. Tabolacci E, Palumbo F, Nobile V, Neri G (2016b) Transcriptional reactivation of the FMR1 Gene. A possible approach to the treatment of the fragile X syndrome. Genes (Basel) 7:1-16.
40. Tran S S, Jun H I, Bahn J H, Azghadi A, Ramaswami G, Van Nostrand E L, Nguyen T B, Hsiao Y H E, Lee C, Pratt G A, Martinez-Cerdeno V, Hagerman R J, Yeo G W, Geschwind D H, Xiao X (2019) Widespread RNA editing dysregulation in brains from autistic individuals. Nat Neurosci.
41. Wang R, Tian B (2020) APAlyzer: A bioinformatics package for analysis of alternative polyadenylation isoforms. Bioinformatics 36:3907-3909.
42. Yu G, Wang L-G, Han Y, He Q-Y (2012) clusterProfiler: an R Package for Comparing Biological Themes Among Gene Clusters. Omi A J Integr Biol.
43. Yukselen O, Turkyilmaz O, Ozturk A, Garber M, Kucukural A (2019) DolphinNext: A distributed data processing platform for high throughput genomics. bioRxiv.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method of diagnosing a subject as having, or having a propensity to develop, a fragile X-associated disorder, comprising assaying at least one RNA biomarker in a biological sample from the subject, wherein the level and/or splicing of the at least one RNA biomarker in the biological sample is indicative of the subject as having, or having a propensity to develop, the fragile X-associated disorder, and wherein the biological sample is a non-neural biological sample.

2. A method of predicting a treatment outcome of a fragile X-associated disorder in a subject, comprising assaying at least one RNA biomarker in a biological sample from the subject, wherein the level and/or splicing of the at least one RNA biomarker in the biological sample is indicative of the subject having a propensity to have a poorer treatment outcome, and wherein the biological sample is a non-neural biological sample.

3. The method of claim 1, wherein:
a) the fragile X-associated disorder is fragile X syndrome (FXS);
b) the biological sample is a bodily fluid sample, a hair sample, buccal swab sample or a skin sample; or
c) both a) and b).

4. The method of claim 3, wherein the bodily fluid sample comprises blood, saliva, tears, urine or semen, and optionally, the bodily fluid sample comprises white blood cells.

5. The method of claim 1, wherein the at least one RNA biomarker is selected from the group consisting of AGAP1, RAB25, FAM3B, XKR3, MAP3K15, LEP, RP11-706O15.3, GCOM1, CXCL6, RGL3, NECAB2, TGM3, LRRC6, MAB21L3, RP11-36B15.1, AC091878.1, RP11-154H23.3, NOV, AC093495.4, RP11-455F5.6, RGPD2, COL9A3, CLEC18A, RP11-256L6.2, LINC01127, SLC38A11, EFCAB12, LA16c-380H5.5, CXCL1, RP11-1334A24.5, AC100793.2, ANKDD1A, AVIL, RP11-44F14.8, RP11-290F20.1, AC116366.5, EPHB4, ST6GALNAC3, PANX2, CREB5, KIAA0319, HECW2, ADCY4, LINC00173, RP11-59D5_B.2, RP11-274B18.2, RP11-213H15.3, CORO7-PAM16, HAL, DPEP3, AC002467.7, MGAM, PNMA8A, FMR1, S100B, RP11-885N19.6, RP11-545I5.3, AC091814.2, KLRC2, L1TD1, PGBD5, MXRA7, CROCC2, SEMA5A, PLA2G4C, RP11-1008C21.1, TANC1, C4orf50, NUAK1, AC104809.4, RGS17, KCNS1, DRAXIN, B3GAT1, ARHGEF28, KIF19, APOL4, GZMH, GAS1, SCD5, GLB1L2, IGHA1, KNDC1, RP11-383H13.1, FGFR2, TFCP2L1, PDGFRB, LAG3, GPR153, PODN, CKB, CERCAM, ZNF365, JUP, TRNP1, JAKMIP1, CPXM1, SLC1A7, LGR6, FCRL6, MORN4, TUBB2A, PRSS23, BFSP1, NCALD, ZNF573, PAK1, MIR4435-2HG, CD8B, PDGFC, TRAPPC2L, AC006504.5, ZNF512, FAM228B, NEIL2, FAM78A, FYB1, RNF216P1, ZCWPW1, DTX2, ATP5MD, MX2, LYRM1, GUF1, DPH7, NSFL1C, MTMR1, GTPBP10, RGS3, DRAM2, RHOH, LAIR2, GBP3, GTF2H1, XPNPEP3, ZNF888, TBC1D5, AC060780.1, SDHAP2, KMT2A, SH3BP2, CSNK1G2, NSUN5P1, LINC01128, RNF19A, SNHG8, TOP1MT, AL135818.1, CR1, CRIM1, NAP1L1, AC004593.2, SEC61A2, PCNX2, TPT1-AS1, HLA-A, LUCAT1, PTPN2, SEC31B, POLR2J3, POLR2J4, CAST, POLR2J4, NUMBL, PRMT7, ATF7IP2, TIMM23B-AGAP6, ADGRE2, GTF2H2B, MICA, SLC29A2, ZBTB10, NLGN3, METTL25, ADAM15, SSH1, SIRPB1, PARP2, PACRGL, ENTPD1-AS1, FUZ, SDR39U1, EPOR, ZSCAN26, SNHG17, GPS2, NECAP1, MRPL11, DNAJC19, ANKZF1, C1orf162, PIGT, SLC25A37, AP1G1, CIC, ITGB7, ATG16L2, BECN1, ARHGEF40, BANP, PIGA, RAD52, IRF3, CEP78, SPINT1, TMEM156, NT5C3B, PLD2, ANKRD12, CASP8, PACS2, HLA-DMA, DHPS, PDCD6, SNX5, MPPE1, AC016394.2, DPM1, E2F5, PTPN7, MTFP1, TOR1AIP1, POT1, JOSD2, NLRX1, FDXR, ZDHHC16, ALKBH4, RPS9, ZNF302, TENT4B, TKT, CARD8, RBM26, WSB1, DDX60L, ATP11A, SRGAP2, CEACAM21, COX18, WDR47, PATZ1, POLM, CC2D1B, CLK4, MIB2, PHF1, KANSL1, TCF3, and combinations thereof.

6. The method of claim 1, wherein the at least one RNA biomarker has an increased expression in the biological sample, relative to a control sample, and optionally, wherein:
a) the expression of at least one RNA biomarker has a log 2 fold increase of ≥0.80 in the biological sample, relative to a control sample;
b) the at least one RNA biomarker is selected from the group consisting of AGAP1, RAB25, FAM3B, XKR3, MAP3K15, LEP, RP11-706O15.3, GCOM1, CXCL6, RGL3, NECAB2, TGM3, LRRC6, MAB21L3, RP11-36B15.1, AC091878.1, RP11-154H23.3, NOV, AC093495.4, RP11-455F5.6, RGPD2, COL9A3, CLEC18A, RP11-256L6.2, LINC01127, SLC38A11, EFCAB12, LA16c-380H5.5, CXCL1, RP11-1334A24.5, AC100793.2, ANKDD1A, AVIL, RP11-44F14.8, RP11-290F20.1, AC116366.5, EPHB4, ST6GALNAC3, PANX2, CREB5, KIAA0319, HECW2, ADCY4, LINC00173, RP11-59D5_B.2, RP11-274B18.2, RP11-213H15.3, CORO7-PAM16, HAL, DPEP3, AC002467.7, MGAM, PNMA8A, and combinations thereof; or
c) both a) and b).

7. The method of claim 1, wherein the at least one RNA biomarker has a reduced expression in the biological sample, relative to a control sample, and optionally, wherein:
a) the expression of the at least one RNA biomarker has a log 2 fold reduction of ≥1.00 in the biological sample, relative to a control sample;
b) the at least one RNA biomarker is selected from the group consisting of FMR1, S100B, RP11-885N19.6, RP11-545I5.3, AC091814.2, KLRC2, L1TD1, PGBD5, MXRA7, CROCC2, SEMA5A, PLA2G4C, RP11-1008C21.1, TANC1, C4orf50, NUAK1, AC104809.4, RGS17, KCNS1, DRAXIN, B3GAT1, ARHGEF28, KIF19, APOL4, GZMH, GAS1, SCD5, GLB1L2, IGHA1, KNDC1, RP11-383H13.1, FGFR2, TFCP2L1, PDGFRB, LAG3, GPR153, PODN, CKB, CERCAM, ZNF365, JUP, TRNP1, JAKMIP1, CPXM1, SLC1A7, LGR6, FCRL6, MORN4, TUBB2A, PRSS23, BFSP1, and combinations thereof; or
c) both a) and b).

8. The method of claim 1, wherein the at least one RNA biomarker has a reduced exon skipping in the biological sample, relative to a control sample, and optionally, wherein:
a) the reduction is ≥13.0%;
b) the at least one RNA biomarker is selected from the group consisting of NCALD, ZNF573, PAK1, MIR4435-2HG, CD8B, PDGFC, TRAPPC2L, AC006504.5, ZNF512, FAM228B, NEIL2, FAM78A, FYB1, RNF216P1, ZCWPW1, DTX2, ATP5MD, MX2, LYRM1, GUF1, DPH7, NSFL1C, MTMR1, GTPBP10, RGS3, and combinations thereof; or
c) both a) and b).

9. The method of claim 1, wherein:
a) the skipped exon is selected from the group consisting of the skipped exons listed in Table 3 or Table 4;
b) the mutually exclusive exon is selected from the group consisting of the mutually exclusive exons listed in Table 5 or Table 6;
c) the alternative 5′ splicing site is selected from the alternative 5′ splicing sites listed in Table 7 or Table 8; or
d) the alternative 3′ splicing site is selected from the alternative 3′ splicing sites listed in Table 9 or Table 10.

10. The method of claim 9, wherein the at least one RNA biomarker has an increased exon skipping in the biological sample, relative to a control sample, and optionally, wherein:
a) the increase is ≥13.0%;
b) the at least one RNA biomarker is selected from the group consisting of NCALD, DRAM2, RHOH, LAIR2, GBP3, GTF2H1, XPNPEP3, ZNF888, TBC1D5, AC060780.1, SDHAP2, KMT2A, SH3BP2, CSNK1G2, ATP5MD, NSUN5P1, LINC01128, RNF19A, SNHG8, TOP1MT, AL135818.1, and combinations thereof, or
c) both a) and b).

11. The method of claim 9, wherein the at least one RNA biomarker has a reduced mutually exclusive exon switching in the biological sample, relative to a control sample, and optionally, wherein:
a) the reduction is ≥10.0%;
b) the at least one RNA biomarker is selected from the group consisting of CR1, CRIM1, ZCWPW1, NAP1L1, TBC1D5, MIR4435-2HG, AC004593.2, GBP3, SEC61A2, PCNX2, TPT1-AS1, HLA-A, LUCAT1, PTPN2, SEC31B, POLR2J3, POLR2J4, CAST, NUMBL, PRMT7, ATF7IP2, TIMM23B-AGAP6, and combinations thereof; or
c) both a) and b).

12. The method of claim 9, wherein the at least one RNA biomarker has an increased mutually exclusive exon switching in the biological sample, relative to a control sample, and optionally, wherein:
a) the increase is ≥12.0%,
b) the at least one RNA biomarker is selected from the group consisting of HLA-A, ADGRE2, PAK1, TBC1D5, GTF2H2B, MICA, SLC29A2, ZBTB10, NLGN3, CAST, METTL25, ADAM15, LUCAT1, SSH1, SIRPB1, GBP3, and combinations thereof; or
c) both a) and b).

13. The method of claim 9, wherein the at least one RNA biomarker has a reduced alternative 5′ splicing in the biological sample, relative to a control sample, and optionally, wherein:
a) the reduction is ≥4.5%;
b) the at least one RNA biomarker is selected from the group consisting of PARP2, PACRGL, ENTPD1-AS1, NEIL2, FUZ, SDR39U1, ADAM15, EPOR, ZSCAN26, SNHG17, GPS2, NECAP1, MRPL11, DNAJC19, ANKZF1, C1orf162, PIGT, SLC25A37, AP1G1, CIC, ITGB7, ATG16L2, BECN1, ARHGEF40, and combinations thereof; or
c) both a) and b).

14. The method of claim 9, wherein the at least one RNA biomarker has an increased alternative 5′ splicing in the biological sample, relative to a control sample, and optionally, wherein:
a) the increase is ≥4.5%;
b) the at least one RNA biomarker is selected from the group consisting of BANP, PIGA, SNHG8, RAD52, IRF3, CEP78, SPINT1, TMEM156, NT5C3B, PLD2, HLA-A, ANKRD12, CASP8, PACS2, HLA-DMA, DHPS, PDCD6, and combinations thereof; or
c) both a) and b).

15. The method of claim 9, wherein the at least one RNA biomarker has a reduced alternative 3′ splicing in the biological sample, relative to a control sample, and optionally, wherein:
a) the reduction is ≥6.5%;
b) the at least one RNA biomarker is selected from the group consisting of SNX5, POLR2J3, MPPE1, AC016394.2, DPM1, E2F5, PTPN7, MTFP1, TOR1AIP1, POT1, JOSD2, NLRX1, FDXR, ZDHHC16, ALKBH4, RPS9, ZNF302, TENT4B, ADGRE2, TKT, CARD8, RBM26, WSB1, and combinations thereof; or
c) both a) and b).

16. The method of claim 9, wherein the at least one RNA biomarker has an increased alternative 3′ splicing in the biological sample, relative to a control sample, and optionally, wherein:
a) the increase is ≥4.5%;
b) the at least one RNA biomarker is selected from the group consisting of DDX60L, ATP11A, SRGAP2, CEACAM21, COX18, WDR47, PATZ1, POLM, CC2D1B, CLK4, MIB2, PHF1, KANSL1, TCF3, and combinations thereof; or
c) both a) and b).

17. The method of claim 1, wherein:
a) assaying the at least one RNA biomarker comprises performing quantitative RT-PCR, microarray, cDNA sequencing (RNA-Seq), or a combination thereof;
b) the method comprises assaying at least 2, 3, 4, 5, 10, 15, 20, 25, 30, 35, 40 or 45 RNA markers in the biological sample from the subject;
c) the subject is a human male;
d) the at least one RNA biomarker comprises fragile X messenger ribonucleoprotein 1 (FMR1), optionally, wherein isoform 12 of FMR1 RNA has an increased expression in the biological sample, relative to a control sample,
e) or any combination of the foregoing.

18. The method of claim 1, further comprising treating the subject.

19. A system, comprising one or more polynucleotide probes and/or one or more polynucleotide primers configured to detect, in a biological sample, the level and/or splicing of the at least one RNA biomarker associated with fragile X-associated disorder.

20. The system of claim 19, wherein:
a) the at least one RNA biomarker is selected from the group consisting of AGAP1, RAB25, FAM13B, XKR3, MAP3K15, LEP, RP11-706O15.3, GCOM1, CXCL6, RGL3, NECAB2, TGM3, LRRC6, MAB21L3, RP11-36B15.1, AC091878.1, RP11-154H23.3, NOV, AC093495.4, RP11-455F5.6, RGPD2, COL9A3, CLEC18A, RP11-256L6.2, LINC01127, SLC38A11, EFCAB12, LA16c-380H5.5, CXCL1, RP11-1334A24.5, AC100793.2, ANKDD1A, AVIL, RP11-44F14.8, RP11-290F20.1, AC116366.5, EPHB4, ST6GALNAC3, PANX2, CREB5, KIAA0319, HECW2, ADCY4, LINC00173, RP11-59D5_B.2, RP11-274B18.2, RP11-213H15.3, CORO7-PAM16, HAL, DPEP3, AC002467.7, MGAM, PNMA8A, FMR1, S100B, RP11-885N19.6, RP11-54515.3, AC091814.2, KLRC2, L1TD1, PGBD5, MXRA7, CROCC2, SEMA5A, PLA2G4C, RP11-1008C21.1, TANC1, C4orf50, NUAK1, AC104809.4, RGS17, KCNS1, DRAXIN, B3GAT1, ARHGEF28, KIF19, APOL4, GZMH, GAS1, SCD5, GLB1L2, IGHA1, KNDC1, RP11-383H13.1, FGFR2, TFCP2L1, PDGFRB, LAG3, GPR153, PODN, CKB, CERCAM, ZNF365, JUP, TRNP1, JAKMIP1, CPXM1, SLC1A7, LGR6, FCRL6, MORN4, TUBB2A, PRSS23, BFSP1, NCALD, ZNF573, PAK1, MIR4435-2HG, CD8B, PDGFC, TRAPPC2L, AC006504.5, ZNF512, FAM228B, NEIL2, FAM78A, FYB1, RNF216P1, ZCWPW1, DTX2, ATP5MD, MX2, LYRM1, GUF1, DPH7, NSFL1C, MTMR1, GTPBP10, RGS3, DRAM2, RHOH, LAIR2, GBP3, GTF2H1, XPNPEP3, ZNF888, TBC1D5, AC060780.1, SDHAP2, KMT2A, SH3BP2, CSNK1G2, NSUN5P1, LINC01128, RNF19A, SNHG8, TOP1MT, AL135818.1, CR1, CRIM1, NAP1L1, AC004593.2, SEC61A2, PCNX2, TPT1-AS1, HLA-A, LUCAT1, PTPN2, SEC31B, POLR2J3, POLR2J4, CAST, POLR2J4, NUMBL, PRMT7, ATF7IP2, TIMM23B-AGAP6, ADGRE2, GTF2H2B, MICA, SLC29A2, ZBTB10, NLGN3, METTL25, ADAM15, SSH1, SIRPB1, PARP2, PACRGL, ENTPD1-AS1, FUZ, SDR39U1, EPOR, ZSCAN26, SNHG17, GPS2, NECAP1, MRPL11, DNAJC19, ANKZF1, C1orf162, PIGT, SLC25A37, AP1G1, CIC, ITGB7, ATG16L2, BECN1, ARHGEF40, BANP, PIGA, RAD52, IRF3, CEP78, SPINT1, TMEM156, NT5C3B, PLD2, ANKRD12, CASP8, PACS2, HLA-DMA, DHPS, PDCD6, SNX5, MPPE1, AC016394.2, DPM1, E2F5, PTPN7, MTFP1, TOR1AIP1, POT1, JOSD2, NLRX1, FDXR, ZDHHC16, ALKBH4, RPS9, ZNF302, TENT4B, TKT, CARD8, RBM26, WSB1, DDX60L, ATP11A, SRGAP2, CEACAM21, COX18, WDR47, PATZ1, POLM, CC2D1B, CLK4, MIB2, PHF1, KANSL1, TCF3, and combinations thereof;
b) the one or more polynucleotide probes are immobilized on a solid substrate, optionally, wherein the system is a microarray; or
c) both a) and b).


SEQUENCE LISTING (10 sequences)

SEQ ID NO: 1 - DNA, 20 bp, synthetic construct
  misc_feature 1..20: note = Iso12_1Forward Primer
  source 1..20: mol_type = other DNA
agaagatggaggagctggtg

SEQ ID NO: 2 - DNA, 20 bp, synthetic construct
  misc_feature 1..20: note = Iso12_1Reverse Primer
  source 1..20: mol_type = other DNA
cagtggagctctccgaagtc

SEQ ID NO: 3 - DNA, 20 bp, synthetic construct
  misc_feature 1..20: note = Iso12_2Forward Primer
  source 1..20: mol_type = other DNA
ccagcagtgcattgaagaag

SEQ ID NO: 4 - DNA, 20 bp, synthetic construct
  misc_feature 1..20: note = Iso12_2Reverse Primer
  source 1..20: mol_type = other DNA
ctgaagcatgtgcattcctg

SEQ ID NO: 5 - DNA, 20 bp, synthetic construct
  misc_feature 1..20: note = Iso1_1 Forward Primer
  source 1..20: mol_type = other DNA
agaagatggaggagctggtg

SEQ ID NO: 6 - DNA, 24 bp, synthetic construct
  misc_feature 1..24: note = Iso1_1 Reverse Primer
  source 1..24: mol_type = other DNA
ttcatgaacatcctttacaaatgc

SEQ ID NO: 7 - DNA, 312 bp, Homo sapiens
  source 1..312: mol_type = genomic DNA
ctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaag

SEQ ID NO: 8 - DNA, 53 bp, Homo sapiens
  source 1..53: mol_type = genomic DNA
gcatttgtaaaggatgttcatgaagattcaataacagttgcatttgaaaacaa

SEQ ID NO: 9 - DNA, 1724 bp, Homo sapiens
  source 1..1724: mol_type = genomic DNA
cattgggacttcggagagctccactgttctgggcgagggctgtgaagaaagagtagtaagaagcggtagtcggcaccaaatcacaatggcaactgatttttagtggcttctctttgtggatttcggaggagattttagatccaaaagtttcaggaagaccctaacatggcccagcagtgcattgaagaagttgatcatcgtgaatattcgcgtccccctttttgttaaacggggtaaattcaggaatgcacatgcttcagcgtctaaaaccattagcagcgctgctacttaaaaattgtgtgtgtgtgtttaagtttccaaagacctaaatatatgccatgaaacttcaggtaattaactgagagtatattattactagggcattttttttttaactgagcgaaaatatttttgtgcccctaagaacttgaccacatttcctttgaatttgtggtgttgcagtggactgaattgttgaggctttatataggcattcatgggtttactgtgctttttaaagttacaccattgcagatcaactaacacctttcagttttaaaaggaagatttacaaatttgatgtagcagtagtgcgtttgttggtatgtaggtgctgtataaattcatctataaattctcatttccttttgaatgtctataacctctttcaataatatcccaccttactacagtattttggcaatagaaggtgcgtgtggaaggaaggctggaaaatagctattagcagtgtccaacacaattcttaaatgtattgtagaatggcttgaatgtttcagacaggacacgtttggctataggaaaataaacaattgactttattctgtgtttaccaattttatgaagacatttggagatcagtatatttcataaatgagtaaagtatgtaaactgttccatactttgagcacaaagataaagccttttgctgtaaaaggaggcaaaaggtaaccccgcgtttatgttcttaacagtctcatgaatatgaaattgtttcagttgactctgcagtcaaaattttaatttcattgattttattgatccataatttcttctggtgagtttgcgtagaatcgttcacggtcctagattagtggttttggtcactagatttctggcactaataactataatacatatacatatatatgtgtgagtaacggctaatggttaggcaagattttgattgacctgtgatataaacttagattggatgccactaaagtttgcttatcacagagggcaagtagcacattatggccttgaagtacttattgttctcttccagcaacttatgatttgctccagtgattttgcttgcacactgactggaatataagaaatgccttctatttttgctattaattccctccttttttgttttgttttgtaacgaagttgtttaacttgaaggtgaatgaagaataggttggttgccccttagttccctgaggagaaatgttaatacttgaacaagtgtgtgtcagacaaattgctgttatgtttatttaattaagtttgatttctaagaaaatctcaaatggtctgcactgatggaagaacagtttctgtaacaaaaaagcttgaaatttttatatgacttataatactgctgtgagttttaaaagtaaagcaaaagtaaactgagttgcttgtccagtgggatggacaggaaagatgtgaaataaaaaccaatgaaaaatgaa

SEQ ID NO: 10 - protein, 31 aa, Homo sapiens
MEELVVEVRGSNGAFYKHWDFGELHCSGRGL